(19) United States
(12) Patent Application Publication
(10) Pub. No.: US 2025/0236886 A1
(43) Pub. Date: Jul. 24, 2025

(54) ARTIFICIAL EXPRESSION CONSTRUCTS FOR SELECTIVELY MODULATING GENE EXPRESSION IN EXCITATORY CORTICAL NEURONS

(71) Applicant: Allen Institute, Seattle, WA (US)

(72) Inventors: Lucas T. Graybuck, Seattle, WA (US); Bosiljka Tasic, Seattle, WA (US); Tanya Daigle, Lake Forest Park, WA (US); Jonathan Ting, Lake Forest Park, WA (US); Hongkui Zeng, Seattle, WA (US); Brian Edward Kalmbach, Poulsbo, WA (US); John K. Mich, Seattle, WA (US); Erik Hess, Issaquah, WA (US); Edward Sebastian Lein, Mercer Island, WA (US); Boaz P. Levi, Seattle, WA (US)

(73) Assignee: Allen Institute, Seattle, WA (US)

(21) Appl. No.: 19/007,263

(22) Filed: Dec. 31, 2024

Related U.S. Application Data
(63) Continuation of application No. 17/291,584, filed on May 5, 2021.
(60) Provisional application No. 62/806,600, filed on Feb. 15, 2019.
(60) Provisional application No. 62/806,684, filed on Feb. 15, 2019.
(60) Provisional application No. 62/872,021, filed on Jul. 9, 2019.
(60) Provisional application No. 62/755,988, filed on Nov. 5, 2018.

(51) Int. Cl. C12N 15/86; A01K 67/0275

(52) U.S. Cl. CPC C12N 15/86; A01K 67/0275; A01K 2217/05; A01K 2227/105; A01K 2267/0393; C12N 2750/14143; C12N 2830/008

(57) ABSTRACT

Artificial expression constructs for selectively modulating gene expression in selected central nervous system cell types are described. The artificial expression constructs can be used to selectively express synthetic genes or modify gene expression in excitatory cortical neurons, such as primarily within cortical layers 2/3, 4, 5, and 6 and including those with extratelencephalic (ET) projections, intratelencephalic (IT) projections, and pyramidal tract (PT) projections, among others.

### CROSS-REFERENCE TO RELATED APPLICATIONS

This application is a continuation of U.S. patent application Ser. No. 17/291,584 filed May 5, 2021, which is a U.S. National Phase Patent Application based on International Patent Application No. PCT/US2019/059927 filed Nov. 5, 2019, which claims priority to U.S. Provisional Patent Application Nos. 62/755,988 filed Nov. 5, 2018; 62/806,600 filed Feb. 15, 2019; 62/806,684 filed Feb. 15, 2019; and 62/872,021 filed Jul. 9, 2019; each of which is incorporated herein by reference in its entirety as if fully set forth herein.

### STATEMENT REGARDING FEDERALLY SPONSORED RESEARCH OR DEVELOPMENT

This invention was made with government support under grant DA036909 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the XML file containing the Sequence Listing is 3CX5319.XML. The text file is 471,040 bytes, was created on Jan. 3, 2025, and is being submitted electronically via Patent Center.

### FIELD OF THE DISCLOSURE

The current disclosure provides artificial expression constructs for selectively driving gene expression in excitatory cortical neurons. The artificial expression constructs can be used to selectively express synthetic genes or modify gene expression in excitatory cortical neurons, such as primarily within cortical layers 2/3, 4, 5, and 6 and including those with extratelencephalic (ET) projections, intratelencephalic (IT) projections, and pyramidal tract (PT) projections, among others.

### BACKGROUND OF THE DISCLOSURE

To fully understand the biology of the brain, different cell types need to be distinguished and defined and, to further study them, vectors that can selectively label and perturb them need to be identified. In mouse, recombinase driver lines have been used to great effect to label cell populations that share marker gene expression. However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans.

Recent advances in single-cell profiling, such as single-cell RNA-seq and surveys of neural electrophysiology and morphology, have revealed that many recombinant driver lines label heterogeneous mixtures of cell types, and often include cells from multiple subclasses. For example, the Rbp4-Cre mouse driver line, which is commonly used to label layer 5 (L5) neurons, labels cells with drastically different connectivity patterns: L5 intratelencephalic (IT, also called cortico-cortical) and pyramidal tract (PT, also called cortico-subcortical) neurons.

### SUMMARY OF THE DISCLOSURE

The current disclosure provides artificial expression constructs that selectively drive gene expression in targeted central nervous system cell populations. Targeted central nervous system cell populations include excitatory cortical neurons, such as those primarily within cortical layers (L) 2/3, 4, 5, and/or 6 and including those with extratelencephalic (ET) projections, intratelencephalic (IT) projections, and/or pyramidal tract (PT) projections. Particular artificial expression constructs disclosed herein target specific excitatory cell types, while others selectively drive gene expression across numerous excitatory neuron types.

For example, artificial expression constructs including a promoter, the eHGT_075h enhancer, and a gene encoding an expression product can lead to selective gene expression in L2/3 IT excitatory cortical neurons.

Artificial expression constructs including a promoter; the Grik1_enhScnn1a-2, eHGT_058h, eHGT_058m, eHGT_439m, and/or eHGT_254h enhancer; and a gene encoding an expression product can lead to selective gene expression in L4 IT excitatory cortical neurons.

Particular examples of artificial expression constructs including a promoter; the mscRE4 enhancer, a concatenated mscRE4, and/or a concatenated mscRE16 enhancer; and a gene encoding an expression product can lead to selective gene expression in L5 PT excitatory cortical neurons. Examples of these expression constructs include T502-057 (vAi3.0), 981 (vAi5.0), 1052 (vAi10.0), CN1818 (vAi128.0), CN2014 (vAi129.0) and vAi130.0.

Artificial expression constructs including a promoter, a concatenated core of the mscRE4 enhancer, and a gene encoding an expression product can lead to selective gene expression in L5 PT and L5 ET excitatory cortical neurons.

Artificial expression constructs including a promoter; the mscRE1, mscRE11, and/or mscRE16 enhancer; and a gene encoding an expression product can lead to selective gene expression in L5 PT and L5 IT excitatory cortical neurons.

Artificial expression constructs including a promoter, the mscRE13 enhancer, and a gene encoding an expression product can lead to selective gene expression in L6 IT excitatory cortical neurons.

Particular examples of artificial expression constructs including a promoter, the mscRE10 enhancer, and a gene encoding an expression product can lead to selective gene expression in L6 CT excitatory cortical neurons. An example includes 995 (vAi15.0).

Artificial expression constructs including a promoter, the eHGT_440h enhancer, and a gene encoding an expression product can lead to selective gene expression in subtypes of L6b excitatory cortical neurons.

Artificial expression constructs including a promoter, the eHGT_078h enhancer; and a gene encoding an expression product can lead to selective gene expression in L2/3 IT, L4 IT, L5 IT, L5 NP, and L5 PT excitatory cortical neurons.

Selective expression of a gene encoding an expression product can be achieved in L2/3 IT, L5 IT, and L6b neurons utilizing the 1036 (vAi16.0) artificial expression construct described herein. This construct includes the mscRE10 enhancer.

Selective expression of a gene encoding an expression product can be achieved in L2/3 IT, L5 PT, L6 CT, and L6b neurons utilizing the 988 (vAi7.1), 1010 (vAi6.1), and/or 1011 (vAi7.2) artificial expression constructs described herein. These constructs include the mscRE4 enhancer.

Pan excitatory and/or broad expression in excitatory cortical neurons can be selectively achieved utilizing artificial expression constructs including a promoter; the eHGT_073h, eHGT_073m, eHGT_077h, and/or eHGT_078m enhancer; and a gene encoding an expression product. In particular embodiments, pan excitatory expression refers to expression in at least four types of cortical excitatory cells with limited to no expression in inhibitory cells and glial cells.

Artificial expression constructs described herein can additionally label other discrete cell types. For example, in addition to L5 PT cells, artificial expression constructs including a promoter, the mscRE4 enhancer, and a gene encoding an expression product can lead to gene expression in subcortical populations in the CEAc, the substantia nigra, compact part (or pars compacta, SNc), and (ProS). Similarly, in addition to L5 PT cells, artificial expression constructs including a promoter, a concatenated core of the mscRE4 enhancer, and a gene encoding an expression product can lead to gene expression in the subiculum, CA1 pyramidal neurons, a subset of dentate gyrus granule cells, scattered striatal neurons, and sparse cerebellar Purkinje cells.

As indicated by the proceeding discussion, certain artificial expression constructs disclosed herein include engineered enhancers, for example, concatenated cores of the mscRE4, eHGT_078h, and eHGT_078m enhancers and concatemers of the mscRE4 and mscRE16 enhancers. In relation to mscRE4, a functional 155 base pair (bp) core of the mscRE4 enhancer (SEQ ID NO: 29) was concatenated (SEQ ID NO: 30) to minimize the size required to drive gene expression. Despite being a 3× concatemer, SEQ ID NO: 30 is shorter in length than the original mscRE4 enhancer (SEQ ID NO: 28, which includes 555 bp). When used to construct an artificial expression construct, such as an rAAV, such concatemers allow more room for cargo genes linked to the enhancer, which is highly desirable, for example, in gene therapy vectors. For instance, many therapeutic cargo genes are too big to fit in an AAV vector design, so space (length of sequence) is at a premium.

As will be described in more detail throughout the disclosure, particular artificial expression constructs disclosed herein include T502-050, T502-054, vAi34.0, vAi33.2, vAi45.0, vAi1.0, T502-057, T502-059, TG978, TG981, TG988, TG995, TG996, TG999, TG1002, TG1010, TG1011, TG1021, TG1036, TG1037, TG1038, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and CN2014.

### BRIEF DESCRIPTION OF THE FIGURES

Many of the drawings submitted herein are better understood in color. Applicant considers the color versions of the drawings as part of the original submission and reserves the right to present color images of the drawings in later proceedings.

FIGS. 1A-1C. TG978 (vAi4.1). Enhancer mscRE4 (eAi3.0). (1A, 1B) Representative epifluorescence images of mscre4-FlpO-WPRE virus induced expression in the brain of a Ai65F reporter mouse (1C) single cell RNA sequencing analysis of tdTomato-positive cells isolated from primary visual cortex (V1) of an mscre4-FlpO infected Ai65F mouse. L2/3, layer 2/3; L5, layer 5; wm, white matter.

FIG. 2. TG981 (vAi5.0) Enhancer mscRE4 (eAi3.0). Representative epifluorescence images of mscre4-EGFP-WPRE virus expression in the brain of a wild type mouse. Brain sections were stained with an anti-GFP antibody to visualize GFP fluorescence.

FIGS. 3A, 3B. TG988 (vAi7.1) Enhancer mscRE4 (eAi3.0). (3A) Representative epifluorescence images of mscre4-tTA2 virus induced expression in the brain of a Ai63 reporter mouse. Brain sections were stained with an anti-dsred antibody to reveal tdTomato fluorescence. (3B) The mscre4-tTA2 virus was directly injected into the brain of an Ai63 mouse and native tdTomato fluorescence was imaged within primary visual cortex (V1 or VISp). Note that imaging parameters between the two images may be different. L2/3, layer 2/3; L5, layer 5; wm, white matter.

FIGS. 4A, 4B. TG1010 (vAi6.1) Enhancer mscRE4 (eAi3.0). Representative epifluorescence images of mscre4-iCre virus induced expression in the brain of a Ai14 reporter mouse. L5, layer 5; L6, layer 6; wm, white matter.

FIGS. 5A, 5B. TG1011 (vAi7.2) Enhancer mscRE4 (eAi3.0). Representative epifluorescence images of mscre4-tTA2 virus induced expression in the brain of a Ai63 reporter mouse.

FIG. 6 TG1021 (vAi8.0Cre) Enhancer mscRE4 (eAi3.0). Representative epifluorescence image of mscre4-Cre-WPRE virus induced expression in the brain of a Ai14 reporter mouse.

FIG. 7. TG1052 (vAi10.0) Enhancer 4XmscRE16 (eAi11.1). Representative epifluorescence image of 4Xmscre16-EGFP-WPRE virus expression in the brain of a wild type mouse. Virus was delivered by stereotaxic injection directly into the brain.

FIGS. 8A, 8B. TG995 (vAi15.0) Enhancer mscRE10 (eAi6.0). Representative epifluorescence images of mscre10-EGFP-WPRE virus expression in the brain of a wild-type mouse.

FIGS. 9A-9C. TG1036 (vAi16.0) Enhancer mscRE10 (eAi6.0). (9A, 9B) Representative epifluorescence images of mscre10-FlpO-WPRE virus induced expression in the brain of a Ai65F reporter mouse (9C) single cell RNA sequencing analysis of tdTomato positive cells isolated from primary visual cortex (V1) of an mscre10-FlpO-WPRE infected Ai65F mouse

FIGS. 10A, 10B. TG1048 (vAi18.0) Enhancer mscRE10 (eAi6.0). Representative epifluorescence images of mscre10-tTA2-WPRE virus induced expression in the brain of a Ai63 reporter mouse.

FIG. 11. TG996 (vAi19.0) Enhancer mscRE11 (eAi7.0). Representative epifluorescence images of mscre11-EGFP-WPRE virus in the brain of a wild-type mouse. Brain sections were stained with an anti-GFP antibody to reveal GFP fluorescence.

FIGS. 12A, 12B. TG999 (vAi21.0) Enhancer mscRE13 (eAi9.0). Representative epifluorescence images of mscre13-EGFP-WPRE virus in the brain of a wild-type mouse. Brain sections were stained with an anti-GFP antibody to reveal GFP fluorescence.

FIGS. 13A, 13B. TG1037 (vAi22.0) Enhancer mscRE13 (eAi9.0). (13A) Representative epifluorescence image of mscre13-FlpO-WPRE virus induced expression in the brain of a Ai65F reporter mouse (13B) single cell RNA sequencing analysis of tdTomato positive cells isolated from primary visual cortex (V1) of an mscre13-FlpO-WPRE infected Ai65F mouse. The Cell types from top to bottom include: Lamp5 Pich2 Dock5, Lamp5 Lsp1, Vip Chat Htr1f, Sst Tac1 Htr1d, Sst Calb2 Pdlm5, Sst Nr2f2 Necab, Pvalb Sema3e Kank4, Pvalb Rein Itm2a, L2/3 IT VISp Rred, L2/3 IT VISp Adamts2, L2/3 IT VISp Agmat, L2/3 IT ALM Sla, L6 IT VISp Penk Col27a1, L6 IT VISp Penk Fst, L6 IT VISp Col18a1, L5 IT VISp Hsd11b1 Endou, L5 IT VISp Whrn Tox2, L5 IT VISp Col27a1, L5 PT VISp C1ql2 Cdh13, L5 PT VISp Krt80, L6 IT VISp Car3, L4 IT VISp Rspo1, High Intronic VISp L5 Endou, L6 CT VISp Gpr139, L6 CT VISp Ctxn3 Brinp3, L6 CT VISp Ctxn3 Sla, and L6b VISp Mup5.

FIG. 14. TG1046 (vAi23.0) Enhancer mscRE13 (eAi9.0). Representative epifluorescence image of mscre13-iCre-WPRE virus induced expression in the brain of a Ai14 reporter mouse.

FIG. 15. TG1049 (vAi24.0) Enhancer mscRE13 (eAi9.0). Representative epifluorescence image of mscre13-tTA2-WPRE virus induced expression in the brain of a Ai63 reporter mouse.

FIGS. 16A, 16B. TG1002 (vAi26.0) Enhancer mscRE16 (eAi11.0). Representative epifluorescence images of mscre16-EGFP-WPRE virus in the brain of a wild-type mouse. Brain sections were stained with an anti-GFP antibody to reveal GFP fluorescence.

FIGS. 17A-17C. TG1038 (vAi27.0) Enhancer mscRE16 (eAi11.0). (17A, 17B) Representative epifluorescence images of mscre16-FlpO-WPRE virus induced expression in the brain of a Ai65F reporter mouse (17C) single cell RNA sequencing analysis of tdTomato positive cells isolated from primary visual cortex (V1) of an mscre16-FlpO-WPRE infected Ai65F mouse. The Cell types from top to bottom include: Lamp5 Pich2 Dock5, Lamp5 Lsp1, Sst Mme Fam114a1, L2/3 IT VISp Agmat, L6 IT VISp Agmat, L6 IT VISp Penk Fst, L6 IT VISp Col23a1, Adamts2, L6 IT VISp Col18a1, L5 IT VISp Hsd11b1 Endou, L5 IT VISp Whrn Tox2, L5 IT VISp Batf3, L5 IT VISp Col6a1 Fezf2, L5 IT ALM Tmem163 Arhgap25, L5 IT ALM Cpa6 Gpr88, L5 PT VISp C1qql2 Cdh13, L5 PT VISp Krt80, High Intronic VISp L5 Endou, L6 CT VISp Ctxn3 Brinp3, L6CT VISp Ctxn3 Sla, and LowAqp4.

FIG. 18. TG1047 (vAi28.0) Enhancer (mscRE16 (eAi11.0). Representative epifluorescence image of mscre16-iCre-WPRE virus induced expression in the brain of a Ai14 reporter mouse.

FIGS. 19A, 19B. TG1050 (vAi29.0) Enhancer mscRE16 (eAi11.0). Representative epifluorescence images of mscre16-tTA2-WPRE virus induced expression in the brain of a Ai63 reporter mouse.

FIG. 20. TG1149/(T502-050; vAi33.0) Enhancer Grik1-enhScnn1a-2 (eAi14.0). Representative confocal image of Hsp68-EGFP-WPRE-Grik1-enhScnn1a-2 virus induced expression in the brain of a wild type mouse.

FIGS. 21A, 21B. TG1108 (vAi34.0) Enhancer Scnn1a (Grik1) (eAi14.0). Representative confocal images of Scnn1a (Grik1)-FlpO-WPRE virus induced expression in the brain of a Ai65 reporter mouse.

FIGS. 22A, 22B. TG1114 (vAi33.2) Enhancer Scnn1a (Grik1) (eAi14.0). Representative epifluorescence images of Scnn1a (Grik1)-EGFP-WPRE virus in the brain of a wild-type mouse. Brain sections were stained with an anti-GFP antibody to reveal GFP fluorescence.

FIG. 23. TG1109 (vAi45.0) Enhancer mscRE12 (eAi8.0). Representative epifluorescence image of mscre12-FlpO-WPRE virus induced expression in the brain of a Ai65F reporter mouse.

FIGS. 24A-24D. CN1402 (vAi106.0) Enhancer eHGT_058h (eAi106.0). (24A) Fluorescence expression of CN1402 shown in whole mouse brain in sagittal section. (24B) High resolution image (left) showing non-overlap of CN1402 SYFP fluorescence (red) and inhibitory marker Gad1 mRNA expression (white). Image on the right shows near compete overlap of CN1402 SYFP fluorescence (red) and cortical excitatory marker Slc17a7 mRNA expression (white). (24C) Quantification of specificity of CN1402 SYFP fluorescence in ALM and V1 mouse cortical areas based on multiplexed FISH data. Single cell transcriptomic characterization of SYFP fluorescent cells isolated from mouse V1. (24D) After single cell gene expression analysis, cells were mapped to an existing taxonomy of mouse cell types. Blue circle location reflects extent of single cell mapping (toward the final leaf), while size of the blue circle reflects the number of single cells that mapped to that point in the hierarchy. Bars projecting down reflect the number of cells that map to that terminal branch of the cell type taxonomy. The cells listed from left to right include: 169 L2/3 IT VISp Rrad, 168 L2/3 IT VISp Adamts2, 167 L2/3 IT VISp Agmat, 164 L4 IT VISp Rspo1, 163 L5 IT VISp Hsd11b1 Endou, 162 L5 IT VISp Whrn Tox2, 160 L5 IT VISp Batf3, 158 L5 IT VISp Col6a1 Fezf2, 157 L5 IT VISp Col27a1, 154 L6 IT VISp Penk Col27a1, 153 L6 IT VISp Penk Fst, 152 L6 IT VISp Col23a1 Adamts2, 149 L6 IT VISp Col18a1, 146 L6 IT VISp Car3, 144 L5 PT VISp Chrna6, 143 L5 PT VISp Lgr5, 142 L5 PT VISp C1ql2 PTgfr, 141 L5 PT VISp C1ql2 Cdh13, 140 L5 PT VISp Krt80, 134 L5 NP VISp Trhr Cpne7, 133 L5 NP VISp Trhr Met, 131 L6 CT Nxph2 Sla, 130 L6 CT VISp Krt80 Sla, 128 L6 CT VISp Nxph2 WIs, 127 L6 CT VISp Ctxn3 Brinp3, 126 L6 CT VISp Ctxn3 Sla, 122 L6 CT VISp Gpr139, 120 L6b Col8a1 Rprm, 119 L6b VISp Mup5, 118 L6b VISp Col8a1 Rxfp1, 115 L6b P2ry12, 114 L6b VISp Crh, 110 Lamp5 Krt73, 109 Lamp5 Fam19a1 Pax6, 108 Lamp5 Fam19a1 Tmem182, 106 Lamp5 Ntn1 Npy2r, 105 Lamp5 PIch2 Dock5, 101 Lamp5 Lsp1, 100 Lamp5 Lhx6, 97 Sncg Slc17a8, 96 Sncg Vip Nptx2, 95 Sncg Gpr50, 93 Vip Itih5, 90 Serpinf1 Clrn1, 89 Serpinf1 Aqp5 Vip, 85 Vip Igfbp6 Car10, 84 Vip Igfbp6 Pltp, 82 Vip Lmo1 Fam159b, 81 Vip Lmo1 Myl1, 79 Vip Igfbp6 Mab21l1, 78 Vip Arhgap36 Hmcn1, 77 Vip Gpc3 Slc18a3, 74 Vip Ptprt Pkp2, 73 Vip Rspo4 Rxfp1 Chat, 71 Vip Lect1 Oxtr, 70 Vip Rspo1 Itga4, 67 Vip Chat Htr1f, 66 Vip Pygm C1ql1, 61 Vip Crispld2 Htr2c, 60 Vip Crispld2 Kcne4, 58 Vip Col15a1 Pde1a, 54 Sst Chodl, 53 Sst Mme Fam114a1, 52 Sst Tac1 Htr1d, 50 Sst Tac1 Tacr3, 49 Sst Calb2 Necab1, 48 Sst Calb2 Pdlim5, 46 Sst Nr2f2 Necab1, 45 Sst Myh8 Etv1, 44 Sst Chrna2 Glra3, 42 Sst Myh8 Pibin, 40 Sst Chrna2 Ptgdr, 39 Sst Tac2 Myh4, 37 Sst Hpse Sema3c, 36 Sst Hpse Cbln4, 34 Sst Crhr2 Efemp1, 33 Sst Crh 4930553C11Rik, 31 Sst Esrn1, 29 Sst Tac2 Tacstd2, 28 Sst Rxfp1 Eya1, 27 Sst Rsfp1 Prdm8, 23 Sst Nts, 21 Pvalb Gabrg1, 20 Pvalb Th Sst, 18 Pvalb Calb1 Sst, 17 Pvalb Akr1c18 Ntf3, 16 Pvalb Sema3e Kank4, 14 Pvalb Gpr149 Islr, 11 Pvalb Reln Itm2a, 10 Pvalb Reln Tac1, 9 Pvalb Tpbg, 4 Pvalb Vipr2, 1 Meis2 Adamts19, 170 Astro Aqp4, 171 OPC Pdgfra Grm5, 173 Oligo Serpinb1a, 174 Oligo Synpr, 175 VLMC Osr1 Cd74, 176 VLMC Osr1 Mc5r, 177 VLMC Spp1 Col15a1, 178 Peri Kcni8, 179 SMC Acta2, 180 Endo Ctla2a, and 181 Microglia Siglech.

FIGS. 25A-25D. CN1457 (vAi107.0) Enhancer eHGT_078h (eAi107.0). (25A) Fluorescence expression of CN1457 shown in whole mouse brain in sagittal section. (25B) High resolution image (left) showing non-overlap of CN1457 SYFP fluorescence (red) and inhibitory marker Gad1 mRNA expression (white). Image on the right shows near compete overlap of CN1457 SYFP fluorescence (red) and cortical excitatory marker Slc17a7 mRNA expression (white). (25C) Quantification of specificity of CN1457 SYFP fluorescence in ALM and V1 mouse cortical areas based on multiplexed FISH data. (25D) Single cell transcriptomic characterization of SYFP fluorescent cells isolated from mouse V1. After single cell gene expression analysis, cells were mapped to an existing taxonomy of mouse cell types. Blue circle location reflects extent of single cell mapping (toward the final leaf), while size of the blue circle reflects the number of single cells that mapped to that point in the hierarchy. Bars projecting down reflect the number of cells that map to that terminal branch of the cell type taxonomy. The cells are the same as the cells listed in the Brief Description of the Figures of FIG. 24D.

FIGS. 26A-26C. CN1416 (vAi108.0) Enhancer eHGT_058m (eAi108.0). (26A) Fluorescence expression of CN1416 shown in whole mouse brain in sagittal section. (26B) High resolution image (left) showing non-overlap of CN1416 SYFP fluorescence (red) and inhibitory marker Gad1 mRNA expression (white). Image on the right shows near compete overlap of CN1416 SYFP fluorescence (red) and cortical excitatory marker Slc17a7 mRNA expression (white). (26C) Quantification of specificity of CN1416 SYFP fluorescence in ALM and V1 mouse cortical areas based on multiplexed FISH data.

FIGS. 27A-27C. CN1452 (vAi111.0) Enhancer eHGT_073h (eAi111.0). (27A) Fluorescence expression of CN1452 shown in whole mouse brain in sagittal section. (27B) Grayscale fluorescent images of DAPI, and mFISH images of Gad1, Pvalb, Sst, SYFP (CN1452) and Vip mRNA in mouse visual cortex. (27C) Co-staining of SYFP (CN1452) and Gad1 showing that only 7% of Gad1+ cells overlap with SYFP. N=43 cells from one animal.

FIGS. 28A-28C. CN1461 (vAi112.0) Enhancer eHGT_073m (eAi112.0). (28A) Fluorescence expression of CN1461 shown in whole mouse brain in sagittal section. (28B) Grayscale fluorescent images of DAPI, and mFISH images of Gad1, Pvalb, Sst, SYFP (CN1461), and Vip mRNA in mouse visual cortex. (28C) Co-staining of SYFP (CN1461) and Gad1 showing that only 1.5% of Gad1+ cells overlap with SYFP. N=130 cells from one animal.

FIGS. 29A-29C. CN1454 (vAi113.0) Enhancer eHGT_075h (eAi113.0). (29A) Fluorescence expression of CN1454 shown in whole mouse brain in sagittal section. (29B) High resolution image (left) showing non-overlap of CN1454 SYFP fluorescence (red) and inhibitory marker Gad1 mRNA expression (white). Image on the right shows near compete overlap of CN1454 SYFP fluorescence (red) and cortical excitatory marker Slc17a7 mRNA expression (white). (29C) Quantification of specificity of CN1454 SYFP fluorescence in V1 mouse cortical areas based on multiplexed FISH data.

FIGS. 30A-30C. CN1456 (vAi114.0) Enhancer eHGT_077h (eAi114.0). (30A) Fluorescence expression of CN1402 shown in whole mouse brain in sagittal section. (30B) High resolution image (left) showing non-overlap of CN1402 SYFP fluorescence (red) and inhibitory marker Gad1 mRNA expression (white). Image on the right shows near compete overlap of CN1402 SYFP fluorescence (red) and cortical excitatory marker Slc17a7 mRNA expression (white). (30C) Quantification of specificity of CN1402 SYFP fluorescence in ALM and V1 mouse cortical areas based on multiplexed FISH data. Single cell transcriptomic characterization of SYFP fluorescent cells isolated from mouse V1.

FIGS. 31A, 31B. CN1818 (vAi128.0) Enhancer mscRE4 (3×Core) (eAi3.2). Expression of construct CN1818 tested by (31A) Native fluorescence microscopy of cells labeled by retro-orbital injection, (31B) Hairpin Chain Reaction (HCR) RNA FISH targeting SYFP2 (from viral expression), Fam84b (expressed in L5 ET cells) and Rorb (expressed in L4 IT and L5 IT cells). FISH revealed a specificity rate of 78% in situ (62 Fam84b+ and SYFP2+/80 total SYFP2+).

FIG. 32A, 32B. CN2014 (vAi129.0) Enhancer mscRE4 (eAi3.0). Expression of construct CN2014 tested by (32A) Native fluorescence microscopy of cells labeled by retro-orbital injection, (32B) Hairpin Chain Reaction (HCR) RNA FISH targeting SYFP2 (from viral expression), Fam84b (expressed in L5 ET cells) and Rorb (expressed in L4 IT and L5 IT cells). FISH revealed a specificity rate of 85% in situ (45 Fam84b+ and SYFP2+/53 total SYFP2+).

FIG. 33. CN1427 (vAi130.0) Enhancer mscRE4 (4×) (eAi3.1). A Native tdTomato fluorescence expression in V1 region of a mouse brain slice. CN1427 serotype PHPeB virus was delivered by retroorbital injection, with analysis of reporter transgene expression at 40 days post injection.

FIGS. 34A, 34B. CN1466 (vAi131.0) Enhancer eHGT_078m (eAi128.0). (34A) Expression of vector CN1466 (green) in mouse neocortical brain slice culture at 25 days in vitro and 15 days post infection. Mutually exclusive labeling CN1466-labeled neurons (green) and GABAergic neurons (red). (34B) Expression of vector CN1466 in human neocortical brain slice cultures at 9 days in vitro and 9 days post infection. Extensive pyramidal neuron labeling is observed.

FIG. 35. CN2139 (vAi134.0) Enhancer eHGT_439m (eAi131.0). Expression of vector CN2139 by retroorbital delivery in mouse brain. Brain slices were subjected to fixed tissue immunohistochemistry with anti-GFP and anti-CTIP2 antibodies. Virus labeled cells were observed in L4 of neocortex.

FIG. 36. CN2137 (vAi135.0) Enhancer eHGT_440h (eAi132.0). Expression of vector CN2137 by retroorbital delivery in mouse brain. Brain slices were subjected to fixed tissue immunohistochemistry with anti-GFP and anti-CTIP2 antibodies. Virus labeled cells were observed in L6b of neocortex.

FIGS. 37A, 37B. (37A) CN1954 (vAi132.0) Enhancer eHGT_078h (3×Core) (eAi129.0). Expression of vector CN1954 in mouse neocortical brain slice culture at 27 days in vitro and 20 days post infection. (37B) CN1955 (vAi133.0) Enhancer eHGT_078m (3×Core) (eAi130.0). Expression of vector CN1955 in mouse neocortical brain slice culture at 27 days in vitro and 20 days post infection.

FIGS. 38A, 38B. (38A) vAi1.0 Enhancer mscRE1 (eAi1.0). Expression of construct mscRE1-SYFP2 tested by A) Native fluorescence imaging of retro-orbital injection. (38B) T502-059 (vAi2.0) Enhancer mscRE1 (eAi2.0). Expression of construct mscRE3-SYFP2 tested by A) Native fluorescence imaging of retro-orbital injection.

FIG. 39. T502-057 (vAi3.0) Enhancer mscRE4 (eAi3.0). Expression of construct mscRE4-SYFP2 tested by native fluorescence imaging of retro-orbital injection.

FIG. 40. Cell sources and Quality Control (QC Statistics). Barplot showing how many cells were flagged with each combination of QC criteria.N, number of cells collected. Unique fragments is the number of uniquely mapped fragments used for analysis, and was used for the first QC cutoff of >1e4 unique fragments. Fraction of fragments overlapping Encyclopedia of DNA Elements (ENCODE) DNase-seq peaks were computed using uniquely mapped fragments and were used for the second QC cutoff of >0.25. Fraction of fragments with length >250 bp was computed using unique fragments and provides the third QC cutoff of >0.1.

FIG. 41. Overview of enhancer discovery for viral tools. To build cell type-specific labeling tools, cells from adult mouse cortex were isolated and a single cell assay for transposase-accessible chromatin using sequencing (scATAC-seq) was performed. Samples were clustered and compared to single cell RNA sequencing (scRNA-seq) datasets to identify the clusters. Single cells matching the same transcriptomic types were then pooled and the genome was searched for type-specific putative enhancers. These regions were cloned upstream of a minimal promoter in an adeno-associated virus (AAV) genomic backbone, which was used to generate self-complementary adeno-associated viral vectors (scAAVs) or recombinant adeno-associated viral vectors (rAAVs). These viral tools were delivered retro-orbitally or stereotaxically to label specific cortical populations. In cells with a matching cell type, enhancers recruit their cognate transcription factors to drive cell type-specific expression. In other cells, viral genomes are present, but transcripts are not expressed. However, it is necessary to test the enhancer constructs for specificity, as not all enhancers behave as expected.

FIG. 42. Fluorescence-activated cell sorting (FACS) Gating examples. (4A) All FACS sorts followed a similar gating strategy: Morphology and debris removal using Forward Scatter Area (FSC-A) and Side Scatter Area (SSC-A); Removal of doublets/multiplets using Forward Scatter Width (FSC-W) x Forward Scatter Height (FSC-H) and Side Scatter Width (SSC-W) x Side Scatter Height (SSC-H) gating; and selection of live cells with or without fluorescent labels using 4′,6-diamidino-2-phenylindole (DAPI) and fluorophore signals. This panel shows example shows gating for direct fluorophore labeling of cells from injection of mscRE4-SYFP2.

FIG. 43. Gm12878 platform comparisons. Comparison of FACS-sorted scATAC-seq libraries to those previously generated using Fluidigm C1 (Buenrostro, et al., Nature 523 (2015)) sci-ATAC-seq (Cusanovich, et al., Science 348 (2015) and Pliner, et al., Mol. Cell 71 (2018)) or droplet-based indexing (10× Genomics) for which data using the common cell line of human Gm12878 cells is available. To use in these comparisons, scATAC-seq data was generated using a FACS-based method for 60 Gm12878 cells. For each published dataset, raw data was obtained from GEO and was aligned and analyzed using the same methods. For 10× Genomics, aligned fragment locations and metadata were obtained from the 10× genomics website. Abbreviations used throughout the plots: bu, Buenrostro, et al., Nature 523 (2015) Fluidigm C1 ATAC-seq; cu, Cusanovich, et al., Science 348 (2015) sci-ATAC-seq (2015); gr, Graybuck, et al. (the data described herein) FACS scATAC-seq; pl, Pliner, et al., Mol. Cell 71 (2018) sci-ATAC-seq (2018), and tx, 10× Genomics, 5k cells 10× ATAC-seq. Gray et al., Elife 1-30 (2017). Two-axis QC criteria plot, showing the QC1 and QC2 cutoffs used for mouse cortical scATAC-seq data.

FIG. 44. Gm12878 platform comparisons. Aggregate fragment length frequency plots. Fragment length is shown on the x-axis, and the fraction of reads with fragments of each bp size was calculated for each sample in each dataset. For this analysis, the median fraction at each fragment size is shown as a solid line, with 25th and 75th percentiles shown in the shaded regions. Abbreviations used throughout the plots: bu, Buenrostro, et al., Nature 523 (2015) Fluidigm C1 ATAC-seq; cu, Cusanovich, et al., Science 348 (2015) sci-ATAC-seq (2015); gr, Graybuck, et al. (the data described herein)

FIG. 45. Samples were clustered in t-SNE space using the Phenograph implementation of Louvain clustering. To identify the cell types within these clusters, cells from each cluster were pooled, and the number of fragments within 20 kb of each TSS were counted. Then, marker genes for transcriptomic clusters from Tasic et al., Nature 563, 72-78 (2018) were selected, and correlations between TSS accessibility scores and log-transformed gene expression were performed. The scRNA-seq cluster with the highest correlation score was assigned as the identity for each Phenograph cluster, and clusters with the same transcriptomic mapping were combined for downstream analyses. The cluster with the highest correlation score was assigned as the identity for each cluster, and clusters with the same transcriptomic mapping were combined for downstream analyses.

FIGS. 46A-46D. scATAC-seq data. The dotplot shows both the fraction of cells in each subclass that express each gene (size of points), and the median expression level within each subclass (color of points). scATAC-seq data were grouped by subclass based on transcriptomic mapping, and aggregated fragment overlaps were plotted near the gene of interest after normalization for fragment count (track plots, right panel). (46A) Subclass-level gene expression profiles (dot-plots, left panel) from Tasic, et al. (2018, Nature) show highly specific expression of the Fam84b gene in the L5 PT subclass. Fam84b (family with sequence similarity 74, member B) is a transcription factor gene that was recently shown to be a highly selective marker gene for L5 PT neurons across two regions of the mouse cortex (Tasic, et al. (2018) Nature). A peak of accessibility specific to L5 PT samples (mscRE4) was identified 113 kb downstream from the Fam84b TSS. (46B) Subclass-level gene expression profiles (dot-plots, left panel) from Tasic, et al. (2018) show enrichment of Hsd11b1 expression in L5 IT and L5 PT cell types. Hsd11b1 (hydroxysteroid 11-beta dehydrogenase 1) is a gene involved in corticosteroid biosynthesis. It has been shown to be selectively expressed in L5 cells, with higher expression in some L5 IT types than in L5 PT cells {Tasic, et al. (2018) Nature}. A peak of accessibility enriched in L5 IT cells but absent in L5 PT cells (mscRE16) was identified 34 kb upstream of the Hsd11b1 TSS. The cell types listed along the side of FIGS. 46A and 46B are (from top to bottom) Lamp5, Sncg, Serpinf1, Vip, Sst Pvalb, L2/3 IT, L4, L5 IT, L6 IT, L5 PT, NP, L6 CT, L6b, Meis2, and CR. (46C) scATAC-seq data showed a peak of accessibility specific to mscRE10 located 34 kb upstream of Car3. (46D) scATAC-seq data showed a peak of accessibility specific to mscRE13 located 86 kb upstream of Osr1.

FIG. 47. mscRE locations and cloning primers.

FIGS. 48A-48C. (48A) Direct enhancer-driven expression of a fluorophore was tested by cloning the putative mscRE4 or mscRE16 enhancer in an scAAV construct with a minimal promoter driving a fluorophore-WPRE3. After packaging, purification, and titering scAAVs were retro-orbitally injected into a wild-type mouse. (48B) Two weeks after retro-orbital injection of an rAAV with mscRE16 driving expression of EGFP (TG1002), cells are selectively labeled in L5 of the mouse cortex by EGFP expression, which is amplified here using antibody staining by immunohistochemistry (IHC). (48C) Two weeks after retro-orbital injection of an scAAV with mscRE4 driving expression of SYFP (T502-057), dim but distinct labeling was seen in L5 PT cells by native fluorescence without antibody amplification.

FIGS. 49A, 49B. Validation of cell type targeting of scAAV-mscRE4-SYFP2 viruses by scRNA-seq. (49A) Enhancer-driven recombinase expression was tested using a scAAV construct with a minimal promoter driving EGFP-WPRE3. After packaging, mice were given retro-orbital injections. After 2 weeks, SYFP-expressing cells were visible in the cortex, which could be isolated by FACS and used for scRNA-seq. (49B) Centroid classifier mapping of labeled cells onto data from Tasic, et al. (2018, Nature) revealed that 91.8% of the cells mapped to L5 PT transcriptomic cell types.

FIGS. 50A-50C. Electrophysiological characterization of mscRE4-labeled cells and demonstration of utility for electrophysiological recording of labeled neurons. (50A) Cortical slices from an animal labeled with the scAAV-mscRE4-SYPF2 (T502-057) virus were used for electrophysiological characterization. Example impedance amplitude profiles obtained from a (Yellow Fluorescent Protein) YFP+ and a YFP-neuron in VISp. For comparison, impedance amplitude profiles from an unlabeled PT-like and an IT-like neuron from somatosensory cortex are also shown. Resonance frequency is plotted as a function of input resistance. (50B) Example voltage responses to a series of hyperpolarizing and depolarizing current injections for a YFP+ and a YFP− neuron. Example voltage responses obtained from unlabeled PT-like and IT-like neurons are also shown for reference. (50C) Input resistance, sag ratio and resonance frequency for three experimental conditions.

FIGS. 51A, 51B. Additional eletrophysiological characteristics of mscRE4-SYFP2 labeled cells. (51A) Microscopy of example cells characterized by patch electrophysiology. Left, a SYFP2-positive cell; right, a SFYP2-negative cell. (51B) Input resistance, sag ratio, and resonance frequency for the four experimental conditions: IT, YFP−, YFP+, and PT.

FIGS. 52A, 52B. Stereotaxic labeling using enhancer-driven viruses. (52A) Native fluorescence imaging of animals with stereotaxic injection of mscRE4-EGFP in primary visual cortex. Enhancer-driven viruses were co-injected with a constitutive dTomato virus, rAAVDJ-EF1a-dTomato at 0.1× of the volumes of the mscRE viruses, to provide injection site location (dotted outlines). (52B) Native fluorescence imaging of animals with stereotaxic injection of mscRE4-SYFP2 into primary visual cortex at the indicated volumes.

FIG. 53. Some enhancer-driven recombinase viruses provide specific, binary labeling. Three different recombinases and one transactivator were inserted downstream of mscRE4 and a promoter in viral constructs. After retro-orbital injection, labeling of L5 was found with various degrees of specificity using tTA2 (TG1011, SEQ ID NO: 88) in an Ai63 reporter mouse (most sparse, most specific), FlpO (TG978, SEQ ID NO: 80) in an Ai65F reporter mouse (most complete and specific), iCre (TG1010, SEQ ID NO: 87) in an Ai14 reporter mouse (complete, but with background in L6), and dgCre in an Ai14 reporter mouse (least specific). Images show native fluorescence in visual cortex 2 weeks post-injection. See FIGS. 68A-68G for depictions.

FIG. 54. Brain-wide imaging of retro-orbitally delivered mscRE4-FlpO-WPRE3 (TG978, SEQ ID NO: 80) viral labeling reveals specific, L5-restricted labeling throughout the cortex, and labeling of specific subcortical populations in the central amygdalar nucleus, capsular part (CEAc), a portion of the CeA, which receives and processes pain signals; the substantia nigra, compact part (or pars compacta, SNc), which is involved in movement control and is affected by Parkinson's disease; and prosubiculum (ProS).

FIGS. 55A, 55B. Validation of cell type targeting of mscRE4-FlpO-WPRE3 viruses by scRNA-seq. (55A) Enhancer-driven recombinase expression was tested using an rAAV construct with a minimal promoter driving FlpO-WPRE3. After packaging, Ai65F mice were given retro-orbital injections. After 2 weeks, tdTomato-expressing cells were visible in the cortex, which could be isolated from L5 dissection, and were sorted by FACS and used for scRNA-seq. (55B) Centroid classifier mapping of labeled cells onto data from Tasic, et al. (2018, Nature) revealed that 90.6% of the cells mapped to L5 PT transcriptomic cell types. The list of cell types along the right, from top to bottom, are: Sst Hpse Cbln4 (3), L5 IT VISp Hsd11b1 Endou (2), L5 PT VISp Chrna6 (2), L5PT VISp Lgr5 (2), L5 PT VISp C1ql2 Ptgfr (40), L5 PT VISp C1ql2 Cdh13 (40), and L5 PT VISp Krt80 (7).

FIGS. 56A-56C. Dual labeling and titration of viral copy number to achieve broad, intersectional labeling (56A, 56B) at high titer, and specific, exclusive labeling (56C) at low titer. These experiments were performed by retro-orbital coinjection of mscRE4-FlpO (TG978, SEQ ID NO: 80) and mscRE16-EGFP (TG1002, SEQ ID NO: 86) viruses into Flp-dependent tdTomato reporter mice (Ai65F). See FIG. 68 for depictions of this dual-labeling strategy. Corner of fluorescence image identifies the fluorophore (Anti-GFP, Native tdTomato, and merge).

FIGS. 57A-57C. Enhancer-driven recombinase viruses as drivers for cell labeling. (57A) Full-section imaging of a mscRE4-FlpO injection shows labeling throughout L5 of the posterior cortex. Inset region on the right corresponds to the white box on the left. Layer overlays from the Allen Brain Reference Atlas shows labeling restricted primarily to L5. tdTomato+ cells were dissected from the full cortical depth and were collected by FACS for scRNA-seq. Transcriptomic profiles were mapped to reference cell types from Tasic, et al. (2018). 87.5% of cells (28 of 32) mapped to L5 PT cell types. (57B) Full-section imaging of mscRE10-FlpO injection shows labeling in layer 6 (L6) of the cortex. Inset region on the right corresponds to the white box on the left. scRNA-seq of tdTomato+ cells shows that layer 6 corticothalamic (L6 CT) and L6b cell types are the most frequently labeled subclasses of neurons at 75% (n=36 of 48). (57C) Full-section imaging of mscRE16-FlpO injection shows labeling in L5 of the cortex. Inset region on the right corresponds to the white box on the left. scRNA-seq of tdTomato+ cells shows that L5 IT cell types are the most frequently labeled subclass of neurons at 42% (n=20 of 48), but other subclasses are also labeled at this titer (Lamp5, 27%; L6 IT, 6%; L5 PT, 15%).

FIG. 58. Retro-orbital mscRE driver screening at multiple titers. Native fluorescence images for reporter mice retro-orbitally (RO) injected with enhancer-driven recombinase viruses at two titers: Low RO, 1×10¹⁰ genome copies, GC; High RO, 1×10¹¹ GC. Fluorescence is tdTomato. Scale bar sizes can be determined by Scale Bar Key where a triangle indicates a scale of 100 μm, the 7-point star indicates a scale of 500 μm, and the 5-point star indicates a scale of 1000 μm. The arrows show where layers are labeled where, in the direction of the arrow, the layers are labeled L1, L2/3, L4, L5, L6, and L6b.

FIGS. 59A-59E. Brain-wide and intersectional labeling of cell type. (59A) Results from full-brain imaging using TissueCyte. Sections throughout the whole brain of an Ai65F mouse after retro-orbital injection of mscRE-FlpO were aligned to the Allen Institute Common Coordinate Framework (CCF) and mapped to the Allen Brain Atlas structural ontology. A high-level overview of cell labeling throughout the structural ontology is represented by the taxonomic plot. “Grey” is the root of the plot, representing all grey-matter regions, and each branching of nodes shows child structures within each region. The size and color of nodes represents the maximum signal found among all children of the nodes, which allows one to follow the tree to the source of high signal within each structure. Insets display selected regions of high or specific signal. Region acronyms correspond to the Allen Brain Adult Mouse Atlas. (59B) Further division of the isocortical regions in the TissueCyte dataset to the level of cortical layers allows brain-wide quantification of layer-specific signal. Representative cortical sections from the TissueCyte dataset are shown along the top, from most anterior to most posterior (left to right). The heatmap shows quantification of the signal in each region and layer. Agranular regions, which lack layer 4, have hashing in the L4 row. From Anterior to Posterior the regions are FRP, ORBv1, ORBm, ORBl, PL, ILA, Aid, Mos, Alv, Mop, SSp-m, GU, ACAd, SSp-n, SSp-un, ACAv, SSp-ul, SSp-II, VISC, Aip, SSs, SSp-bfd, SSp-tr, AUDv, AUDd, AUDp, PTLp, RSPv, RSPd, PERI, VISam, TE, ECT, AUDpo, VISI, VISpm, and VISpl. (59C) Diagrams showing the use of co-injected recombinase viruses in a dual-reporter system for co-labeling or intersectional labeling of cell types. In this experiment, one virus driving FlpO and a second driving iCre are co-injected into a mouse with genetically-encoded Flp-dependent and Cre-dependent reporters. In target cell types, enhancers will drive the recombinases, which will permanently label their target cell types. If the enhancers selected are mutually exclusive, distinct populations will be labeled. If they overlap, intersectional labeling is possible. (59D) Native fluorescence imaging of an Ai65F;Ai140 dual-reporter mouse line retro-orbitally injected with mscRE16-FlpO (red fluorescence) and mscRE4-iCre (green fluorescence). These enhancers are expected to label mutually-exclusive cell types in L5 of the cortex. The region in the white box corresponds the inset image, showing strong labeling of cells in L5. (59E) Cell counts within each layer for all cortical regions labeled with EGFP (mscRE4; L5 PT), tdTomato (mscre16; L5 IT), or both in the image in (59D).

FIGS. 60A, 60B. Whole-brain characterization of mscRE16-FlpO. (60A) TissueCyte imaging of an mscRE16-FlpO;Ai65F mouse 2 weeks after retro-orbital injection was registered to the Allen Institute Common Coordinate Framework (CCF), and each structure in the adult mouse structural ontology was scored. As for FIGS. 59A-59E, these panels provide a high-level overview of cell labeling throughout the structural ontology. The size and color of nodes represents the maximum signal found among all children of the nodes, which allows one to follow the tree to the source of high signal within each structure. Insets display selected regions of high or specific signal. The inset at the bottom-left shows projection of IT neurons across the corpus callosum. (60B) Layer quantification for the same TissueCyte image registered to the CCF for all isocortical regions. Agranular regions that lack L4 are shown with a white box in the L4 row. All acronyms correspond to the Allen Institute for Brain Science Adult Mouse 3D atlas.

FIGS. 61A-61D. mscRE4 AAV vectors target rare L5 PT neurons in the human cortex. Human acute slice cultures resected from the middle temporal gyrus (MTG) were infected with a quartet of viruses: two mscRE4-driven rAAVs expressing Cre or Flp recombinase and two fluorescent reporter viruses, one expressing SYFP and the other expressing an RFP. This strategy enables high specificity by selection of only colabeled neurons. (61A) Biocytin fills of colabeled cells that were used for patch electrophysiology reveals morphology consistent with human L5 PT neurons; (61B) dual fluorescent labeling of a L5 PT neuron in human cortex (scale bar is 100 microns); (61C) transcriptomic validation was performed by mapping RNA extracted from a labeled cell using Patch-seq. The RNA was reverse-transcribed, amplified, sequenced, and mapped to a human MTG reference dataset, and matched the human L4/5 PT cell type in 100 of 100 trials using a bootstrapped centroid classifier; (61D) electrophysiology of a colabeled human L5 PT cell is consistent with previous studies of L5 PT cells, and demonstrates the utility of this method for selective electrophysiological targeting.

FIG. 62. Annotated sequence of CN1818

FIG. 63. 3×Core-mscRE4-SYFP2 viruses (CN1818, SEQ ID NO: 109) were injected retro-orbitally into adult mice. 3 weeks after injection, brains from injected mice were sectioned and imaged to assess targeted expression of SYFP2 fluorophore labeling. Robust expression of SYFP2 reporter gene in the adult mouse brain was observed following retroorbital injection of CN1818. Labeled cells are predominantly in layer 5 and have electrophysiological properties consistent with L5 PT neurons.

FIGS. 64A, 64B. (64A) Nissl stain of the M1 region in a macaque brain slice showing neocortical layers, and higher magnification view of the boxed region showing numerous magnopyramidal Betz cells (white arrows). (64B) Native YFP expression detected in a Betz cell (white arrow) 4 days post infection with CN1818, and corresponding Nissl stain of the same field of view.

FIGS. 65A-65C. (66A) Prospective viral labeling (green) and targeted patch clamp recording of a putative Betz cell in a cultured macaque M1 brain slice infected with CN1818, with Alexa dye filling from the patch pipette (red). (66B) Firing in response to a 1s, 3 nA current injection step, showing narrow action potential width. (66C) Summary plot showing high firing rate in response to escalating current injection steps.

FIGS. 66A-66C. (66A) Spike frequency acceleration and subthreshold membrane potential oscillations in the gamma band shown for a CN1818 virus labeled macaque M1 putative Betz cell. (66B) Prominent fast sag, low input resistance (19MOhms) and (66C) subthreshold membrane resonance with a peak resonance frequency of 5.3 Hz.

FIG. 67. 3×Core-mscRE4-SYFP2 viruses (CN1818, SEQ ID NO: 109) was applied to human surgical ex vivo cortical slice cultures. After incubation, the cortical slices were imaged by microscopy to assess targeted expression of SYFP2 fluorophore labeling. It was found that CN1818 labels L5 PT neurons in human ex vivo neocortical brain slice cultures. Scale bars are 1 mm in length.

FIGS. 68A-68G. (68A) and (68B) show the traditional Cre/lox and Flp/FRT systems, respectively, to generate cell type-specific labels by breeding. (68C) Shows the traditional TET Transactivator/TET Responsive element (tTA2/TRE) system used to generate cell type-specific labels. (68D), (68E), and (68F) show mechanisms to bypass breeding by substituting a viral Cre, Flp, or tTA driver. (68F) also shows an additional layer of regulation via doxycycline treatment, which can reduce or inactivate tTA2 activity. (68G) shows bypassing these systems altogether for direct labeling. A strong advantage of the Cre or Flp-dependent reporters is that they can be much brighter and are permanently on after recombination to remove the STOP sites. The tTA2/TRE system is an additional mechanism for selective labeling that may also be tunable by doxycycline treatment.

FIG. 69. Diagrammatic overview of a multi-virus labeling system. Here, two different viruses driven by the same or different enhancers drive either a recombinase or a fluorophore. If injected into a reporter mouse, enhancer-driven recombinases will cause excision of a STOP site in the target cell type, and the enhancer-driven fluorophore will be expressed directly in another target cell type. If these cell types overlap in their use of the viral enhancer elements, intersectional colabeling can be observed.

FIG. 70. Enhancer ID, labeled cell types, and validation methods.

FIG. 71. Summary of vector components. Sequence names, associated length, enhancer, promoter, product class, primary product and other components of expression constructs described herein.

FIG. 72. Taxonomy and clustering of selected central nervous system cells.

FIGS. 73A, 73B. (73A) Enhancer targeting validation data. FM stands for fluorescence microscopy. (73B) Cell type specificity of enhancers and vectors described herein. S=subset of types in group; A=all types in group; *=validated in mouse, RNA-seq, and a third modality; ˜=validated in mouse, RNA-seq, primate/human, and a fourth modality.

FIG. 74. Schematic of cortical layers, with particular relevance to the primate visual cortex. This schematic is provided as an illustration of intracortical layers.

FIGS. 75A, 75B. A database of human neocortical cell subclass-specific accessible chromatin elements. (75A) Workflow for human neocortical epigenetic characterization. (75B-75D) High-quality nuclei (2858 from 14 specimens) visualized by tSNE and colored according to mapped transcriptomic cell type (75B), sort strategy (775C), or specimen (75D). L, layer. (75E) Transcriptomic abundances of eleven known cell subclass-specific marker genes across 75 cell types identified in human temporal cortex middle temporal gyrus (Hodge et al., bioRxiv, 384826, 2018).

FIG. 76. Mapping ATAC-seq clusters to RNA-seq cell types. Transcriptomic cell types within subclasses were summed across for clusterwise mapping, to yield clusterwise mapping to subclasses. This plot represents the final mapped subclass assigned as the most frequent mapping for each cluster, and these subclass identities are used for the pileups and calculations in FIGS. 75B, 77, and 78.

FIG. 77. Properties of human neocortical cell subclass-specific accessible genomic elements. Percent overlap of ATAC-seq peaks with previously identified DMRs (Lister et al., Science. 341, 1237905, 2013, Luo et al., Science. 357, 600-604, 2017), comparing real peaks to randomized peak positions. Absolute numbers of detected peaks and peak-DMR overlaps are shown

FIG. 78. Accessible chromatin elements furnish human genetic tools. Multiple enhancer-AAV vectors yield distinct subclass selectivities. Seven gene loci and ATAC-seq read pileups are shown, as well as expression pattern in mouse V1 for those seven AAV reporter vectors. Scale 200 μm.

FIG. 79. Sequences supporting the disclosure. Sequences for Enhancer Grik1-enhScnn1a-1 short form (SEQ ID NO: 188), Enhancer Grik1-enhScnn1a-1 (eAi14.0) (SEQ ID NO: 25), Enhancer mscRE1 (eAi1.0) (SEQ ID NO: 26), Enhancer mscRE3 (eAi2.0) (SEQ ID NO: 27), Enhancer mscRE4 (eAi3.0) (SEQ ID NO: 28), Enhancer mscRE4 core (SEQ ID NO: 29), Enhancer 3× mscRE4 core (eAi3.2) (SEQ ID NO: 30), Enhancer mscRE4 (4×) (eAi3.1) (SEQ ID NO: 31), Enhancer mscRE10 (eAi6.0) (SEQ ID NO: 32), Enhancer mscRE11 (eAi7.0) (SEQ ID NO: 33), Enhancer mscRE12 long form (SEQ ID NO: 34), Enhancer mscre12 (eAi8.0) (SEQ ID NO: 35), Enhancer mscRE13 (eAi9.0) (SEQ ID NO: 36), Enhancer mscRE16 (eAi11.0) (SEQ ID NO: 37), Enhancer 4XmscRE16 (eAi11.1) (SEQ ID NO: 38), Enhancer eHGT_078h (eAi107.0) (SEQ ID NO: 39), Enhancer eHGT_078h Core (SEQ ID NO: 177), Enhancer eHGT_078h (3×Core) (eAi129.0) (SEQ ID NO: 40), Enhancer eHGT_058h (eAi106.0) (SEQ ID NO: 41), Enhancer eHGT_058m (eAi108.0) (SEQ ID NO: 42), Enhancer eHGT_073h (eAi111.0) (SEQ ID NO: 43), Enhancer eHGT_073m (eAi112.0) (SEQ ID NO: 44), Enhancer eHGT_075h (eAi113.0) (SEQ ID NO: 45), Enhancer eHGT_077h (eAi114.0) (SEQ ID NO: 46), Enhancer eHGT_254h (eAi127.0) (SEQ ID NO: 47), Enhancer eHGT_078m (eAi128.0) (SEQ ID NO: 48), Enhancer eHGT_078m Core (SEQ ID NO: 178), Enhancer eHGT_078m (3×Core) (eAi130.0) (SEQ ID NO: 49), Enhancer eHGT_439m (eAi131.0) (SEQ ID NO: 50), Enhancer eHGT_440h (eAi132.0) (SEQ ID NO: 51), Beta-globin minimal promoter (SEQ ID NO: 52), minCMV (SEQ ID NO: 53), mutated minCMV promoter (SEQ ID NO: 54), Hsp68 minimal Promoter (SEQ ID NO: 55), SYFP2 (SEQ ID NO: 56), EGFP (SEQ ID NO: 57), Optimized Flp recombinase (SEQ ID NO: 58), Improved Cre recombinase (SEQ ID NO: 59), WPRE3 (SEQ ID NO: 60), BGHpA (SEQ ID NO: 61), HA tag (SEQ ID NO: 62), HA tag (SEQ ID NO: 63), P2A (SEQ ID NO: 64), T2A (SEQ ID NO: 65), E2A (SEQ ID NO: 66), F2A (SEQ ID NO: 67), tet-Transactivator (SEQ ID NO: 68), PHP.eB capsid (SEQ ID NO: 69), AAV9 VP1 capsid (SEQ ID NO: 70), Plasmid backbone 1 (SEQ ID NO: 71), Plasmid backbone 2 (SEQ ID NO: 72), T502-050 (vAi33.0) (SEQ ID NO: 73), T502-054 (vAi33.1) (SEQ ID NO: 179), T502-057 (vAi3.0) (SEQ ID NO: 74), T502-059 (vAi2.0) (SEQ ID NO: 75), vAi1.0 (SEQ ID NO: 76), vAi33.2 (TG1114) (SEQ ID NO: 77), vAi34.0 (TG1108) (SEQ ID NO: 78), vAi45.0 (TG1109) (SEQ ID NO: 79), TG975 (vAi4.0) (SEQ ID NO: 180), TG978 (vAi4.1) (SEQ ID NO: 80), TG979 (vAi4.2) (SEQ ID NO: 181), TG981 (vAi5.0) (SEQ ID NO: 81), TG982 (vAi6.0) (SEQ ID NO: 182), TG987 (vAi7.0) (SEQ ID NO: 183), TG988 (vAi7.1) (SEQ ID NO: 82), TG995 (vAi15.0) (SEQ ID NO: 83), TG996 (vAi19.0) (SEQ ID NO: 84), TG997 (vAi20.0) (SEQ ID NO: 184), TG999 (vAi21.0) (SEQ ID NO: 85), TG1002 (vAi26.0) (SEQ ID NO: 86), TG1009 (vAi8.0dgCre) (SEQ ID NO: 185), TG1010 (vAi6.1) (SEQ ID NO: 87), TG1011 (vAi7.2) (SEQ ID NO: 88), TG1021 (vAi8.0Cre) (SEQ ID NO: 89), TG1022 (vAi9.0) (SEQ ID NO: 186), TG1036 (vAi16.0) (SEQ ID NO: 90), TG1037 (vAi22.0) (SEQ ID NO: 91), TG1038 (vAi27.0) (SEQ ID NO: 92), TG1045 (vAi17.0) (SEQ ID NO: 187), TG1046 (vAi23.0) (SEQ ID NO: 93), TG1047 (vAi28.0) (SEQ ID NO: 94), TG1048 (vAi18.0) (SEQ ID NO: 95), TG1049 (vAi24.0) (SEQ ID NO: 96), TG1050 (vAi29.0) (SEQ ID NO: 97), TG1052 (vAi10.0) (SEQ ID NO: 98), CN1402 (vAi106.0) (SEQ ID NO: 99), CN1416 (vAi108.0) (SEQ ID NO: 100), CN1427 (vAi130.0) (SEQ ID NO: 101), CN1452 (vAi111.0) (SEQ ID NO: 102), CN1454 (vAi113.0) (SEQ ID NO: 103), CN1456 (vAi114.0) (SEQ ID NO: 104), CN1457 (vAi107.0) (SEQ ID NO: 105), CN1461 (vAi112.0) (SEQ ID NO: 106), CN1466 (vAi131.0) (SEQ ID NO: 107), CN1772 (vAi127.0) (SEQ ID NO: 108), CN1818 (vAi128.0) (SEQ ID NO: 109), CN1954 (vAi132.0) (SEQ ID NO: 110), CN1955 (vAi133.0) (SEQ ID NO: 111), CN2014 (vAi129.0) (SEQ ID NO: 112), CN2137 (vAi135.0) (SEQ ID NO: 113), CN2139 (vAi134.0) (SEQ ID NO: 114), Myosin light chain kinase, Green fluorescent protein, Calmodulin chimera (SEQ ID NO: 115), Genetically-encoded green calcium indicator NTnC (SEQ ID NO: 116), Calcium indicator TN-XXL (SEQ ID NO: 117), BRET-based auto-luminescent calcium indicator (SEQ ID NO: 118), Calcium indicator protein OeNL (Ca2+)-18u (SEQ ID NO: 119), GCaMP6m (SEQ ID NO: 120), GCaMP6s (SEQ ID NO: 121), GCaMP6f (SEQ ID NO: 122), Channelopsin 1 (SEQ ID NOs: 123 and 124), Channelrhodopsin-2 (SEQ ID NOs: 125 and 126), CRISPR-associated protein (Cas) (SEQ ID NO: 127), Cas9 (SEQ ID NO: 128), CRISPR-associated endonuclease Cpf1 (SEQ ID NO: 129), Ribonuclease 4 or Ribonuclease L (SEQ ID NO: 130), Deoxyribonuclease II beta (SEQ ID NO: 131), Sodium channel protein type 1 subunit alpha (SEQ ID NO: 132), Potassium voltage-gated channel subfamily KQT member 2 (SEQ ID NO: 133), Voltage-dependent L-type calcium channel subunit alpha-1C (SEQ ID NO: 134), Lactase (SEQ ID NO: 135), Lipase (SEQ ID NO: 136), Helicase (SEQ ID NO: 137), Amylase (SEQ ID NO: 138), Alpha-glucosidase (SEQ ID NO: 139), Transcription factor SP1 (SEQ ID NO: 140), Transcription factor AP-1 (SEQ ID NO: 141), Heat shock factor protein 1 (SEQ ID NO: 142), CCAAT/enhancer-binding protein (C/EBP) beta isoform a (SEQ ID NO: 143), Octamer-binding protein 1 (Oct-1) (SEQ ID NO: 144), Transforming growth factor receptor beta 1 (SEQ ID NO: 145), Platelet-derived growth factor receptor (SEQ ID NO: 146), Epidermal growth factor receptor (SEQ ID NO: 147), Vascular endothelial growth factor receptor (SEQ ID NO: 148), Interleukin 8 receptor alpha (SEQ ID NO: 149), Caveolin (SEQ ID NO: 150), Dynamin (SEQ ID NO: 151), Clathrin heavy chain 1 isoform 1 (SEQ ID NO: 152), Clathrin heavy chain 2 isoform 1 (SEQ ID NO: 153), Clathrin light chain A isoform a (SEQ ID NO: 154), Clathrin light chain B isoform a (SEQ ID NO: 155), Ras-related protein Rab-4A isoform 1 (SEQ ID NO: 156), Ras-related protein Rab-11A (SEQ ID NO: 157), Platelet-derived growth factor (SEQ ID NO: 158), Transforming growth factor-beta3 (SEQ ID NO: 159), Nerve growth factor (SEQ ID NO: 160), Epidermal growth factor (EGF) (SEQ ID NO: 161), GTPase HRas (SEQ ID NO: 162), Cocaine And Amphetamine Regulated Transcript (Chain A) (SEQ ID NO: 163), Protachykinin-1 (SEQ ID NO: 164), Substance P is position 58-68 of Protachykinin-1 (SEQ ID NO: 165), Oxytocin-neurophysin 1 (SEQ ID NO: 166), Oxytocin is position 20-28 of Oxytocin-neurophysin 1 (SEQ ID NO: 167), and Somatostatin (SEQ ID NO: 168) . . . . The nucleic acid sequences described herein are shown using standard letter abbreviations for nucleotide bases, as defined in 37 C.F.R. § 1.822. Only one strand of each nucleic acid sequence is shown, but the complementary strand is understood as included in embodiments where it would be appropriate.

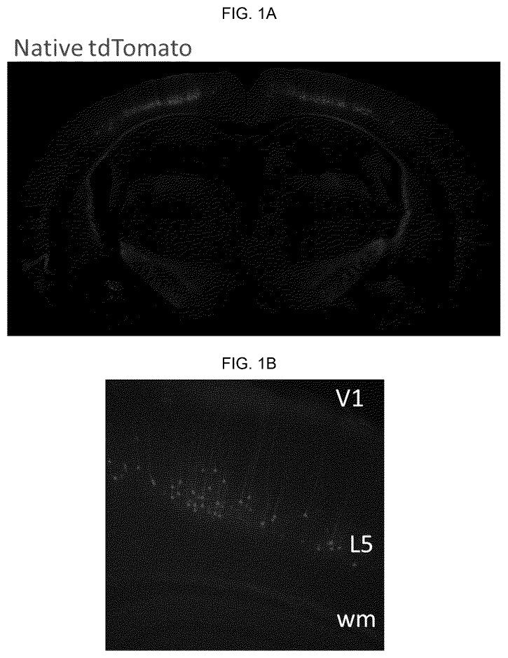

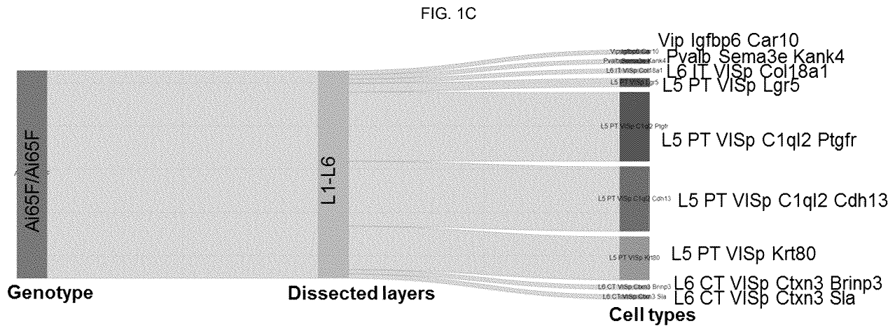

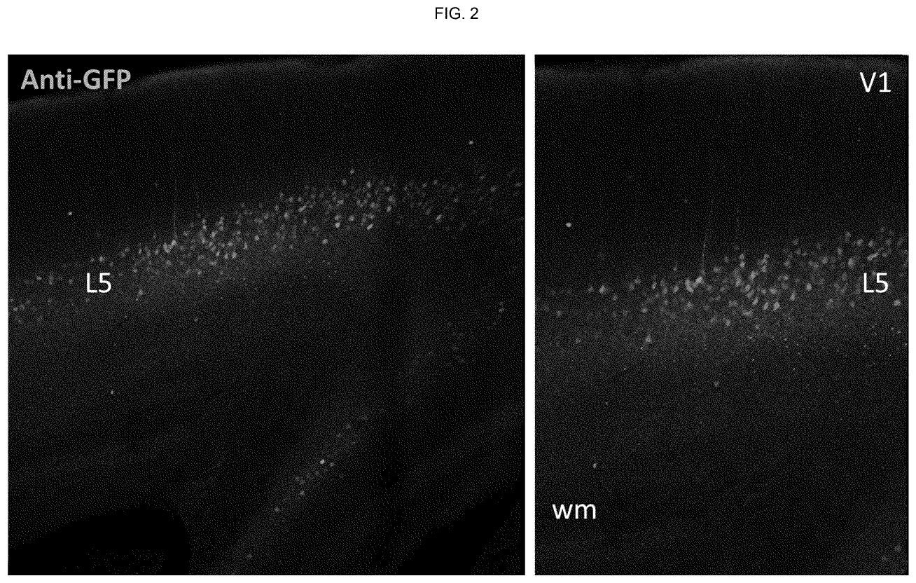

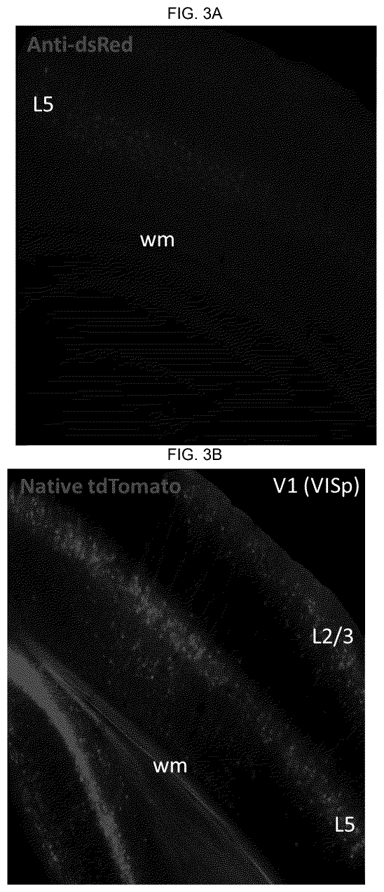

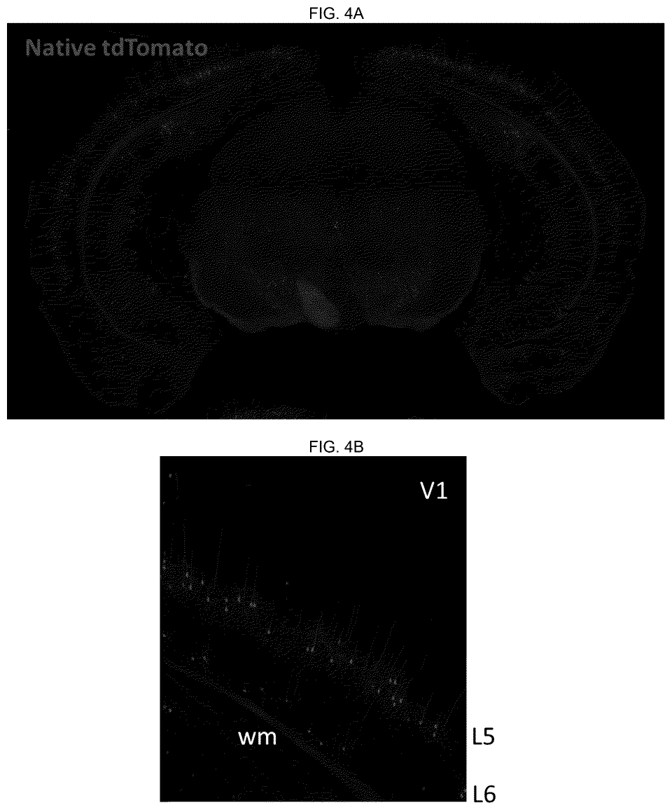

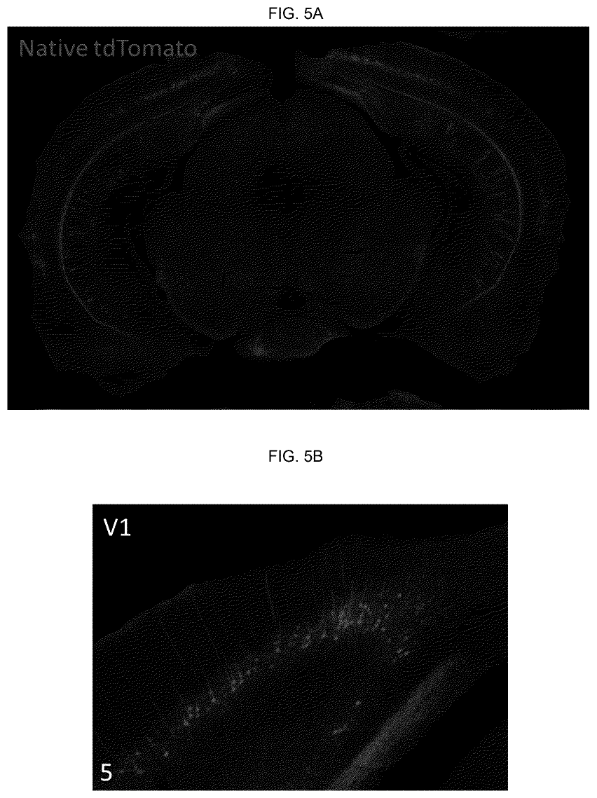

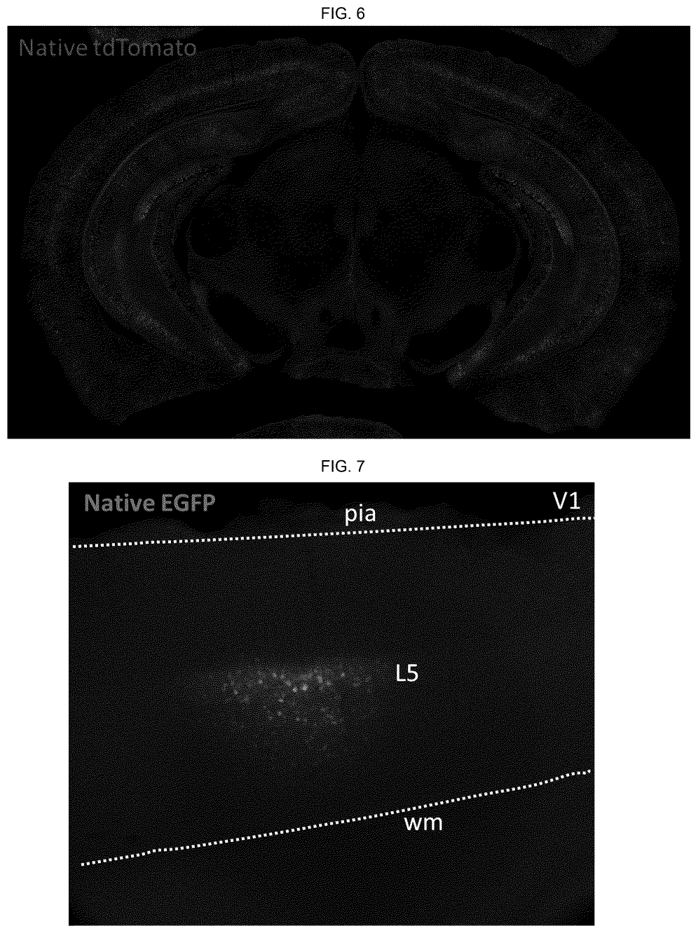

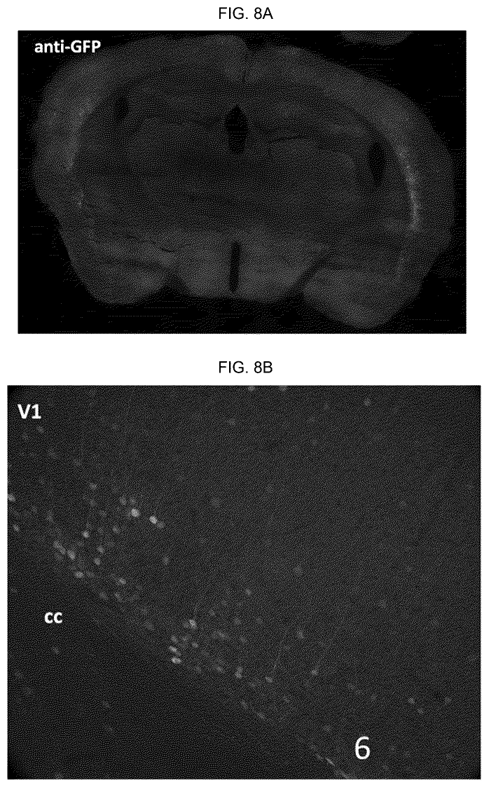

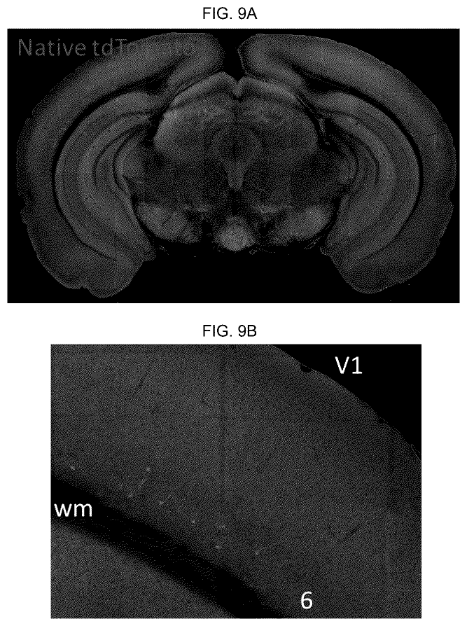

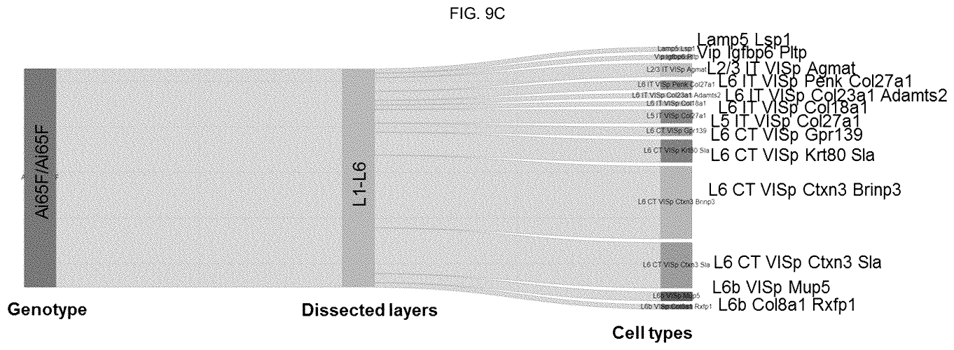

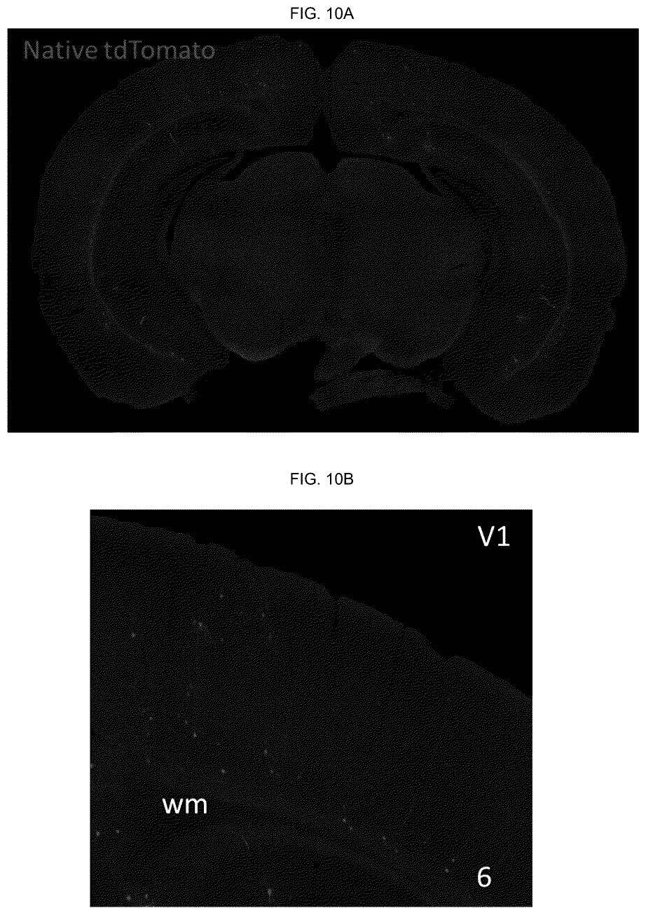

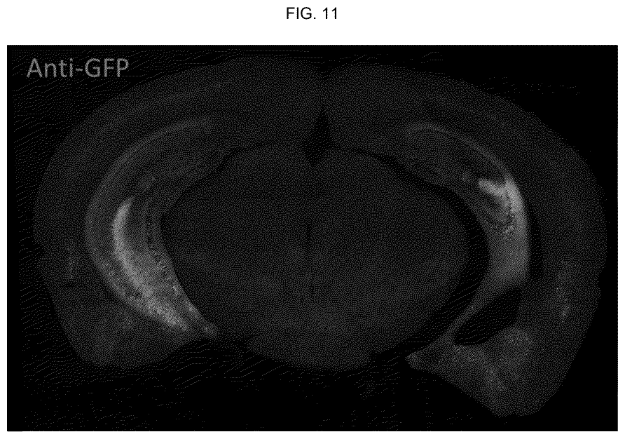

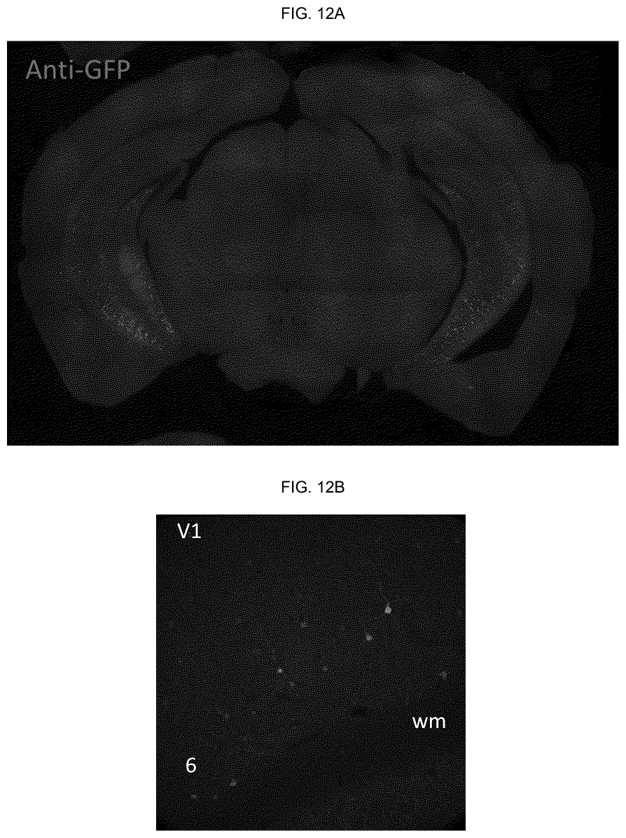

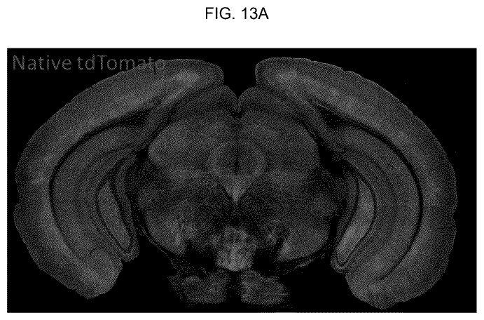

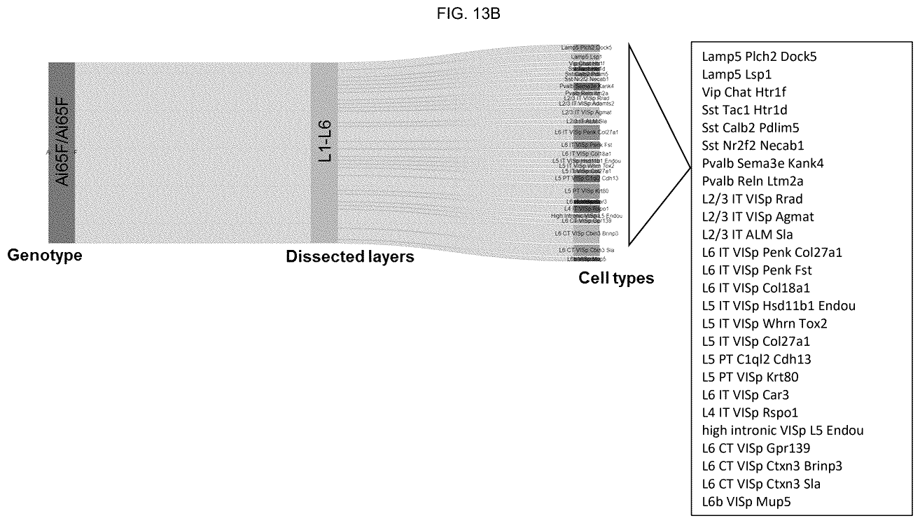

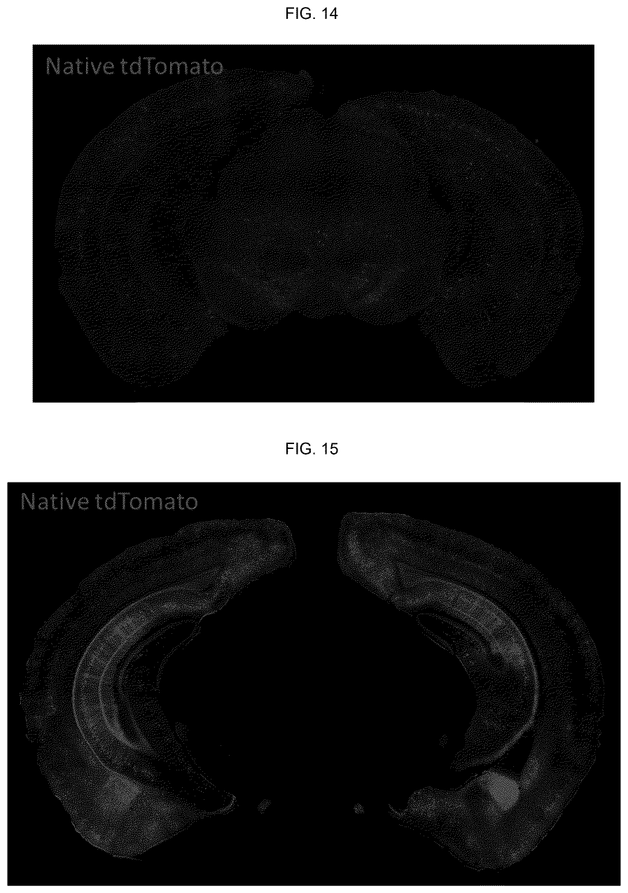

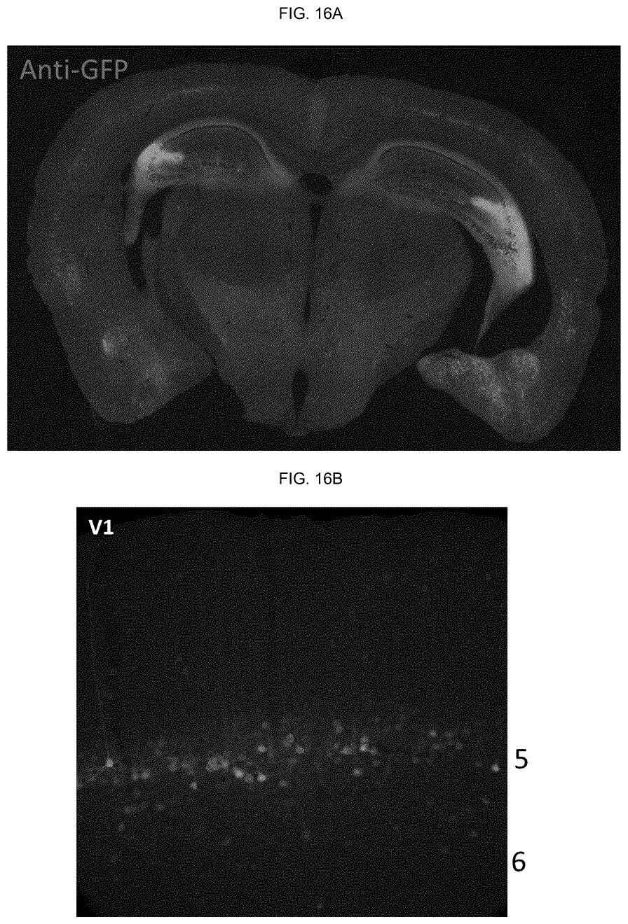

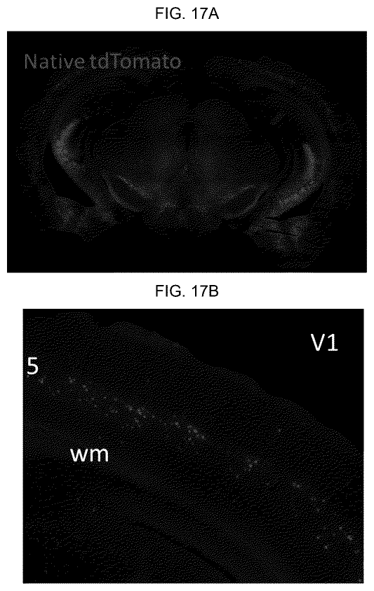

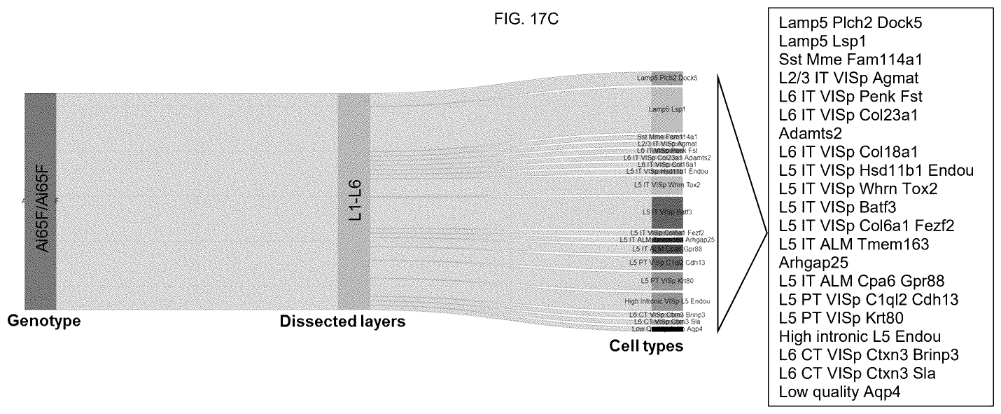

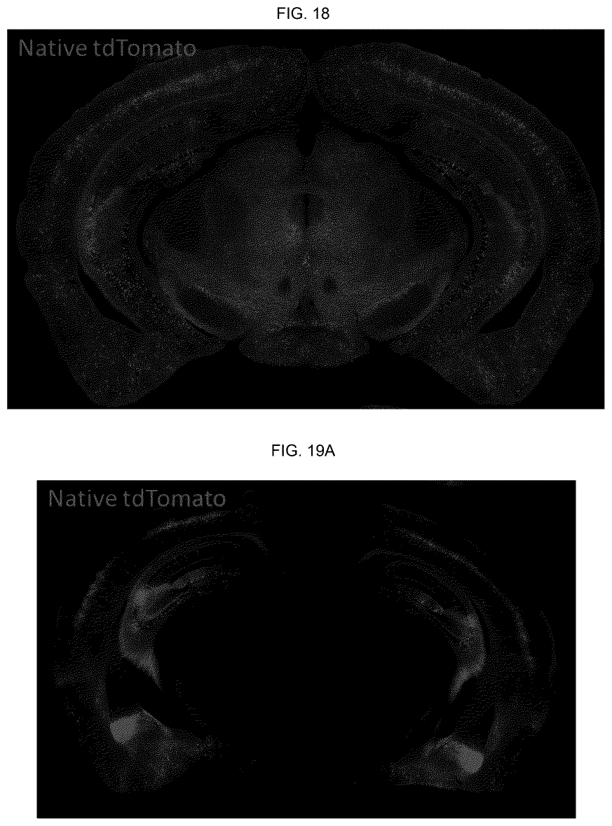

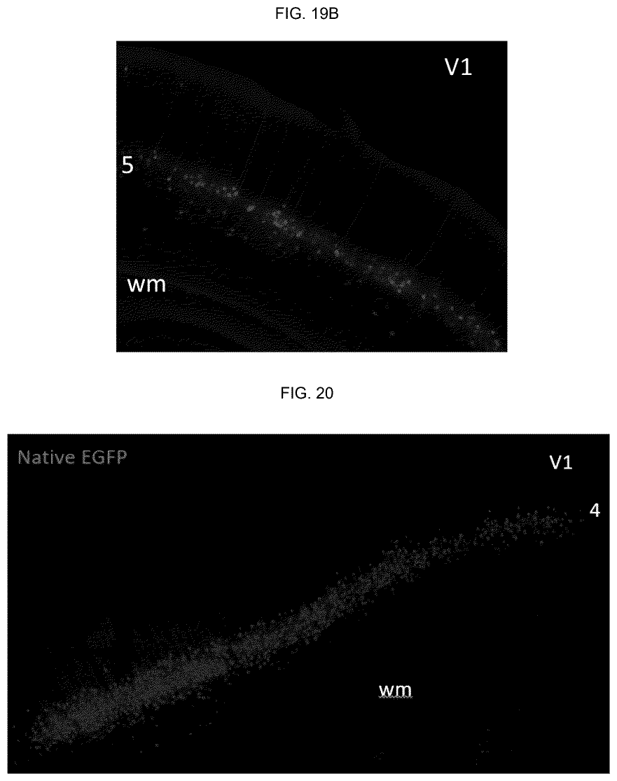

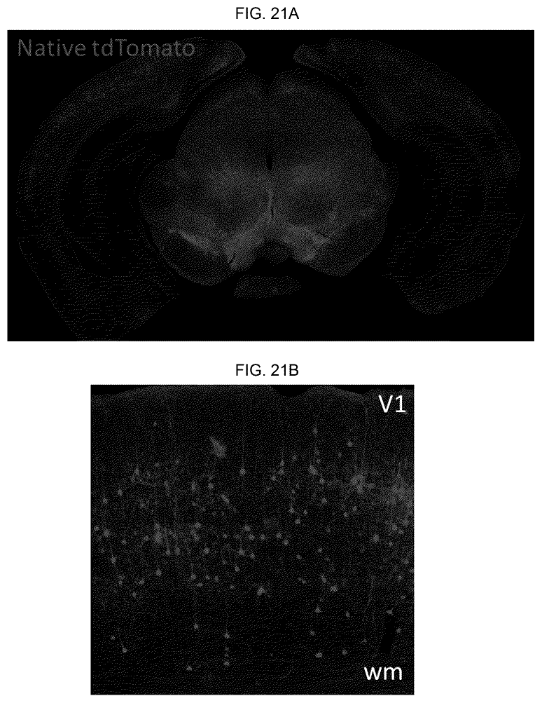

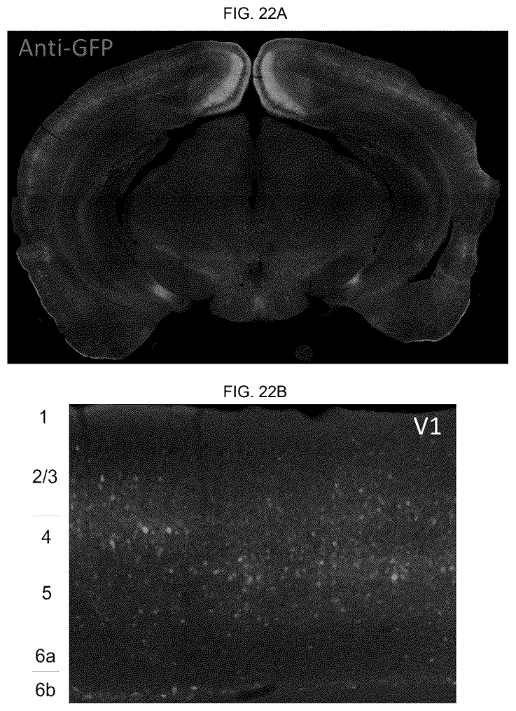

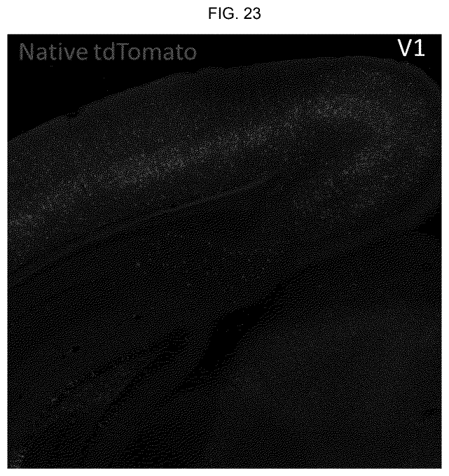

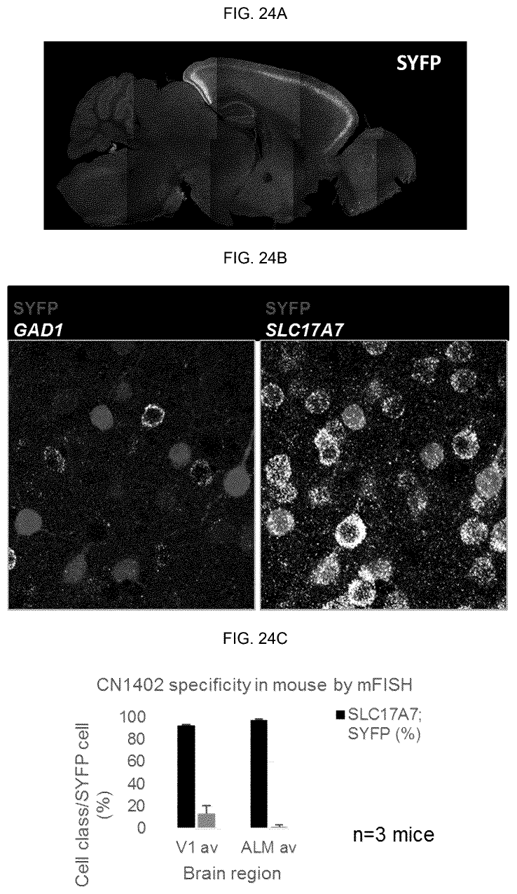

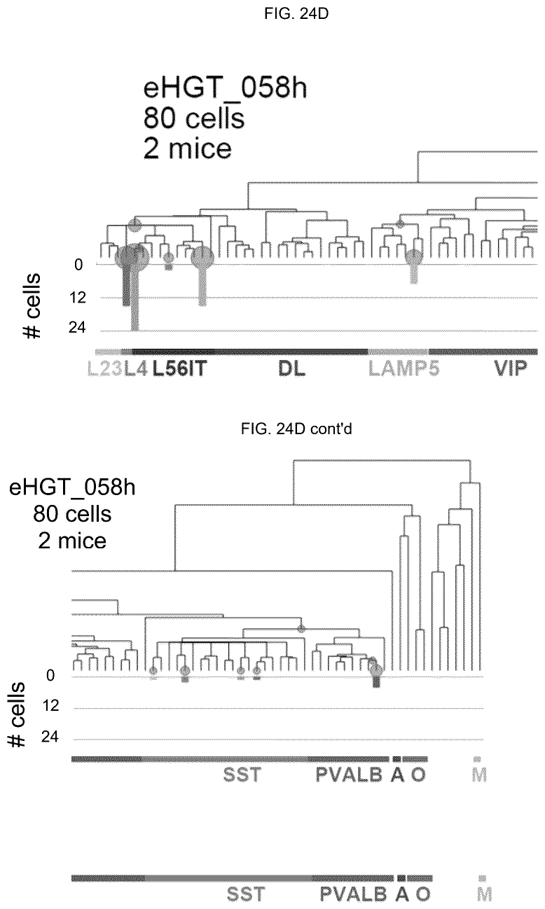

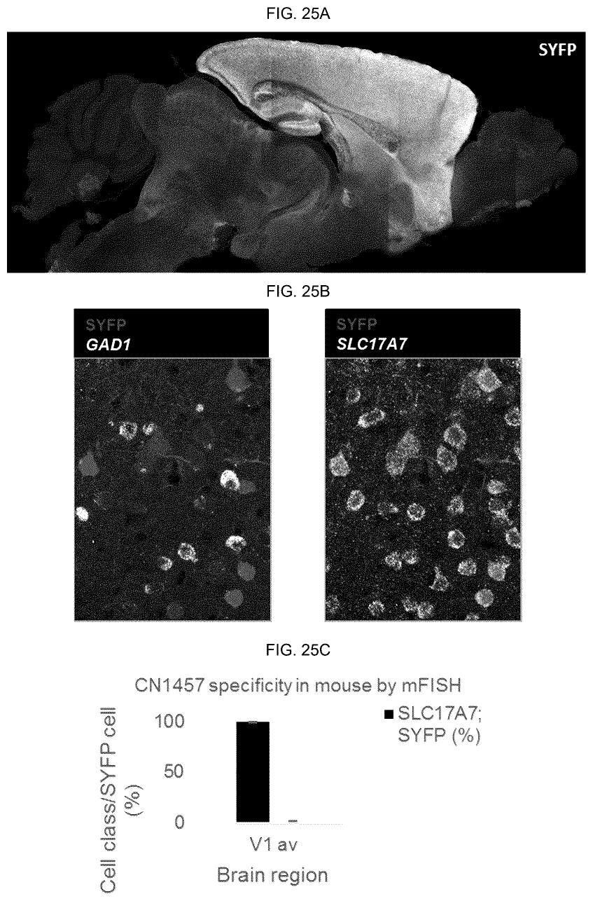

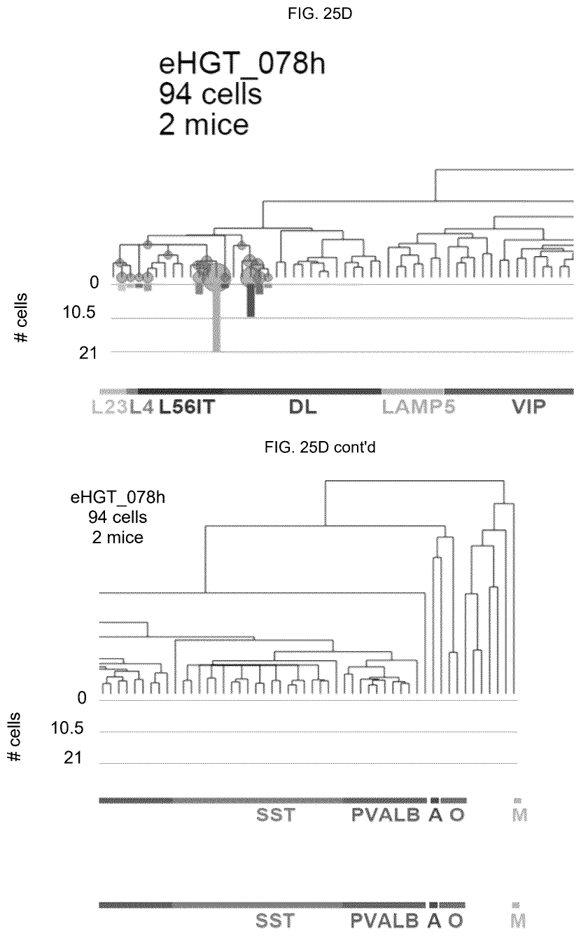

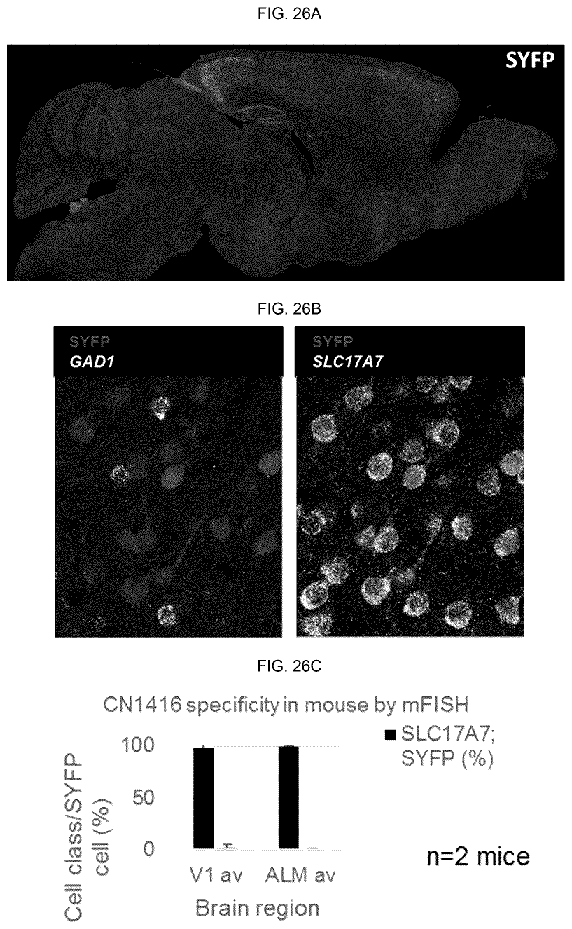

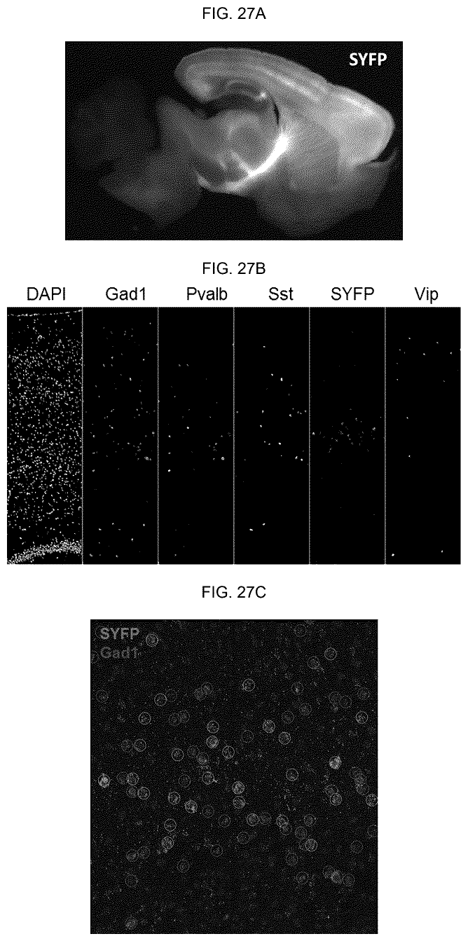

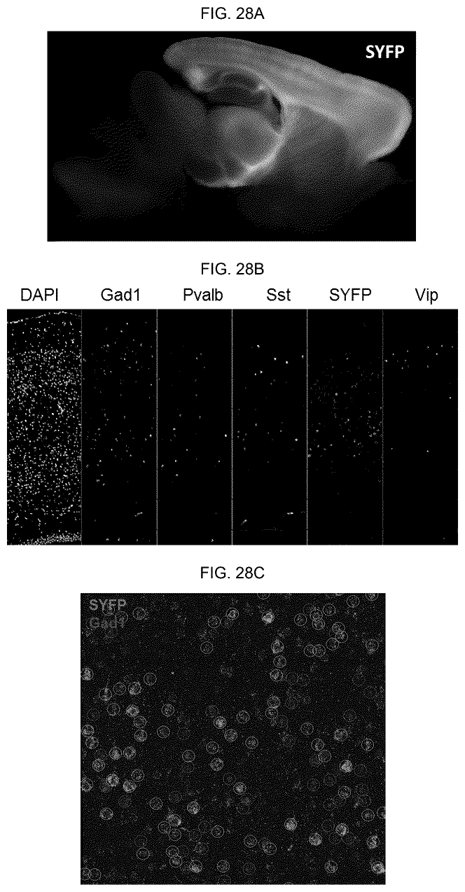

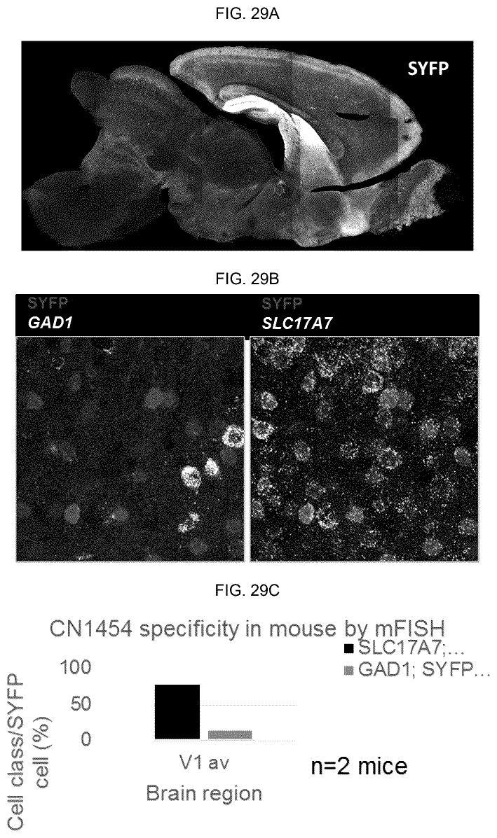

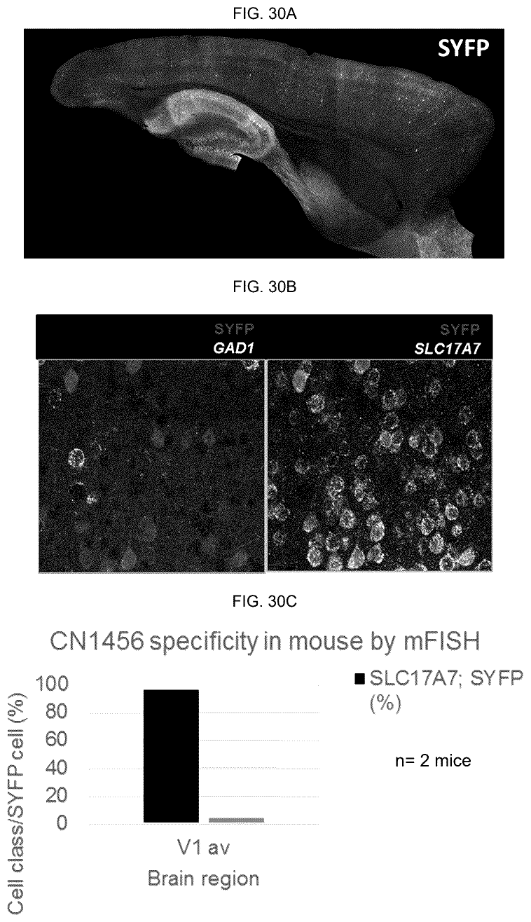

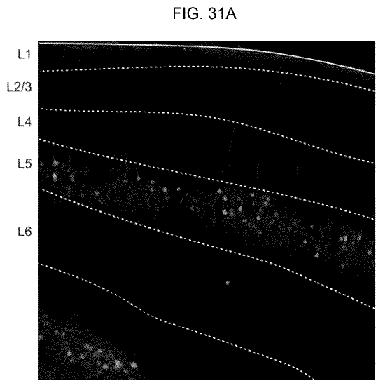

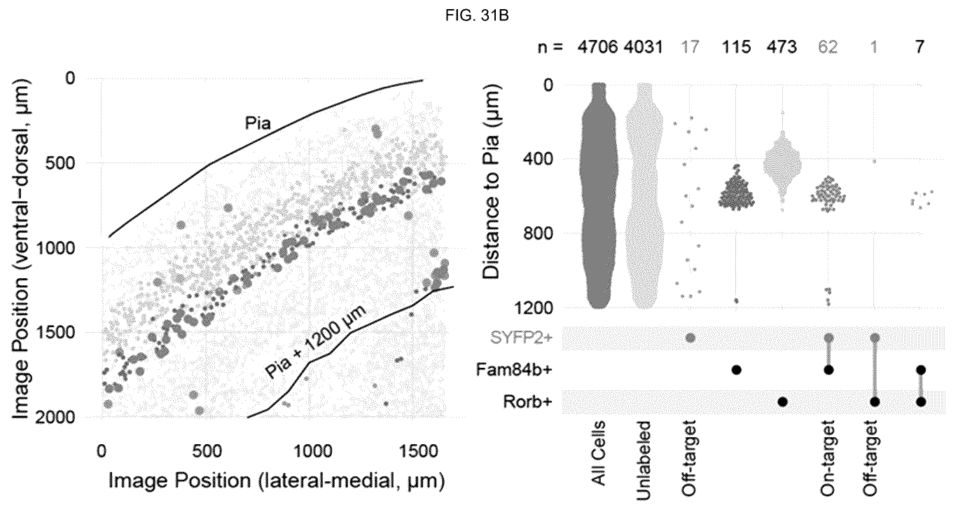

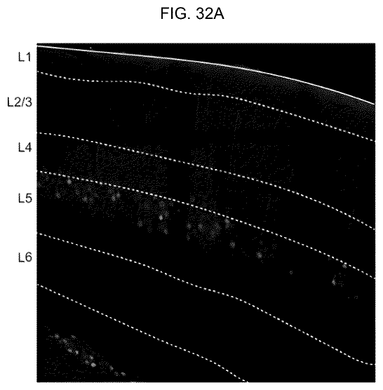

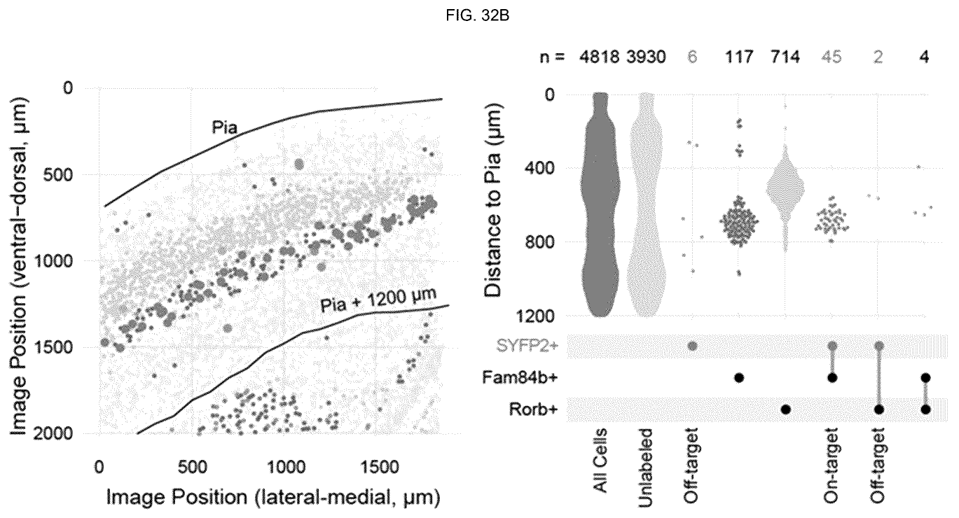

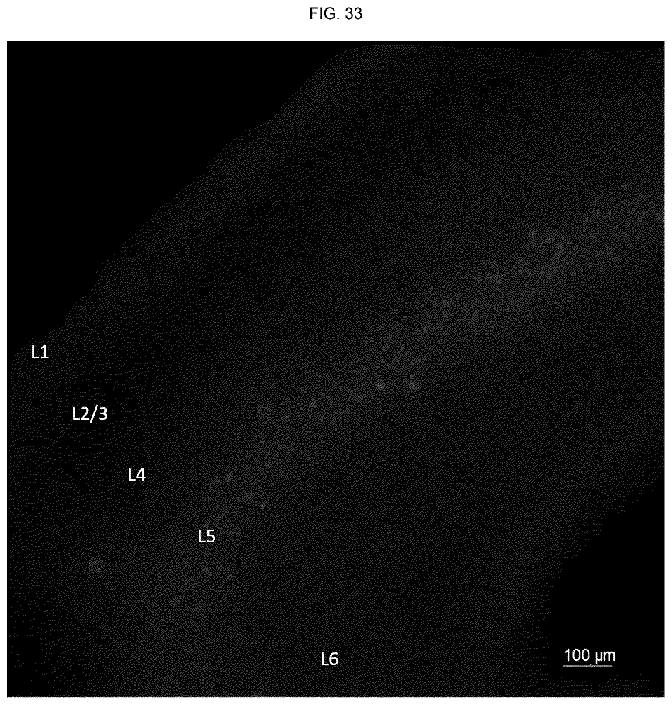

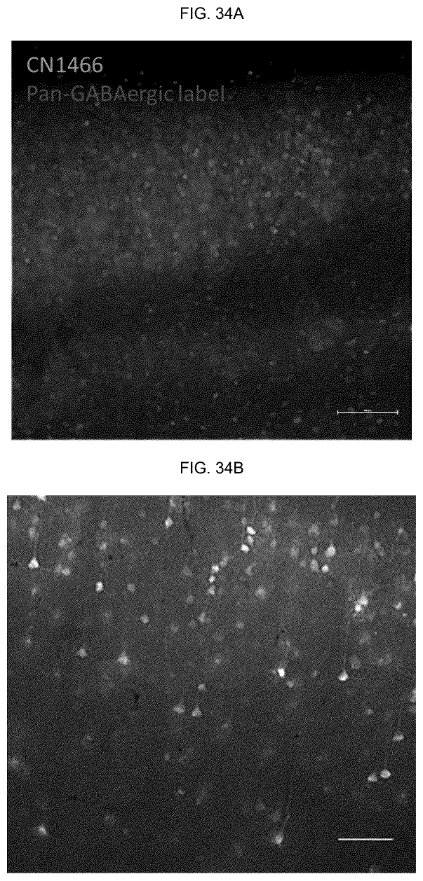

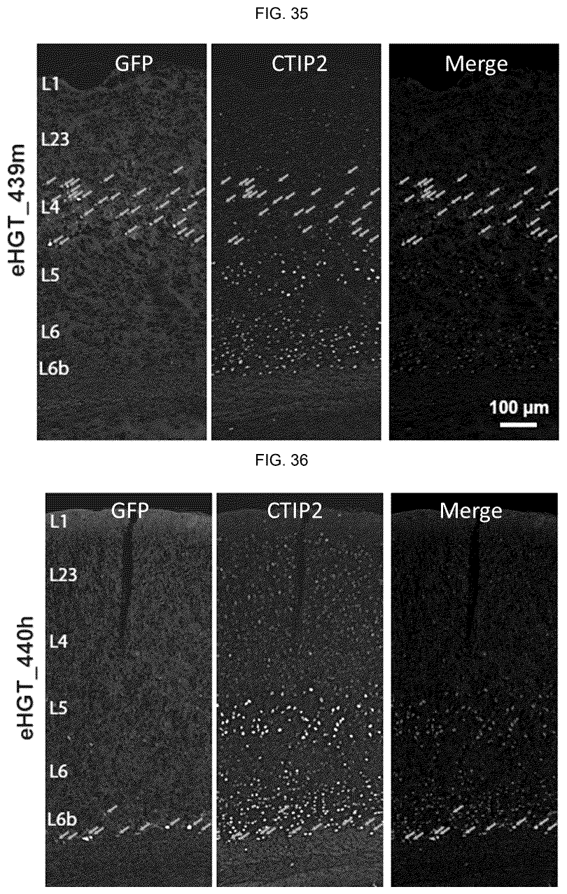

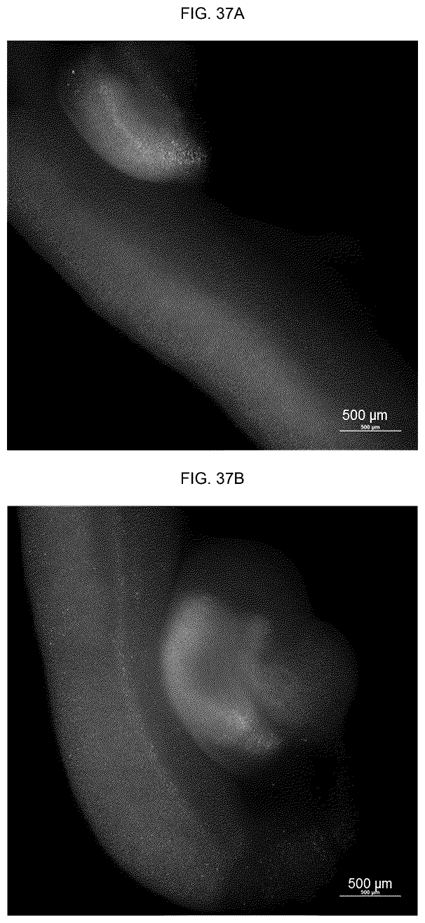

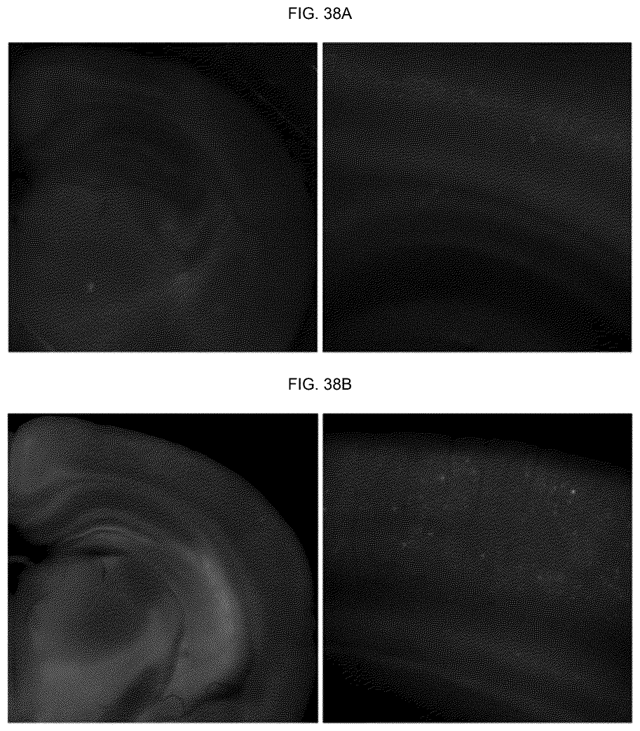

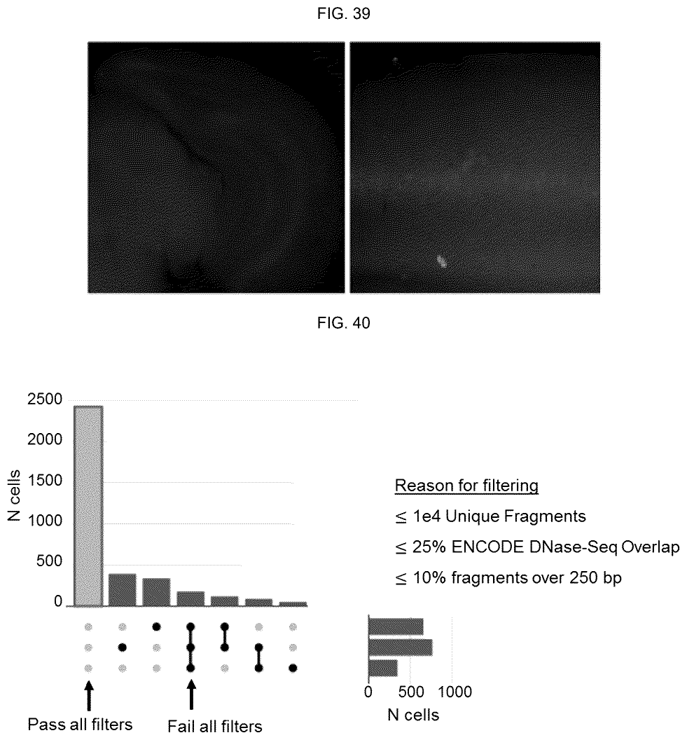

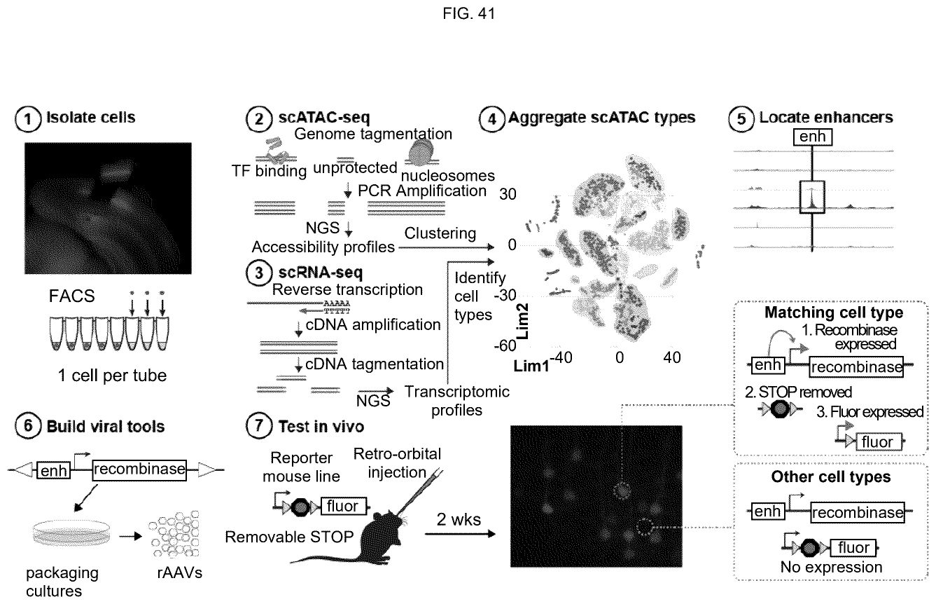

### DETAILED DESCRIPTION

To fully understand the biology of the brain, different cell types need to be distinguished and defined and, to further study them, vectors that can selectively label and perturb them need to be identified. Tasic, Curr. Opin. Neurobiol. 50, 242-249 (2018); Zeng & Sanes, Nat. Rev. Neurosci. 18, 530-546 (2017). In mouse, recombinase driver lines have been used to great effect to label cell populations that share marker gene expression. Daigle et al., Cell 174, 465-480.e22 (2018); Taniguchi, et al., Neuron 71, 995-1013 (2011); Gong et al., J. Neurosci. 27, 9817-9823 (2007). However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans.

Recent advances in single-cell profiling, such as single-cell RNA-seq (Tasic et al., Nature 563, 72-78 (2018); Tasic 2016, Nat Neurosci 19, 335-346) and surveys of neural electrophysiology and morphology (Gouwens 2019, Nat Neurosci 22, 1182-1195), have revealed that many recombinant driver lines label heterogeneous mixtures of cell types, and often include cells from multiple subclasses. For example, the Rbp4-Cre mouse driver line, which is commonly used to label layer 5 (L5) neurons, also labels cells with drastically different connectivity patterns: L5 intratelencephalic (IT, also called cortico-cortical) and pyramidal tract (PT, also called cortico-subcortical) neurons.

The current disclosure provides artificial expression constructs that selectively drive gene expression in targeted central nervous system cell populations. Targeted central nervous system cell populations include: L2/3 IT excitatory cortical neurons; L4 IT excitatory cortical neurons; L5 PT excitatory cortical neurons; L5 PT and L5 ET excitatory cortical neurons; L5 PT and L5 IT excitatory cortical neurons; L6 IT excitatory cortical neurons; L6 CT excitatory cortical neurons; L2/3 and 5 excitatory cortical neurons; L2/3 IT, L4 IT, L5 IT, L5 NP, L5 PT, and CR excitatory cortical neurons; pan excitatory and/or broad expression in excitatory cortical neurons; L5 PT excitatory cortical neurons in combination with subcortical populations in the CEAc, the substantia nigra, compact part (or pars compacta, SNc), and (ProS); and L5 PT excitatory cortical neurons in combination with cells within the subiculum, CA1 pyramidal neurons, a subset of dentate gyrus granule cells, scattered striatal neurons, and sparse cerebellar Purkinje cells.

Artificial expression constructs including a promoter; the Grik1_enhScnn1a-2, eHGT_058h, eHGT_058m, eHGT_439m, and/or eHGT_254h enhancer; and a gene encoding an expression product can lead to selective gene expression in L4 IT excitatory cortical neurons.

Particular examples of artificial expression constructs including a promoter; the mscRE4 enhancer, a concatenated mscRE4, and/or a concatenated mscRE16 enhancer; and a gene encoding an expression product can lead to selective gene expression in L5 PT excitatory cortical neurons. Examples of these expression constructs include T502-057 (vAi3.0), 981 (vAi5.0), 1052 (vAi10.0), CN1818 (vAi128.0), CN2014 (vAi129.0) and vAi130.0.

Artificial expression constructs including a promoter, a concatenated core of the mscRE4 enhancer, and a gene encoding an expression product can lead to selective gene expression in L5 PT and L5 ET excitatory cortical neurons.

Artificial expression constructs including a promoter; the mscRE1, mscRE11, and/or mscRE16 enhancer; and a gene encoding an expression product can lead to selective gene expression in L5 PT and L5 IT excitatory cortical neurons.

Artificial expression constructs including a promoter, the mscRE13 enhancer, and a gene encoding an expression product can lead to selective gene expression in L6 IT excitatory cortical neurons.

Particular examples of artificial expression constructs including a promoter, the mscRE10 enhancer, and a gene encoding an expression product can lead to selective gene expression in L6 CT excitatory cortical neurons. An example includes 995 (vAi15.0).

Artificial expression constructs including a promoter, the eHGT_440h enhancer, and a gene encoding an expression product can lead to selective gene expression in subtypes of L6b excitatory cortical neurons.

Artificial expression constructs including a promoter, the eHGT_078h enhancer; and a gene encoding an expression product can lead to selective gene expression in L2/3 IT, L4 IT, L5 IT, L5 NP, and L5 PT excitatory cortical neurons.

Selective expression of a gene encoding an expression product can be achieved in L2/3 IT, L5 IT, and L6b neurons utilizing the 1036 (vAi16.0) artificial expression construct described herein. This construct includes the mscRE10 enhancer.

Selective expression of a gene encoding an expression product can be achieved in L2/3 IT, L5 PT, L6 CT, and L6b neurons utilizing the 988 (vAi7.1), 1010 (vAi6.1), and/or 1011 (vAi7.2) artificial expression constructs described herein. These constructs include the mscRE4 enhancer.

Pan excitatory and/or broad expression in excitatory cortical neurons can be selectively achieved utilizing artificial expression constructs including a promoter; the eHGT_073h, eHGT_073m, eHGT_077h, and/or eHGT_078m enhancer; and a gene encoding an expression product. In particular embodiments, pan excitatory expression refers to expression in at least four types of cortical excitatory cells with limited to no expression in inhibitory cells and glial cells.

Artificial expression constructs described herein can additionally label other discrete cell types. For example, in addition to L5 PT cells, artificial expression constructs including a promoter, the mscRE4 enhancer, and a gene encoding an expression product can lead to gene expression in subcortical populations in the CEAc, the substantia nigra, compact part (or pars compacta, SNc), and (ProS). Similarly, in addition to L5 PT cells, artificial expression constructs including a promoter, a concatenated core of the mscRE4 enhancer, and a gene encoding an expression product can lead to gene expression in the subiculum, CA1 pyramidal neurons, a subset of dentate gyrus granule cells, scattered striatal neurons, and sparse cerebellar Purkinje cells.

As indicated by the proceeding discussion, certain artificial expression constructs disclosed herein include engineered enhancers, for example, concatenated cores of the mscRE4, eHGT_078h, and eHGT_078m enhancers as well as concatemers of the mscRE4 and mscRE16 enhancers. In relation to mscRE4, a functional 155 base pair (bp) core of the mscRE4 enhancer (SEQ ID NO: 29) was concatenated (SEQ ID NO: 30) to minimize the size required to drive gene expression. Despite being a 3× concatemer, SEQ ID NO: 30 is shorter in length than the original mscRE4 enhancer (SEQ ID NO: 28, which includes 555 bp). When used to construct an artificial expression construct, such as an rAAV, such concatemers allow more room for cargo genes linked to the enhancer, which is highly desirable, for example, in gene therapy vectors. For instance, many therapeutic cargo genes are too big to fit in an AAV vector design, so space (length of sequence) is at a premium.

As will be described in more detail throughout the disclosure, particular artificial expression constructs disclosed herein include T502-050, T502-054, vAi34.0, vAi33.2, vAi45.0, vAi1.0, T502-057, T502-059, TG978, TG981, TG988, TG995, TG996, TG997, TG999, TG1002, TG1010, TG1011, TG1021, TG1036, TG1037, TG1038, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and CN2014.

Aspects of the disclosure are now described with the following additional options and detail: (i) Artificial Expression Constructs & Vectors for Selective Expression of Genes in Selected Cell Types; (ii) Compositions for Administration (iii) Cell Lines Including Artificial Expression Constructs; (iv) Transgenic Animals; (v) Methods of Use; (vi) Kits and Commercial Packages; (vii) Exemplary Embodiments; (viii) Experimental Examples; and (ix) Closing Paragraphs.

(i) Artificial Expression Constructs & Vectors for Selective Expression of Genes in Selected Cell Types. Artificial expression constructs disclosed herein include (i) an enhancer sequence that leads to selective expression of a coding sequence within a targeted central nervous system cell type, (ii) a coding sequence that is expressed, and (iii) a promoter. The expression construct can also include other regulatory elements if necessary or beneficial.

In particular embodiments, an “enhancer” or an “enhancer element” is a cis-acting sequence that increases the level of transcription associated with a promoter and can function in either orientation relative to the promoter and the coding sequence that is to be transcribed and can be located upstream or downstream relative to the promoter or the coding sequence to be transcribed. There are art-recognized methods and techniques for measuring function(s) of enhancer element sequences. Particular examples of enhancer sequences utilized within artificial expression constructs disclosed herein include mscRE1, mscRE3, mscRE4, a concatemer of the mscRE4 core, mscRE10, mscRE11, mscRE12, mscRE13, mscRE16, a concatemer of mscRE16, Grik1_enhScnn1a-2, eHGT_058h, eHGT_058m, eHGT_073h, eHGT_073m, eHGT_075h, eHGT_077h, eHGT_078h, a concatemer of eHGT_078h core, eHGT_078m, a concatemer of eHGT_078m core, eHGT_439m, eHGT_440h, and eHGT_254h.

In particular embodiments, a targeted central nervous system cell type enhancer is an enhancer that is uniquely or predominantly utilized by the targeted central nervous system cell type. A targeted central nervous system cell type enhancer enhances expression of a gene in the targeted central nervous system cell type but does not substantially direct expression of genes in other non-targeted cell types, thus having neural specific transcriptional activity.

When a coding sequence is selectively expressed in selected neural cells and is not substantially expressed in other neural cell types, the product of the coding sequence is preferentially expressed in the selected cell type. In particular embodiments, preferential expression is greater than 50% expression as compared to a reference cell type; greater than 60% expression as compared to a reference cell type; greater than 70% expression as compared to a reference cell type; greater than 80% expression as compared to a reference cell type; or greater than 90% expression as compared to a reference cell type. In particular embodiments, a reference cell type refers to non-targeted neural cells. The non-targeted neural cells can be within the same anatomical structure as the targeted cells and/or can project to a common anatomical area. In particular embodiments, a reference cell type is within an anatomical structure that is adjacent to an anatomical structure that includes the targeted cell type. In particular embodiments, a reference cell type is a non-targeted neural cell with a different gene expression profile than the targeted cells.

In particular embodiments, the product of the coding sequence may be expressed at low levels in non-selected cell types, for example at less than 1% or 1%, 2%, 3%, 5%, 10%, 15% or 20% of the levels at which the product is expressed in selected neural cells. In particular embodiments, the targeted central nervous system cell type is the only cell type that expresses the right combination of transcription factors that bind an enhancer disclosed herein to drive gene expression. Thus, in particular embodiments, expression occurs exclusively within the targeted cell type.

In particular embodiments, targeted cell types (e.g. neural, neuronal, and/or non-neuronal) can be identified based on transcriptional profiles, such as those described in Tasic et al., 2018 Nature, and Hodge et al., Nature 573, 61-68 (2019). Human cell types are further defined in an ontological framework defined at bioontology.org. For reference, the following description of neural cell types and distinguishing features is also provided:

The cortical glutamatergic neuron class. Glutamatergic neurons (also called excitatory neurons) generate the neurotransmitter glutamate, which is excitatory (promotes firing) when received by neurons with ionotropic receptors and is modulatory when received by neurons expressing metabotropic receptors. Most cortical glutamatergic neurons project outside of their resident area (defined as the location of the primary cell body, including the nucleus), and genetic markers have been correlated with these projection properties.

Cortical glutamatergic neuron subclasses. Subclasses of glutamatergic neurons are defined both by the layer in which the neuronal cell body (including the nucleus) resides, as well as the major projection pattern of these neurons. In mouse, glutamatergic neurons are found in layer (L) 1, L2/3, L4, L5, L6, and in the cortical subplate (also called L6b). In human, glutamatergic neurons are found in L2, L3, L4, L5, L6, and L6b. In mouse, L2/3 is often considered a single layer, while in the human cortex layers 2 and 3 are distinct. Intratelencephalic (IT, also called cortico-cortical) neurons project primarily from cortical cell bodies to other adjacent or distant cortical regions. Corticothalamic (CT) neurons project primarily from the cortex to the thalamus. Pyramidal tract (PT, also called corticofugal or extratelencephalic neurons) project primarily from cortex to a variety of subcortical targets, usually from Layer 5 of the cortex. Near-projecting (NP) neurons appear to have only local projections within their cortical region of residence.

In the mouse, the projection and layer categories intersect in specific patterns that define glutamatergic neuron subclasses: For IT neurons: L2/3 IT, L4 IT, L5 IT, L6 IT; for CT neurons, L6 CT; for PT neurons, L5 PT; and for NP neurons, L5/6 NP (found in both layers in some regions). Projections of the L6b subclass of cells are not yet clearly defined, although projections from L6b to local targets as well as cortico-cortical projections to the anterior cingulate and subcortical projections to the thalamus have been observed. In mouse, there is also a highly distinct type of neurons that stands on its own: CR-Lhx5 cells correspond to Cajal-Retzius (CR) cells based on their location in L1 and expression of known Cajal-Retzius markers, such as Trp73, Lhx5 and Reln.

In the human cortex, long range cortical and subcortical projections are difficult to ascertain directly. However, similar patterns of cell types are observed based on layer position and molecular correspondence to the projection classes seen in the mouse. Layer 4 cells tend to receive input from other cortical structures through the expression of specific genes such as RORB, by the lack of projection neurons, and through a granular cytoarchitecture usually visualized by nuclear markers such as DAPI.

Summary of Cortical Glutamatergic Subclasses:
All: Express glutamate transmitters Slc17a6 and/or Slc17a7. They all express Snap25 and lack expression of Gad1/Gad2 and lack expression of Slc1A3.
L2/3 IT: Primarily reside in Layer 2/3 and have mainly intratelencephalic (cortico-cortical) projections.
L4 IT: Primarily reside in Layer 4 and mainly have either local or intratelencephalic (cortico-cortical) projections.
L5 IT: Primarily reside in Layer 5 and have mainly intratelencephalic (cortico-cortical) projections. Also called L5a.
L5 PT: Primarily reside in Layer 5 and have mainly cortico-subcortical (pyramidal tract or corticofugal) projections. Also called L5b or L5 CF (corticofugal) or L5 ET (extratelencephalic). This subclass includes cells that are located in the primary motor cortex and neighboring areas and are corticospinal projection neurons, which are associated with motor neuron/movement disorders, such as ALS. This subclass includes thick-tufted pyramidal neurons, including distinctive subtypes found only in specialized regions, e.g. Betz cells, Meynert cells, and von Economo cells.
L5 NP: Primarily reside in Layer 5 and have mainly nearby projections.
L6 CT: Primarily reside in Layer 6 and have mainly cortico-thalamic projections.
L6 IT: Primarily reside in Layer 6 and have mainly intratelencephalic (cortico-cortical) projections. Included in this subclass are L6 IT Car3 cells, which are highly similar to intracortical-projecting cells in the claustrum.
L6b: Primarily reside in the cortical subplate (L6b), with local (near the cell body) projections and some cortico-cortical projections from VISp to anterior cingulate, and cortico-subcortical projections to the thalamus.
CR: A distinct subclass defined by a single type in L1, Cajal-Retzius cells express distinct molecular markers Lhx5 and Trp73.

Within each subclass, differentially expressed genes define multiple distinct and experimentally targetable cell types. For example, within L2/3 IT cells in the primary visual cortex, 3 distinct cell types have been observed: L2/3 IT VISp Rrad, L2/3 IT VISp Adamts2, and L2/3 IT VISp Agmat, which are identified by the expression of the Rrad, Adamts2, and Agmat genes, respectively. These gene labels are mainly used to distinguish each cell type from related cell types within the cell subclass (in this case, L2/3 IT), and may not represent a single gene that distinguishes the cell type from all other cells in the cortex. Marker genes may need to be applied in a combinatorial fashion to uniquely identify a given cell type.

The cortical GABAergic neuron class. GABAergic neurons (also called inhibitory neurons) generate the neurotransmitter gamma aminobutyric acid (GABA), which inhibits firing of downstream neurons. All cortical GABAergic neurons except one (called Meis2-Adamts19) share many gene expression markers including Thy1 and Scn2b. Meis2-Adamts19 type corresponds to the Meis2-expressing GABAergic neuronal type largely confined to white matter that originates from the embryonic pallial-subpallial boundary. Among GABAergic types, this is the only type that reliably expresses the transcription factor Meis2 mRNA, transcribes the smallest number of genes, and does not express Thy1 and Scn2b.

Summary of Cortical GABAergic Subclasses:
All: Express GABA synthesis genes Gad1/GAD1 and Gad2/GAD2.
Lamp5, Sncg, Serpinf1, and Vip: Developmentally derived from neuronal progenitors from the caudal ganglionic eminence (CGE) or preoptic area (POA).
Sst and Pvalb: Developmentally derived from neuronal progenitors in the medial ganglionic eminence (MGE).
Lamp5: Found in many cortical layers, especially upper (L1-L2/3), and have mainly neurogliaform and single bouquet morphology.
Sncg: Found in many cortical layers, and have molecular overlaps with Lamp5 and Vip cells, but inconsistent expression of Lamp5 or Vip, with more consistent expression of Sncg.
Serpinf1: Found in many cortical layers, and have molecular overlaps with Sncg and Vip cells, but inconsistent expression of Sncg or Vip, with more consistent expression of Serpinf1.
Vip: Found in many cortical layers, but especially frequent in upper layers (L1-L4), and highly express the neurotransmitter vasoactive intestinal peptide (Vip).
Sst: Found in many cortical layers, but especially frequent in lower layers (L5-L6). They highly express the neurotransmitter somatostatin (Sst), and frequently block dendritic inputs to postsynaptic neurons. Included in this subclass are sleep-active Sst Chodl neurons (which also express Nos1 and Tacr1) that are highly distinct from other Sst neurons but express some shared marker genes including Sst. In human, SST gene expression is often detected in layer 1 LAMP5+ cells.
Pvalb: Found in many cortical layers, but especially frequent in lower layers (L5-L6). They highly express the calcium-binding protein parvalbumin (Pvalb), express neuropeptide Tac1, and frequently dampen the output of postsynaptic neurons. Most fast-spiking inhibitory cells express Pvalb strongly. Included in this subclass are chandelier cells, which have distinct, chandelier-like morphology and express the markers Cpne5 and Vipr2 in mouse, and NOG and UNC5B in human.
Meis2: A distinct subclass defined by a single type, only cortical GABAergic type that expresses Meis2 gene, and does not express some other genes that are expressed by all other cortical GABAergic types (for example, Thy1 and Scn2b). This type is found in L6b and subcortical white matter.

Cells located in the central nucleus of the amygdala (CEA, which includes CEAc) are involved in pain, anxiety, and fear processing. Cells in the substantia nigra compact part (SNc, also called pars compacta) are located in the midbrain, are involved in motor control, and are adversely affected in Parkinson's disease. Cells in the prosubiculum (ProS) are located between the hippocampus CA1 region and the subiculum.

The subiculum is the most inferior component of the hippocampal formation. It lies between the entorhinal cortex and the CA1 subfield of the hippocampus proper. CA1 pyramidal neurons send their axons to the subiculum and deep layers of the entorhinal cortex. Granule cells within the dentate gyrus receive excitatory neuron input from the entorhinal cortex and send excitatory output to the hippocampal CA3 region via mossy fibers. Cell bodies of striatal neurons are located within the subcortical basal ganglia of the forebrain. Purkinje cells send inhibitory projections to the deep cerebellar nuclei, and constitute the dominant, if not sole output of all motor coordination in the cerebellar cortex.

Non-neuronal Subclasses:
Astrocytes: Neuroectoderm-derived glial cells which express the marker Aqp4 and often GFAP, but do not express neuronal marker SNAP25. They can have a distinct star-shaped morphology and are involved in metabolic support of other cells in the brain. Multiple astrocyte morphologies are observed in mouse and human
Oligodendrocytes: Neuroectoderm-derived glial cells, which express the marker Sox10. This category includes oligodendrocyte precursor cells (OPCs). Oligodendrocytes are the subclass that is primarily responsible for myelination of neurons.
VLMCs: Vascular leptomeningeal cells (VLMCs) are part of the meninges that surround the outer layer of the cortex and express the marker genes Lum and Col1a1.
Pericytes: Blood vessel-associated cells, also called mural cells, that express the marker genes Konj8 and Abcc9. Pericytes wrap around endothelial cells and are important for regulation of capillary blood flow and are involved in blood-brain barrier permeability.
SMCs: Specialized smooth-muscle cells, also called mural cells, which are blood vessel-associated cells that express the marker gene Acta2. SMCs cover arterioles in the brain and are involved in blood-brain barrier permeability.
Endothelial: Cells that line blood vessels of the brain. Endothelial cells express the markers Tek and PDGF-B.
Microglia: hematopoietic-derived immune cells, which are brain-resident macrophages, and perivascular macrophages (PVMs) that may be transitionally associated with brain tissue, or included as a biproduct of brain dissection methods. Microglia are known to express Cx3cr1, Tmem119, and PTPRC (CD45).

In particular embodiments, a coding sequence is a heterologous coding sequence that encodes an effector element. An effector element is a sequence that is expressed to achieve, and that in fact achieves, an intended effect. Examples of effector elements include reporter genes/proteins and functional genes/proteins.

Exemplary reporter genes/proteins include those expressed by Addgene ID #s 83894 (pAAV-hDIx-Flex-dTomato-Fishell_7), 83895 (pAAV-hDIx-Flex-GFP-Fishell_6), 83896 (pAAV-hDIx-GiDREADD-dTomato-Fishell-5), 83898 (pAAV-mDIx-ChR2-mCherry-Fishell-3), 83899 (pAAV-mDIx-GCaMP6f-Fishell-2), 83900 (pAAV-mDIx-GFP-Fishell-1), and 89897 (pcDNA3-FLAG-mTET2 (N500)). Exemplary reporter genes particularly can include those which encode an expressible fluorescent protein, or expressible biotin; blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato, dTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

GFP is composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostability and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ¿), also known as its optical cross section of 9.13×10-21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm. Exemplary functional molecules include functioning ion transporters, cellular trafficking proteins, enzymes, transcription factors, neurotransmitters, calcium reporters, channel rhodopsins, guide RNA, nucleases, or designer receptors exclusively activated by designer drugs (DREADDs).

Ion transporters are transmembrane proteins that mediate transport of ions across cell membranes. These transporters are pervasive throughout most cell types and important for regulating cellular excitability and homeostasis. Ion transporters participate in numerous cellular processes such as action potentials, synaptic transmission, hormone secretion, and muscle contraction. Many important biological processes in living cells involve the translocation of cations, such as calcium (Ca2+), potassium (K+), and sodium (Na+) ions, through such ion channels. In particular embodiments, ion transporters include voltage gated sodium channels (e.g., SCN1A), potassium channels (e.g., KCNQ2), and calcium channels (e.g. CACNA1C)).

Exemplary enzymes, transcription factors, receptors, membrane proteins, cellular trafficking proteins, signaling molecules, and neurotransmitters include enzymes such as lactase, lipase, helicase, alpha-glucosidase, amylase; transcription factors such as SP1, AP-1, Heat shock factor protein 1, C/EBP (CCAA-T/enhancer binding protein), and Oct-1; receptors such as transforming growth factor receptor beta 1, platelet-derived growth factor receptor, epidermal growth factor receptor, vascular endothelial growth factor receptor, and interleukin 8 receptor alpha; membrane proteins, cellular trafficking proteins such as clathrin, dynamin, caveolin, Rab-4A, and Rab-11A; signaling molecules such as nerve growth factor (NGF), platelet-derived growth factor (PDGF), transforming growth factor β (TGFβ), epidermal growth factor (EGF), GTPase and HRas; and neurotransmitters such as cocaine and amphetamine regulated transcript, substance P, oxytocin, and somatostatin.

In particular embodiments, functional molecules include reporters of neural function and states such as calcium reporters. Intracellular calcium concentration is an important predictor of numerous cellular activities, which include neuronal activation, muscle cell contraction and second messenger signaling. A sensitive and convenient technique to monitor the intracellular calcium levels is through the genetically encoded calcium indicator (GECI). Among the GECIs, green fluorescent protein (GFP) based calcium sensors named GCaMPs are efficient and widely used tools. The GCaMPs are formed by fusion of M13 and calmodulin protein to N- and C-termini of circularly permutated GFP. Some GCaMPs yield distinct fluorescence emission spectra (Zhao et al., Science, 2011, 333 (6051): 1888-1891). Exemplary GECIs with green fluorescence include GCaMP3, GCaMP5G, GCaMP6s, GCaMP6m, GCaMP6f, jGCaMP7s, jGCaMP7c, jGCaMP7b, and jGCaMP7f. Furthermore, GECIs with red fluorescence include jRGECO1a and jRGECO1b. AAV products containing GECIs are commercially available. For example, Vigene Biosciences provides AAV products including AAV8-CAG-GCaMP3 (Cat. No: BS4-CX3AAV8), AAV8-Syn-FLEX-GCaMP6s-WPRE (Cat. No: BS1-NXSAAV8), AAV8-Syn-FLEX-GCaMP6s-WPRE (Cat. No: BS1-NXSAAV8), AAV9-CAG-FLEX-GCaMP6m-WPRE (Cat. No: BS2-CXMAAV9), AAV9-Syn-FLEX-jGCaMP7s-WPRE (Cat. No: BS12-NXSAAV9), AAV9-CAG-FLEX-jGCaMP7f-WPRE (Cat. No: BS12-CXFAAV9), AAV9-Syn-FLEX-jGCaMP7b-WPRE (Cat. No: BS12-NXBAAV9), AAV9-Syn-FLEX-jGCaMP7c-WPRE (Cat. No: BS12-NXCAAV9), AAV9-Syn-FLEX-NES-jRGECO1a-WPRE (Cat. No: BS8-NXAAAV9), and AAV8-Syn-FLEX-NES-jRCaMP1b-WPRE (Cat. No: BS7-NXBAAV8).

In particular embodiments calcium reporters include the genetically encoded calcium indicators GECI, NTnC; Myosin light chain kinase, GFP, Calmodulin chimera; Calcium indicator TN-XXL; BRET-based auto-luminescent calcium indicator; and/or Calcium indicator protein OeNL (Ca2+)-18u).

In particular embodiments, functional molecules include modulators of neuronal activity like channel rhodopsins (e.g., channelopsin-1, channelrhodopsin-2, and variants thereof). Channelrhodopsins are a subfamily of retinylidene proteins (rhodopsins) that function as light-gated ion channels. In addition to channelrhodopsin 1 (ChR1) and channelrhodopsin 2 (ChR2), several variants of channelrhodopsins have been developed. For example, Lin et al. (Biophys J, 2009, 96 (5): 1803-14) describe making chimeras of the transmembrane domains of ChR1 and ChR2, combined with site-directed mutagenesis. Zhang et al. (Nat Neurosci, 2008, 11 (6): 631-3) describe VChR1, which is a red-shifted channelrhodopsin variant. VChR1 has lower light sensitivity and poor membrane trafficking and expression. Other known channelrhodopsin variants include the ChR2 variant described in Nagel, et al., Proc Natl Acad Sci USA, 2003, 100 (24): 13940-5), ChR2/H134R (Nagel, G., et al., Curr Biol, 2005, 15 (24): 2279-84), and ChD/ChEF/ChIEF (Lin, J. Y., et al., Biophys J, 2009, 96 (5): 1803-14), which are activated by blue light (470 nm) but show no sensitivity to orange/red light. Additional variants are described in Lin, Experimental Physiology, 2010, 96.1:19-25 and Knopfel et al., The Journal of Neuroscience, 2010, 30 (45): 14998-15004).

In particular embodiments, functional molecules include DNA and RNA editing tools such CRISPR/CAS (e.g., guide RNA and a nuclease, such as Cas, Cas9 or cpf1). Functional molecules can also include engineered Cpf1s such as those described in US 2018/0030425, US 2016/0208243, WO/2017/184768 and Zetsche et al. (2015) Cell 163:759-771; single gRNA (see e.g., Jinek et al. (2012) Science 337:816-821; Jinek et al. (2013) eLife 2: e00471; Segal (2013) eLife 2: e00563) or editase, guide RNA molecules or homologous recombination donor cassettes.

Additional effector elements include Cre, iCre, dgCre, FlpO, and tTA2. iCre refers to a codon-improved Cre. dgCre refers to an enhanced GFP/Cre recombinase fusion gene with an N terminal fusion of the first 159 amino acids of the Escherichia coli K-12 strain chromosomal dihydrofolate reductase gene (DHFR or folA) harboring a G67S mutation and modified to also include the R12Y/Y100I destabilizing domain mutation. FlpO refers to a codon-optimized form of FLPe that greatly increases protein expression and FRT recombination efficiency in mouse cells. Like the Cre/LoxP system, the FLP/FRT system has been widely used for gene expression (and generating conditional knockout mice, mediated by the FLP/FRT system). tTA2 refers to tetracycline transactivator.

Exemplary expressible elements are expression products that do not include effector elements, for example, a non-functioning or defective protein. In particular embodiments, expressible elements can provide methods to study the effects of their functioning counterparts. In particular embodiments, expressible elements are non-functioning or defective based on an engineered mutation that renders them non-functioning. In these aspects, non-expressible elements are as similar in structure as possible to their functioning counterparts.

Exemplary self-cleaving peptides include the 2A peptides which lead to the production of two proteins from one mRNA. The 2A sequences are short (e.g., 20 amino acids), allowing more use in size-limited constructs. Particular examples include P2A, T2A, E2A, and F2A. In particular embodiments, the expression constructs include an internal ribosome entry site (IRES) sequence. IRES allow ribosomes to initiate translation at a second internal site on a mRNA molecule, leading to production of two proteins from one mRNA.

Coding sequences encoding molecules (e.g., RNA, proteins) described herein can be obtained from publicly available databases and publications. Coding sequences can further include various sequence polymorphisms, mutations, and/or sequence variants wherein such alterations do not affect the function of the encoded molecule. The term “encode” or “encoding” refers to a property of sequences of nucleic acids, such as a vector, a plasmid, a gene, cDNA, mRNA, to serve as templates for synthesis of other molecules such as proteins.

The term “gene” may include not only coding sequences but also regulatory regions such as promoters, enhancers, and termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include minBglobin, CMV, minCMV, a mutated minCMV, SV40 immediately early promoter, the Hsp68 minimal promoter (proHSP68), and the Rous Sarcoma Virus (RSV) long-terminal repeat (LTR) promoter. Minimal promoters have no activity to drive gene expression on their own but can be activated to drive gene expression when linked to a proximal enhancer element.

In particular embodiments, expression constructs are provided within vectors. The term vector refers to a nucleic acid molecule capable of transferring or transporting another nucleic acid molecule, such as an expression construct. The transferred nucleic acid is generally linked to, e.g., inserted into, the vector nucleic acid molecule. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors.

Viral vector is widely used to refer to a nucleic acid molecule that includes virus-derived nucleic acid elements that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adenovirus. “Adenovirus vectors” refer to those constructs containing adenovirus sequences sufficient to (a) support packaging of an expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in selected cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retrovirus. Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In particular embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In particular embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In particular embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and U5 regions. The R region plays a role during reverse transcription in permitting the transfer of nascent DNA from one end of the genome to the other.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al., 1999, J. Virol., 73:2886); the posttranscriptional regulatory element present in hepatitis B virus (HPRE) (Smith et al., Nucleic Acids Res. 26 (21): 4818-4827, 1998); and the like (Liu et al., 1995, Genes Dev., 9:1766). In particular embodiments, vectors include a posttranscriptional regulatory element such as a WPRE or HPRE. In particular embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation sequence 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40 pA. In particular embodiments, a preferred embodiment of an expression construct includes a terminator element. These elements can serve to enhance transcript levels and to minimize read through from the construct into other plasmid sequences.

In particular embodiments, a viral vector further includes one or more insulator elements. Insulators elements may contribute to protecting viral vector-expressed sequences, e.g., effector elements or expressible elements, from integration site effects, which may be mediated by cis-acting elements present in genomic DNA and lead to deregulated expression of transferred sequences (i.e., position effect; see, e.g., Burgess-Beusse et al., PNAS., USA, 99:16433, 2002; and Zhan et al., Hum. Genet., 109:471, 2001). In particular embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33:198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

TABLE 1
Particular embodiments of vectors disclosed herein include:

| Expression Construct Name | Features |
|---|---|
| T502-050 | rAAV: Grik1_enhScnn1a-2-Hsp68-EGFP-WPRE3-BGHpA |
| T502-054 | rAAV: Grik1_enhScnn1a-2-pBGmin-EGFP-WPRE3-BGHpA |
| vAi34.0 | rAAV: Grik1_enhScnn1a-2-pBGmin-FlpO-WPRE3 |
| vAi33.2 | rAAV: Grik1_enhScnn1a-2-pBGmin-EGFP-WPRE3 |
| vAi45.0 | rAAV: mscRE12-pBGmin-FlpO-WPRE-BGHpA |
| vAi1.0 | rAAV: mscRE1-pBGmin-SYFP2-WPRE3-BGHpA |
| T502-057 | scAAV: mscRE4-pBGmin-SYFP2-WPRE3-bGHpA |
| T502-059 | rAAV: mscRE3-pBGmin-SYFP2-WPRE3-BGHpA |
| TG975 | rAAV: mscRE4-pBGmin-IRES2-FlpO-WPRE3 |
| TG978 | rAAV: mscRE4-pBGmin-FlpO-WPRE3 |
| TG979 | rAAV: mscRE4-pBGmin-FlpO-bGHpA |
| TG981 | rAAV: mscRE4-pBGmin-EGFP-WPRE3-bGHpA |
| TG982 | rAAV: mscRE4-pBGmin-IRES2-iCre-bGHpA |
| TG987 | rAAV: mscRE4-pBGmin-IRES2-tTA2-bGHpA |
| TG988 | rAAV: mscRE4-pBGmin-tTA2-bGHpA |
| TG995 | rAAV: mscRE10-pBGmin-EGFP-WPRE3-BGHpA |
| TG996 | rAAV: mscRE11-pBGmin-EGFP-WPRE3-BGHp |
| TG997 | rAAV: mscRE12-pBGmin-EGFP-WPRE3-BGHpA |
| TG999 | rAAV: mscRE13-pBGmin-EGFP-WPRE3-BGHpA |
| TG1002 | rAAV: mscRE16-pBGmin-EGFP-WPRE3-bGHpA |
| TG1009 | rAAV: mscRE4-pBGmin-dgCre-WPRE3-bGHpA |
| TG1010 | rAAV: mscRE4-pBGmin-iCre-WPRE3-bGHpA |
| TG1011 | rAAV: mscRE4-pBGmin-IRES2-tTA2-WPRE3-bGHpA |
| TG1021 | rAAV: mscRE4-pBGmin-Cre-WPRE3-bGHpA |
| TG1022 | rAAV: mscRE4-pBGmin-Cre-i-Cre-WPRE3-bGHpA |
| TG1036 | rAAV: mscRE10-pBGmin-FlpO-WPRE3-BGHpA |
| TG1037 | rAAV: mscRE13-pBGmin-FlpO-WPRE3-BGHpA |
| TG1038 | rAAV_mscRE16- pBGmin-FlpO-WPRE3-bGHpA |
| TG1045 | rAAV: mscRE10-pBGmin-iCre-WPRE3-BGHpA |
| TG1046 | rAAV: mscRE13-pBGmin-iCre-WPRE3-BGHpA |
| TG1047 | rAAV: mscRE16-pBGmin-iCre-WPRE3-bGHpA |
| TG1048 | rAAV: mscRE10-pBGmin-tTA2-WPRE3-BGHpA |
| TG1049 | rAAV: mscRE13-pBGmin-tTA2-WPRE3-BGHpA |
| TG1050 | rAAV: mscRE16- pBGmin-tTA2-WPRE3-bGHpA |
| TG1052 | rAAV: 4XmscRE16-pBGmin-EGFP-WPRE3-bGHpA |
| CN1402 | rAAV: eHGT_058h-minBglobin-SYFP2-WPRE3-BGHpA |
| CN1416 | rAAV: eHGT_058m-minBglobin-SYFP2-WPRE3-BGHpA |
| CN1427 | rAAV: mscRE4(4x)-minBglobin-tdTomato-WPRE3-BGHpA |
| CN1452 | rAAV: eHGT_073h-minBglobin-SYFP2-WPRE3-BGHpA |
| CN1454 | rAAV: eHGT_075h-minBglobin-SYFP2-WPRE3-BGHpA |
| CN1456 | rAAV: eHGT_077h-minBglobin-SYFP2-WPRE3-BGHpA |
| CN1457 | rAAV: eHGT_078h-minBglobin-SYFP2-WPRE3-BGHpA |
| CN1461 | rAAV: eHGT_073m-minBglobin-SYFP2-WPRE3-BGHpA |
| CN1466 | rAAV: eHGT_078m-minBglobin-SYFP2-WPRE3-BGHpA |
| CN1772 | rAAV: hsA2-eHGT_254h-minRho-SYFP2-WPRE3-BGHpA |
| CN1818 | rAAV: 3xCore-mscRE4-minCMV-SYFP2-WPRE3-bGHpA |
| CN1954 | rAAV: hsA2-eHGT 078h(3xCore)-minRho-SYFP2-WPRE3-BGHpA |
| CN1955 | rAAV: hsA2-eHGT 078m(3xCore)-minRho-SYFP2-WPRE3-BGHpA |
| CN2014 | rAAV: mscRE4-minCMV-SYFP2-WPRE3-BGHpA |
| CN2137 | rAAV: eHGT_440h-minBglobin-SYFP2-WPRE3-BGHpAv |
| CN2139 | rAAV: eHGT_439m-minBglobin-SYFP2-WPRE3-BGHpA |

In particular embodiments vectors (e.g., AAV) with capsids that cross the blood-brain barrier (BBB) are selected. In particular embodiments, vectors are modified to include capsids that cross the BBB. Examples of AAV with viral capsids that cross the blood brain barrier include AAV9 (Gombash et al., Front Mol Neurosci. 2014; 7:81), AAVrh.10 (Yang, et al., Mol Ther. 2014; 22 (7): 1299-1309), AAV1R6, AAV1R7 (Albright et al., Mol Ther. 2018; 26 (2): 510), rAAVrh.8 (Yang, et al., supra), AAV-BR1 (Marchio et al., EMBO Mol Med. 2016; 8 (6): 592), AAV-PHP.S (Chan et al., Nat Neurosci. 2017; 20 (8): 1172), AAV-PHP.B (Deverman et al., Nat Biotechnol. 2016; 34 (2): 204), AAV-PPS (Chen et al., Nat Med. 2009; 15:1215), and the PHP.eB capsid. The PHP.eB capsid differs from AAV9 such that, using AAV9 as a reference, amino acids starting at residue 586: S-AQ-A (SEQ ID NO: 169) are changed to S-DGTLAVPFK-A (SEQ ID NO: 170).

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31 (4): 317), for example, as described in relation to clinical trials for the treatment of spinal muscular atrophy (SMA) syndrome by AveXis (AVXS-101, NCT03505099) and the treatment of CLN3 gene-Related Neuronal Ceroid-Lipofuscinosis (NCT03770572).

AAVrh.10, was originally isolated from rhesus macaques and shows low seropositivity in humans when compared with other common serotypes used for gene delivery applications (Selot et al., Front Pharmacol. 2017; 8:441) and has been evaluated in clinical trials LYS-SAF302, LYSOGENE, and NCT03612869.

AAV1R6 and AAV1R7, two variants isolated from a library of chimeric AAV vectors (AAV1 capsid domains swapped into AAVrh.10), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 171) epitope that was isolated during in vivo screening of a random AAV display peptide library. It shows high specificity accompanied by high transgene expression in the brain with minimal off-target affinity (including for the liver) (Körbelin et al., EMBO Mol Med. 2016; 8 (6): 609).

AAV-PHP.S (Addgene, Watertown, MA) is a variant of AAV9 generated with the CREATE method that encodes the 7-mer sequence QAVRTSL (SEQ ID NO: 172), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 173). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 174) epitope into the capsid of AAV2, shows a dramatically improved brain tropism relative to AAV2.

For additional information regarding capsids that cross the blood brain barrier, see Chan et al., Nat. Neurosci. 2017 August: 20 (8): 1172-1179.

(ii) Compositions for Administration. Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in particular embodiments, when administered intravenously (e.g. at the retro-orbital plexus).

In particular embodiments, compositions can be formulated for intravenous, intraparenchymal, intraocular, intravitreal, parenteral, subcutaneous, intracerebro-ventricular, intramuscular, intrathecal, intraspinal, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

The formation and use of liposomes is generally known to those of skill in the art. Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24 (12): 1113-1128, 1998; Quintanar-Guerrero et al., Pharm Res. 15 (7): 1056-1062, 1998; Quintanar-Guerrero et al., J. Microencapsul. 15 (1): 107-119, 1998; Douglas et al., Crit Rev Ther Drug Carrier Syst 3 (3): 233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69 (2): 199-202, 1980; Couvreur et al., Crit Rev Ther Drug Carrier Syst. 5 (1) 1-20, 1988; zur Muhlen et al., Eur J Pharm Biopharm, 45 (2): 149-155, 1998; Zambaux et al., J Control Release 50 (1-3): 31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17 (1): 33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.

(iii) Cell Lines Including Artificial Expression Constructs. The present disclosure includes cells including an artificial expression construct described herein. A cell that has been transformed with an artificial expression construct can be used for many purposes, including in neuroanatomical studies, assessments of functioning and/or non-functioning proteins, and drug screens that assess the regulatory properties of enhancers.

A variety of host cell lines can be used, but in particular embodiments, the cell is a mammalian neural cell. In particular embodiments, the enhancer sequence of the artificial expression construct is mscRE1, mscRE3, mscRE4, a concatemer of the mscRE4 core, mscRE10, mscRE11, mscRE12, mscRE13, mscRE16, a concatemer of mscRE16, Grik1_enhScnn1a-2, eHGT_058h, eHGT_058m, eHGT_073h, eHGT_073m, eHGT_075h, eHGT_077h, eHGT_078h, a concatemer of eHGT_078h core, eHGT_078m, a concatemer of eHGT_078m core, eHGT_439m, eHGT_440h, and eHGT_254h and/or the artificial expression construct includes T502-050, T502-054, vAi34.0, vAi33.2, vAi45.0, vAi1.0, T502-057, T502-059, TG975, TG978, TG979, TG981, TG982, TG987, TG988, TG995, TG996, TG997, TG999, TG1002, TG1009, TG1010, TG1011, TG1021, TG1022, TG1036, TG1037, TG1038, TG1045, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and/or CN2014, and the cell line is a human, primate, or murine neural cell. Cell lines which can be utilized for transgenesis in the present disclosure also include primary cell lines derived from living tissue such as rat or mouse brains and organotypic cell cultures, including brain slices from animals such as rats or mice. The PC12 cell line (available from the American Type Culture Collection, ATCC, Manassas, VA) has been shown to express a number of neuronal marker proteins in response to Neuronal Growth Factor (NGF). The PC12 cell line is considered to be a neuronal cell line and is applicable for use with this disclosure. JAR cells (available from ATCC) are a platelet derived cell-line that express some neuronal genes, such as the serotonin transporter gene, and may be used with embodiments described herein.

WO 91/13150 describes a variety of cell lines, including neuronal cell lines, and methods of producing them. Similarly, WO 97/39117 describes a neuronal cell line and methods of producing such cell lines. The neuronal cell lines disclosed in these patent applications are applicable for use in the present disclosure.

In particular embodiments, a “neural cell” refers to a cell or cells located within the central nervous system, and includes neurons and glia, and cells derived from neurons and glia, including neoplastic and tumor cells derived from neurons or glia. A “cell derived from a neural cell” refers to a cell which is derived from or originates or is differentiated from a neural cell.

In particular embodiments, “neuronal” describes something that is of, related to, or includes, neuronal cells. Neuronal cells are defined by the presence of an axon and dendrites. The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

In particular embodiments, non-neuronal cell lines may be used, including mouse embryonic stem cells. Cultured mouse embryonic stem cells can be used to analyze expression of genetic constructs using transient transfection with plasmid constructs. Mouse embryonic stem cells are pluripotent and undifferentiated. These cells can be maintained in this undifferentiated state by Leukemia Inhibitory Factor (LIF). Withdrawal of LIF induces differentiation of the embryonic stem cells. In culture, the stem cells form a variety of differentiated cell types. Differentiation is caused by the expression of tissue specific transcription factors, allowing the function of an enhancer sequence to be evaluated. (See for example Fiskerstrand et al., FEBS Lett 458:171-174, 1999.)

Methods to differentiate stem cells into neuronal cells include replacing a stem cell culture media with a media including basic fibroblast growth factor (bFGF) heparin, an N2 supplement (e.g., transferrin, insulin, progesterone, putrescine, and selenite), laminin and polyornithine. A process to produce myelinating oligodendrocytes from stem cells is described in Hu, et al., 2009, Nat. Protoc. 4:1614-22. Bibel, et al., 2007, Nat. Protoc. 2:1034-43 describes a protocol to produce glutamatergic neurons from stem cells while Chatzi, et al., 2009. Exp. Neurol. 217:407-16 describes a procedure to produce GABAergic neurons. This procedure includes exposing stem cells to all-trans-RA for three days. After subsequent culture in serum-free neuronal induction medium including Neurobasal medium supplemented with B27, bFGF and EGF. 95% GABA neurons develop

U.S. Publication No 2012/0329714 describes use of prolactin to increase neural stem cell numbers while U.S. Publication No 2012/0308530 describes a culture surface with amino groups that promotes neuronal differentiation into neurons, astrocytes and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No. 5,766,948; FGF-1, FGF-2); Neurotrophin-3 (NT-3) and Neurotrophin-4 (NT-4); Caldwell, et al., 2001, Nat. Biotechnol. 1; 19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-α and TGF-β (U.S. Pat. Nos. 5,851,832 and 5,753,506).

In particular embodiments, yeast one-hybrid systems may also be used to identify compounds that inhibit specific protein/DNA interactions, such as transcription factors for the mscRE1, mscRE3, mscRE4, mscRE10, mscRE11, mscRE12, mscRE13, mscRE16, Grik1_enhScnn1a-2, eHGT_058h, eHGT_058m, eHGT_073h, eHGT_073m, eHGT_075h, eHGT_077h, eHGT_078h, a concatemer of eHGT_078h core, eHGT_078m, a concatemer of eHGT_078m core, eHGT_439m, eHGT_440h, and/or eHGT_254h enhancer.

Transgenic animals are described below. Cell lines may also be derived from such transgenic animals. For example, primary tissue culture from transgenic mice (e.g., also as described below) can provide cell lines with the expression construct already integrated into the genome. (for an example see Mackenzie & Quinn, Proc Natl Acad Sci USA 96:15251-15255, 1999).

(iv) Transgenic Animals. Another aspect of the disclosure includes transgenic animals, the genome or cells of which contain an artificial expression construct including mscRE1, mscRE3, mscRE4, a concatemer of the mscRE4 core, mscRE10, mscRE11, mscRE12, mscRE13, mscRE16, a concatemer of mscRE16, Grik1_enhScnn1a-2, eHGT_058h, eHGT_058m, eHGT_073h, eHGT_073m, eHGT_075h, eHGT_077h, eHGT_078h, a concatemer of eHGT_078h core, eHGT_078m, a concatemer of eHGT_078m core, eHGT_439m, eHGT_440h, and/or eHGT_254h operatively linked to a heterologous coding sequence. In particular embodiments, the genome or cells of a transgenic animal includes an artificial expression construct including T502-050, T502-054, vAi34.0, vAi33.2, vAi45.0, vAi1.0, T502-057, T502-059, TG975, TG978, TG979, TG981, TG982, TG987, TG988, TG995, TG996, TG997, TG999, TG1002, TG1009, TG1010, TG1011, TG1021, TG1022, TG1036, TG1037, TG1038, TG1045, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and/or CN2014. In particular embodiments, when a non-integrating vector is utilized, a transgenic animal includes an artificial expression construct including mscRE1, mscRE3, mscRE4, a concatemer of the mscRE4 core, mscRE10, mscRE11, mscRE12, mscRE13, mscRE16, a concatemer of mscRE16, Grik1_enhScnn1a-2, eHGT_058h, eHGT_058m, eHGT_073h, eHGT_073m, eHGT_075h, eHGT_077h, eHGT_078h, a concatemer of eHGT_078h core, eHGT_078m, a concatemer of eHGT_078m core, eHGT_439m, eHGT_440h, eHGT_254h and/or T502-050, T502-054, vAi34.0, vAi33.2, vAi45.0, vAi1.0, T502-057, T502-059, TG975, TG978, TG979, TG981, TG982, TG987, TG988, TG995, TG996, TG997, TG999, TG1002, TG1009, TG1010, TG1011, TG1021, TG1022, TG1036, TG1037, TG1038, TG1045, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and/or CN2014 within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In particular embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to heterologous expression of the construct. A disadvantage of this approach is that the expression of the introduced DNA may be affected by the specific genetic background of the host animal.

As indicated above in relation to cell lines, the artificial expression constructs of this disclosure can be used to genetically modify mouse embryonic stem cells using techniques known in the art. Typically, the artificial expression construct is introduced into cultured murine embryonic stem cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice be crossed with an appropriate strain to produce offspring that will carry the transgene.

In addition to the methods of delivery described above, the following techniques are also contemplated as alternative methods of delivering artificial expression constructs to target cells or selected tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17 (1): 33-58, 1998); transdermal matrices (U.S. Pat. Nos. 5,770,219 and 5,783,208); and feedback-controlled delivery (U.S. Pat. No. 5,697,899).

(v) Methods of Use. In particular embodiments, a composition including a physiologically active component described herein is administered to a subject to result in a physiological effect.

In particular embodiments, the disclosure includes the use of the artificial expression constructs described herein to modulate expression of a heterologous gene which is either partially or wholly encoded in a location downstream to that enhancer in an engineered sequence. Thus, there are provided herein methods of use of the disclosed artificial expression constructs in the research, study, and potential development of medicaments for preventing, treating or ameliorating the symptoms of a disease, dysfunction, or disorder.

Particular embodiments include methods of administering to a subject an artificial expression construct that includes SEQ ID NOs: 25-51, 177-178, and/or 188 and/or SEQ ID NOs: 73-114, and/or 179-187 as described herein to drive selective expression of a gene in a selected neural cell type.

Particular embodiments include methods of administering to a subject an artificial expression construct that includes SEQ ID NOs: 25-51, 177-178, and/or 188 and/or SEQ ID NOs: 73-114, and/or 179-187 as described herein to drive selective expression of a gene in a selected neural cell type wherein the subject can be an isolated cell, a network of cells, a tissue slice, an experimental animal, a veterinary animal, or a human.

As is well known in the medical arts, dosages for any one subject depends upon many factors, including the subject's size, surface area, age, the particular compound to be administered, sex, time and route of administration, general health, and other drugs being administered concurrently. Dosages for the compounds of the disclosure will vary, but, in particular embodiments, a dose could be from 10⁵ to 10¹⁰⁰ copies of an artificial expression construct of the disclosure. In particular embodiments, a patient receiving intravenous, intraparenchymal, intraspinal, retro-orbital, or intrathecal administration can be infused with from 10⁶ to 10²² copies of the artificial expression construct.

An “effective amount” is the amount of a composition necessary to result in a desired physiological change in the subject. Effective amounts are often administered for research purposes. Effective amounts disclosed herein can cause a statistically-significant effect in an animal model or in vitro assay.

The amount of expression constructs and time of administration of such compositions will be within the purview of the skilled artisan having benefit of the present teachings. It is likely, however, that the administration of effective amounts of the disclosed compositions may be achieved by a single administration, such as for example, a single injection of sufficient numbers of infectious particles to provide an effect in the subject. Alternatively, in some circumstances, it may be desirable to provide multiple, or successive administrations of the artificial expression construct compositions or other genetic constructs, either over a relatively short, or a relatively prolonged period of time, as may be determined by the individual overseeing the administration of such compositions. For example, the number of infectious particles administered to a mammal may be 10⁷, 10⁸, 10⁹, 10¹⁰, 10¹¹, 10¹², 10¹³, or even higher, infectious particles/ml given either as a single dose or divided into two or more administrations as may be required to achieve an intended effect. In fact, in certain embodiments, it may be desirable to administer two or more different expression constructs in combination to achieve a desired effect.

In certain circumstances it will be desirable to deliver the artificial expression construct in suitably formulated compositions disclosed herein either by pipette, retro-orbital injection, subcutaneously, intraocularly, intravitreally, parenterally, subcutaneously, intravenously, intraparenchymally, intracerebro-ventricularly, intramuscularly, intrathecally, intraspinally, orally, by oral or nasal inhalation, intraperitoneally, or by direct application or injection to one or more cells, tissues, or organs. The methods of administration may also include those modalities as described in U.S. Pat. Nos. 5,543,158; 5,641,515 and 5,399,363.

(vi) Kits and Commercial Packages. Kits and commercial packages contain an artificial expression construct described herein. The expression construct can be isolated. In particular embodiments, the components of an expression product can be isolated from each other. In particular embodiments, the expression product can be within a vector, within a viral vector, within a cell, within a tissue slice or sample, and/or within a transgenic animal. Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition.

The Exemplary Embodiments and Experimental Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (vii) Exemplary Embodiments

1. A concatenated core of an enhancer disclosed herein.

2. A concatenated core of embodiment 1, wherein the core is selected from SEQ ID NOs: 29, 177, and/or 178.

3. The concatenated core of embodiment 1 or 2, wherein the concatenated core includes 2, 3, 4, 5, 6, 7, 8, 9, or 10 copies of SEQ ID NOs: 29, 177, and/or 178.

4. The concatenated core of embodiment 3, including 3 copies of SEQ ID NO: 29.

5. The concatenated core of embodiment 4, including SEQ ID NO: 30.

6. The concatenated core of embodiment 3, including 3 copies of SEQ ID NO: 177.

7. The concatenated core of embodiment 6 including SEQ ID NO: 40.

8. The concatenated core of embodiment 3, including 3 copies of SEQ ID NO: 178.

9. The concatenated core of embodiment 8 including SEQ ID NO: 49.

10. An artificial expression construct including (i) an enhancer selected from mscRE1, mscRE3, mscRE4, mscRE10, mscRE11, mscRE12, mscRE13, mscRE16, Grik1_enhScnn1a-2, eHGT_058h, eHGT_058m, eHGT_073h, eHGT_073m, eHGT_075h, eHGT_077h, eHGT_078h, eHGT_078m, eHGT_439m, eHGT_440h, eHGT_254h, and/or a concatemer of any of embodiments 1-8; (ii) a promoter; and (iii) a heterologous encoding sequence.

11. The artificial expression construct of embodiment 10, wherein the heterologous encoding sequence encodes an effector element or an expressible element.

12. The artificial expression construct of embodiment 11, wherein the effector element includes a reporter protein or a functional molecule.

13. The artificial expression construct of embodiment 12, wherein the reporter protein includes a fluorescent protein.

14. The artificial expression construct of embodiment 12, wherein the functional molecule includes a functional ion transporter, enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or a designer receptor exclusively activated by designer drug (DREADD).

15. The artificial expression construct of embodiment 11, wherein the expressible element includes a non-functional molecule.

16. The artificial expression construct of embodiment 15, wherein the non-functional molecule includes a non-functional ion transporter, enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or a DREADD.

17. The artificial expression construct of any of embodiments 10-16 including a concatemer of an enhancer selected from mscRE1, mscRE3, mscRE4, mscRE10, mscRE11, mscRE12, mscRE13, mscRE16, Grik1_enhScnn1a-2, eHGT_058h, eHGT_058m, eHGT_073h, eHGT_073m, eHGT_075h, eHGT_077h, eHGT_078h, eHGT_078m, eHGT_439m, eHGT_440h, and eHGT_254h.

18. The artificial expression construct of embodiment 17 wherein the concatemer includes 2, 3, 4, 5, 6, 7, 8, 9, or 10 copies of the selected enhancer.

19. The artificial expression construct of embodiment 18 wherein the concatemer includes 3 or 4 copies of mscRE4 or 3 or 4 copies of mscRE16.

20. The artificial expression construct of any of embodiments 10-19, wherein the artificial expression construct is associated with a capsid that crosses the blood brain barrier.

21. The artificial expression construct of embodiment 20, wherein the capsid includes PHP.eB, AAV-BR1, AAV-PHP.S, AAV-PHP.B, or AAV-PPS.

22. The artificial expression construct of any of embodiments 10-21, wherein the expression construct includes or encodes a skipping element.

23. The artificial expression construct of embodiment 22, wherein the skipping element includes a 2A peptide and/or an internal ribosome entry site (IRES).

24. The artificial expression construct of embodiment 23, wherein the 2A peptide includes selected from T2A, P2A, E2A, or F2A.

25. The artificial expression construct of any of embodiments 10-24, wherein the artificial expression construct includes a set of features selected from: an enhancer selected from mscRE1, mscRE3, mscRE4, mscRE10, mscRE11, mscRE12, mscRE13, mscRE16, Grik1_enhScnn1a-2, eHGT_058h, eHGT_058m, eHGT_073h, eHGT_073m, eHGT_075h, eHGT_077h, eHGT_078h, eHGT_078m, eHGT_439m, eHGT_440h, or eHGT_254h, and/or a concatemer of any of embodiments 1-9; a promoter selected from pBGmin or minBglobin; an expression product selected from EGFP, SYFP2, IRES2, FlpO, Cre, iCre, dgCre, or tTA2; and a post-regulatory element selected from WPRE3 and/or BGHpA

26. A vector including a concatenated core and/or artificial expression construct of any of embodiments 1-25.

27. A vector including features selected from T502-050, T502-054, vAi34.0, vAi33.2, vAi45.0, vAi1.0, T502-057, T502-059, TG975, TG978, TG979, TG981, TG982, TG987, TG988, TG995, TG996, TG997, TG999, TG1002, TG1009, TG1010, TG1011, TG1021, TG1022, TG1036, TG1037, TG1038, TG1045, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and CN2014.

28. The vector of embodiment 27, wherein the vector includes a viral vector.

29. The vector of embodiment 28, wherein the viral vector includes a recombinant adeno-associated viral (AAV) vector.

30. An adeno-associated viral (AAV) vector including at least one heterologous encoding sequence, wherein the heterologous encoding sequence is under control of a promoter and an enhancer selected from mscRE1, mscRE3, mscRE4, mscRE10, mscRE11, mscRE12, mscRE13, mscRE16, Grik1_enhScnn1a-2, eHGT_058h, eHGT_058m, eHGT_073h, eHGT_073m, eHGT_075h, eHGT_077h, eHGT_078h, eHGT_078m, eHGT_439m, eHGT_440h, eHGT_254h, and/or a concatemer of any of embodiments 1-9.

31. The AAV vector of embodiment 30, wherein the AAV vector is replication-competent.

32. A transgenic cell including a concatenated core, artificial expression construct and/or vector of any of the preceding embodiments.

33. The transgenic cell of embodiment 32, wherein the transgenic cell is an excitatory cortical neuron.

34. The transgenic cell of embodiment 32 or 33, wherein the transgenic cell is a layer (L) 2, L3, L4, L5, or L6 excitatory cortical neuron.

35. The transgenic cell of any of embodiments 32-34, wherein the transgenic cell is an L4 IT excitatory cortical neuron, an L5 PT excitatory cortical neuron, an L5 ET excitatory cortical neuron, an L5 IT excitatory cortical neuron, an L5 NP excitatory cortical neuron, an L6 IT excitatory cortical neuron, an L6 CT excitatory cortical neuron, or a CR excitatory cortical neuron.

36. The transgenic cell of embodiment 32, wherein the transgenic cell is derived from a subcortical population in the CEAc, the substantia nigra, compact part, the subiculum, or the prosubiculum (ProS).

37. The transgenic cell of embodiment 32, wherein the transgenic cell is a CA1 pyramidal neuron, a dentate gyrus granule cell, a striatal neuron, or a cerebellar Purkinje cell.

38. A non-human transgenic animal including a concatenated core enhancer, an artificial expression construct, vector, and/or transgenic cell of any of the preceding embodiments.

39. The non-human transgenic animal of embodiment 38, wherein the non-human transgenic animal is a mouse or a non-human primate.

40. An administrable composition including a concatenated core, an artificial expression construct, vector, or transgenic cell of any of the preceding embodiments.

41. A kit including a concatenated core, an artificial expression construct, vector, transgenic cell, transgenic animal, and/or administrable compositions of any of the preceding embodiments.

42. A method for selectively expressing a heterologous gene within a population of neural cells in vivo or in vitro, the method including providing the administrable composition of embodiment 40 in a sufficient dosage and for a sufficient time to a sample or subject including the population of neural cells thereby selectively expressing the gene within the population of neural cells.

43. The method of embodiment 42, wherein the heterologous gene encodes an effector element or an expressible element.

44. The method of embodiment 43, wherein the effector element includes a reporter protein or a functional molecule.

45. The method of embodiment 44, wherein the reporter protein includes a fluorescent protein.

46. The method of embodiment 44, wherein the functional molecule includes a functional ion transporter, enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or a DREADD.

47. The method of embodiment 43, wherein the expressible element includes a non-functional molecule.

48. The method of embodiment 47, wherein the non-functional molecule includes a non-functional ion transporter, enzyme, transcription factor, receptor, membrane protein, cellular trafficking protein, signaling molecule, neurotransmitter, calcium reporter, channel rhodopsin, CRISPR/CAS molecule, editase, guide RNA molecule, homologous recombination donor cassette, or DREADD.

49. The method of any of embodiments 42-48, wherein the providing includes pipetting.

50. The method of embodiment 49, wherein the pipetting is to a brain slice.

51. The method of embodiment 50, wherein the brain slice includes an excitatory neuron.

52. The method of embodiment 50 or 51, wherein the brain slice includes a layer (L) 2, L3, L4, L5, and/or a L6 excitatory cortical neuron.

53. The method of any of embodiments 50-52, wherein the brain slice includes an L4 IT excitatory cortical neuron, an L5 PT excitatory cortical neuron, an L5 ET excitatory cortical neuron, an L5 IT excitatory cortical neuron, an L5 NP excitatory cortical neuron, an L6 IT excitatory cortical neuron, an L6 CT excitatory cortical neuron, and/or a CR excitatory cortical neuron.

54. The method of any of embodiments 50-53, wherein the brain slice includes a subcortical population in the CEAc, the substantia nigra, compact part, the subiculum, and/or the prosubiculum (ProS).

55. The method of any of embodiments 50-54, wherein the brain slice includes a CA1 pyramidal neuron, a dentate gyrus granule cell, a striatal neuron, and/or a cerebellar Purkinje cell.

56. The method of any of embodiments 50-55, wherein the brain slice is murine, human, or non-human primate.

57. The method of embodiment 48, wherein the providing includes administering to a living subject.

58. The method of embodiment 57, wherein the living subject is a human, non-human primate, or a mouse.

59. The method of embodiments 56 or 57, wherein the administering to a living subject is through injection.

60. The method of embodiment 59, wherein the injection includes intravenous injection, intraparenchymal injection, intracerebroventricular (ICV) injection, intra-cisterna magna (ICM) injection, or intrathecal injection.

61. An artificial expression construct including T502-050, T502-054, vAi34.0, vAi33.2, vAi45.0, vAi1.0, T502-057, T502-059, TG975, TG978, TG979, TG981, TG982, TG987, TG988, TG995, TG996, TG997, TG999, TG1002, TG1009, TG1010, TG1011, TG1021, TG1022, TG1036, TG1037, TG1038, TG1045, TG1046, TG1047, TG1048, TG1049, TG1050, TG1052, CN1402, CN1457, CN1818, CN1416, CN1452, CN1461, CN1454, CN1456, CN1772, CN1427, CN1466, CN1954, CN1955, CN2137, CN2139, and CN2014.

(viii) Experimental Examples. Example 1. Individual neuronal or non-neuronal cells were isolated from the mouse cortex by FACS and examined using the Assay for Transposase-Accessible Chromatin with next generation sequencing (ATAC-seq). This strategy allowed interrogation of both abundant and very rare cell types with the same method. 25 individual or combinatorial Cre or Flp-driver lines were utilized in combination with reporter lines, many of which have been characterized using single-cell RNA-seq (Tasic, et al., 2018, Nature 563:72-78), as well as retrograde labeling to selectively sample cell populations in adult mouse brain. Shared GABAergic cell types across two distant poles of mouse cortex, but divergent glutamatergic cell types from different cortical regions have been observed (Tasic, et al., 2018, Nature 563:72-78). Therefore, dissections focused on visual cortex for glutamatergic cell types, but allowed broader cortical sampling for GABAergic cell types. Retrogradely labeled cells were collected only from visual cortex. In total, 3,381 single cells from 25 driver-reporter combinations in 60 mice, 126 retrogradely labeled cells from injections into 3 targets across 7 donors, and 96 samples labeled in 1 retro-orbital injection from a viral tool generated were collected. After FACS, individual cells were subjected to ATAC-seq, and were sequenced in 60-96 sample batches using a MiSeq (Materials and Methods of Example 1). Quality control filtering was performed to select 2,416 samples with >10,000 uniquely mapped paired-end fragments, >10% of which had a fragment size longer than 250 bp, and with >25% of fragments overlapping high-depth cortical DNAse-seq peaks generated by ENCODE (Yue, et al., 2014, Nature, 515:355-364).

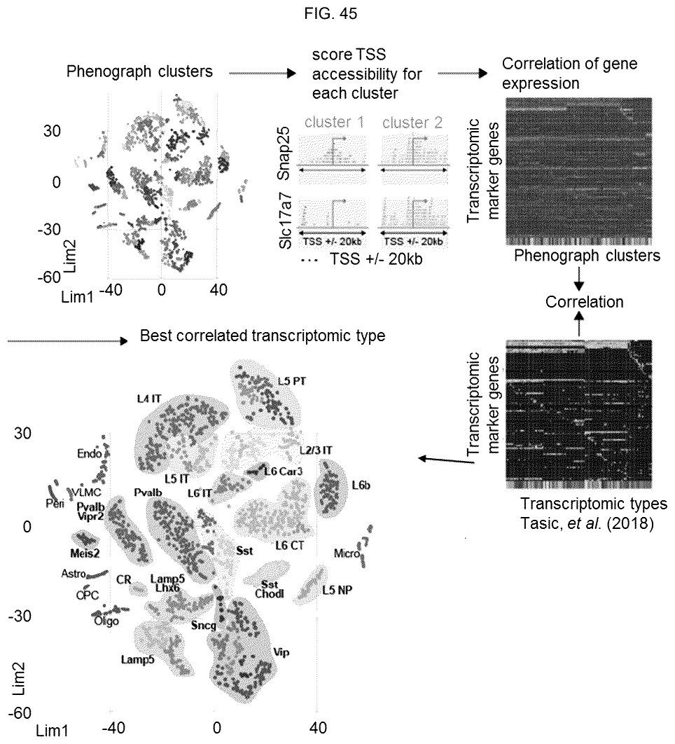

Previous studies have shown that most recombinase driver lines label more than one transcriptomic cell type (Tasic, et al., 2018, Nature 563:72-78; and Tasic, et al., 2016, Nat Neurosci 19:335-346). To increase the resolution of chromatin accessibility profiles beyond that provided by driver lines, the scATAC-seq data was clustered using a novel feature-free method for computation of pairwise Jaccard distances. These distances were used for clustering by t-stochastic neighborhood embedding (t-SNE), followed by phenograph clustering (FIG. 45). Cluster identity was then assigned by comparison of accessibility near transcription start sites (TSS±20 kb) to scRNA-seq dataset for VISp (Tasic, et al., 2018, Nature 563:72-78) using median correlation.

Layer 5 of visual cortex contains L5 IT neurons that project to other cortical regions, near-projecting (L5 NP) neurons that have only local projections, and L5 PT neurons that have long axonic projections to subcortical brain regions such as thalamus (Tasic, et al., 2018, Nature 563:72-78; Harris, et al., 2018, biorXiv, 292961). The driver line Rbp4-Cre labels both L5 IT and L5 PT neurons in cortex (Tasic, et al., 2016, Nat Neurosci 19:335-346). To deconvolute these populations, L5 PT and L5 IT neurons were identified in the scATAC-seq dataset based on correlation with scRNA-seq cell types, labeling of these cells by Rbp4-Cre, and by retrograde labeling from a known L5 PT target region, the lateral posterior nucleus of the thalamus (LP). Populations of L5 PT and L5 IT scATAC-seq samples were pooled into subclass-specific tracks, and searches were performed near transcriptomic marker genes for 500 bp putative enhancer elements that were specific to L5 PT or L5 IT cells, and which had strong sequence conservation. These regions are referred to as mouse single-cell regulatory elements (mscREs).

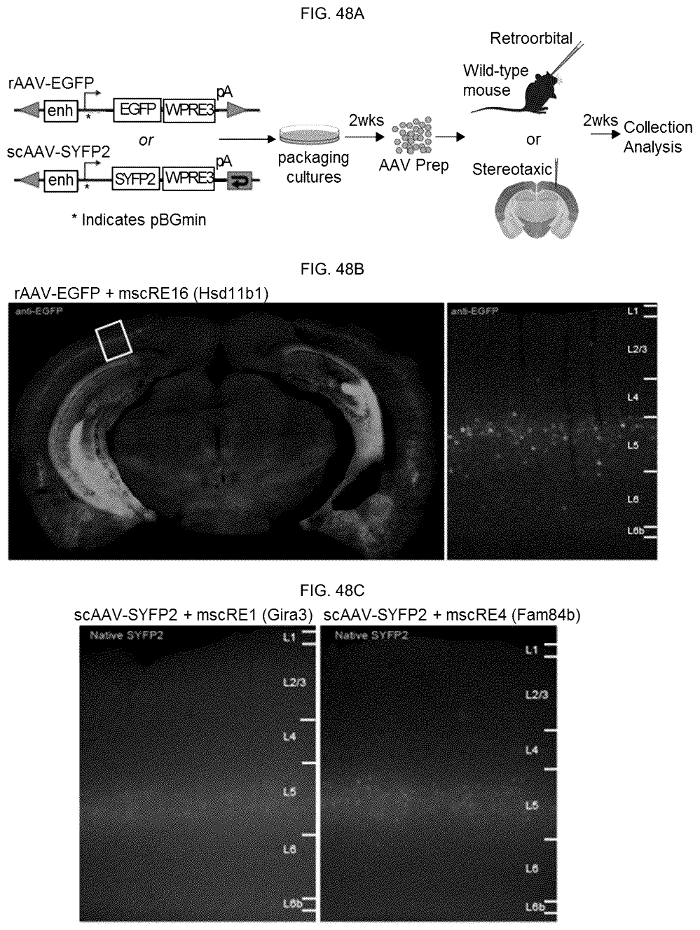

Putative mscREs were cloned upstream of a minimal beta-globin promoter driving SYFP2 or EGFP expression in a viral construct to generate AAVs (FIG. 48A). These constructs were packaged for retro-orbital injection into wild-type mice in a PHP.eB-serotype virus, which can cross the blood-brain barrier (Chan, et al., 2017, Nat. Neurosci 20:1172-1179). In total, 4 mscREs for L5 PT cells, and 2 mscREs for L5 IT were screened. Two weeks after retro-orbital injection, brains of infected mice were collected and screened expression by visual inspection of native fluorescence and immunohistochemistry to enhance SYFP2 and EGFP signal. Three of the enhancers provided labeling of cells in L5, while others showed off-target or no detectable labeling.

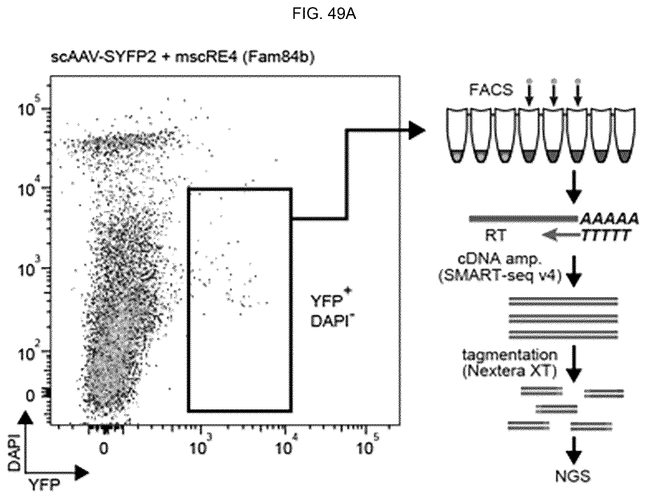

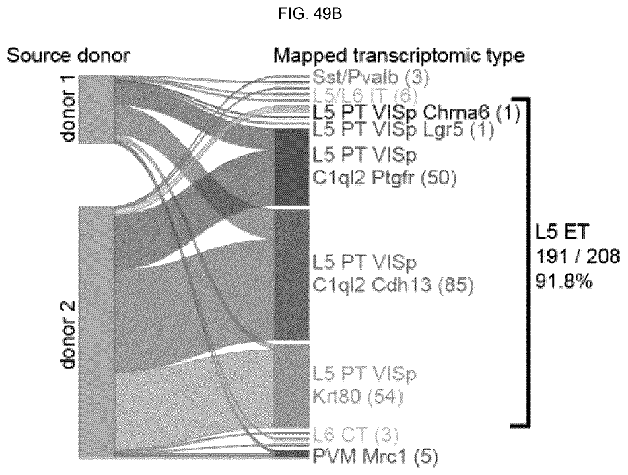

To assess the specificity of cell type labeling, stereotaxic injection of these viruses in VISp was performed, labeled cells were sorted by FACS, and scRNA-seq was performed as described previously (Tasic, et al., 2018, Nature 563:72-78). scRNA-seq expression profiles were compared to a VISp reference dataset using centroid classification of cell types (Materials and Methods of Example 1). The mscRE4 element yielded specificity for L5 PT cells, (FIG. 48B), mscRE1 yielded specificity for L5 PT cells, and mscRE16 yielded specificity for L5 IT cells. scRNA-seq of FACS-sorted cells was also performed from retro-orbital labeling of the mscRE4 and mscRE1 viruses, with similarly specific results (>92% for mscRE4). Direct labeling of cells by stereotaxic injection induced an innate immune response similar to anterograde labeling, but retrograde injections caused no significant upregulation of immune-related pathways at the time of collection. For mscRE4, labeling of L5 PT cells was confirmed by electrophysiological characterization of labeled vs unlabeled cells in the cortex. Cells labeled by mscRE4 had characteristics of L5 PT neurons, whereas cells that were label-negative did not (FIG. 49A). This demonstrates the utility of these viral tools for electrophysiology experiments targeted to specific subclasses for which driver lines are not available.

L5 PT cells are often difficult to isolate from single-cell suspensions when in a heterogeneous mixture with other cell types due to differential cell survival (Tasic, et al., 2016, Nat Neurosci 19:335-346; and Tasic, et al., 2018, Nature 563:72-78). Retro-orbital injection of the mscRE4-driven virus (T502-057) was used to bootstrap the scATAC-seq dataset by sorting cells labeled by mscRE4 for FACS. As expected, based on scRNA-seq analysis, 55 of 61 high-quality mscRE4 scATAC-seq profiles clustered together with other L5 PT samples (90.2%).

Although the direct fluorophore labeling provided enough signal to sort cells by FACS or perform patch-clamp experiments, use of an enhancer to drive expression of a recombinase could allow for expression of previously generated mouse reporter lines that drive fluorophores, activity reporters, opsins, or genes that are too large to package in AAVs (Daigle, et al., 2018, Cell 174 (2): 465-480 and Madisen, et al., 2015, Neuron 85 (5): 942-958). To test the specificity of enhancer-driven recombinase expression, mscRE4 was cloned into constructs containing a minimal beta-globin enhancer driving dgCre (TG1009), iCre (TG1010), FlpO (TG978) or tTA2 (TG1011), and packaged them in PHP.eB viruses. These viruses were delivered by retro-orbital injection into mice with genetically encoded reporters for each recombinase (Ai14 for dgCre and iCre; Ai65F for FlpO; and Ai63 for tTA2). Labeling was characterized by sectioning and microscopy of native fluorescence (Materials and Methods of Example 1). FlpO, dgCre, and tTA2 yielded highly specific labeling of cells in L5 of the mouse cortex. For the FlpO virus, whole-brain microscopy was also performed using a TissueCyte system, and strong, specific labeling of L5 cells was found throughout the cortex, with bright labeling of pyramidal tract projections to subcortical targets. Finally, brain-wide colabeling of both L5 IT and L5 PT populations by retro-orbital injection of mscRE4-FlpO (to label L5 PT cells, red, TG978) and mscRE16-EGFP (to label L5 IT cells, green, TG1002) was tested in the same Ai65F animal. Distinct labeling of these two cell populations in L5 by microscopy was found, demonstrating that multiple enhancer-driven viruses can be used to simultaneously label populations of transcriptomically defined cell types in the same animal.

Materials and Methods of Example 1. Mouse breeding and husbandry. Mice were housed under Institutional Care and Use Committee protocols 1508 and 1802 at the Allen Institute for Brain Science, with no more than five animals per cage, maintained on a 12 hr day/night cycle, with food and water provided ad libitum. Animals with anophthalmia or microphthalmia were excluded from experiments. Animals were maintained on a C57BL/6J genetic background.

Retrograde labeling. Stereotaxic injection of CAV-Cre (Hnasko et al., 2006, Proc. Natl. Acad. Sci. USA 103:8858-8863) was performed into brains of heterozygous or homozygous Ai14 mice using coordinates obtained from Paxinos adult mouse brain atlas (Paxinos & Franklin, The Mouse Brain in Stereotaxic Coordinates Compact 3rd Ed., Academic Press, NY, 2008}. TdT+ single cells were isolated from VISp by FACS.

Single cell ATAC. Single-cell suspensions of cortical neurons were generated as described previously (Gray, et al., 2017, eLife 6: e21883}, with the exception of use of Papain in place of Proteinase K for dissociation of some samples. Individual cells with high fluorophore labeling (tdTomato or SYFP2) were then sorted for neuronal sorting or low fluorophore labeling for non-neuronal cell labeling, and low DAPI into 200 μL 8-well strip tubes containing 1.5 μL tagmentation reaction mix (0.75 μL Nextera Reaction Buffer, 0.2 μL Nextera Tn5 Enzyme, 0.55 μL water). After collection, cells were briefly spun down in a bench-top centrifuge, then immediately tagmented at 37° C. for 30 minutes in a PCR machine. After tagmentation, 0.6 μL Proteinase K stop solution was added to each tube (5 mg/mL Proteinase K solution (Qiagen), 50 mM EDTA, 5 mM NaCl, 1.25% SDS) followed by incubation at 40° C. for 30 minutes in a PCR machine. The tagmented DNA was then purified using AMPure XP beads (Beckman Coulter) at a ratio of 1.8:1 resuspended beads to reaction volume (3.8 μL added to 2.1 μL), with a final elution volume of 11 μL. Libraries were indexed and amplified by the addition of 15 μL 2× Kapa HiFi HotStart ReadyMix and 2 μL Nextera i5 and i7 indexes to each tube, followed by incubation at 72° C. for 3 minutes and PCR (95° C. for 1 min, 22 cycles of 98° C. for 20 sec, 65° C. for 15 sec, and 72° C. for 15 sec, then final extension at 72° C. for 1 min). After amplification, sample concentrations were measured using a Quant-iT PicoGreen assay (Thermo Fisher) in duplicate. For each sample, the mean concentration was calculated by comparison to a standard curve, and the mean and standard deviation of concentrations was calculated for all samples. Samples with a concentration greater than 2 standard deviations above the mean were not used for downstream steps, as these were found in early experiments to dominate sequencing runs. All other samples were pooled by combining 5 μL of each sample in a 1.5 mL tube. The combined library was then purified by adding Ampure XP beads in a 1.8:1 ratio, with final elution in 50 μL. The mixed library was then quantified using a BioAnalyzer High Sensitivity DNA kit (Agilent).

scATAC sequencing, alignment, and filtering. Mixed libraries, containing 60 to 96 samples each, were sequenced on an Illumina MiSeq at a final concentration of 20-30 pM. After sequencing, raw FASTQ files were aligned to the GRCm38 (mm10) mouse genome using Bowtie v1.1.0 as described previously (Gray, et al., 2017, eLife 6: e21883). After alignment, duplicate reads were removed using samtools rmdup, which yielded only single copies of uniquely mapped paired reads in BAM format. For analysis, samples were filtered to remove the ones with fewer than 10,000 paired-end fragments (20,000 reads), and with at least 10% of sequenced fragments longer than 250 bp. An additional filter was created using ENCODE whole cortex DNase-seq HotSpot peaks (sample ID ENCFF651EAU from experiment ID ENCSROOCOF). Samples with less than 25% of paired-end fragments that overlapped DNase-seq peaks were removed from downstream analysis. Cells passing these criteria both had sufficient unique reads for downstream analysis and had high-quality chromatin accessibility profiles as assessed by fragment size analysis. As an additional QC check, aggregate scATAC-seq data was compared to bulk ATAC-seq data from matching Cre-driver lines, where available. Aggregate single-cell datasets were found to match well to previously published bulk datasets.

Jaccard distance calculation, PCA and tSNE embedding, and density-based clustering. To compare scATAC-seq samples, all cells were downsampled to an equal number of uniquely aligned fragments (10,000 per sample). These fragments were extended to a length of 10 kb, then any overlapping fragments within each sample were collapsed into regions based on the outer boundaries of overlapping fragments. Then, the number of overlapping regions between every pair of samples was counted and divided by the total number of regions in both samples to obtain a Jaccard similarity score. These scores were converted to Jaccard distances (1-Jaccard similarity), and the resulting matrix was used as input for t-stochastic neighbor embedding (t-SNE). After t-SNE, samples were clustered in t-SNE space using the RPhenograph package with settings that yielded >100 clusters to obtain small groups of similar neighbors (Levine, et al., 2015, Cell 162:184-197).

Correlation with single-cell transcriptomics. Phenograph-defined neighborhoods were assigned to cell subclasses and clusters by comparison of accessibility scores of regions within 20 kb of each transcription start site (TSS) to median expression values of scRNA-seq clusters from mouse primary visual cortex (Tasic, et al., 2018, Nature 563:72-78) (Materials and Methods of Example 1). This strategy of neighbor assignment and correlation allowed resolution of cell types within the scATAC-seq data close to the resolution of the scRNA-seq data, as types that were split too far would resolve to the same transcriptomic type by correlation. To assess the robustness of these assignments, a bootstrapped clustering method was used, in which 20% of the scATAC-seq samples were randomly discarded, t-SNE was performed, clusters assigned, and comparison to scRNA-seq clusters were performed 100 times. As an alternative to Phenograph clustering, these analyses were also performed by selecting the 5 nearest neighbors of each sample in t-SNE space and performing the same count and correlation analysis described above.

Merging cell classes and peak calling. Aligned reads from single cell subclasses/clusters were used to create Tag Directories and call chromatin accessible peaks using HOMER (findPeaks -region-o auto). The resulting peaks were transformed to BED format and used as input for DiffBind/differential enrichment analyses.

Viral genome cloning. Enhancers were cloned from C57BI/6J genomic DNA using enhancer-specific primers and Phusion high-fidelity polymerase (M0530S; NEB). Individual enhancers were then inserted into an rAAV or self-complementary adeno-associated virus (scAAV) backbone that contained a minimal beta-globin promoter, gene, and bovine growth hormone polyA using standard molecular cloning approaches. Plasmid integrity was verified via Sanger sequencing and restriction digests to confirm intact inverted terminal repeat (ITR) sites.

Viral packaging and tittering. Before transfection, 105 μg of AAV viral genome plasmid, 190 μg pHelper, and 105 μg AAV-PHP.eB were mixed with 5 mL of Opti-MEM I media (Reduced Serum, GlutaMAX; ThermoFisher Scientific) and 1.1 mL of a solution of 1 mg/mL 25 kDa linear Polyethylenimine (Polysciences) in PBS at pH 4-5. This cotransfection mixture was incubated at room temperature for 10 minutes. Recombinant AAV of the PHP.eB serotype was generated by adding 0.61 mL of this cotransfection mixture to each of ten 15-cm dishes of HEK293T cells (ATCC) at 70-80% confluence. 24 hours post-transfection, cell medium was replaced with DMEM (with high glucose, L-glutamine and sodium pyruvate; ThermoFisher Scientific) with 4% FBS (Hyclone) and 1% Antibiotic-Antimycotic solution. Cells were collected 72 hours post transfection by scraping in 5 mL of medium, and were pelleted at 1500 rpm at 4° C. for 15 minutes. Pellets were suspended in a buffer containing 150 mM NaCl, 10 mM Tris, and 10 mM MgCl2, pH 7.6, and were frozen in dry ice. Cell pellets were thawed quickly in a 37° C. water bath, then passed through a syringe with a 21-23G needle 5 times, followed by 3 more rounds of freeze/thaw, and 30 minutes of incubation with 50 U/ml Benzonase (Sigma-Aldrich) at 37° C. The suspension was then centrifuged at 3,000×g and purified using a layered iodixanol step gradient (15%, 25%, 40%, and 60%) by centrifugation at 58,000 rpm in a Beckman 70Ti rotor for 90 minutes at 18° C. by extraction of a volume below the 40-60% gradient layer interface. Viruses were concentrated using Amicon Ultra-15 centrifugal filter unit by centrifugation at 3,000 rpm at 4° C., and reconstituted in PBS with 5% glycerol and 35 mM NaCl before storage at −80° C.

Retro-orbital injections. To introduce AAV viruses into the blood stream, 21 day old or older C57BI/6J, Ai14, Ai65F, or Ai63 mice (Madisen, et al., 2015, Neuron 85 (5): 942-958) were briefly anesthetized by isoflurane and 1×10¹⁰-1×10¹¹ viral genome copies (gc) was delivered into the retro-orbital sinus in a maximum volume of 50 μL or less. This approach has been utilized previously to deliver AAV viruses across the blood brain barrier and into the murine brain with high efficiency (Chan., et al., 2017, Nat Neurosci 20 (8): 1172-1179). For delivery of multiple AAVs, the viruses were mixed beforehand and then delivered simultaneously into the retro-orbital sinus. Animals were allowed to recover and then sacrificed 1-3 weeks post-infection in order to analyze virally-introduced transgenes within the brain.

Stereotaxic injections and tissue processing. Viral DNA was packaged in a PHP.eB serotype to produce recombinant adeno-associated virus (rAAV) for mscRE4-minBGprom-EGFP-WPRE3 (TG981), mscRE4-minBGprom-IRES2-tTa2-WPRE3 (TG1011), and mscRE4-minBGprom-FlpO-WPRE3 (TG978) viruses (titers: 1.64×1014, 5.11×1013, 6.00×1013, respectively), or self-complementary AAV (scAAV) for mscRE4-minBGprom-SYFP2-WPRE3-BGHpA (T502-057) virus (titer 1.34×10¹³) (Chan, et al., Nat. Neurosci 20:1172-1170, 2017). Each virus was delivered bilaterally at 250 nL and 50 nL into the primary visual cortex (VISp; coordinates: A/P:-3.8, ML:-2.5, DV: 0.6) of male and female C57BI6/J and wild-type transgenic mice (Htr2a-Cre (-), SST-IRES-Cre; Ai67 (-), Cck-IRES-Cre (-)) for rAAV-mscRE4-minBGprom-EGFP-WPRE3 and scAAV-mscRE4-minBGprom-SYFP2-WPRE3 viruses, or heterozygous Ai65F and Ai63 mice for rAAV-mscRE4-minBGprom-FlpO-WPRE3 and rAAV-mscRE4-minBGprom-tTa2-WPRE3 viruses, respectively, using a pressure injection system (Nanoject II, Drummond Scientific Company, Catalog #3-000-204). To mark the injection site, rAAV-EF1a-tdTomato or rAAV-EF1a-EGFP was co-injected at a dilution of 1:10 with experimental virus. The expression for all viruses was analyzed at 14 days post-injection. For tissue processing, mice were transcardially perfused with 4% paraformaldehyde (PFA) and post-fixed in 30% sucrose for 1-2 days. 50 μm sections were prepared using a freezing microtome and fluorescent images of the injections were captured from mounted sections using a Nikon Eclipse TI epi-fluorescent microscope.

Immunohistochemistry. Mice were transcardially perfused with 0.1M phosphate buffered saline (PBS) followed by 4% paraformaldehyde (PFA). Brains were removed, post-fixed in PFA overnight, followed by an additional incubation overnight in 30% sucrose. Coronal sections (50 um) were cut using a freezing microtome and native fluorescence or antibody-antibody enhanced was analyzed in mounted sections. To enhance the Enhanced Green Fluorescent Protein (EGFP) fluorescence, a rabbit anti-GFP antibody was used to stain free floating brain sections. Briefly, sections were rinsed three times in PBS, blocked for 1 hour in phosphate buffered saline (PBS) containing 5% donor donkey serum, 2% bovine serum albumin (BSA) and 0.2% Triton X-100, and incubated overnight at 4° C. in the anti-GFP primary antibody (1:2000; Abcam ab6556). The following day, sections were washed three times in PBS and incubated in blocking solution containing an Alexa® 488 conjugated secondary antibody (1:1500; Invitrogen), washed in PBS, and mounted in Vectashield containing DAPI (H-1500, Vector Labs). Epifluorescence images of native or antibody-enhanced fluorescence were acquired on a Nikon Eclipse Ti microscope or on a TissueCyte 1000 (Tissue Vision) system.

Virus titers were measured using quantitative PCR (qPCR) with a primer pair that recognizes a region of 117 bp in the AAV2 ITRs (Forward: GGAACCCCTAGTGATGGAGTT (SEQ ID NO: 175); Reverse: CGGCCTCAGTGAGCGA (SEQ ID NO: 176)). QPCR reactions were performed using QuantiTect SYBR Green PCR Master Mix (Qiagen) and 500 nM primers. To determine virus titers, a positive control AAV with known titer and newly produced viruses with unknown titers were treated with DNAse I. Serial dilutions (1/10, 1/100, 1/500, 1/2500, 1/12500, and 1/62500) of both positive control and newly generated viruses were loaded on the same qPCR plate. A standard curve of virus particle concentrations vs Cq values was generated based on the positive control virus, and the titers of the new viruses were calculated based on the standard curve.

Single cell RNA sequencing and cell type mapping. scRNA-seq was performed using the SMART-Seq v4 kit (Takara Cat #634894) as described previously (Tasic, et al., 2018, Nature 563:72-78). In brief, single cells were sorted into 8-well strips containing SMART-Seq lysis buffer with RNase inhibitor (0.17 U/μL), and were immediately frozen on dry ice for storage at −80 C. SMART-Seq reagents were used for reverse transcription and cDNA amplification. Samples were tagmented and indexed using a NexteraXT DNA Library Preparation kit (Illumina FC-131-1096) with NexteraXT Index Kit V2 Set A (FC-131-2001) according to manufacturer's instructions except for decreases in volumes of all reagents, including cDNA, to 0.4× recommended volume. Full documentation for the scRNA-seq procedure is available in the ‘Documentation’ section of the Allen Institute data portal at http://celltypes.brain-map.org/. Samples were sequenced on an Illumina HiSeq 2500 or Illumina MiSeq as 50 bp paired-end reads to a median depth of XX reads per cell. Reads were aligned to GRCm38 (mm10) using STAR v2.5.3 (Dobin, et al., 2013, Bioinformatics 29:15-21) in towpassMode, and exonic read counts were quantified using the GenomicRanges package for R as described in Tasic, et al., (2018, Nature 563:72-78). To determine the corresponding cell type for each scRNA-seq dataset, the scrattch.hicat package for R was utilized (Tasic, et al., 2018, Nature 563:72-78). Marker genes that distinguished each cluster were selected, then this panel of genes was used in a bootstrapped centroid classifier which performed 100 rounds of correlation using 80% of the marker panel selected at random in each round.

Physiology. Coronal mouse brain slices were prepared using the NMDG protective recovery method (Ting, et al., 2014, Methods Mol. Biol. 1183:221-242). Mice were deeply anesthetized by intraperitoneal administration of Advertin (20 mg/kg) and were perfused through the heart with an artificial cerebral spinal (ACSF) solution containing (in mM): 92 NMDG, 2.5 KCl, 1.25 NaH₂PO₄, 30 NaHCO₃, 20 HEPES, 25 glucose, 2 thiourea, 5 Na-ascorbate, 3 Na-pyruvate, 5 CaCl₂): 4H₂O and 10 MgSO₄: 7H₂O. Slices (300 μm) were sectioned on a Compresstome VF-200 (Precisionary Instruments) using a zirconium ceramic blade (EF-INZ10, Cadence). After sectioning, slices were transferred to a warmed (32-34° C.) recovery chamber filled with NMDG ACSF under constant carbogenation. After 12 minutes, slices were transferred to a holding chamber containing an ACSF made of (in mM) 92 NaCl, 2.5 KCl, 1.25 NaH₂PO₄, 30 NaHCO₃, 20 HEPES, 25 glucose, 2 thiourea, 5 Na-ascorbate, 3 Na-pyruvate, 128 CaCl₂) 4H₂O and 2 MgSO₄·7H₂O continuously bubbled with 95/5 O₂/CO₂.

For patch clamp recordings, slices were placed in a submerged, heated (32-34° C.) recording chamber that was continuously perfused with ACSF under constant carbogenation containing (in mM): 119 NaCl, 2.5 KCl, 1.25 NaH₂PO₄, 24 NaHCO₃, 12.5 glucose, 2 CaCl₂·4H₂O and 2 MgSO₄·7H₂O (pH 7.3-7.4). Neurons were viewed with an Olympus BX51WI microscope and infrared differential contrast optics and a 40× water immersion objective. Patch pipettes (3-6 MΩ) were pulled from borosilicate glass using a horizontal pipette puller (P1000, Sutter Instruments). Electrical signals were acquired using a Multiclamp 700B amplifier and PClamp 10 data acquisition software (Molecular Devices). Signals were digitized (Axon Digidata 1550B) at 10-50 kHz and filtered at 2-10 kHz. Pipette capacitance was compensated and the bridge balanced throughout whole-cell current clamp recordings. Access resistance was 8-25 MΩ).

Data was analyzed using custom scripts written in Igor Pro (Wavemetrics). All measurements were made at resting membrane potential. Input resistance (R_{N}) was calculated from the linear portion of the voltage-current relationship generated in response to a series of 1s current injections. The maximum and steady state voltage deflections were used to determine the maximum and steady state of R_{N}, respectively. Voltage sag was fined as the ratio of maximum to steady-state R_{N}. Resonance frequency (f_{R}) was determined from the voltage response to a constant amplitude sinusoidal current injection that either linearly increased from 1-15 Hz over 15 seconds or increased logarithmically from 0.2-40 Hz over 20 seconds. Impedance amplitude profiles were constructed from the ratio of the fast Fourier transform of the voltage response to the fast Fourier transform of the current injection. f_{R} corresponded to the frequency at which maximum impedance was measured. While the majority of neurons included in the examples currently described were located in primary visual cortex (n=10 YFP+, 10 YFP−), recordings from motor cortex (n=1 YFP+) and primary somatosensory cortex (n=4 YFP) were also made. For illustrative purposes, the properties of YFP+ and YFP− neurons to 32 L5 pyramidal neurons located in somatosensory cortex from an uninfected mouse were also compared. To classify these neurons as IT-like or PT-like, Divisive Analysis of Clustering (diana) from the cluster package in R was used (Maechler and Rousseeuw, 2012, R package version 1 (2), 56). Iₕ-related membrane properties are known to differentiate IT and PT neurons across many brain regions (Baker, et al., 2018, J. Neurosci. 38:5441-5455. As such, features included in clustering were restricted to the Ih-related membrane properties—sag ratio, R_{N} and f_{R}. To assess statistical significance of clustering, the sigclust package in R (Huang, et al., 2015, J Comput Graph Stat 24 (4): 975-993) was used.

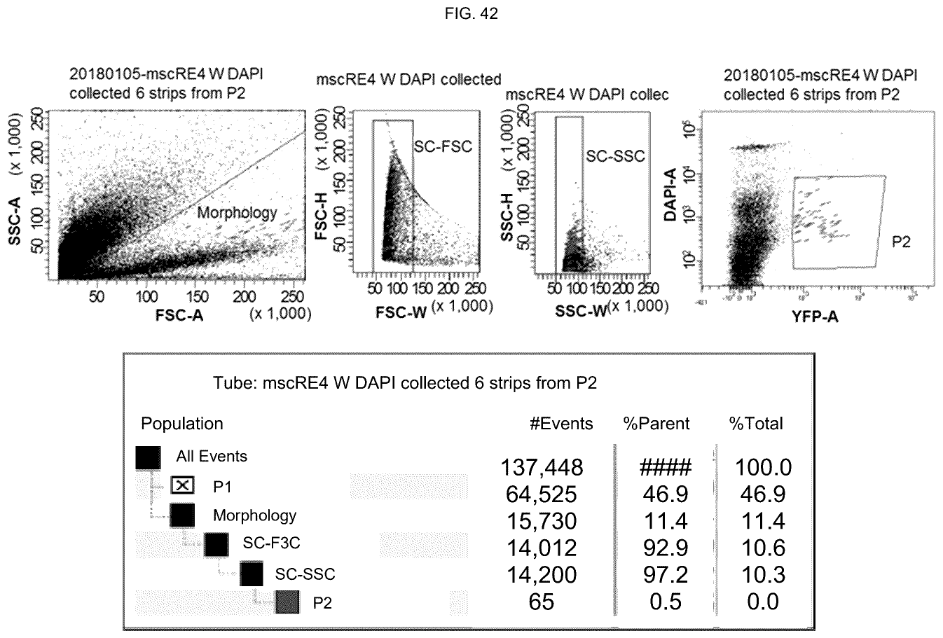

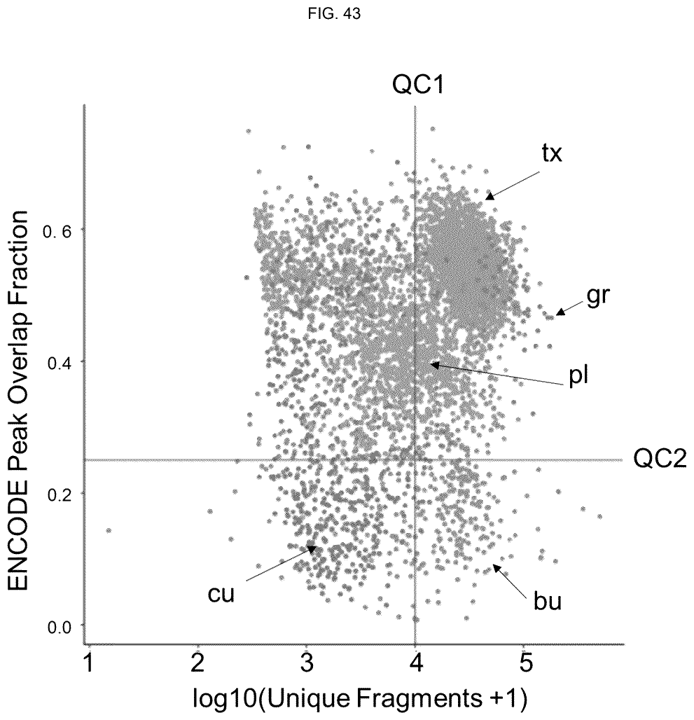

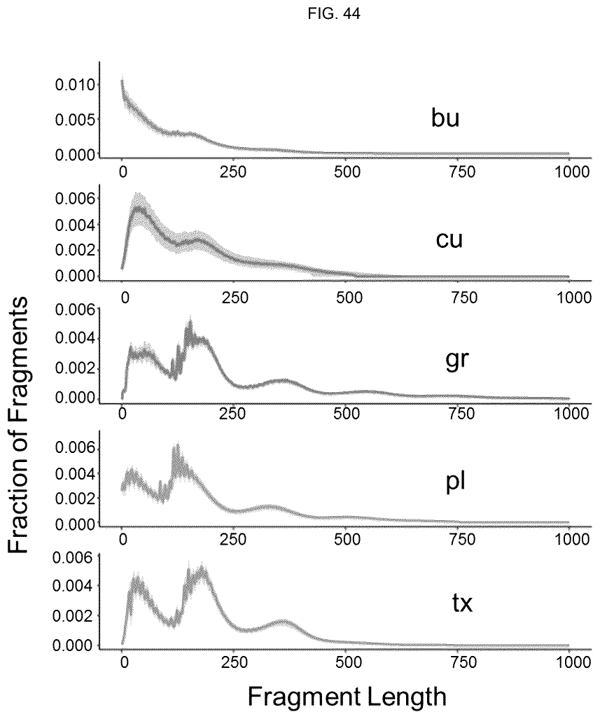

Example 2. Prospective, brain-wide labeling of neuronal subclasses with enhancer-driven adeno-associated virus (AAVs). Individual neuronal and non-neuronal cells from transgenically-labeled mouse cortex were isolated by Fluorescent Activated Cell Sorting (FACS) and examined using the Assay for Transposase-Accessible Chromatin with next generation sequencing (scATAC-seq). Buenrostro, et al., 2015, Nature 523:486-90); Cusanovich, et al., 2015, Science (80): 348, 910-914. This strategy allows for interrogation of both abundant (e.g. layer 4 intratelencephalic L4 IT neurons, 17% of primary visual area of the cortex, VISp, neurons) and very rare cell types (e.g. Sst Chodl neurons, 0.1% of VISp neurons) with the same method. To sample cells both broadly and specifically in the mouse brain, 25 different Cre or Flp-driver lines, or their combinations crossed to appropriate reporter lines, were utilized (FIG. 42). Many of the same lines were previously characterized by single-cell RNA-seq. Tasic, et al., 2018, Nature 563, 72-78. In addition, retrograde labeling by recombinase-expressing viruses was employed to selectively sample cells with specific projections (Retro-ATAC-seq). This method yielded scATAC-seq libraries of comparable quality to previously published scATAC-seq studies (FIGS. 43, 44). Buenrostro, et al., 2015, Nature 523, 486-90; Pliner et al., 2018, Mol. Cell 71, 858-871.e8; Cusanovich, et al., 2015, Science 348, 910-4.

To generate scATAC-seq data that would be directly comparable to the scRNA-seq dataset (Tasic, et al., 2018, Nature 563:72-78), the dissections were focused on visual cortex for glutamatergic cell types, but allowing broader cortical sampling for GABAergic cell types. This strategy is rooted in the observation that GABAergic cell types are shared across two distant poles of mouse cortex, whereas the glutamatergic cell types are distinct among different cortical regions. Tasic, et al., 2018, Nature 563:72-78. Retro-ATAC-seq cells were collected only from the visual cortex. In total, 3,381 single cells from 25 driver-reporter combinations in 60 mice, 126 retrogradely labeled cells from injections into 3 targets across 7 donors, and 96 samples labeled by one retro-orbital injection of a viral tool generated according to the current disclosure were collected. After FACS, individual cells were processed using ATAC-seq, and were sequenced in 60-96 sample batches using a MiSeq (Materials and Methods of Example 2). Quality control (QC) was performed by filtering to select 2,416 samples with >10,000 uniquely mapped paired-end fragments, >10% of which had a fragment size longer than 250 bp, and with >25% of fragments overlapping high-depth cortical DNAse-seq peaks generated by Encyclopedia of DNA Elements (ENCODE) (FIG. 42). Yue, et al., 2014, Nature 515:355-64.

Previous studies have shown that most recombinase driver lines label more than one transcriptomic cell type. Tasic, et al., 2018, Nature 563:72-78; Tasic, et al., 2016, Nat. Neurosci. 19, 335-346. To increase the cell type resolution of chromatin accessibility profiles beyond that provided by driver lines, the scATAC-seq data was clustered using a novel, feature-free method for computation of pairwise Jaccard distances. These distances were used for principal component analysis (PCA) and t-stochastic neighbor embedding (t-SNE), followed by Phenograph clustering (FIG. 45, Materials and Methods of Example 2). Levine, et al., 2015, Cell 162:184-197. This clustering method clearly grouped cells from class-specific driver lines together, and segregated them into multiple clusters as expected based on transcriptomic analyses. Cluster identity was then assigned by comparison of accessibility near transcription start sites (TSS+20 kb) to the scRNA-seq dataset generated for VISp using median correlation

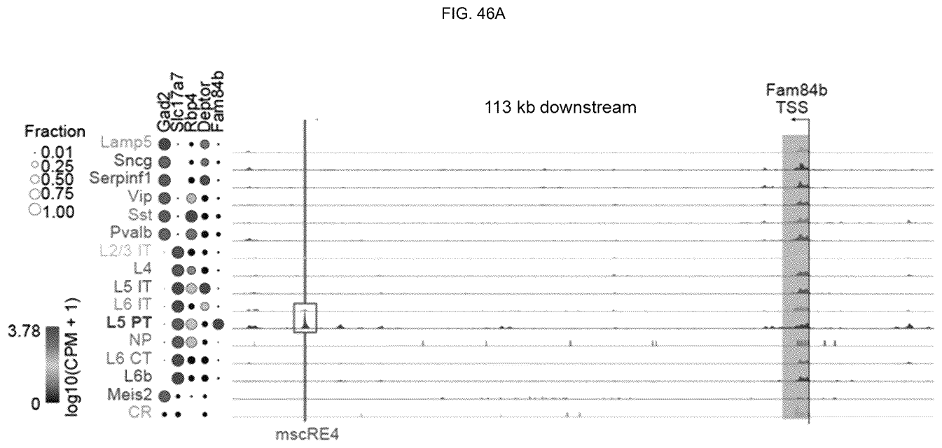

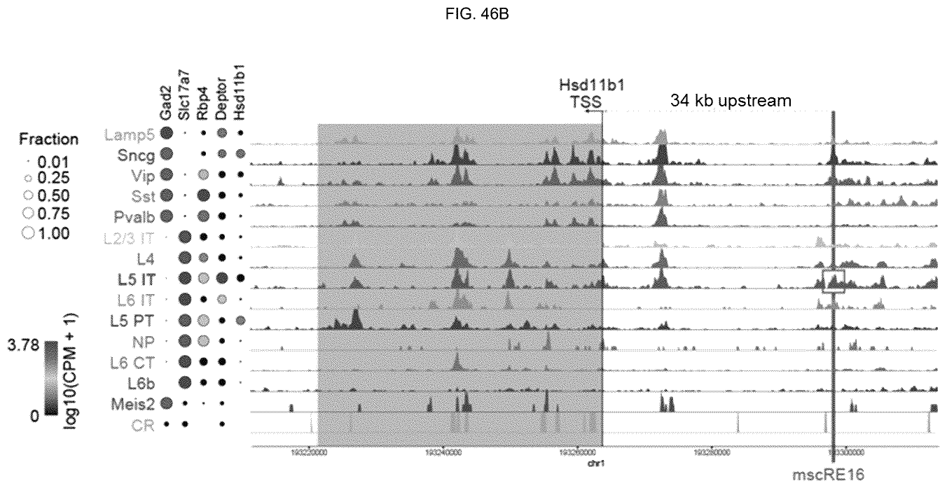

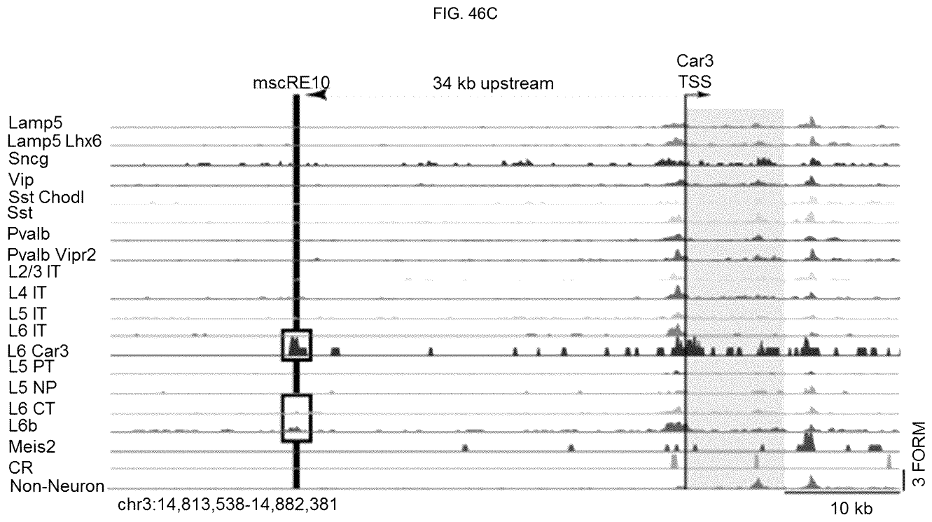

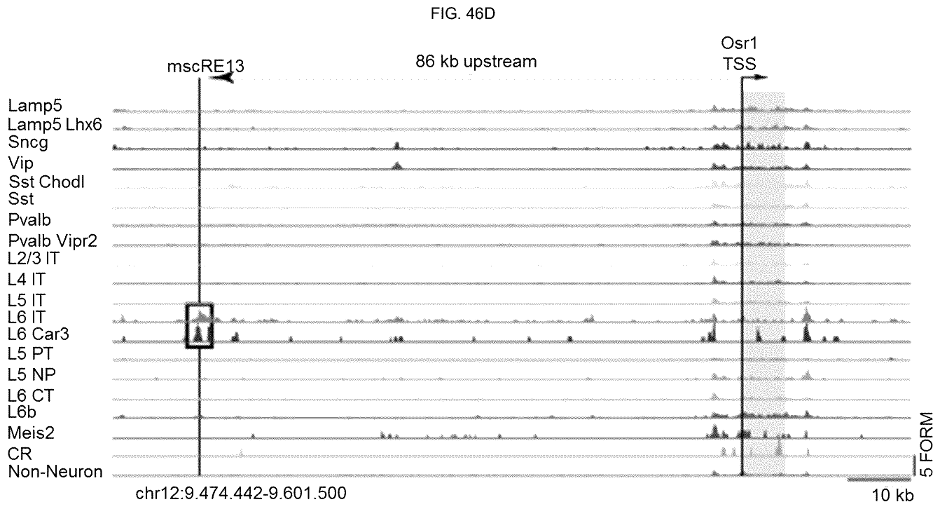

(FIG. 45, Materials and Methods of Example 2). Tasic, et al., 2018, Nature 563:72-78. Subclass-level assignments for each driver line were found to match closely with those observed for the same driver lines by scRNA-seq. Once assigned, clusters from the same subclass (e.g. Vip or layer 5, L5, IT) or distinct cell type (e.g. Pvalb Vipr2) were aggregated for peak calling and examination of accessibility patterns (FIGS. 46A-46D). Comparisons of these scATAC-seq aggregate profiles to previously published ATAC-seq from cortical populations showed strong correspondence between aggregate profiles and populations, and comparisons to previously published cortical scATAC-seq data demonstrate an increase in cell type resolution using the current dataset generated by this lab. Cusanovich, et al., 2018, Cell 174, 1309-1324.e18; Preissl, et al., 2018, Nat. Neurosci. 21:432-439.

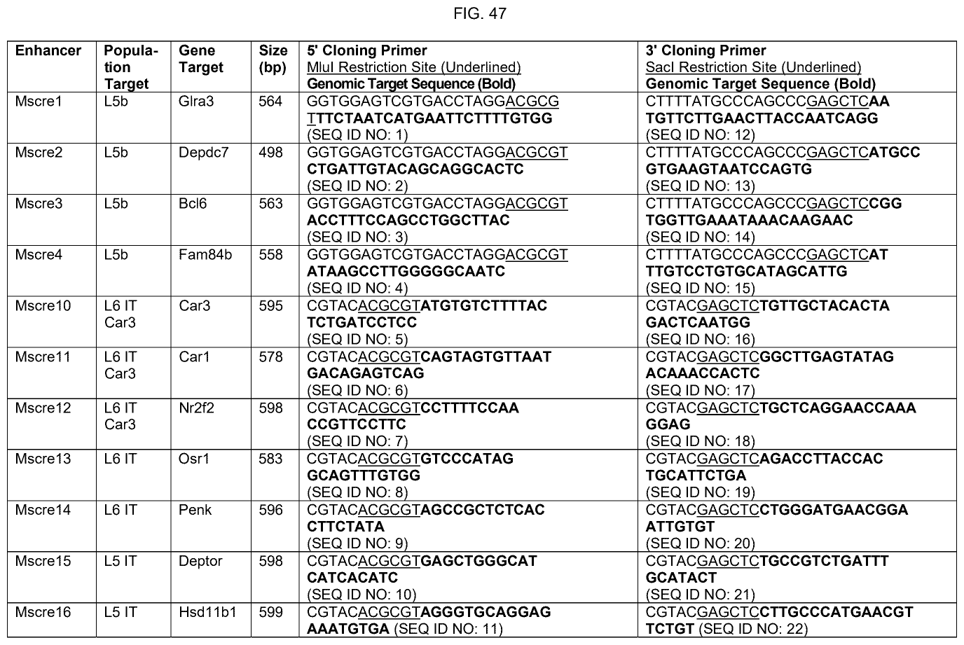

L5 of mouse cortex contains three major subclasses of excitatory neurons: intertelencephalic (IT) neurons that project to other cortical regions, near-projecting (L5 NP) neurons that have mostly local projections, and cortico-fugal (a subset of which is called pyramidal tract, L5 PT) neurons that project to subcortical brain regions such as the thalamus. Tasic, et al., 2018, Nature 563:72-78; Harris et al., bioRxiv, 2018 doi: 10.1101/292961. The driver line Rbp4-Cre labels both L5 IT and L5 PT neurons in cortex, but not L5 NP. Tasic, et al., 2018, Nature 563:72-78. The scATAC-seq clustering identified L5 PT and L5 IT neurons in the generated dataset based on correlation with scRNA-seq cell types (FIG. 45). Labeling of these cells by Rbp4-Cre and retrograde labeling from a known L5 PT target region, the lateral posterior nucleus of the thalamus (LP), validated that these cells are likely L5 IT (Rbp4-Cre+ only) and L5 PT neurons (Rbp4 and LP Retro-ATAC-seq). A search was performed near transcriptomic marker genes for 500 bp putative enhancer regions that were specific to L5 PT or L5 IT cells, and which had strong sequence conservation (FIG. 46A-46D). These regions are referred to as mouse single-cell regulatory elements (mscREs, FIG. 47).

To functionally test mscREs, their genomic sequences were cloned upstream of a minimal beta-globin promoter driving fluorescent proteins SYFP2 or EGFP in a recombinant adeno-associated virus (rAAV) genome (FIG. 48A). These constructs were packaged using a PHP.eB serotype, which can cross the blood-brain barrier, to enable delivery by retro-orbital injection. Four mscREs were screened for L5 PT cells and two for L5 IT (FIG. 47). Chan, et al., 2017, Nat. Neurosci. 20:1172-1179. Two weeks after retro-orbital injection, the brains of infected mice were collected and screened for expression by visual inspection of native fluorescence and immunohistochemistry to enhance SYFP2 and EGFP signal. Two of these enhancers provided specific labeling of cells in L5 (FIG. 48C, right) and were selected for further validation.

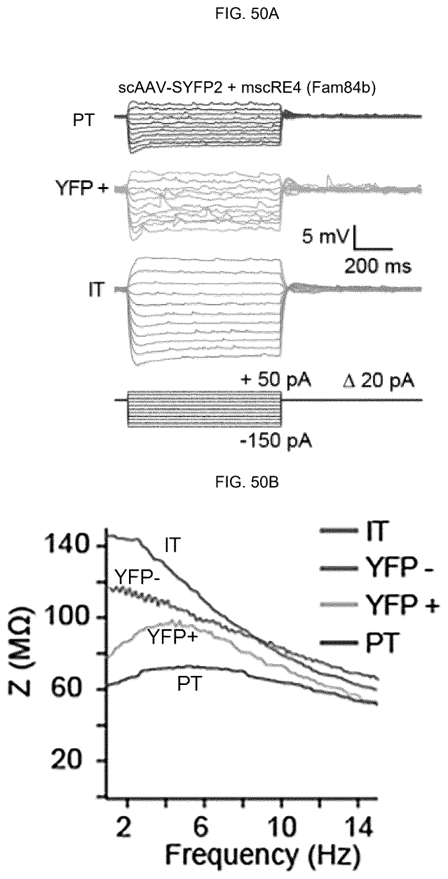

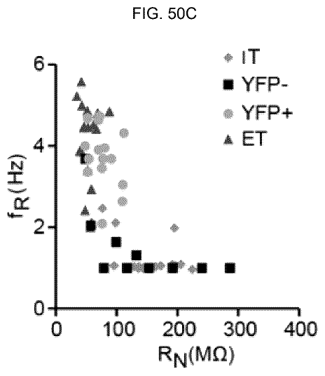

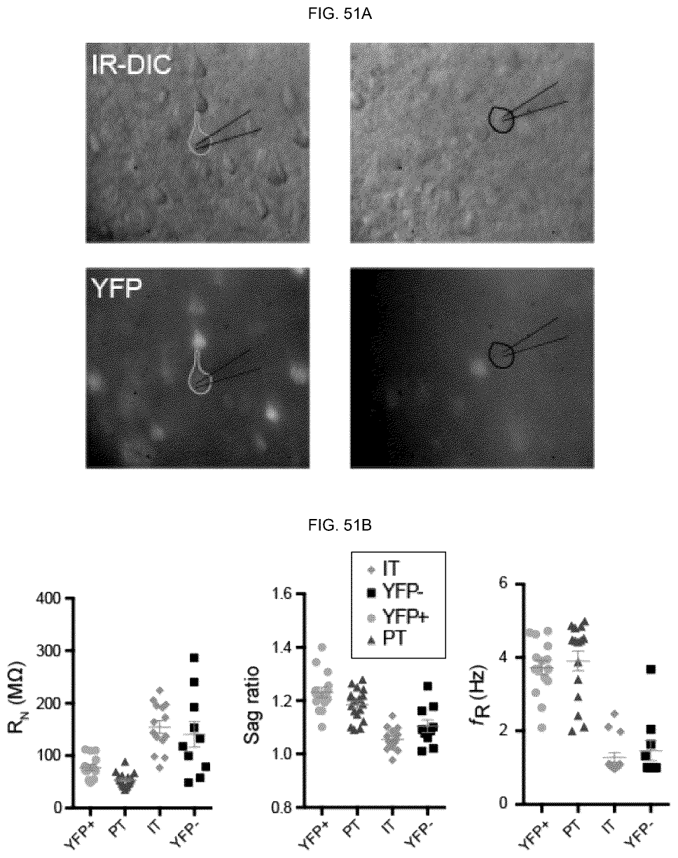

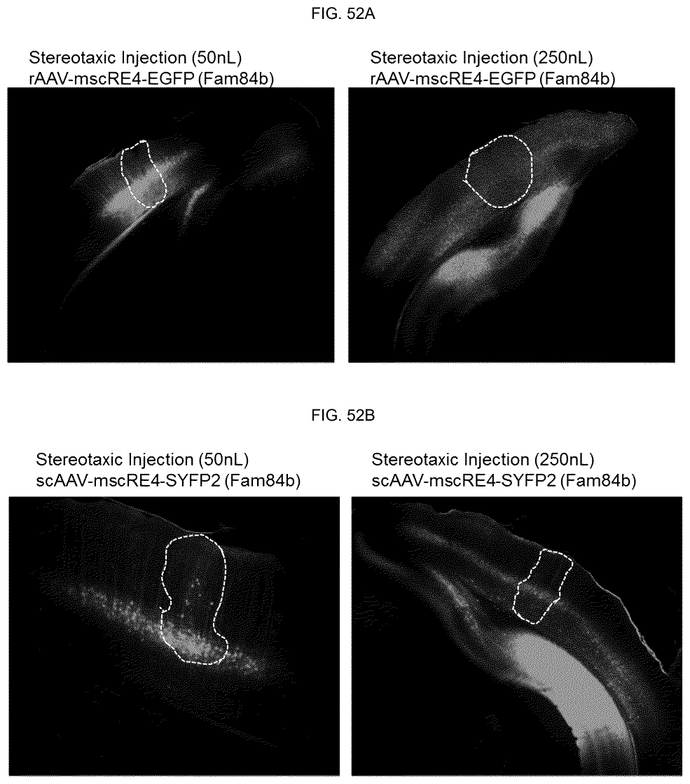

To assess the utility of enhancer-driven fluorophores as viral tools, a retro-orbital injection of the mscRE4-SYFP2 virus was performed in additional animals. From two of these, L5 of VISp was dissected, labeled cells were sorted by FACS, and scRNA-seq was performed as described previously. Tasic, et al., 2018, Nature 563:72-78. scRNA-seq expression profiles were compared to a VISp reference dataset using centroid classification of cell types (Materials and Methods of Example 2). Tasic, et al., 2018, Nature 563:72-78. The mscRE4-SYFP2 virus was found to yield >91% specificity for L5 PT cells within L5 (FIG. 49B). Labeling of L5 PT cells was confirmed by electrophysiological characterization of labeled vs unlabeled cells in the cortex (FIGS. 49B, 50A, 51). Cells labeled by mscRE4 had characteristics of L5 PT neurons, whereas cells that were label-negative more closely matched L5 IT neurons. Baker, et al., J. Neurosci. 38, 5441-5455, 2018. This experiment demonstrates the utility of these viral tools for electrophysiology experiments targeted to specific neuronal subclasses for which driver lines are not available. Finally, stereotaxic injection of the mscRE4 fluorophore viruses directly into VISp was tested. It was found that an extremely bright and specific labeling could be achieved by using stereotaxic injection, although the specificity depended on the volume of injection, likely reflecting a loss of specificity at high numbers of viral genome copies per cell (FIGS. 52A, 52B).

L5 PT cells are often difficult to isolate from single-cell suspensions when in a heterogeneous mixture with other cell types due to differential cell survival, and there is currently no reliable driver line to selectively label L5 PT cells. Tasic, et al., 2018, Nature 563:72-78; Tasic, et al., Nat. Neurosci. 19, 335-346, 2016. Retro-orbital injection of the mscRE4-SYFP2 virus was used to enhance the scATAC-seq generated dataset by sorting cells labeled by mscRE4 for FACS. As expected based on scRNA-seq analysis, 55 of 61 high-quality mscRE4 scATAC-seq profiles clustered together with other L5 PT samples (90.2%).

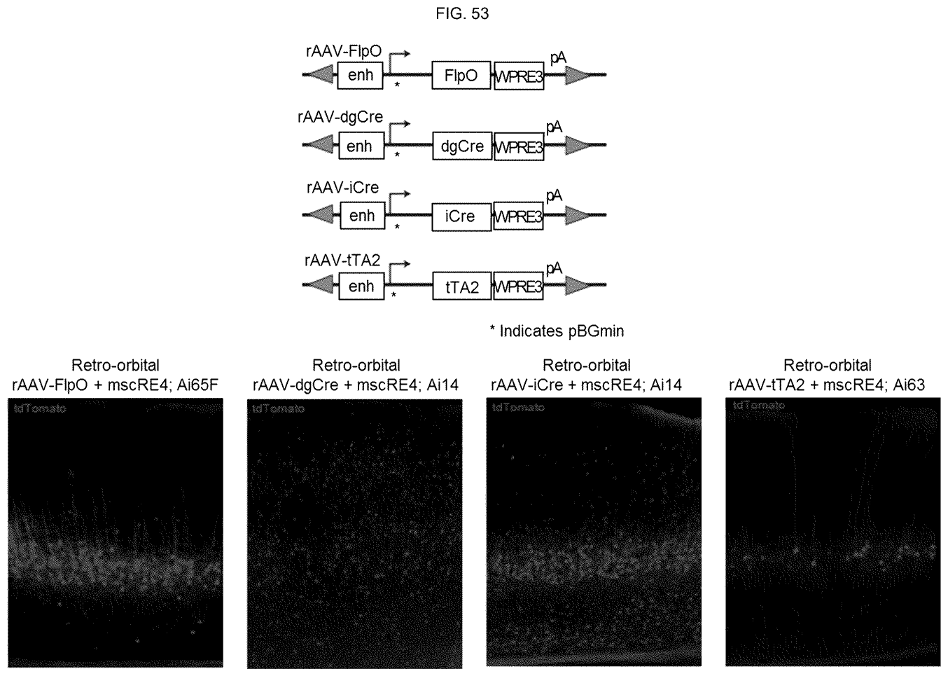

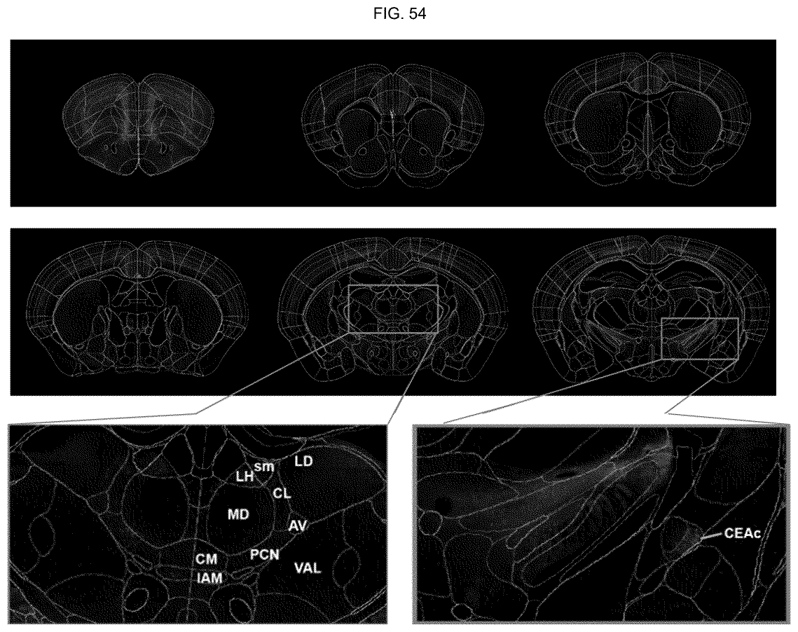

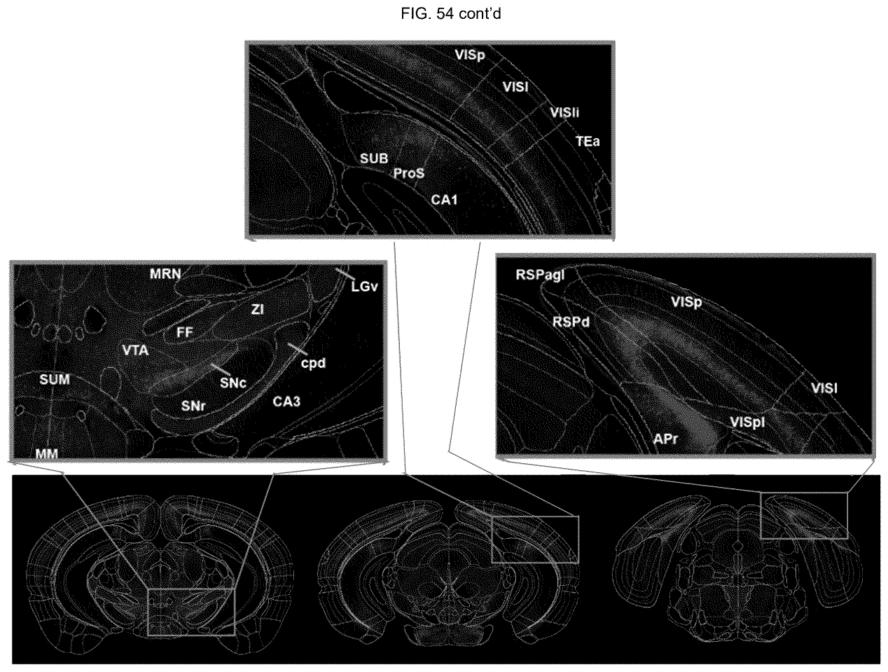

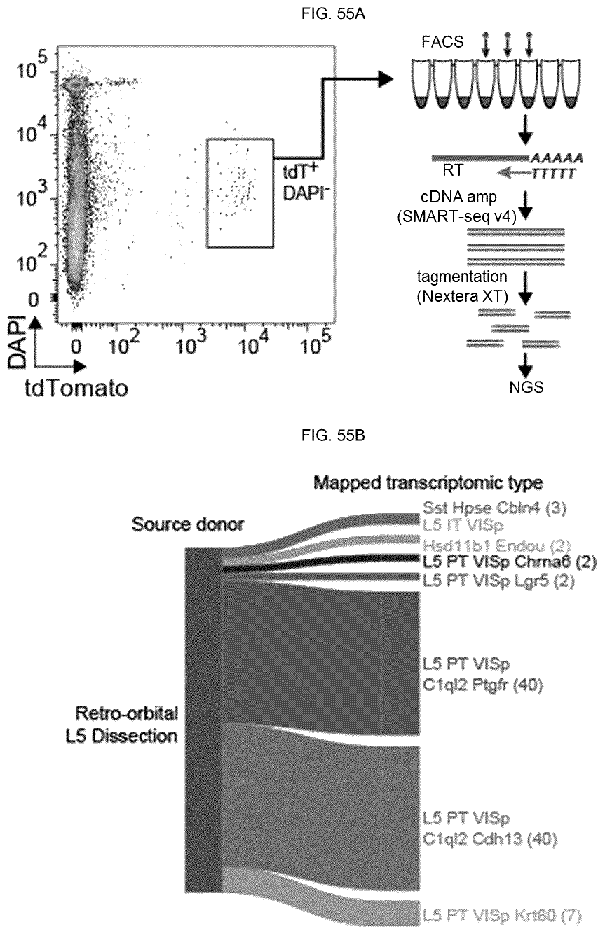

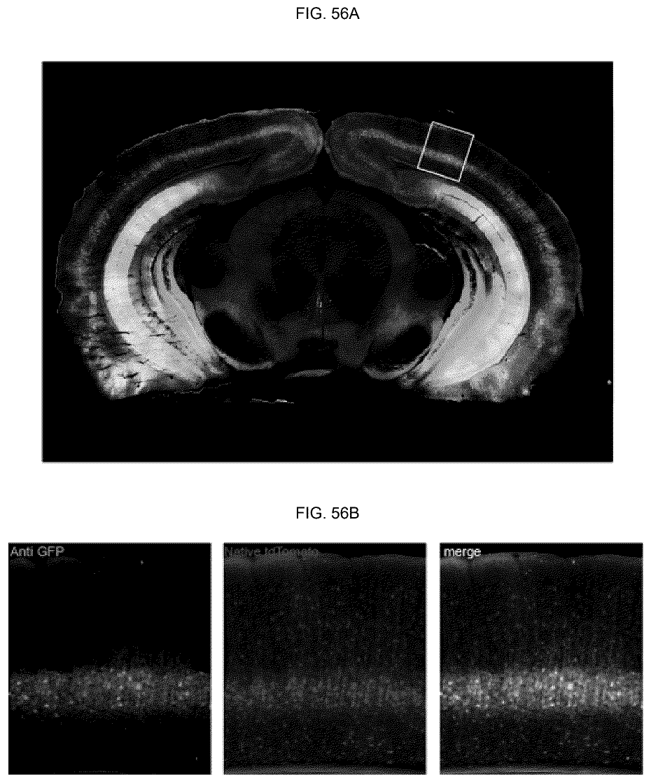

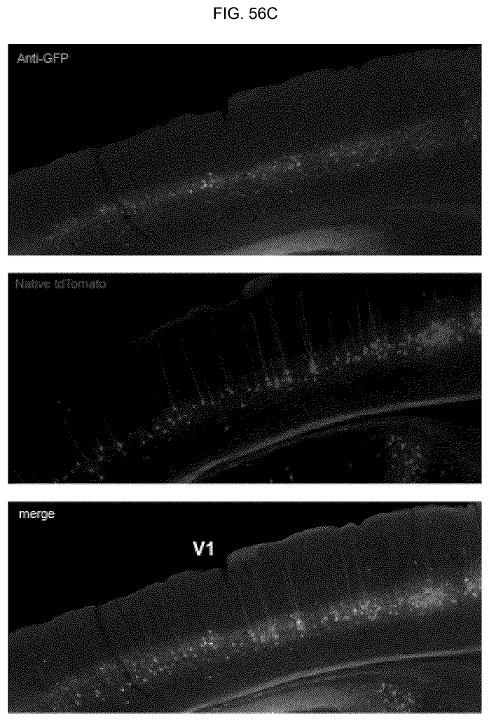

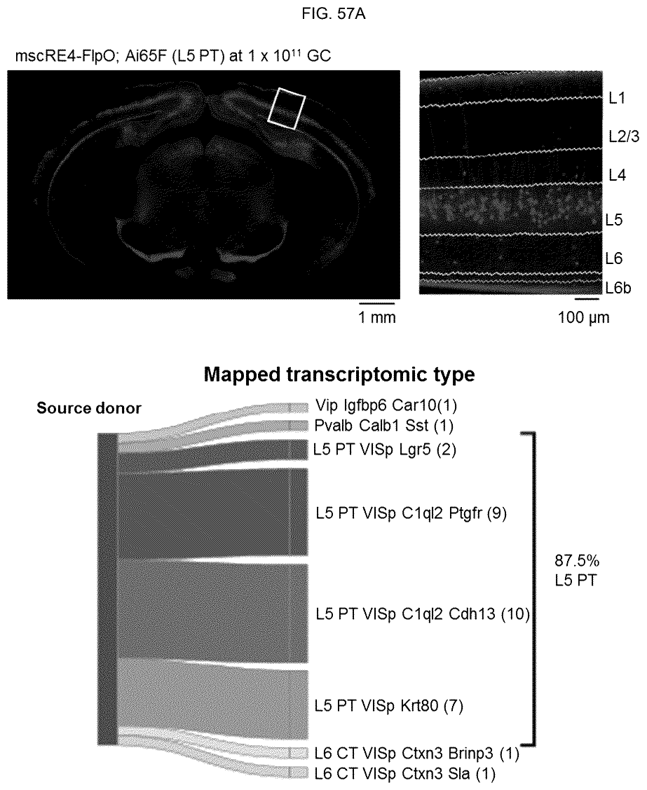

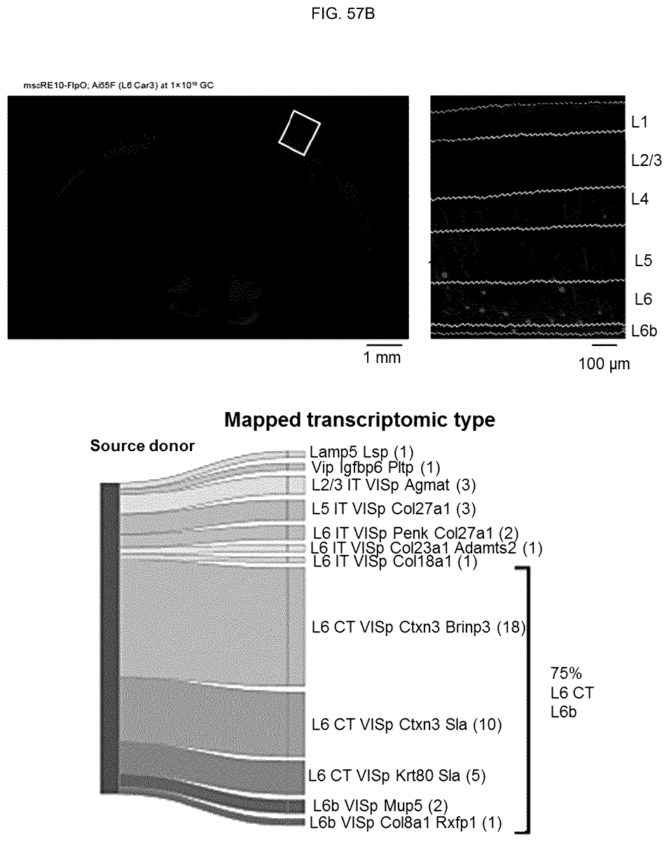

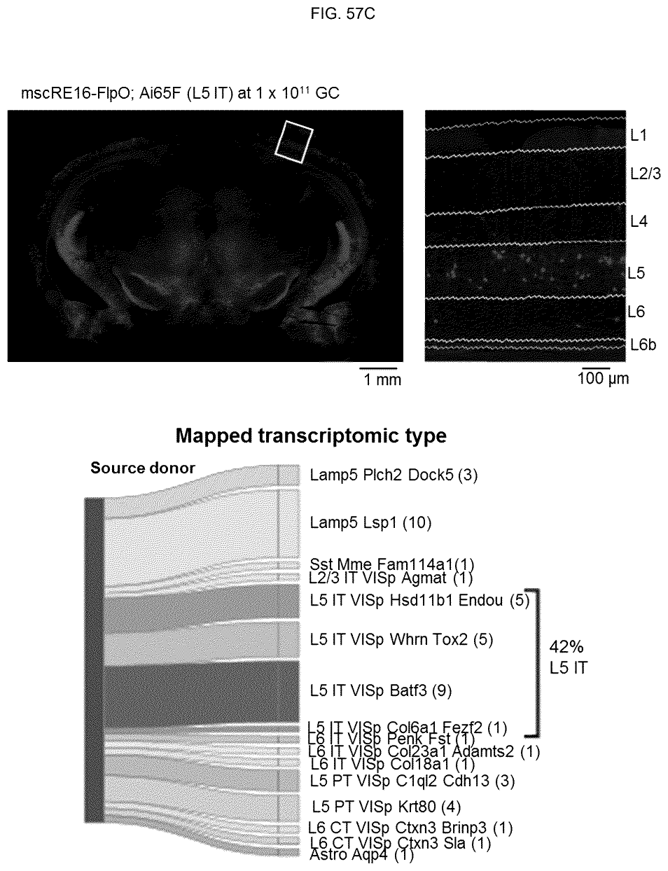

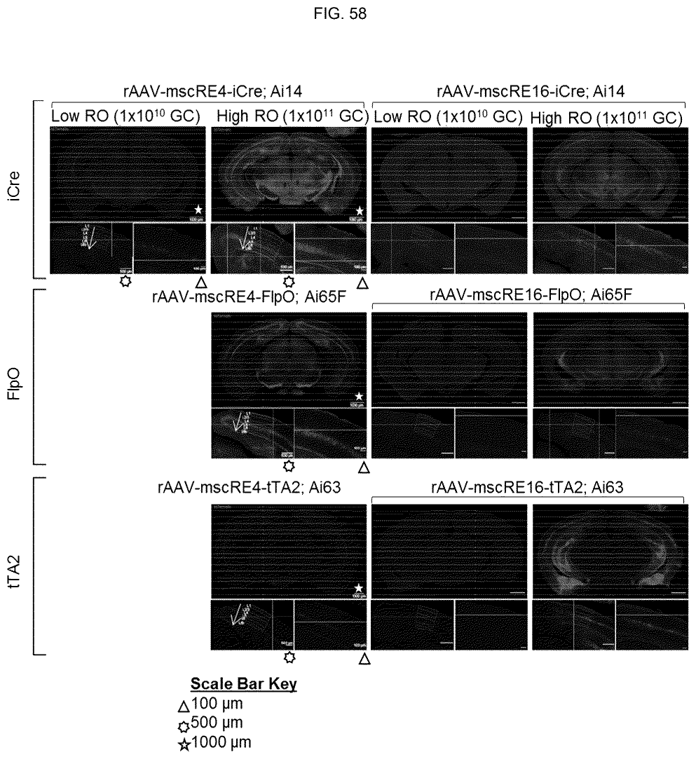

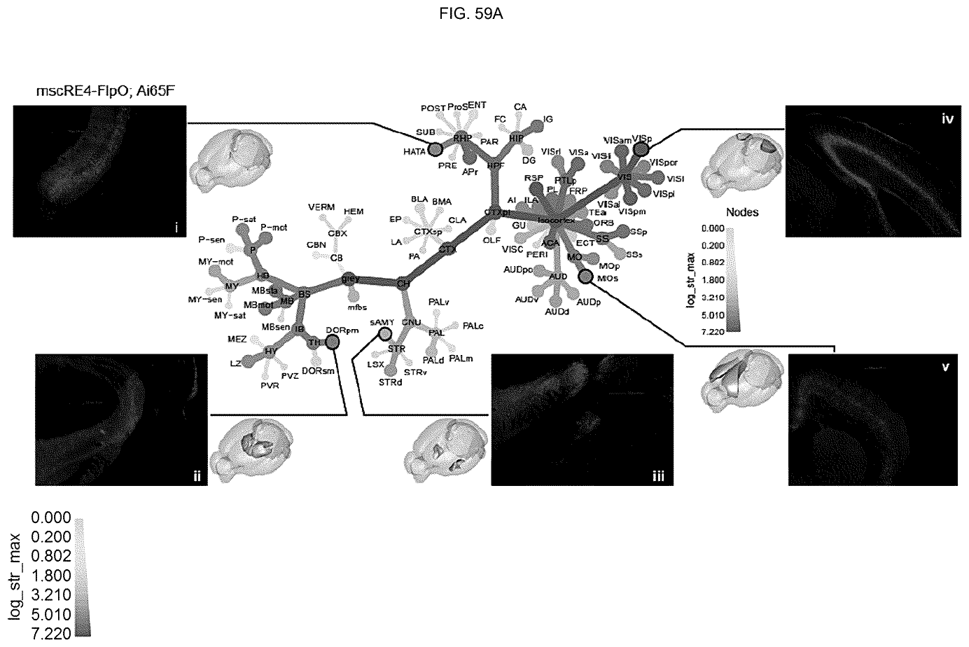

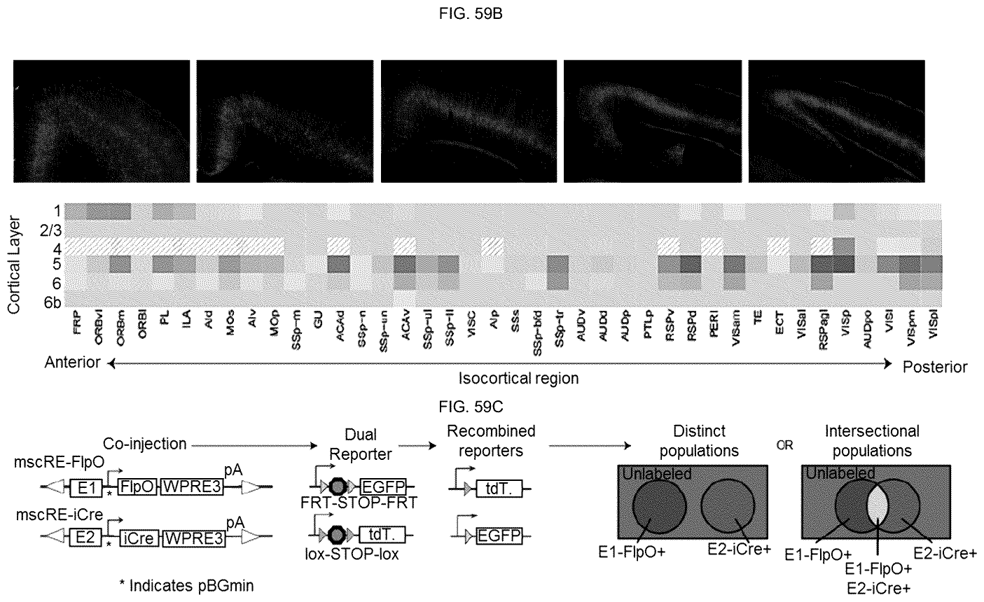

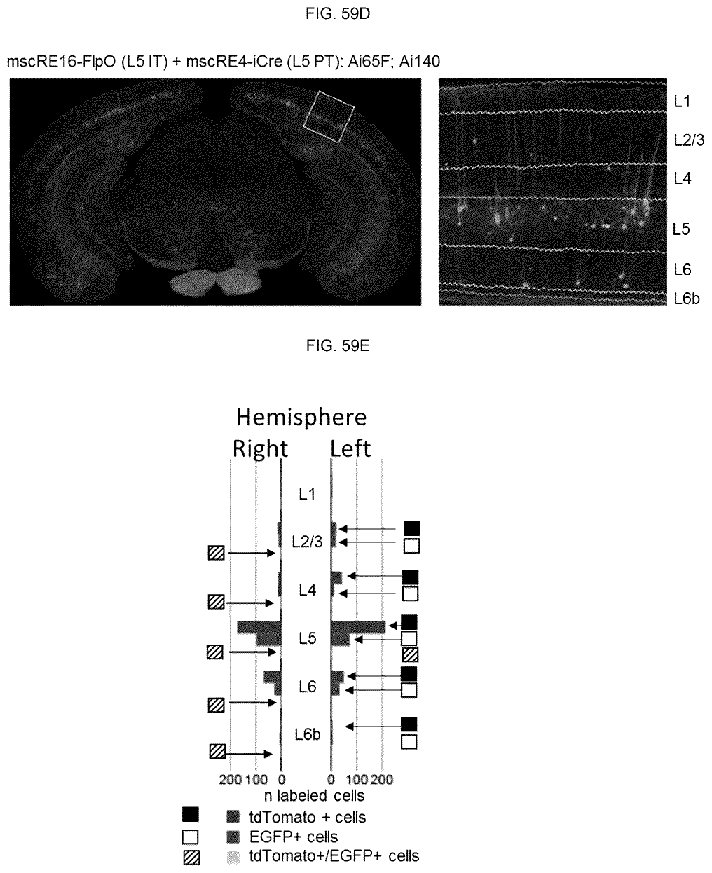

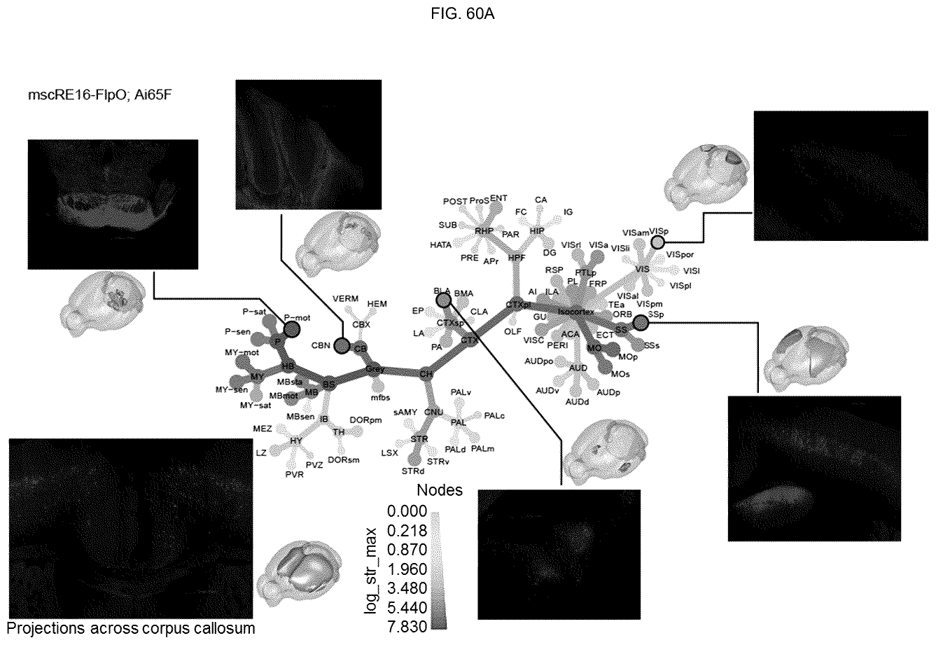

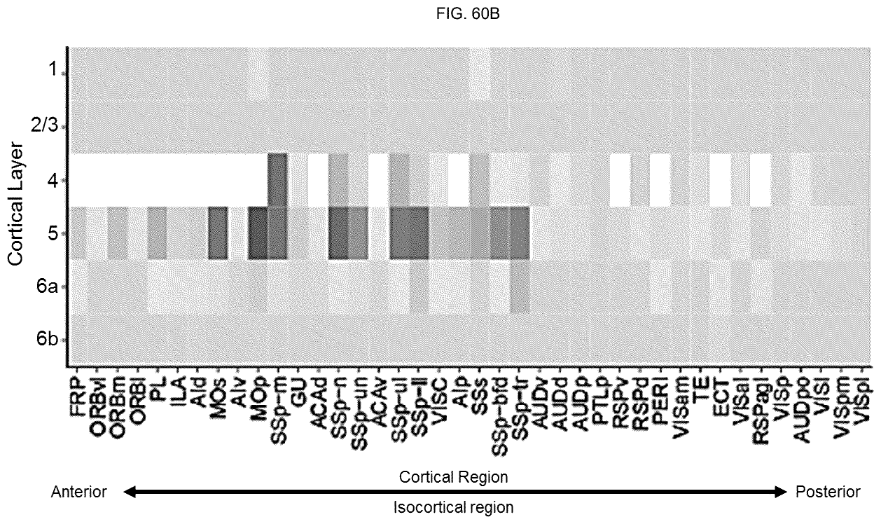

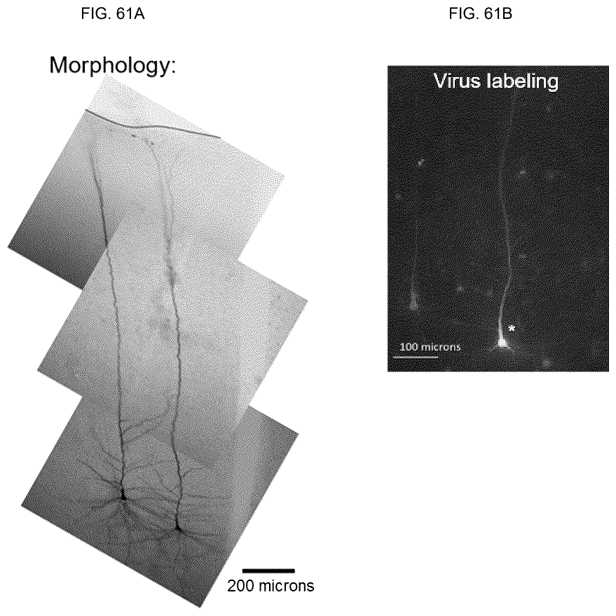

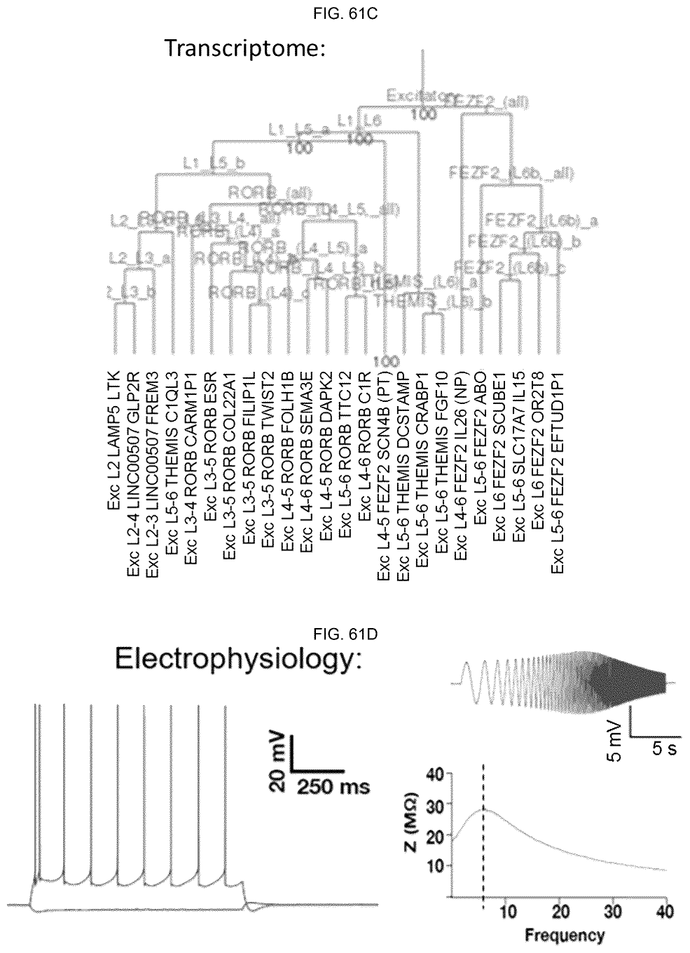

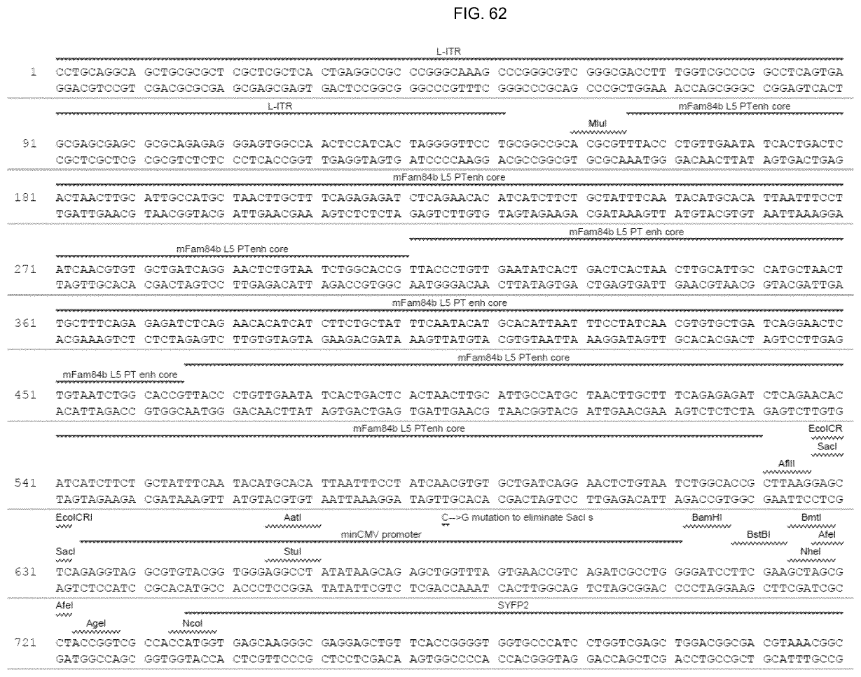

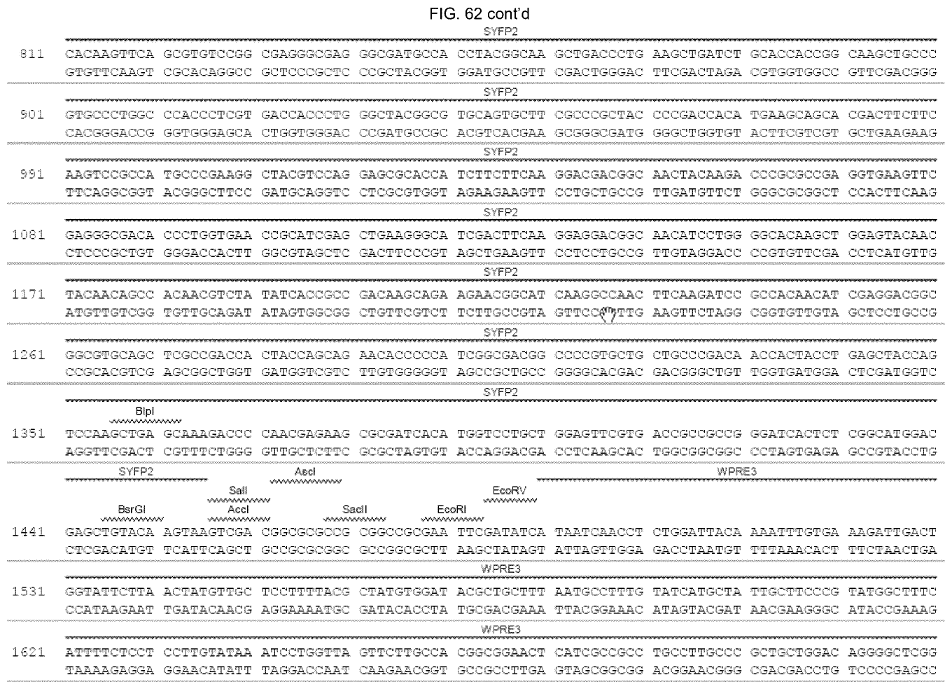

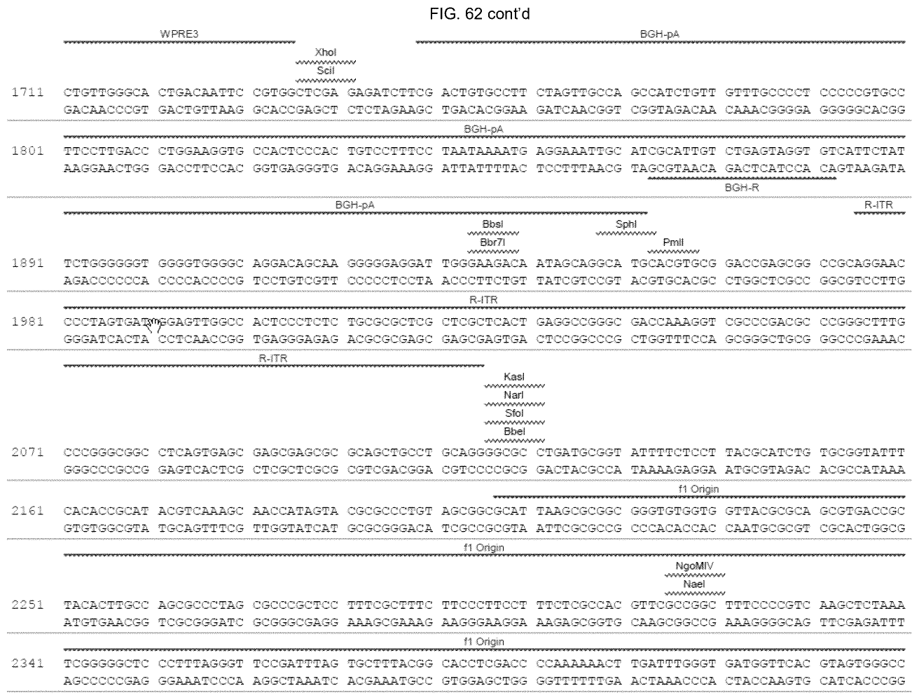

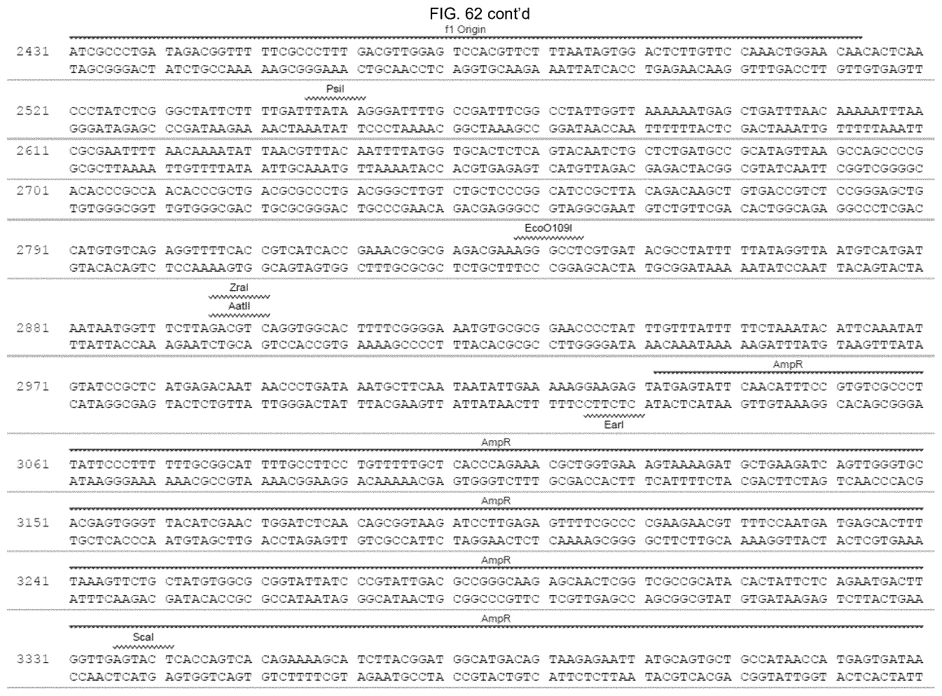

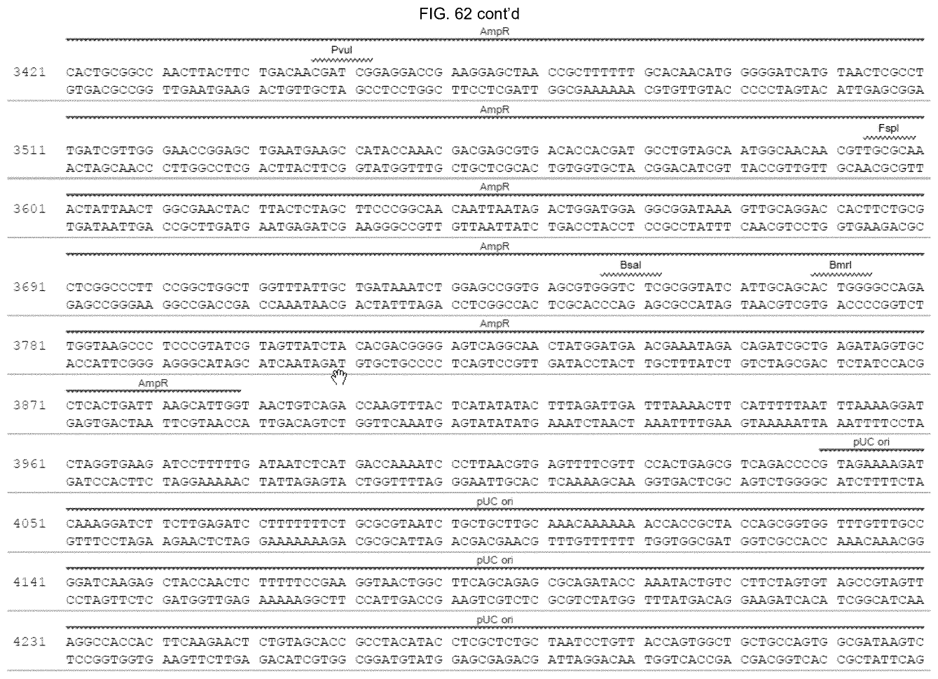

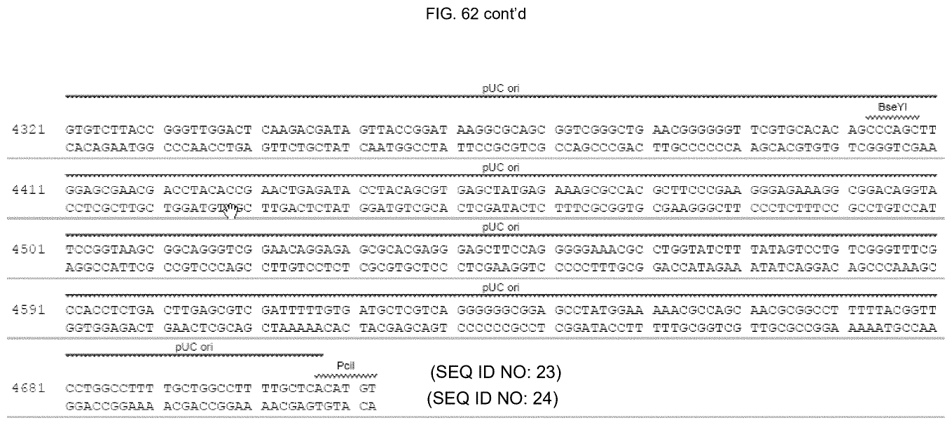

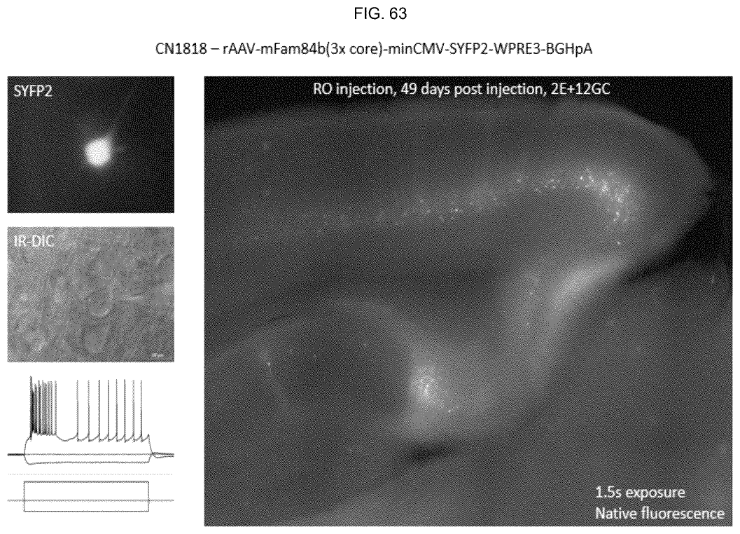

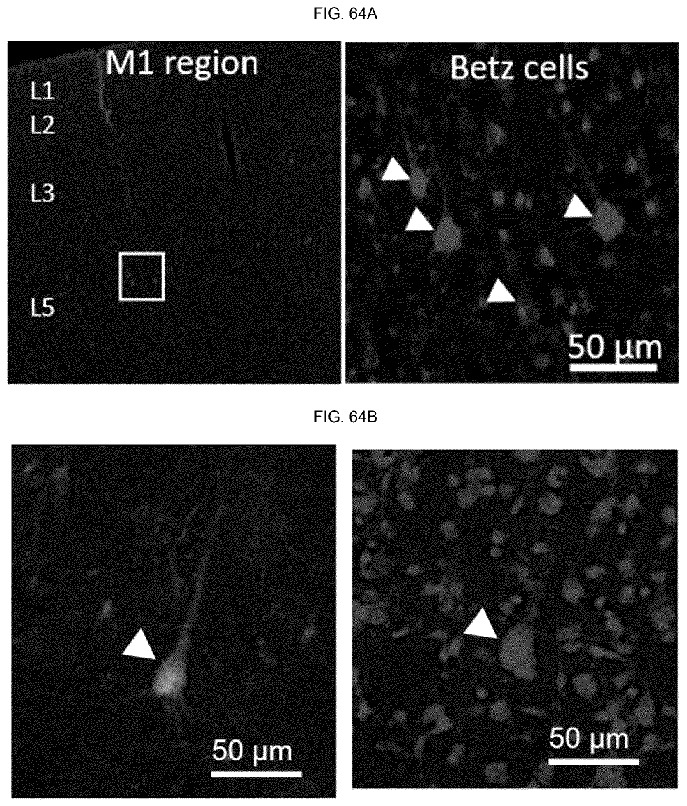

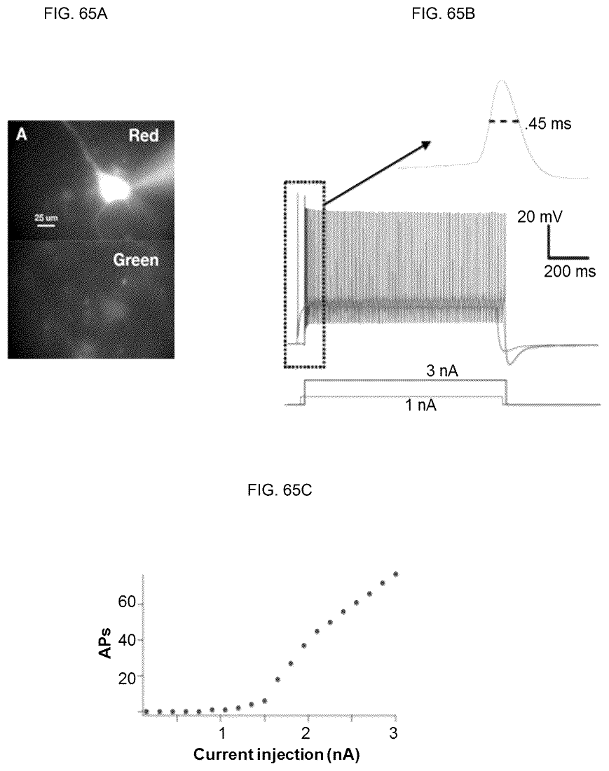

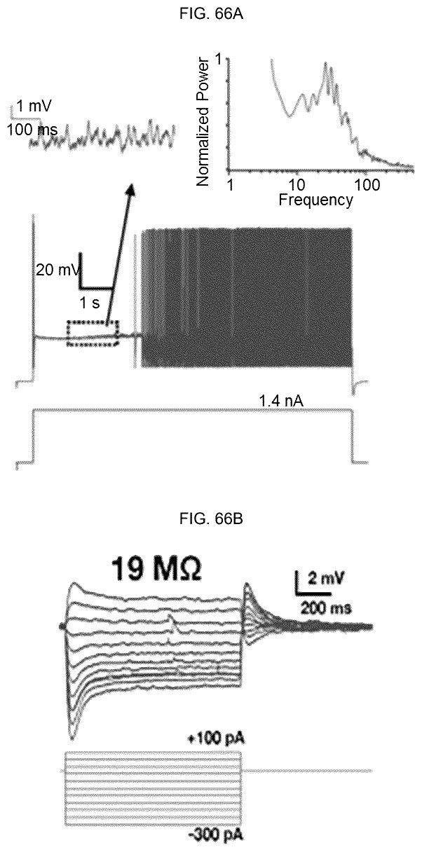

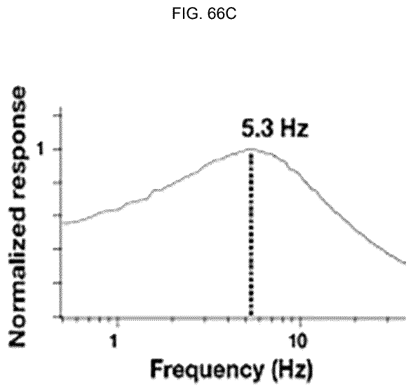

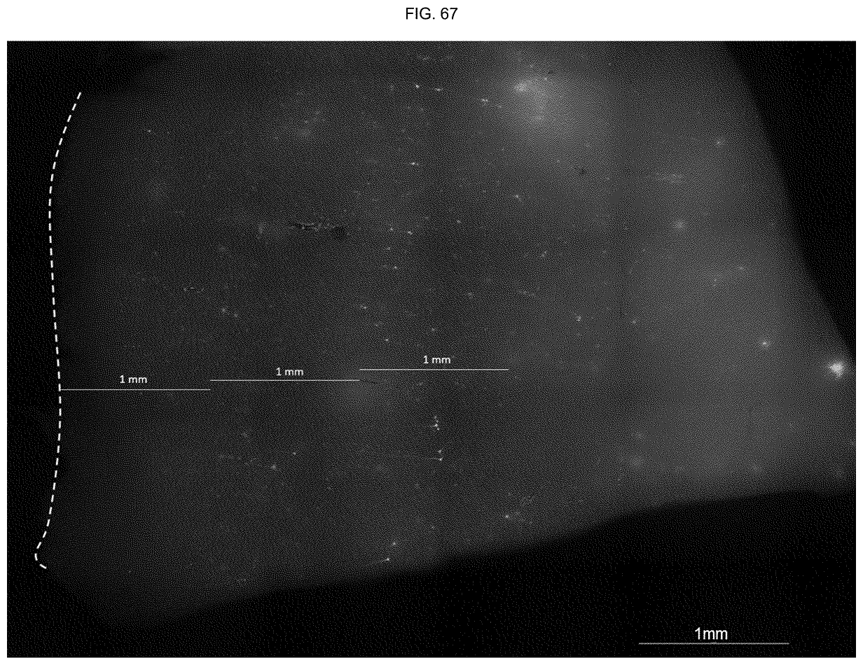

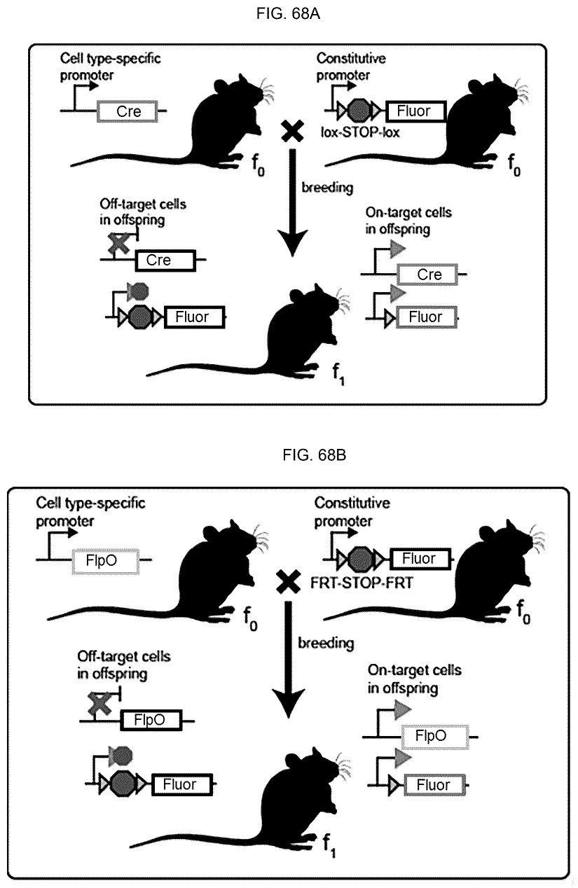

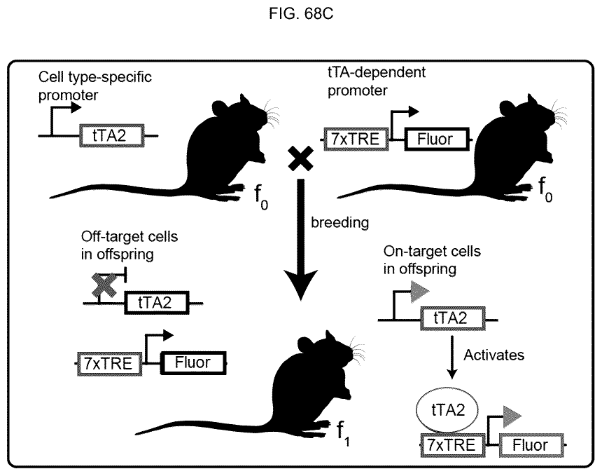

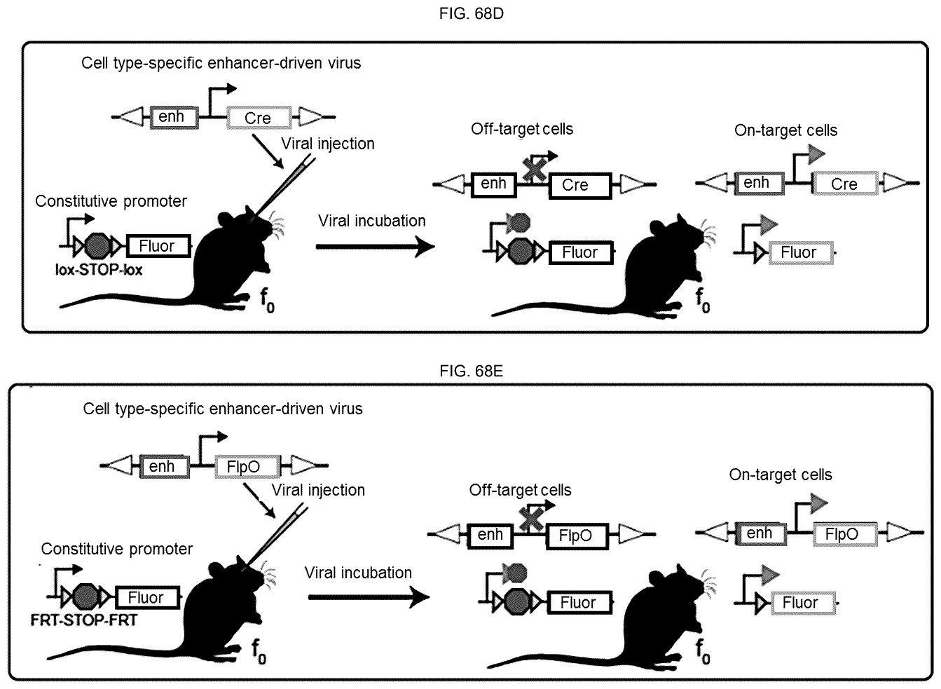

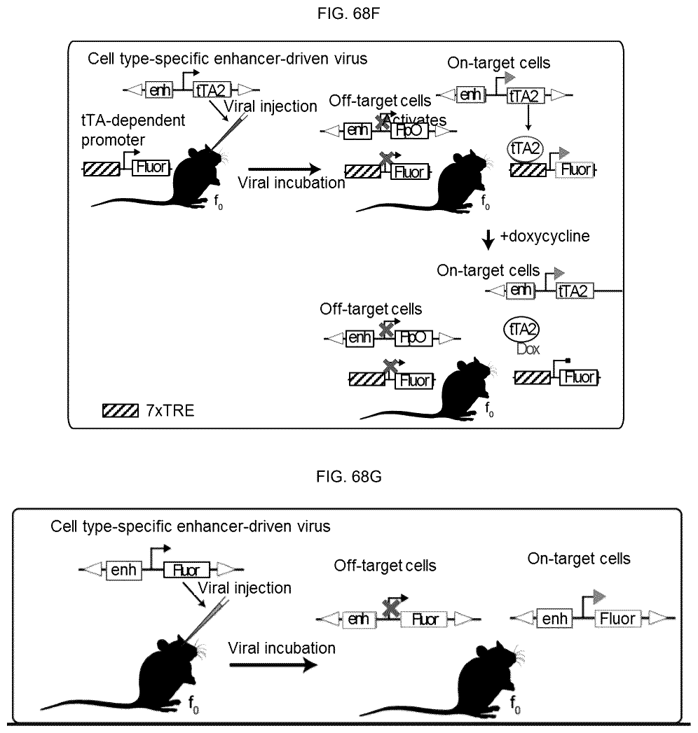

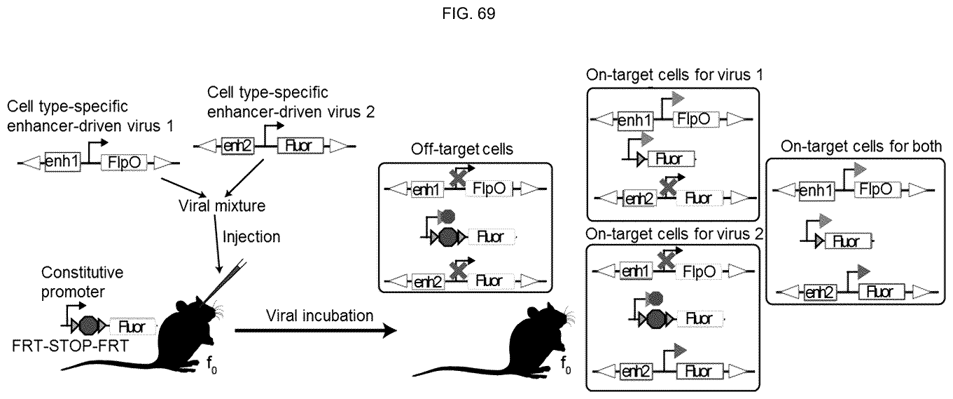

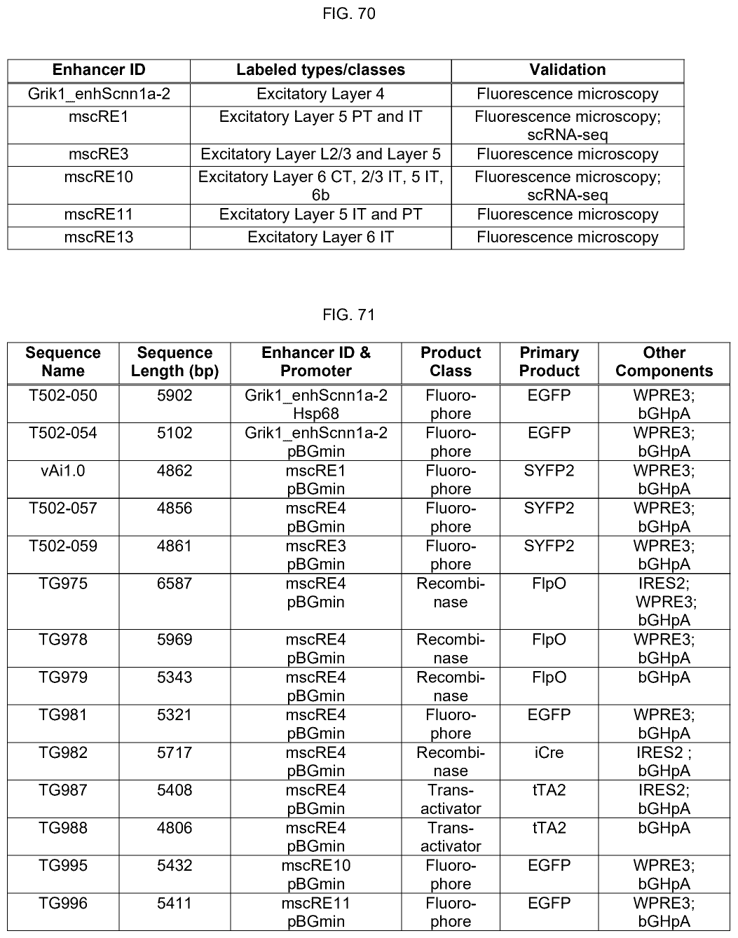

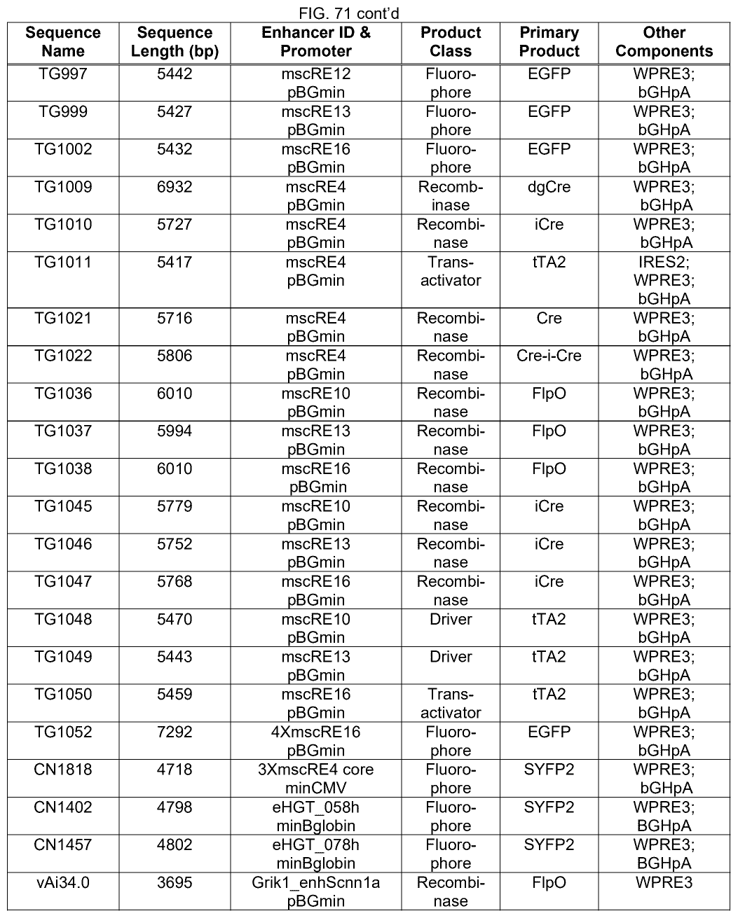

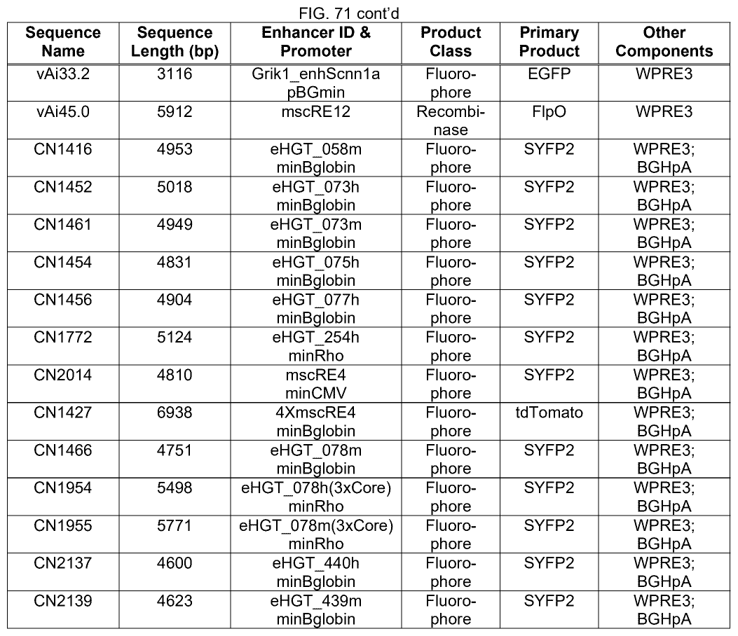

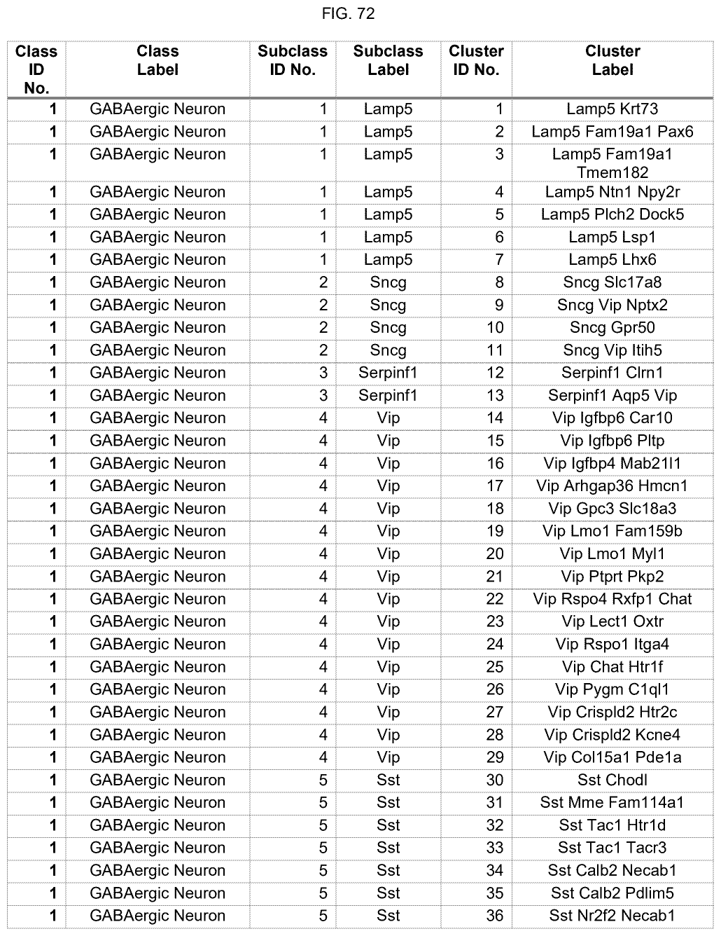

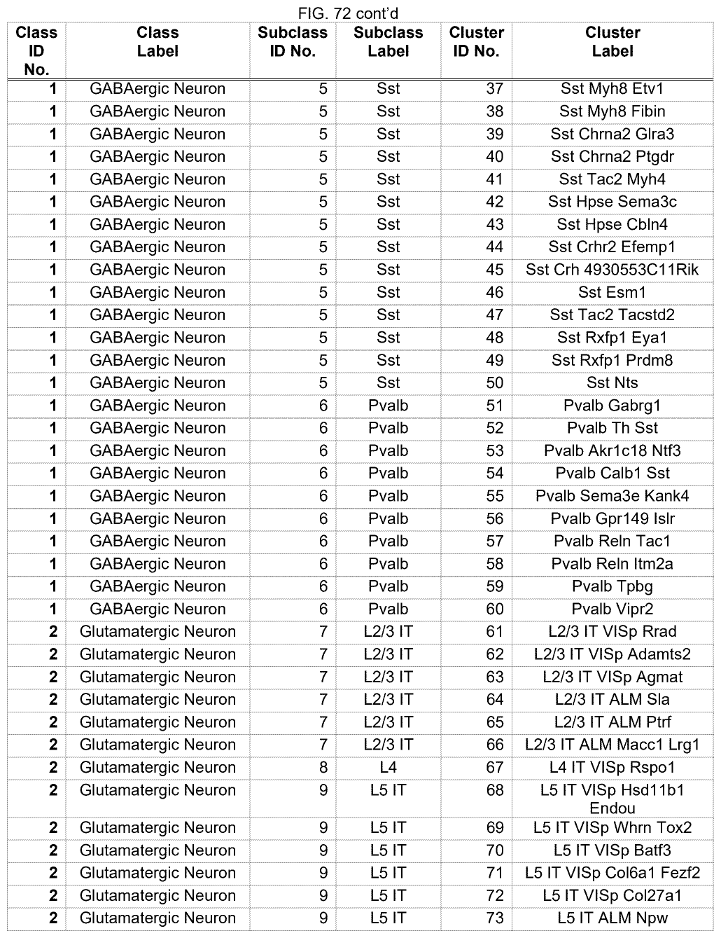

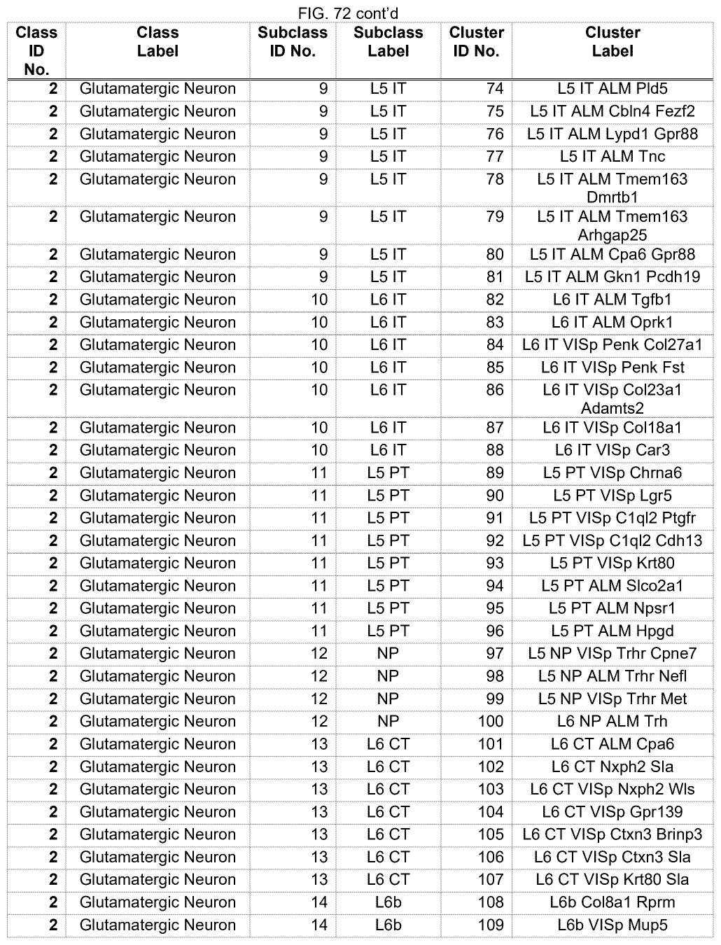

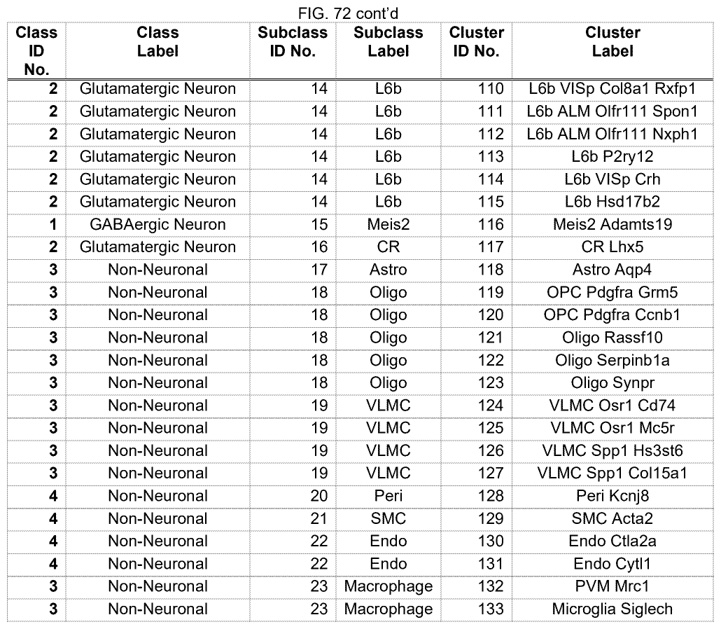

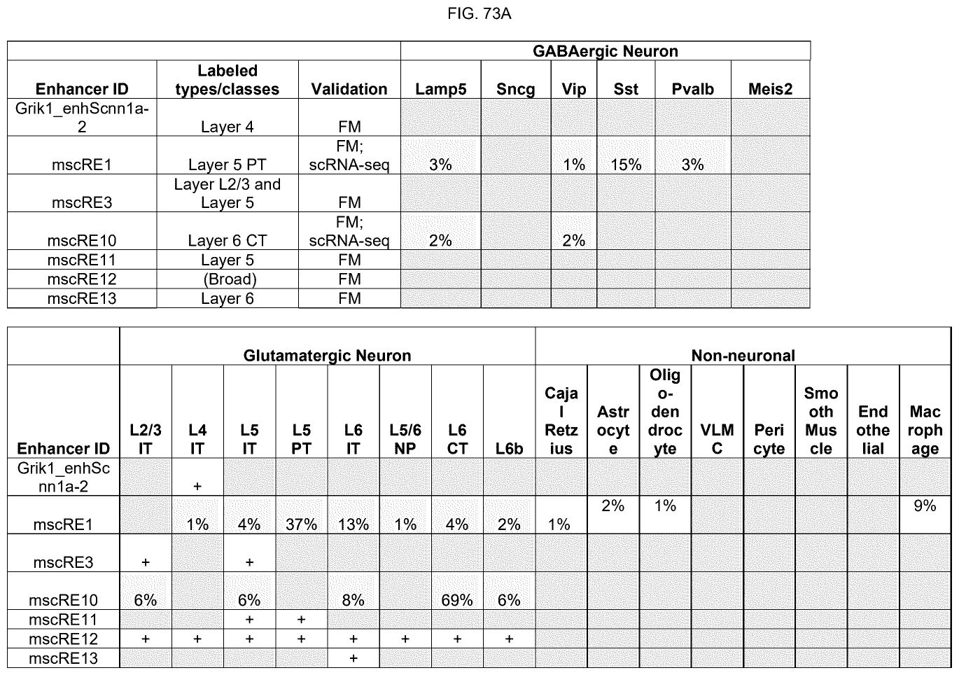

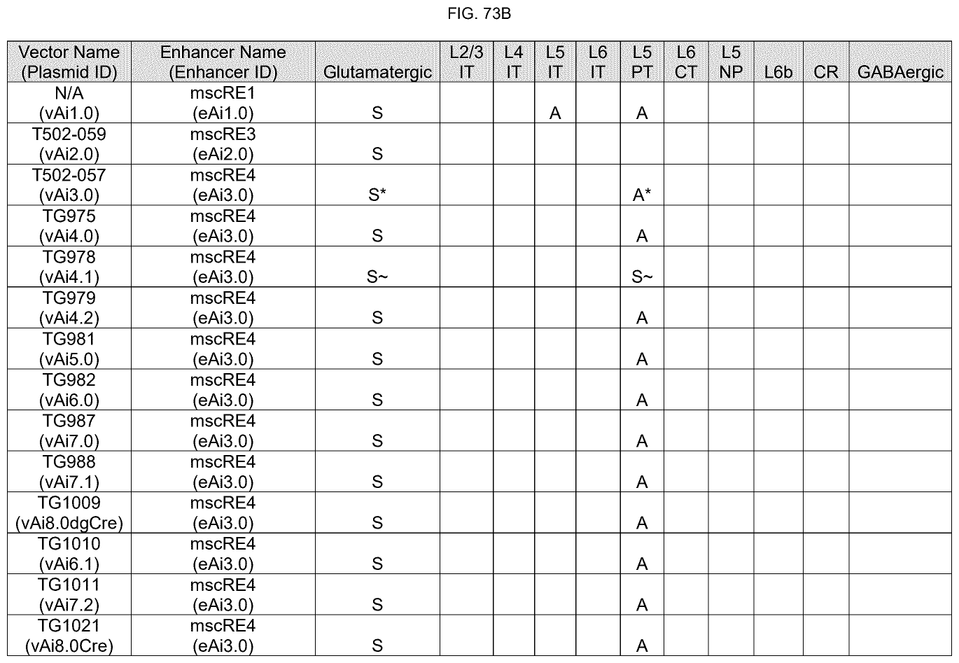

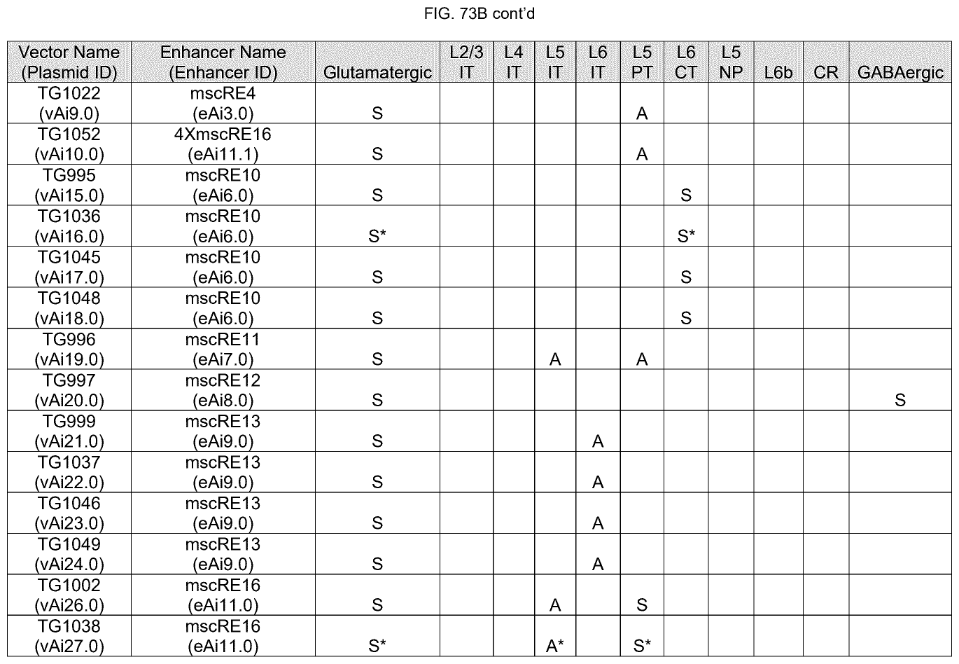

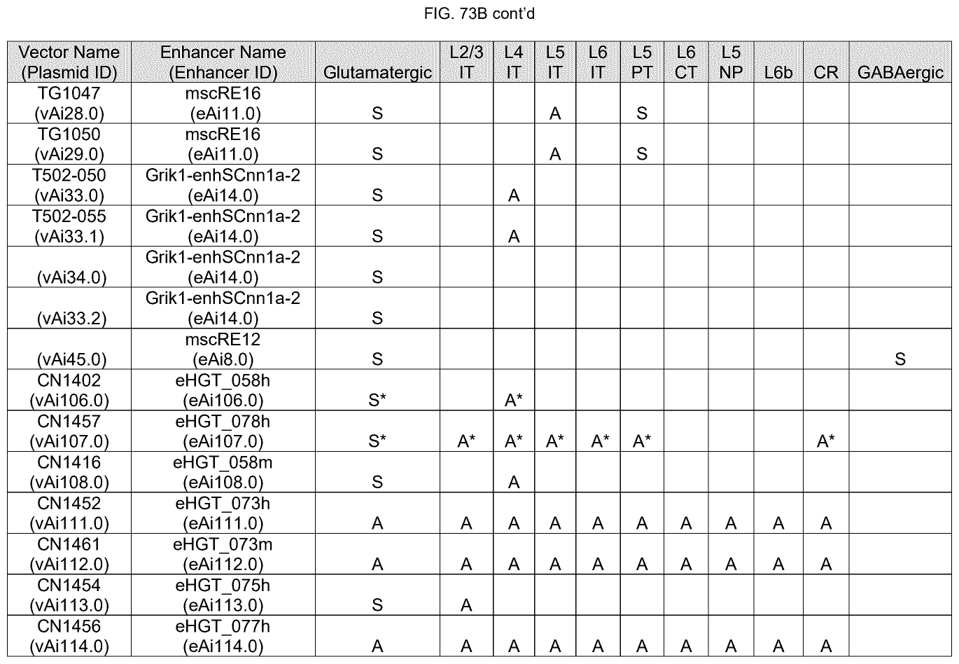

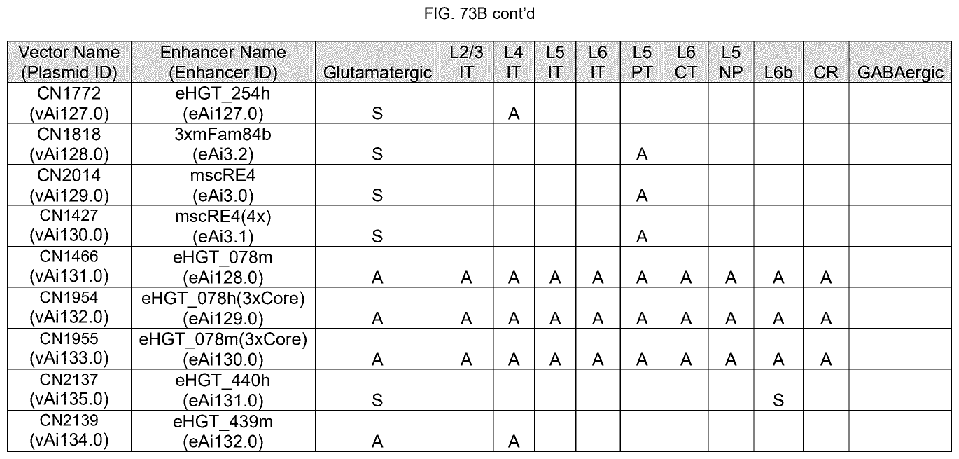

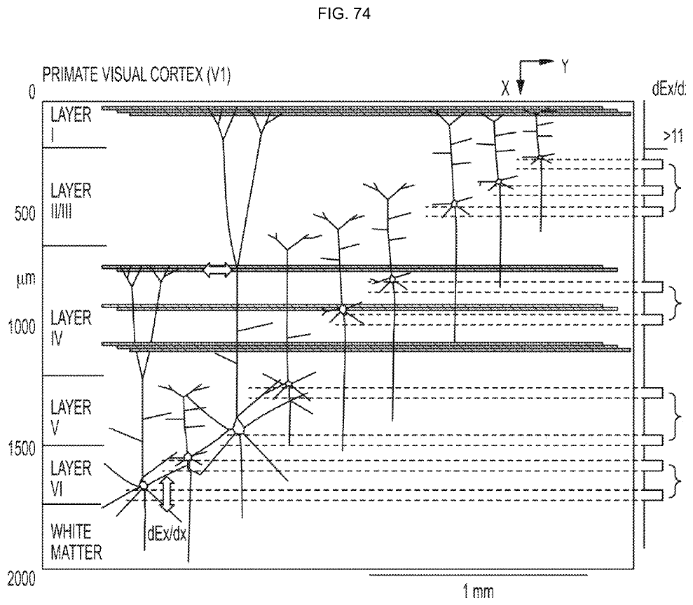

Although fluorophore expression provided enough signal to sort cells by FACS or perform patch-clamp experiments, expression of a recombinase from a specific enhancer virus would expand the utility of these tools as drivers for reporter lines that express fluorophores, activity reporters, opsins, or other genes that are too large to package in AAVs. Daigle, et al., 2018, Cell 174 (2): 465-480; Madisen, et al., Neuron 85, 942-958, 2015. To test the specificity of enhancer-driven recombinase expression, mscRE4 was cloned into constructs containing a minimal beta-globin promoter driving destabilized Cre (dgCre), iCre, FlpO, or tTA2, and the constructs were packaged into PHP.eB viruses (FIG. 53). These viruses were delivered by retro-orbital injection into mice with genetically encoded reporters for each recombinase (Ai14 for dgCre and iCre; Ai65F for FlpO; and Ai63 for tTA2). Madisen, et al., Nat. Neurosci. 13, 133-40, 2010; Madisen, et al., Neuron 85, 942-958, 2015; Daigle, et al., Cell 174, 465-480.e22, 2018. Labeling was assessed by sectioning and microscopy of native fluorescence (FIG. 53). FlpO, iCre, and tTA2 viral constructs yielded labeling of cells in L5 of the mouse cortex with varying levels of specificity, while dgCre showed non-specific labeling of cortical layers. The same strategy was applied to screen both mscRE4 and mscRE16 drivers of FlpO, iCre, and/or tTA2 by retro-orbital injection at two different titers (1×10¹⁰ and 1×10¹¹ total genome copies, GC). The specificity and completeness of labeling was found to depend heavily on both the injected titer and the recombinase-reporter combination used in these experiments (FIG. 58). Based on these experiments, a single titer for each FlpO virus was chosen for in-depth characterization, and additional animals were injected for scRNA-seq and whole-brain two-photon tomography by TissueCyte (FIGS. 57A-57C, 59A, 59B, and 60). Each of these viruses was found to have a high degree of layer and subclass specificity in the cortex, with 87.5% of cells labeled by mscRE4-FlpO corresponding to L5 PT cells (FIG. 57A) and 42% of cells labeled by mscRE16-FlpO corresponding to L5 IT cells (FIG. 57C), with little overlap. TissueCyte imaging revealed that two viruses labeled additional subcortical populations (mscRE4 in APr, CEa, and HIP, FIG. 59A; and mscRE16 in pons, BLA, and HIP, FIG. 60).

Viruses can also be co-administered to label multiple populations of cells, either exclusively or intersectionally (FIG. 59C). This strategy reduces the need for triple- or quadruple crosses to obtain co-labeled populations of cells. Brain-wide co-labeling of both L5 IT and L5 PT populations was tested by retro-orbital injection of mscRE4-iCre (to label L5 PT cells, green) and mscRE16-FlpO (to label L5 IT cells, red) in the same Ai65F;Ai140 animal (FIG. 59D). Distinct labeling of these two cell populations was found in L5 by microscopy (FIG. 59E), demonstrating that multiple enhancer-driven viruses can be used to simultaneously label or perturb populations of prospectively defined subclasses in the same animal.

Materials and Methods of Example 2. Mouse breeding and husbandry and retrograde labeling were performed as described in the Materials and Methods section of Example 1.

Single cell ATAC. Single-cell suspensions of cortical neurons were generated as described previously, with the exception of use of papain in place of pronase for some samples, and the addition of trehalose to the dissociation and sorting medium for some samples. Gray, et al., Elife 1-30, 2017 doi: 10.7554/eLife.21883. Then individual cells were sorted using FACS with gating of negative-DAPI (and positive-fluorophore labeling (tdTomato, EGFP, or SYFP2) to select for live neuronal cells or negative-DAPI and negative-fluorophore labeling for live non-neuronal cells.

For GM12878 scATAC, cells were obtained from Coriell Institute, and were grown in T25 culture flasks in RPMI 1640 Medium (Gibco, Thermo Fisher Cat #11875093) supplemented with 10% fetal bovine serum (FBS) and Penn/Strep. At 80% confluence, cells were transferred to a 15 ml conical tube, centrifuged, and washed with PBS containing 1% FBS. Cells were then resuspended in PBS with 1% FBS and 2 ng/ml DAPI (DAPI*2HCl, Life Technologies Cat #D1306) for FACS sorting.

Single cells were sorted into 200 μL 8-well strip tubes containing 1.5 μL tagmentation reaction mix (0.75 μL Nextera Reaction Buffer, 0.2 μL Nextera Tn5 Enzyme, 0.55 μL water). After collection, cells were briefly spun down in a bench-top centrifuge, then immediately tagmented at 37° C. for 30 minutes in a thermocycler. After tagmentation, 0.6 μL of Proteinase K stop solution was added to each tube (5 mg/mL Proteinase K solution (Qiagen), 50 mM EDTA, 5 mM NaCl, 1.25% SDS) followed by incubation at 40° C. for 30 minutes in a thermocycler. Then, the tagmented DNA was purified using AMPure XP beads (Beckman Coulter) at a ratio of 1.8:1 resuspended beads to reaction volume (3.8 μL added to 2.1 μL), with a final elution volume of 11 μL. Libraries were indexed and amplified by the addition of 15 μL 2× Kapa HiFi HotStart ReadyMix and 2 μL Nextera i5 and i7 indexes to each tube, followed by incubation at 72° C. for 3 minutes and PCR (95° C. for 1 minute, 22 cycles of 98° C. for 20 seconds, 65° C. for 15 seconds, and 72° C. for 15 seconds, then final extension at 72° C. for 1 minute). After amplification, sample concentrations were measured using a Quant-iT PicoGreen assay (Thermo Fisher) in duplicate. For each sample, the mean concentration was calculated by comparison to a standard curve, and the mean and standard deviation of concentrations was calculated for each batch of samples. Samples with a concentration greater than 2 standard deviations above the mean were not used for downstream steps, as these were found in early experiments to dominate sequencing runs. All other samples were pooled by combining 5 μL of each sample in a 1.5 ml tube. Then, the combined library was purified by adding Ampure XP beads in a 1.8:1 ratio, with final elution in 50 μL. The mixed library was then quantified using a BioAnalyzer High Sensitivity DNA kit (Agilent).

scATAC sequencing, alignment, and filtering was performed as described in the Materials and Methods section of Example 1. Jaccard distance calculation, PCA and tSNE embedding, and density-based clustering were also performed as described in the Materials and Methods section of Example 1, except that in comparing scATAC-seq samples, fragments were extended to a length of 1 kb and samples were clustered in t-SNE space using the RPhenograph package with k=6.

Correlation with single-cell transcriptomics. Phenograph-defined neighborhoods were assigned to cell subclasses and clusters by comparison of accessibility near transcription start site (TSS) to median expression values of scRNA-seq clusters at the cell type (e.g. L5 PT Chrna6) and at the subclass level (e.g. Sst) from mouse primary visual cortex. Tasic, et al., Nature 563, 72-78, 2018. To score each transcription start site (TSS), TSS locations were retrieved from the RefSeq Gene annotations provided by the UCSC Genome Browser database, and windows from TSS+/−20 kb were generated. Then, the number of fragments for all samples within each cluster that overlapped these windows were counted. For comparison, differentially expressed marker genes were selected from the Tasic, et al., Nature 563, 72-78, 2018 scRNA-seq dataset using the scrattch.hicat package for R. Then, Phenograph cluster scores were correlated with the log-transformed median exon read count values for this set of marker genes for each scRNA-seq cluster from primary visual cortex, and the transcriptomic cell type with the highest-scoring correlation was assigned. This strategy of neighbor assignment and correlation allowed resolution of cell types within the scATAC-seq data close to the resolution of the scRNA-seq data, as types that were split too far would resolve to the same transcriptomic subclass or type by correlation.

scATAC-seq grouping and peak calling. For downstream analysis, cell type assignments were grouped to the subclass level, with the exception of highly distinct cell types (Lamp5 Lhx6, Sst Chodl, Pvalb Vipr2, L6 IT Car3, CR, and Meis2). Unique fragments for all cells within each of these subclass/distinct type groups were aggregated to BAM files for analysis. Aligned reads from single cell subclasses/clusters were used to create Tag Directories and peaks of chromatin accessibility were called using HOMER with settings “findPeaks-region-o auto”. The resulting peaks were converted to BED format. Heinz, et al., Mol. Cell 38, 576-589, 2010.

Population ATAC of Sst neurons. Population ATAC-seq of neurons from Sst-IRES2-Cre;Ai14 mice was performed as described previously. Gray, et al., Elife 1-30, 2017. doi: 10.7554/eLife.21883. Briefly, cells from the visual cortex of an adult mouse were microdissected and FACS sorted into 8-well strips as described above, but with 500 cells per well instead of single cells as for scATAC-seq. Cell membranes were lysed, and nuclei were pelleted before resuspension in the same tagmentation buffer described above at a higher volume (25 μL). Tagmentation was carried out at 37 C for 1 hour, followed by addition of 5 μL of Cleanup Buffer (900 mM NaCl, 300 mM EDTA), 2 μL 5% SDS, and 2 μL Proteinase K and incubation at 40° C. for 30 minutes, and cleanup with AMPure XP beads (Beckman Coulter) at a ratio of 1.8:1 beads to reaction volume. Samples were amplified using KAPA HotStart Ready Mix (Kapa Biosystems, Cat #KK2602) and 2 μL each of Nextera i5 and i7 primers (Illumina), quantified using a Bioanalyzer, and sequenced on an Illumina MiSeq.

Comparisons to bulk ATAC-seq data. For comparison to previously published studies, data was used from GEO accession GSE63137 from Mo, et al., Neuron 86, 1369-1384, 2015 for Camk2a, Pvalb, and Vip neuron populations, GEO accession GSE87548 from Gray, et al. (Elife 1-30, 2017) for Cux2, Scnn1a-Tg3, Rbp4, Ntsr1, Gad2, mES, and genomic controls. Mo, et al., Neuron 86, 1369-1384, 2015; Gray et al., Elife 1-30, 2017 doi: 10.7554/eLife.21883. For these comparisons, population ATAC-seq of Sst neurons, described above, were also included. For each population, reads from all replicates were merged and each region was downsampled to 6.4 million reads. Then, peaks were called using HOMER as described above for aggregated scATAC-seq. The BED-formatted peaks for scATAC-seq aggregates with or without bulk ATAC-seq datasets were used as input for comparisons using the DiffBind package for R as described previously. Gray, et al., Elife 1-30, 2017 doi: 10.7554/eLife.21883.

Identification of mouse single-cell regulatory elements. A targeted search for mouse single cell regulatory elements (mscREs) was done by performing pairwise differential expression analysis of scRNA-seq clusters to identify uniquely expressed genes in L5 PT and L5 IT subclasses across all glutamatergic subclasses. Then, unique peaks were searched for within 1 Mbp of each marker gene, and these peaks were manually inspected for low or no accessibility in off-target cell types and for conservation. If a region of high conservation overlapped the peak region, but the peak was not centered on the highly conserved region, the peak selection was adjusted to include neighboring highly conserved sequence. For cloning, primer search was centered on 500 bp regions centered at the middle of the selected peak regions and included up to 100 bp on either side. Final region selections and PCR primers are shown in FIG. 47.

The following techniques were performed as described in the Materials and Methods Section of Example 1: viral genome cloning; viral packaging, titering, and titer measurement; retro-orbital injections; stereotaxic injections (except that each virus was delivered bilaterally at 250 nL, 50 nL, and 25 nL); immunohistochemistry; and single cell RNA sequencing and cell type mapping.

Comparisons to previous scATAC-seq studies. For comparisons to GM12878 datasets, raw data from Cusanovich, et al. (Science 348, 910-4, 2015) was downloaded from GEO accession GSE67446, Salav, et al. (2015) from GEO accession GSE65360, and Pliner, et al. (Mol. Cell 71, 858-871.e8, 2018) from GEO accession GSE109828. Buenrostro et al., Nature 523, 486-90, 2015. Processed 10× Genomics data was retrieved from the 10× Genomics website. Samples from Buenrostro, Cusanovich, Pliner, and the Gm12878 from this lab were aligned to the hg38 human genome using the same bowtie pipeline described above for mouse samples to obtain per-cell fragment locations. 10× Genomics samples were analyzed using fragment locations provided by 10× Genomics. For comparison to TSS regions, the RefSeq Genes tables provided by the UCSC Genome Browser database for hg19 (for 10× data) and for hg38 (for other datasets) were used. To compare to ENCODE peaks, ENCODE Gm12878 DNA-seq HotSpot results from ENCODE experiment ID ENCSROOOEJD aligned to hg19 (ENCODE file ID ENCFF206HYT) or hg38 (ENCODE file ID ENCFF773SPT) were used.

For comparisons to previously published mouse cortex datasets, raw FASTQ files were downloaded from GEO accession GSE111586 for Cusanovich, et al. (Cell 174, 1309-1324.e18, 2018) and from GEO accession GSE100033 for Preissl, et al. Nat. Neurosci. 21, 1, 2018. Multiplexed files were aligned to the mm10 genome using Bowtie v1.1.0 and were demultiplexed using an R script prior to removal of duplicate location alignments. Only barcodes with >1,000 mapped reads were retained for analysis. Per-barcode statistics were computed using the same algorithms used for per-cell statistics from the dataset generated by this lab, and samples from the Cusanovich, et al., Cell 174, 2018 dataset that passed the established QC criteria, were subjected to the same analysis pipeline as the data generated by this lab after demultiplexing and duplicate read removal. Metadata from Cusanovich, et al., (Cell 174, 2018) were obtained from the Mouse sci-ATAC-seq Atlas website at http://atlas.gs.washington.edu/mouse-atac/.

Physiology, patch clamp recordings, and data analysis was performed as described in the Materials and Methods section of Example 1.

TissueCyte imaging and analysis. TissueCyte images were collected, registered, and segmented as described previously. Oh et al., (Nature 508, 207-214, 2014). After registration, 3D arrays of signal binned to 25 um voxels were analyzed in R by subtraction of background, and averaging the signal in the finest structure in the Allen Brain Atlas structural ontology. To propagate signals from fine to coarse structure in the ontology, hierarchical calculations that assigned the maximum value of child nodes in the ontology to each parent from the bottom to the top of the ontology were performed. Then, the ontology was filtered to remove very fine structures, and the taxa and metacodeR packages for R were used to display the resulting ontological relationships and structure scores. Foster et al., bioRxiv 071019, 2016 doi: 10.1101/071019.

Software for analysis and visualization. Analysis and visualization of scATAC-seq and transcriptomic datasets were performed using R v.3.5.0 and greater in the Rstudio IDE (Integrated Development Environment for R) or using the Rstudio Server Open Source Edition as well as the following packages: for general data analysis and manipulation, data.table, dplyr, Matrix, matrixStats, purrr, and reshape2; for analysis of genomic data, GenomicAlignments, GenomicRanges, and rtracklayer; for plotting and visualization, cowplot, ggbeeswarm, ggExtra, ggplot2, and rgl; for clustering and dimensionality reduction, Rphenograph and Rtsne; for analysis of transcriptomic datasets: scratch.hicat and scrattch.io; for taxonomic analysis and visualization, metacodeR and taxa; and plater for management of plate-based experimental results and metadata.

### Example 3. Human single neuron epigenetic evaluation of neocortical cell classes. The

primate and especially human neocortex is greatly expanded in size and complexity relative to that of other mammals like the rodent (Zeng, et al., Cell. 149, 483-496, 2012; Rakic, Nat Rev Neurosci. 10, 724-735, 2009). Neocortical expansion enables human-centric abilities such as language and reasoning, which are disrupted in human diseases like schizophrenia and autism (King, et al., JAMA Netw Open. 1, e184777-e184777, 2018; van den Heuvel et al., JAMA Psychiatry. 70, 783-792, 2013). This structure contains of billions of cells, grouped into dozens if not hundreds of molecularly defined cell types (Zeisel, et al., Science. 347, 1138-1142, 2015; Tasic, et al., Nat Neurosci. 19, 335-346, 2016; Tasic et al., Nature. 563, 72, 2018; Hodge, et al., bioRxiv, 384826, 2018).

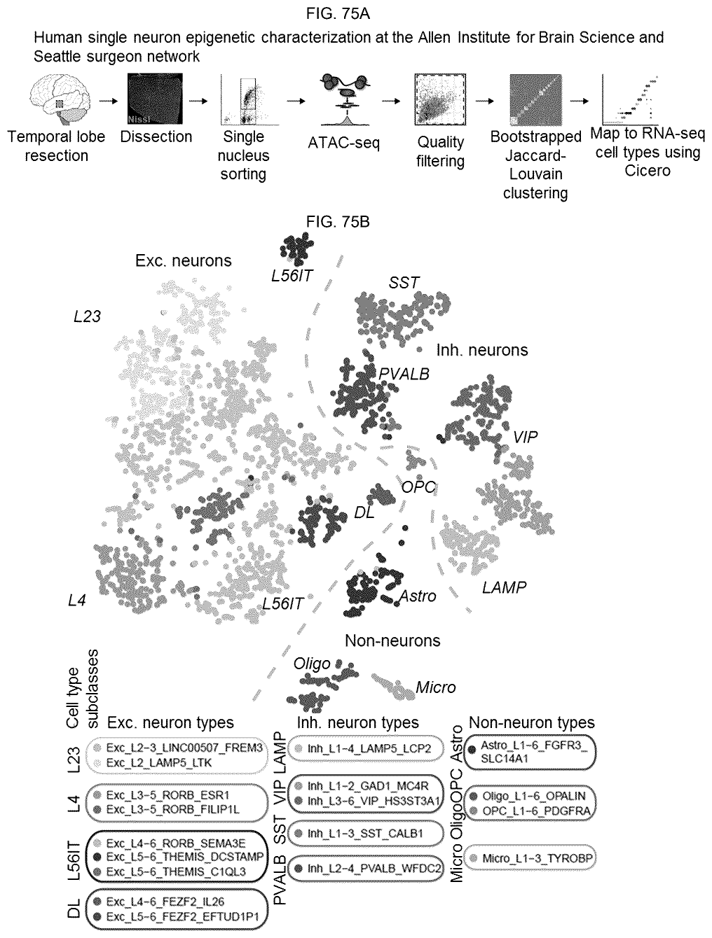

To understand these cells and their regulation, from multiple fresh neurosurgical specimens (bulk n=5, single n=14) a high-quality dataset of accessible chromatin was generated using both bulk and single human brain nuclei via ATAC-seq (Buenrostro et al., Nature. 523, 486-490, 2015; Graybuck et al., bioRxiv, 525014, 2019; Gray et al., eLife Sciences. 6, e21883, 2017). 3660 single nucleus ATAC-seq libraries (median 48542 unique mapped reads) were prepared and 2858 quality-filtered nuclei were used for clustering and mapping (FIG. 75A, and Materials and Methods of Example 3). 27 ATAC-seq clusters were identified that mapped to 18 human brain temporal lobe transcriptomically defined cell types (Hodge et al., bioRxiv, 384826, 2018) (FIG. 75B). These cell types spanned three major classes of brain cell types: excitatory, inhibitory, and non-neurons; and eleven cell type subclasses: excitatory layer 2/3 (L23), layer 4 (L4), layer 5/6 intra-telencephalic (L56IT), and deep layer non-intratelencephalic neurons (DL); inhibitory LAMP5, VIP, SST, and PVALB neurons, and non-neuronal Astrocytes, Microglia, and Oligodendrocytes/OPCs. The identified cell types were typically identified in the expected sort strategy (FIG. 75B), and all cell types were populated by multiple specimens.

To identify putative regulatory elements within each subclass, data was aggregated for all nuclei within each subclass, and subclass-specific peaks were called with Homer (Heinz et al., Molecular Cell. 38, 576-589, 2010), revealing peaks proximal to recently identified transcriptomic subclass-specific marker genes (Hodge et al., bioRxiv, 384826, 2018), confirming the clustering and mapping strategy. Furthermore, within peaks chromVAR (Schep et al., Nature Methods. 14, 975-978, 2017) identified expected cell type-distinguishing transcription factor motifs, including DLX1 in inhibitory neurons and NEUROD6 in lower-layer excitatory neurons, whose accessibilities correlated with their transcript abundances (Hodge et al., bioRxiv, 384826, 2018) across subclasses (paired t-tests for correlation; DLX1 t=3.0 p<0.01; NEUROD6 t=5.4 p<0.001). These observations indicate strong concordance between RNA-seq and ATAC-seq data modalities.

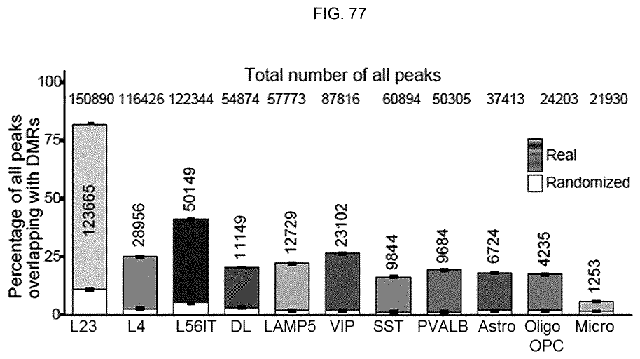

To assess the correspondence among accessibility and epigenetic modifications and primary sequence, the overlap between subclass snATAC-seq peaks and differentially methylated regions (DMRs) as previously identified (Lister, et al., Science. 341, 1237905, 2013; Luo, et al., Science. 357, 600-604, 2017) was calculated and aggregated by subclass. For every cell subclass, a greater overlap of ATAC-seq peaks was observed with DMRs than would be expected by chance alone (FIG. 77E), furnishing thousands of independently validated human neocortical cell subclass epigenetic elements.

To explore the relationships of these elements to genes, cell subclass peaks were also subset to sets of all peaks, subclass-specific peaks, transcription start site (TSS)-distal peaks (farther than 20 kb from any RefSeq TSS), and the intersection of subclass-specific and TSS-distal peaks; this analysis revealed a particularly strong DMR overlap in TSS-distal peaks (ANOVA F=3.6; all peaks versus TSS-distal p<0.05; all peaks versus TSS-distal and subclass-specific; p<0.01 [Sidak post-hoc corrected probabilities]). To further characterize ATAC-seq peaks, their primary sequence conservation was next calculated by phyloP scores (Pollard et al., Genome Res. 20, 110-121, 2010). All cell subclass peak sets were on average more conserved than random DNA stretches. In particular, it was observed that TSS-distal peaks have greater conservation scores than all peaks (paired t-test, p<. 001, t=5.4, df=10), and inhibitory neuron subclass peaks had significantly greater conservation than those of excitatory neuron subclasses (heteroscedastic t-test, p<0.05, t=2.6, df=5.6 for the all peak sets; p<0.05, t=2.5, df=5.9 for the TSS-distal peak sets), agreeing with previous observations by Luo et al. (Science. 357, 600-604, 2017).

Taken as a whole, high conservation and confirmation via molecularly independent techniques together suggest that ATAC-seq identifies authentic functional genomic elements that bestow human neocortical cell type identity.

In order to count human accessible chromatin regions shared with mouse (“conserved”), and those unique to human (“divergent”), Jaccard similarity coefficients among human peaks and human genome-mapped mouse peaks were computed for all cell subclasses. All mouse subclasses display highest Jaccard similarity enrichment to their orthologous human subclasses, and all but one human subclass map as expected reciprocally. In addition, non-neurons displayed the strongest cross-species epigenetic similarities, followed by inhibitory neurons, and excitatory neurons displayed the weakest but still greater than random similarities. Quantifying conserved and divergent peaks in each species revealed thousands in each category, with many more conserved peaks than expected by chance alone. Furthermore, much greater primary sequence conservation is observed in conserved peaks than divergent peaks in both species (heteroscedastic t-test; human t=10.3, p<0.001; mouse t=6.6, p<0.001), suggesting that these elements perform important evolutionarily shared functions. Across 11 cortical subclasses, it was observed that 34±10% (mean±sd) of all human accessible chromatin elements are conservedly detected in mouse. In conclusion, many functional genomic elements are conserved between human and mouse, across all major neocortical cell subclasses.

Having established a high-quality and high-resolution catalog of human neocortical accessible genomic elements, these data were used as a tool to associate cell subclasses with brain diseases and traits. Linkage disequilibrium score regression (LDSC; Bulik-Sullivan et al., Nature Genetics. 47, 291-295, 2015; Finucane et al., Nat Genet. 47, 1228-1235, 2015) was used to find significant associations between human brain cell subclass ATAC-seq peaks and SNPs identified in 15 genome-wide association study brain diseases or traits with sufficient power (see Materials and Methods of Example 3). Overall similar association patterns were observed using either ATAC-seq peaks or DMRs (Lister et al., Science. 341, 1237905, 2013; Luo et al., Science. 357, 600-604, 2017), and generally weak associations for the outgroup trait (Crohn's disease) and outgroup peakset (The ENCODE Project Consortium, Nature. 489, 57-74, 2012), together suggesting that these analyses are robust to experimental technique.

Subclass peaksets were split into conserved and divergent subsets, and generally stronger associations between brain diseases/traits and conserved peaks were found. Significant associations (passing Bonferroni-corrected p-value significance cutoffs) between multiple neuronal (but not non-neuronal) subclass peaksets and educational attainment and schizophrenia were observed, similar to previous analyses of RNA-seq data (Skene et al., Nature Genetics. 50, 825, 2018; Girdhar et al., Nature Neuroscience. 21, 1126-1136, 2018; Cusanovich et al., Cell. 174, 1309-1324.e18, 2018), and it was found that these associations are stronger in conserved regions than in divergent regions. The strongest association was also observed between microglial peaks and Alzheimer's disease as in previous reports (Skene et al., Nature Genetics. 50, 825, 2018; Girdhar et al., Nature Neuroscience. 21, 1126-1136, 2018; Cusanovich et al., Cell. 174, 1309-1324.e18, 2018), although these results did not pass significance cutoffs, possibly due to low overall total heritability and hence power in Alzheimer's studies. Interestingly, this microglial-Alzheimer's association is stronger in divergent peaks than in conserved peaks, suggesting human-specific modes of microglial gene expression contribute to Alzheimer's pathology.

Since human divergent peaks outnumber conserved peaks, it was speculated whether overall heritability of neuron-associated traits (educational attainment and schizophrenia) is largely conserved or divergent. Summing total subclass-associated heritabilities revealed that the conserved peaks contain the majority of heritability, and significantly more than divergent peaks. Taken as a whole, these analyses suggest that that cross-species epigenetic analysis enables the discovery of conserved functional genomic elements that illuminate human health and disease.

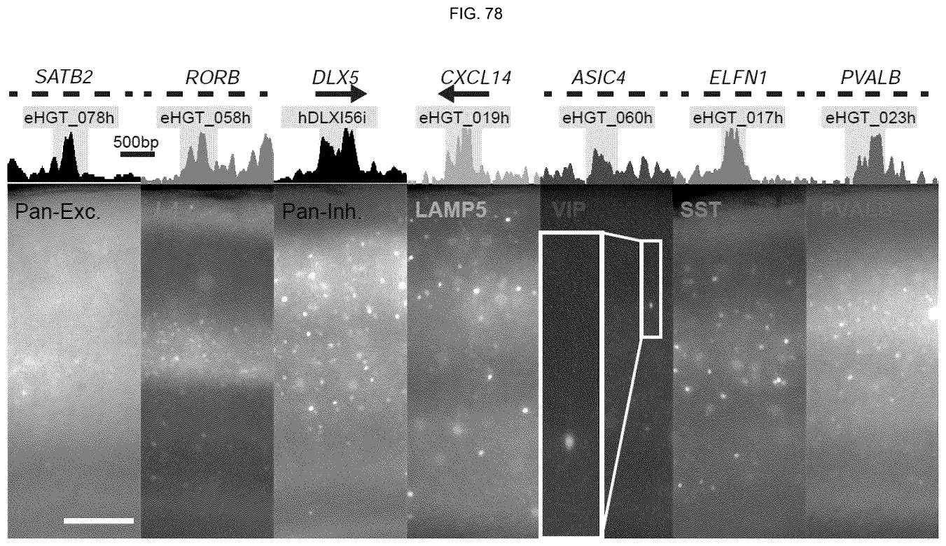

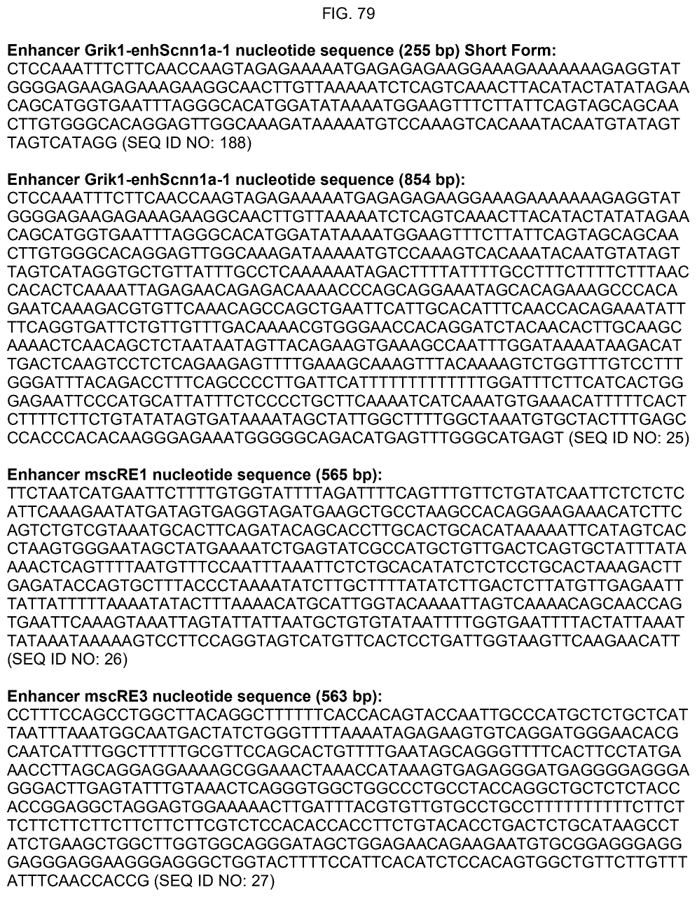

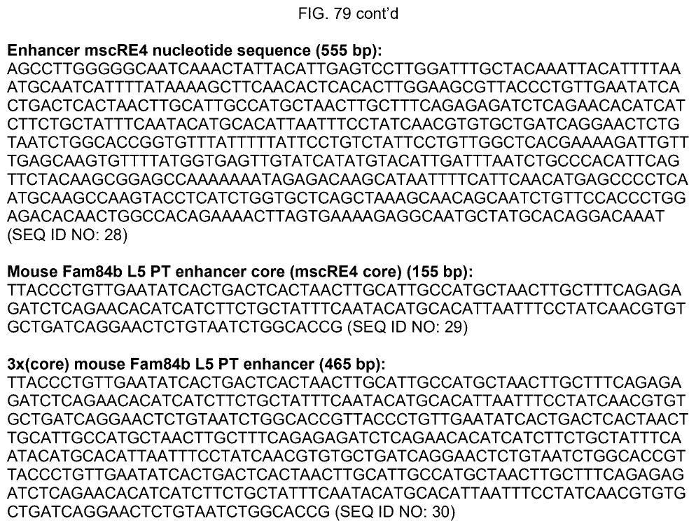

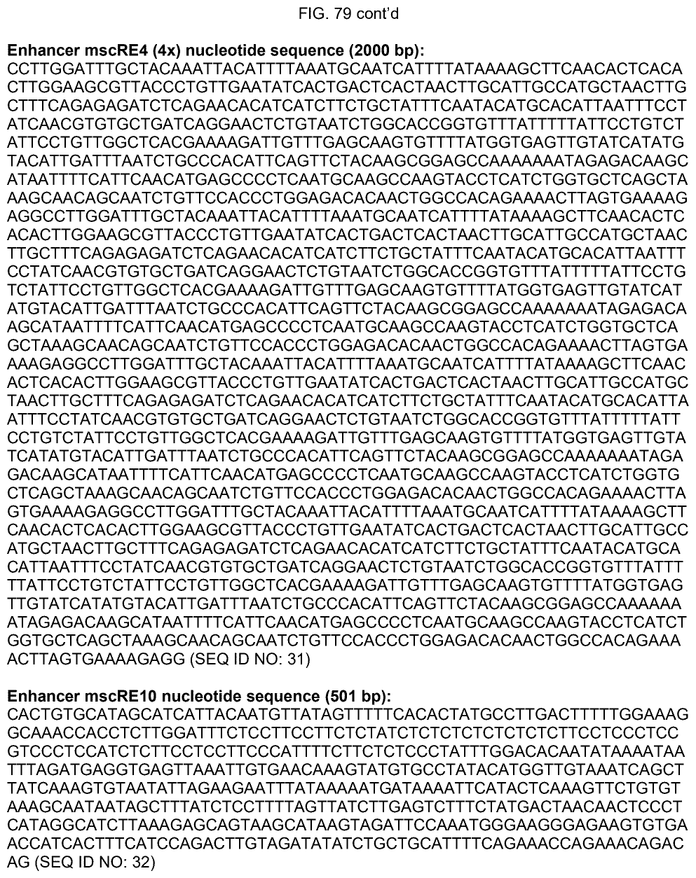

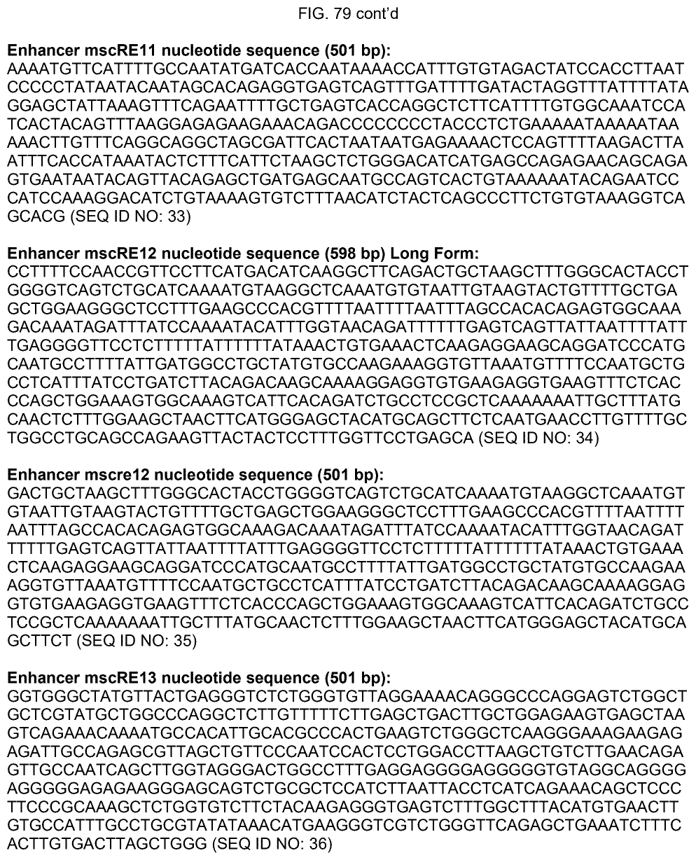

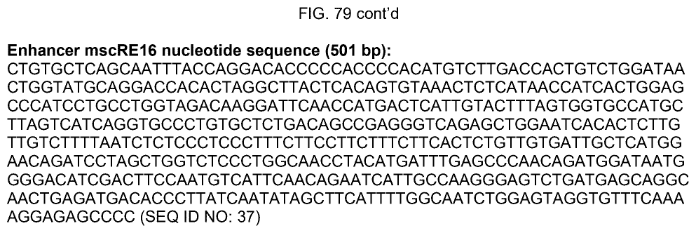

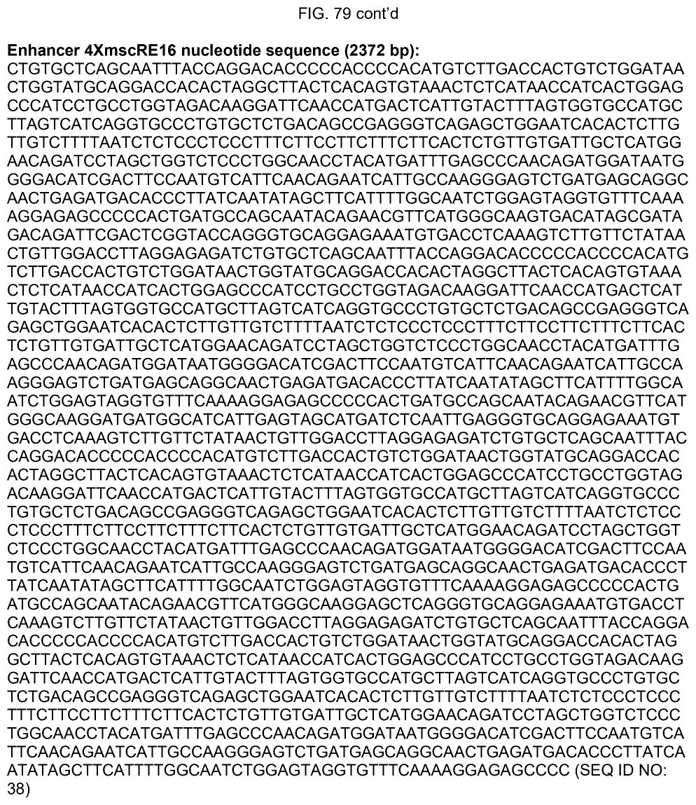

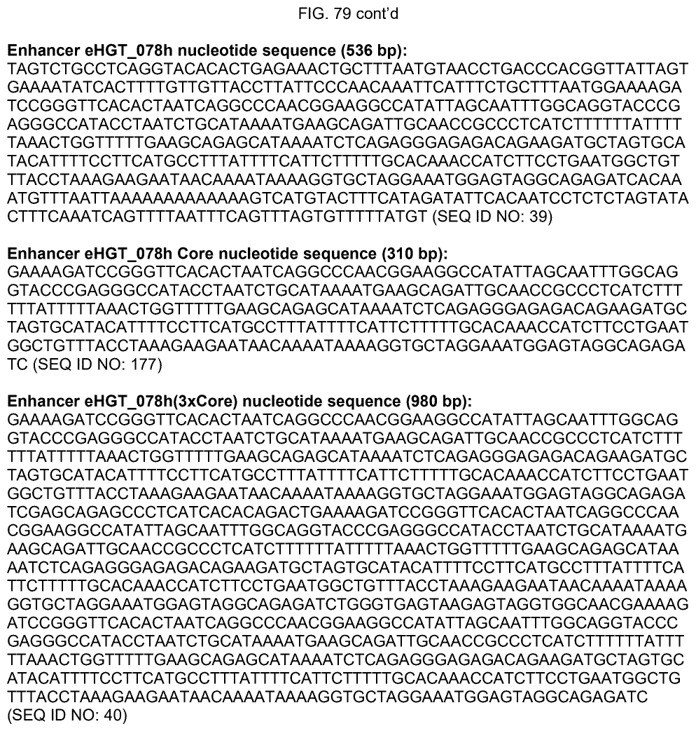

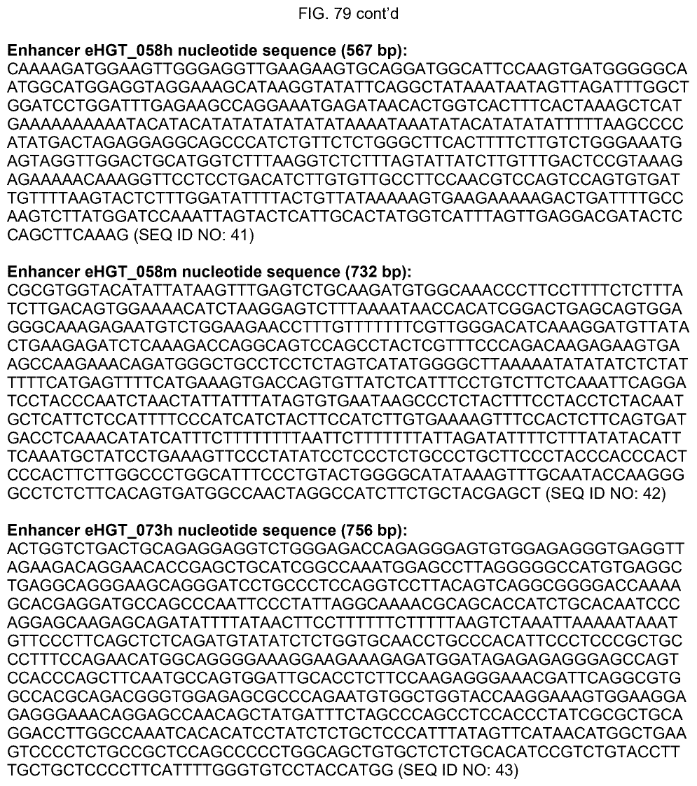

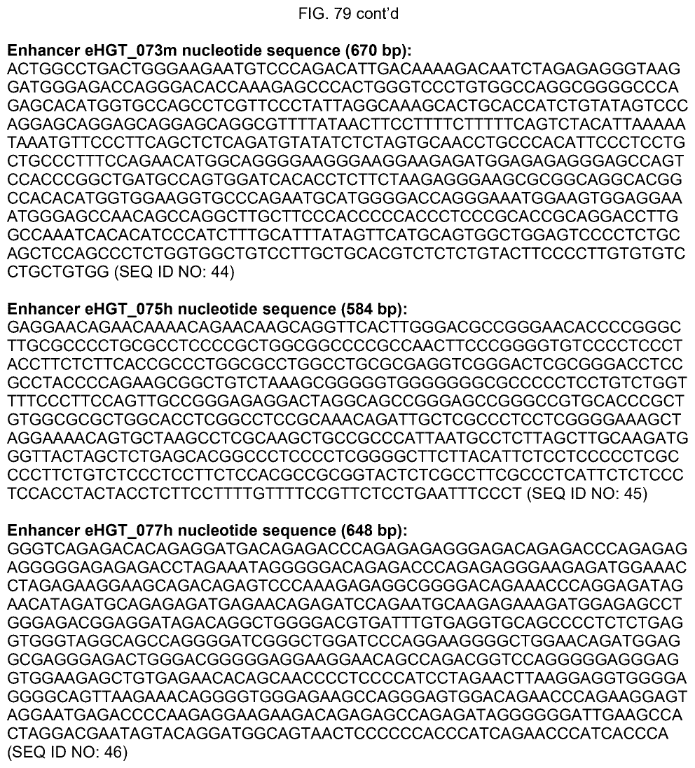

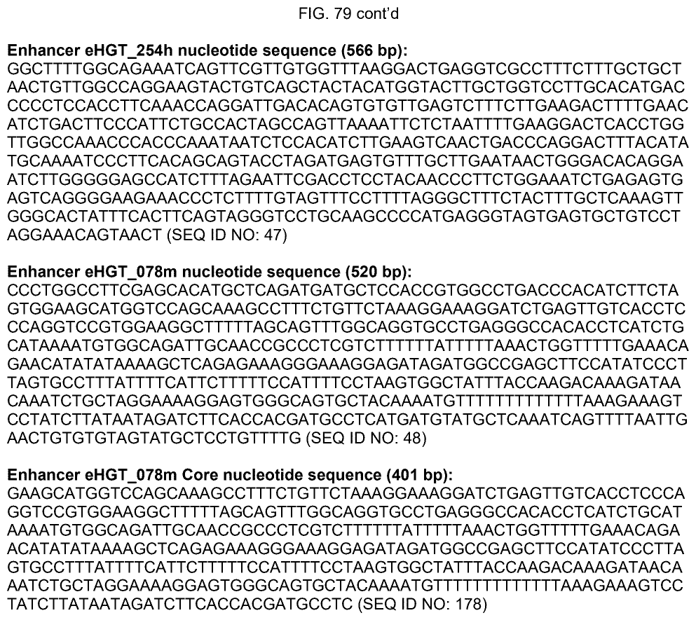

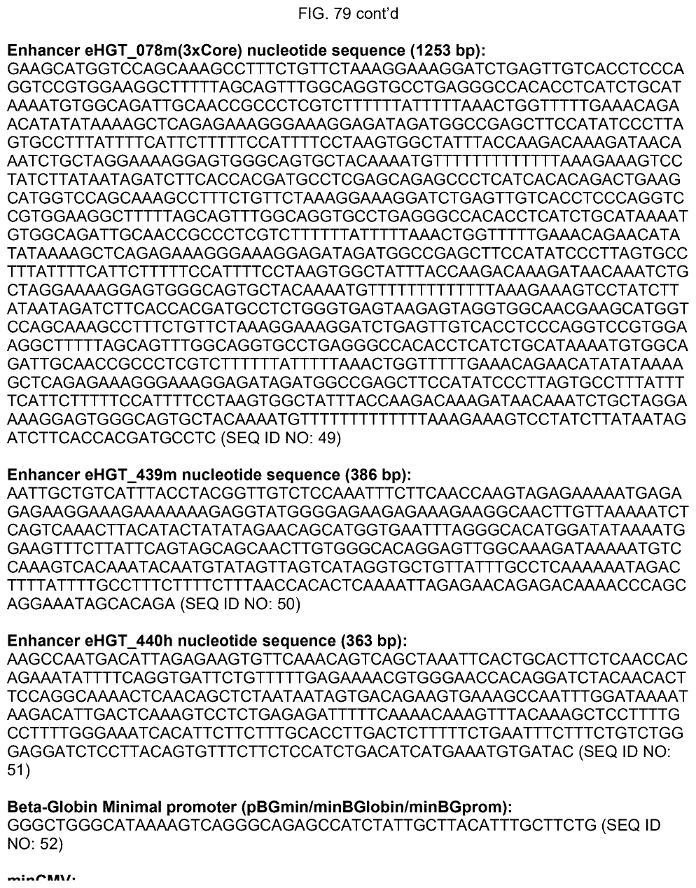

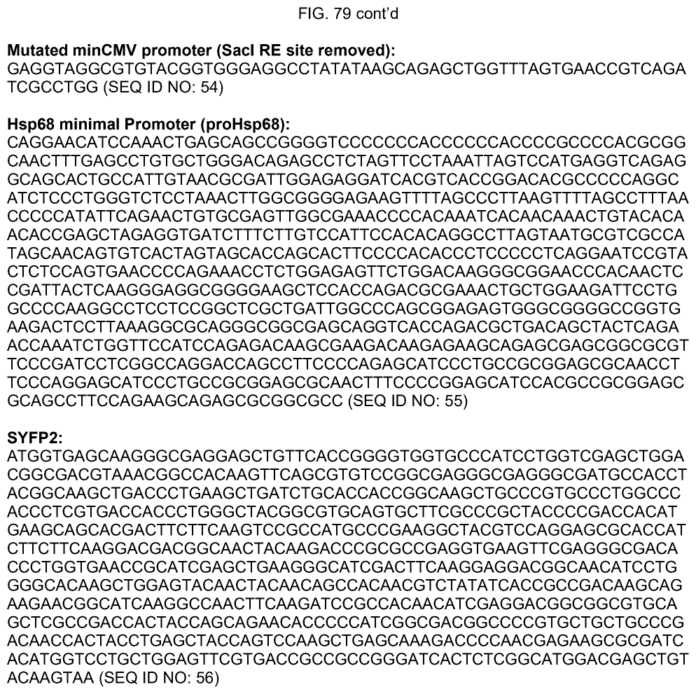

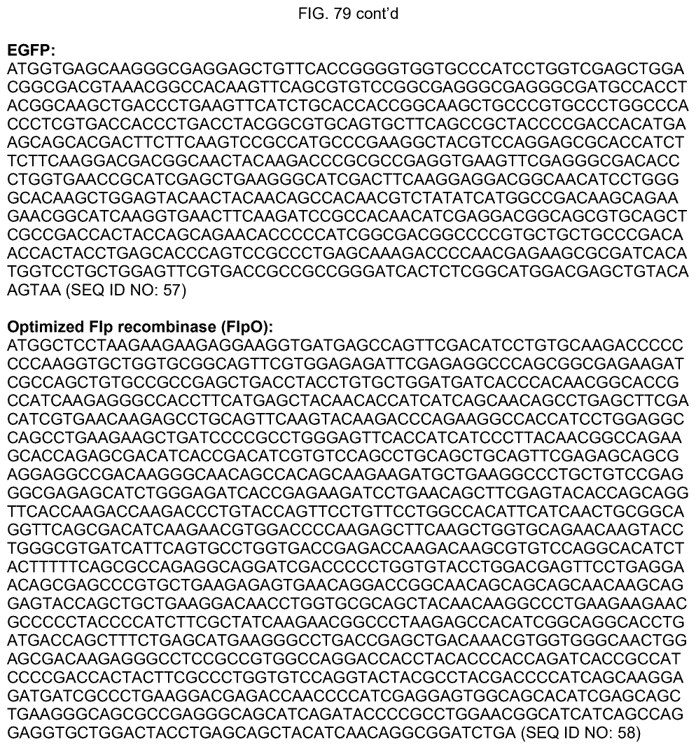

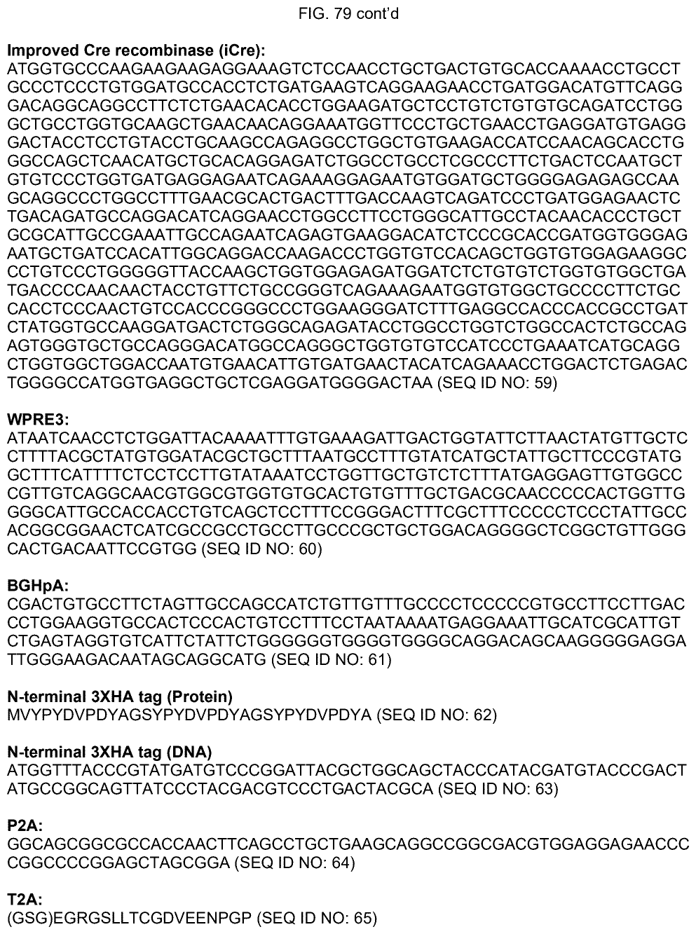

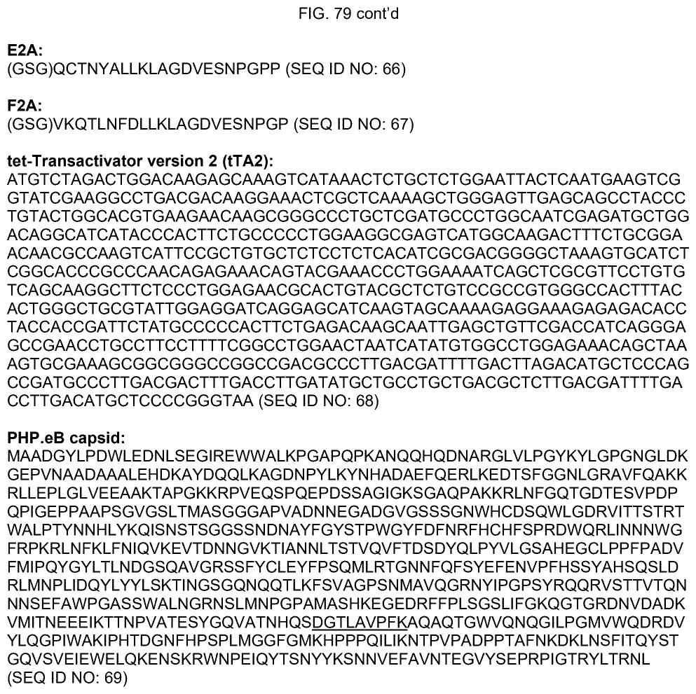

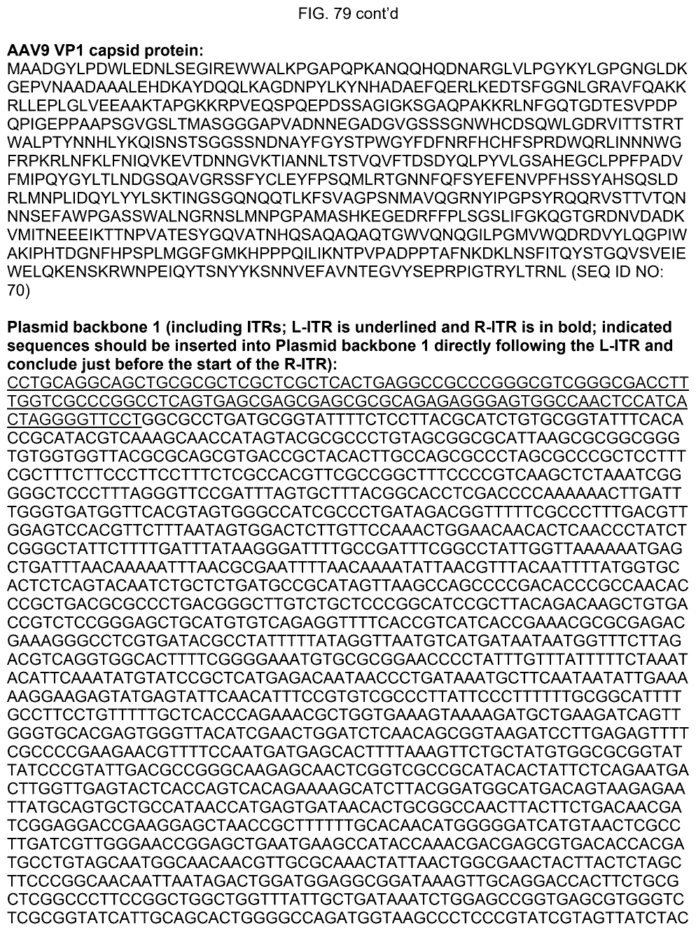

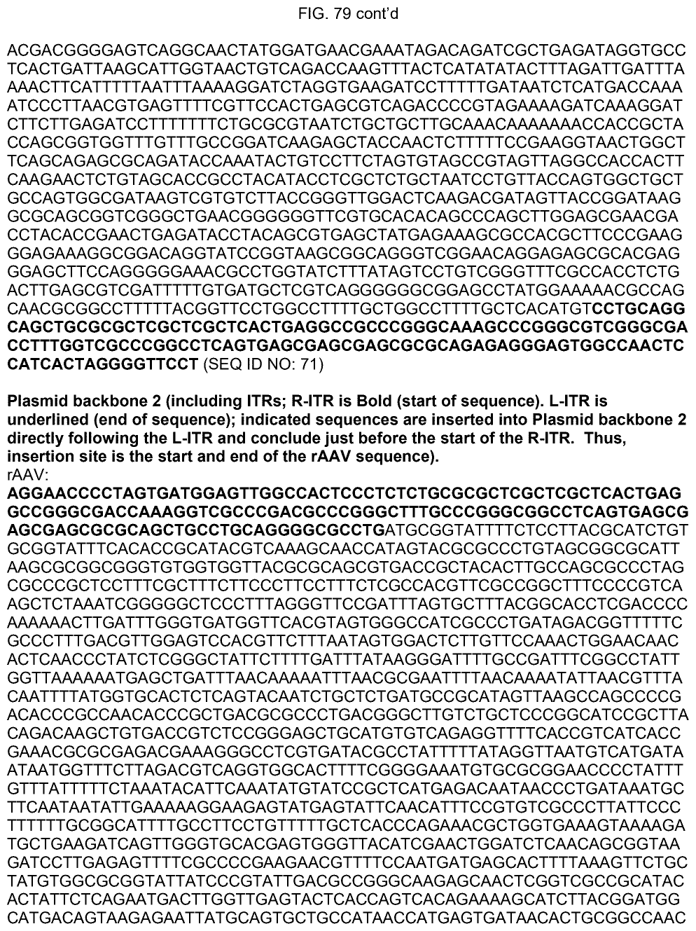

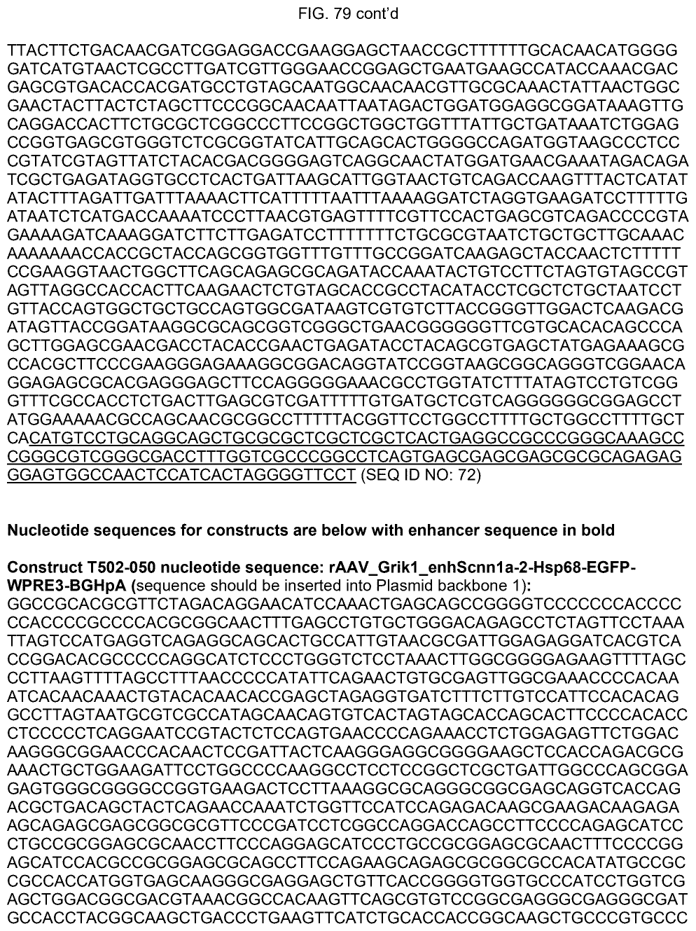

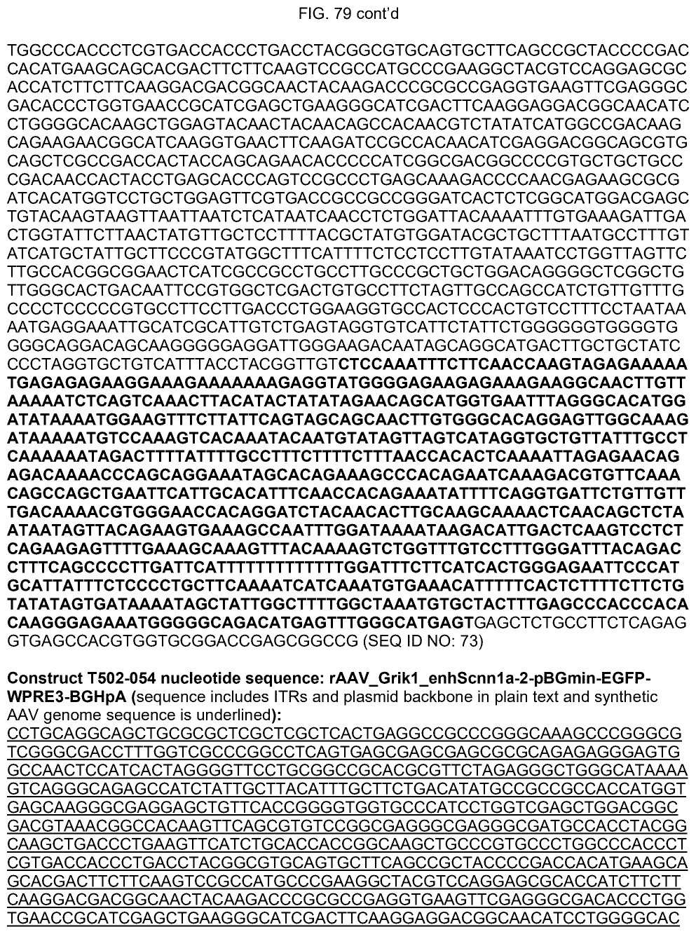

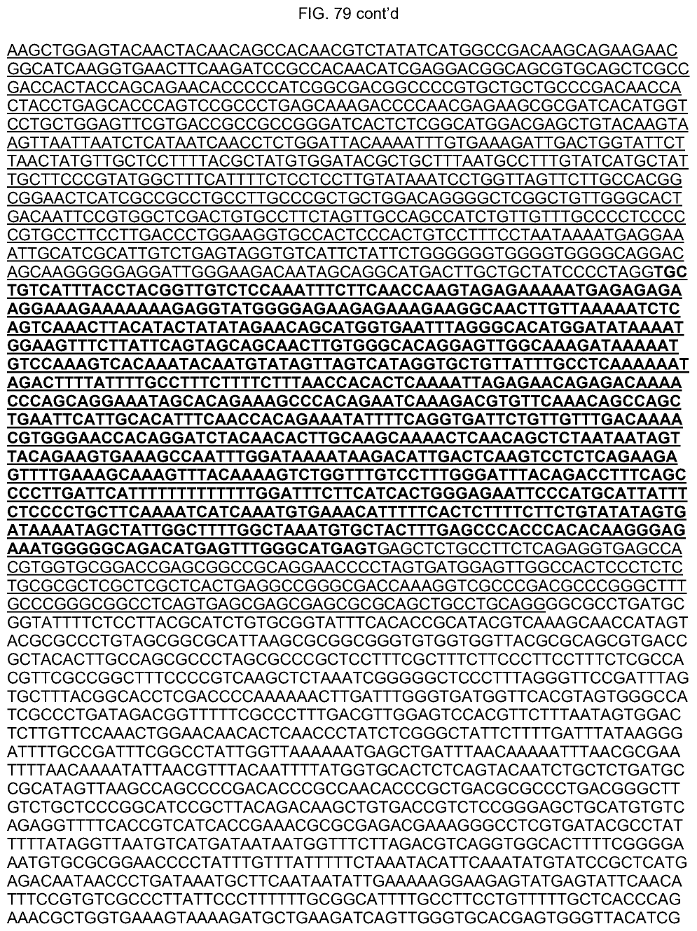

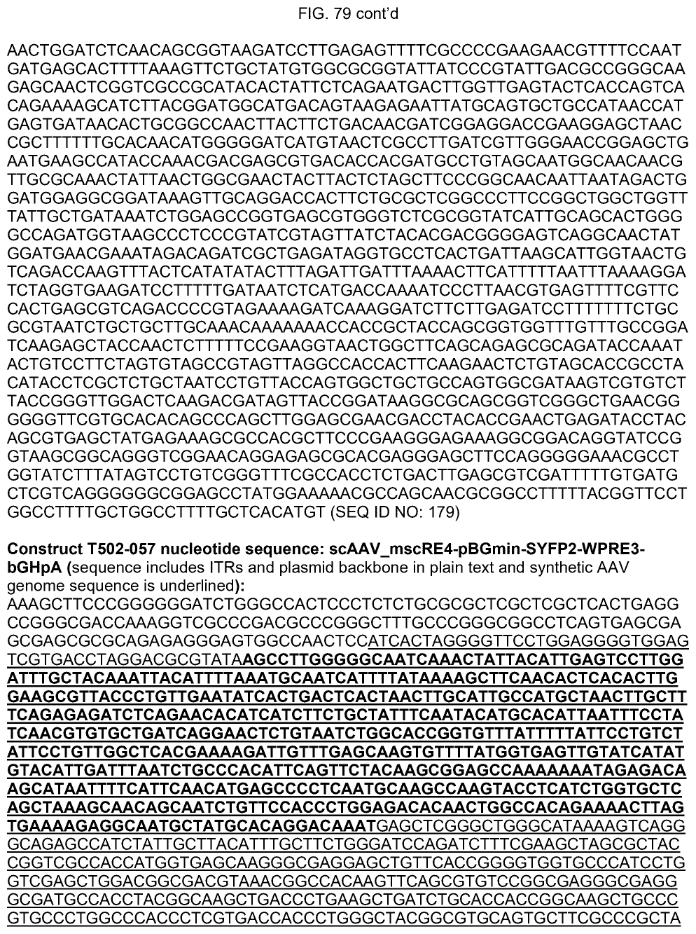

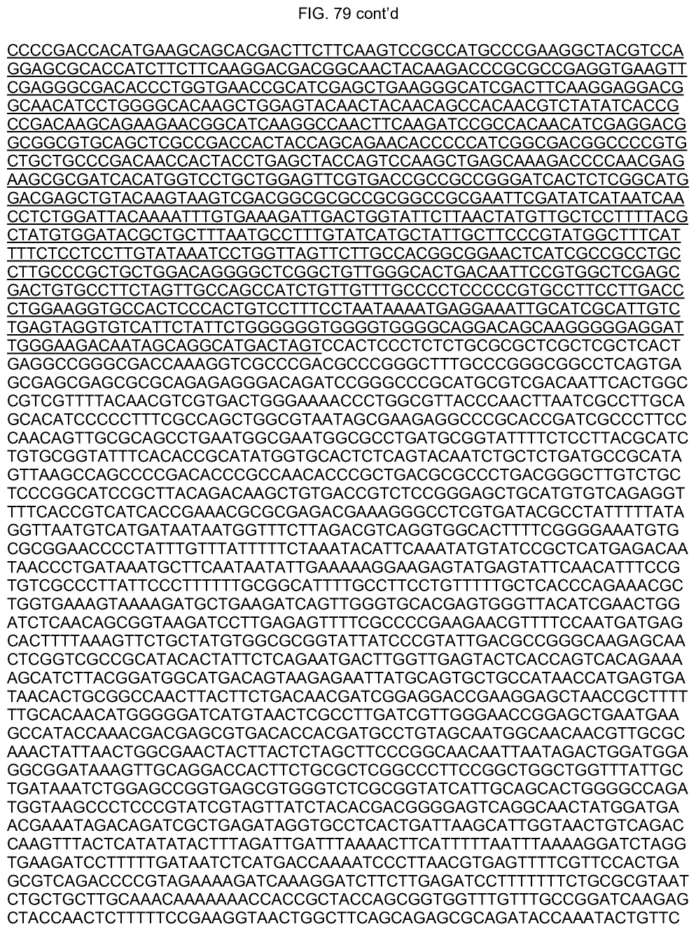

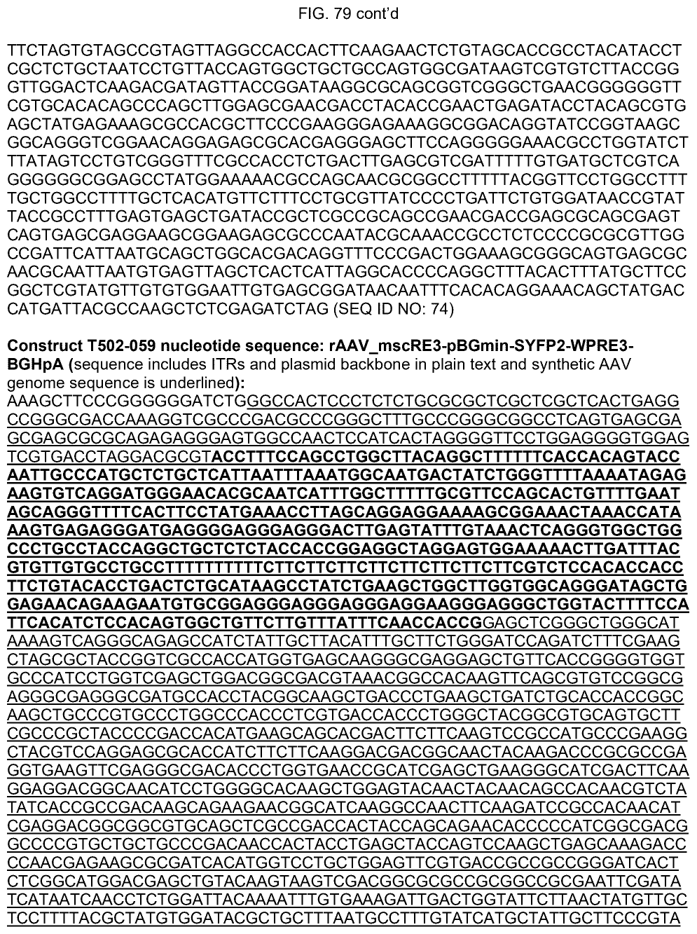

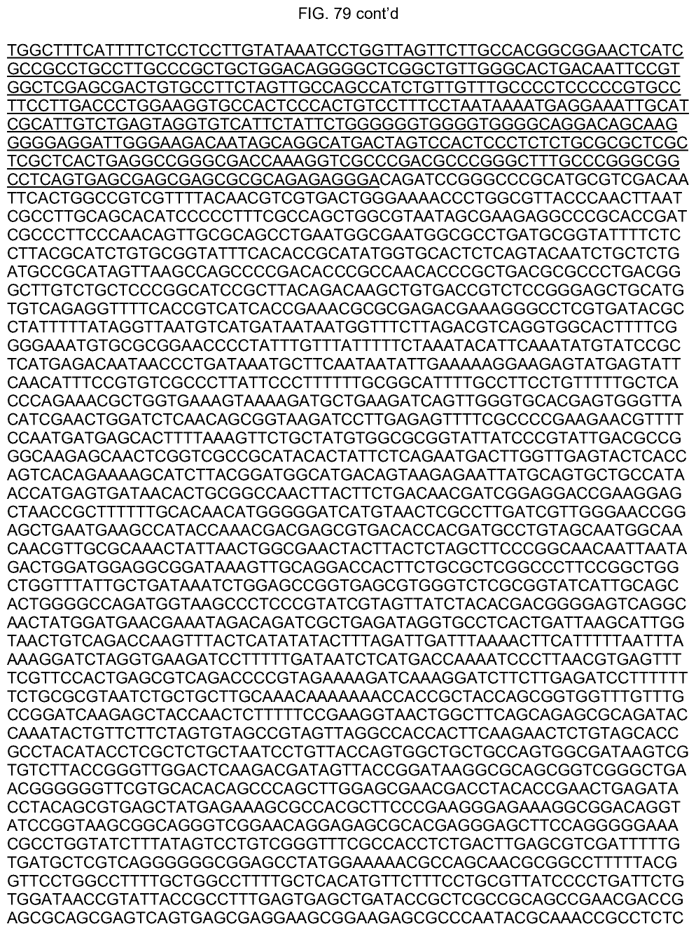

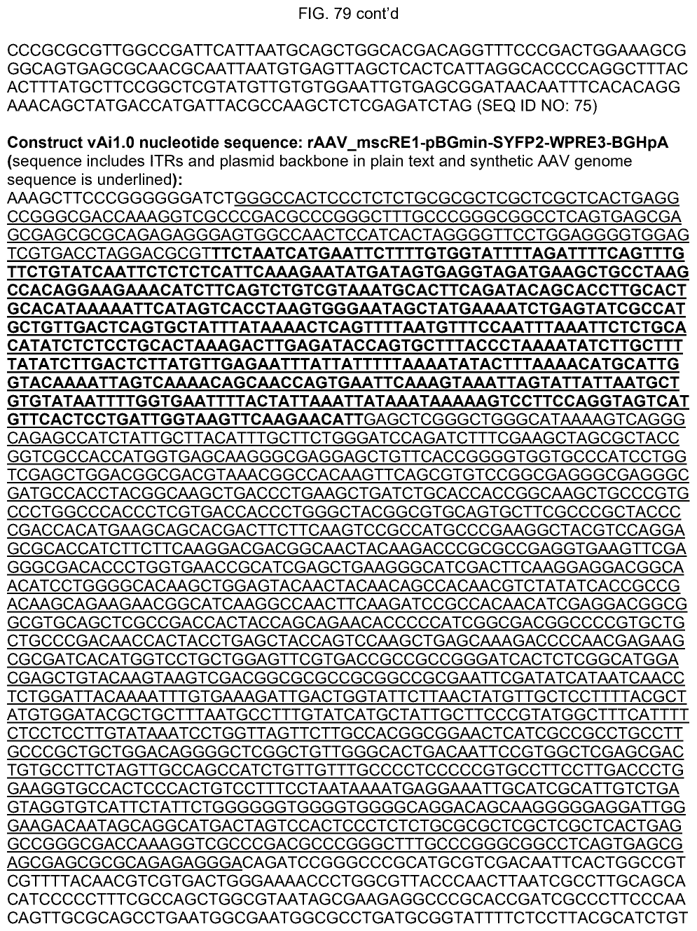

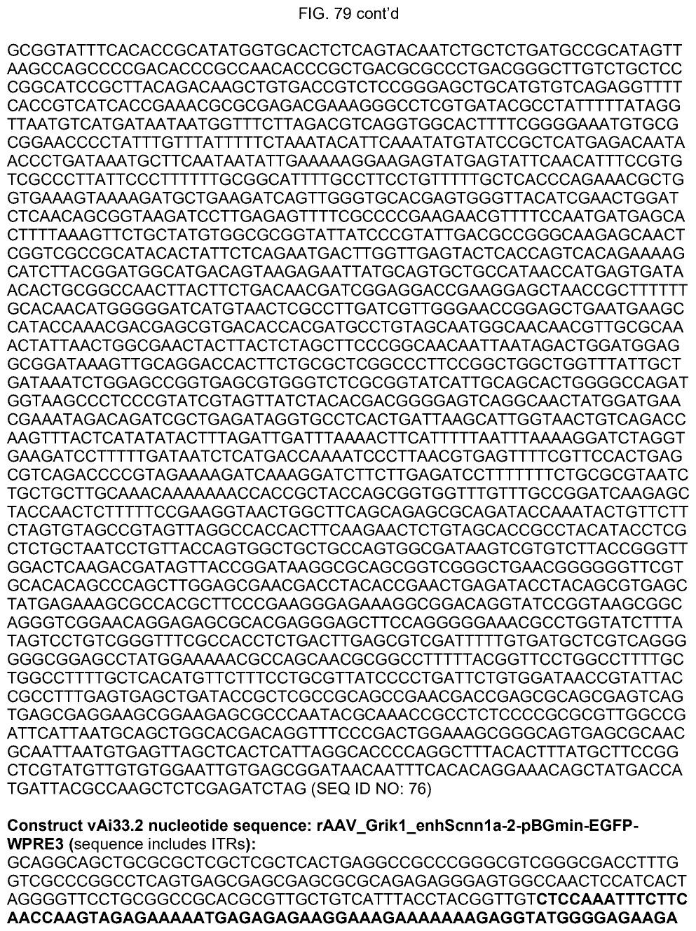

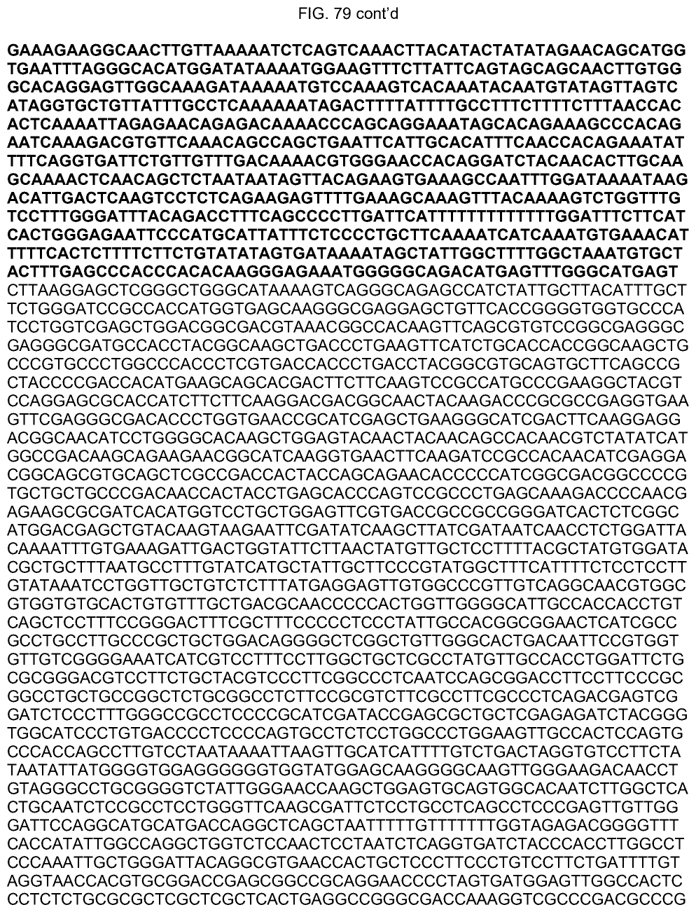

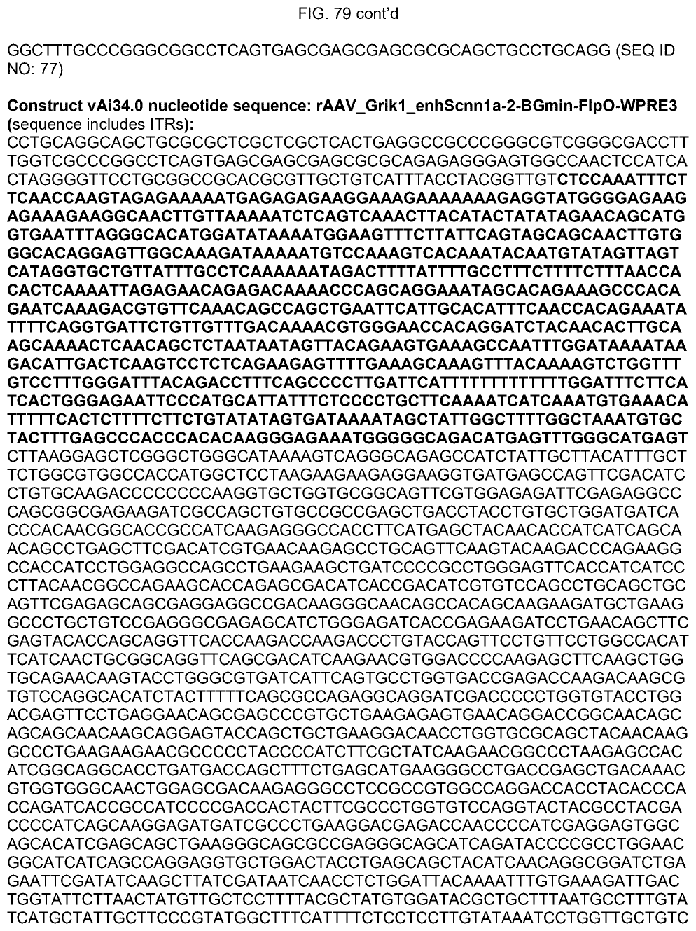

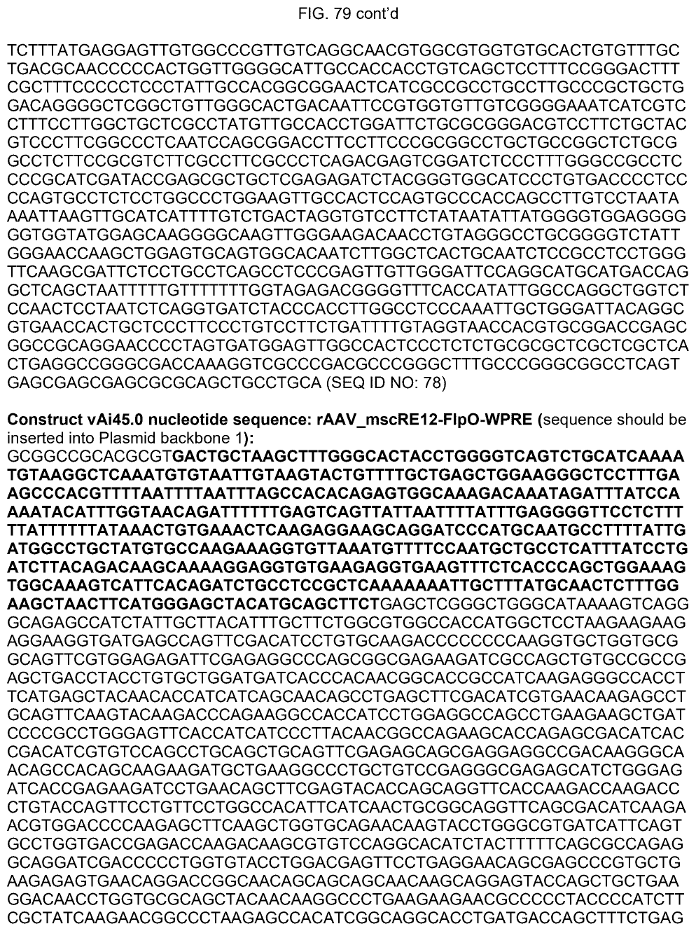

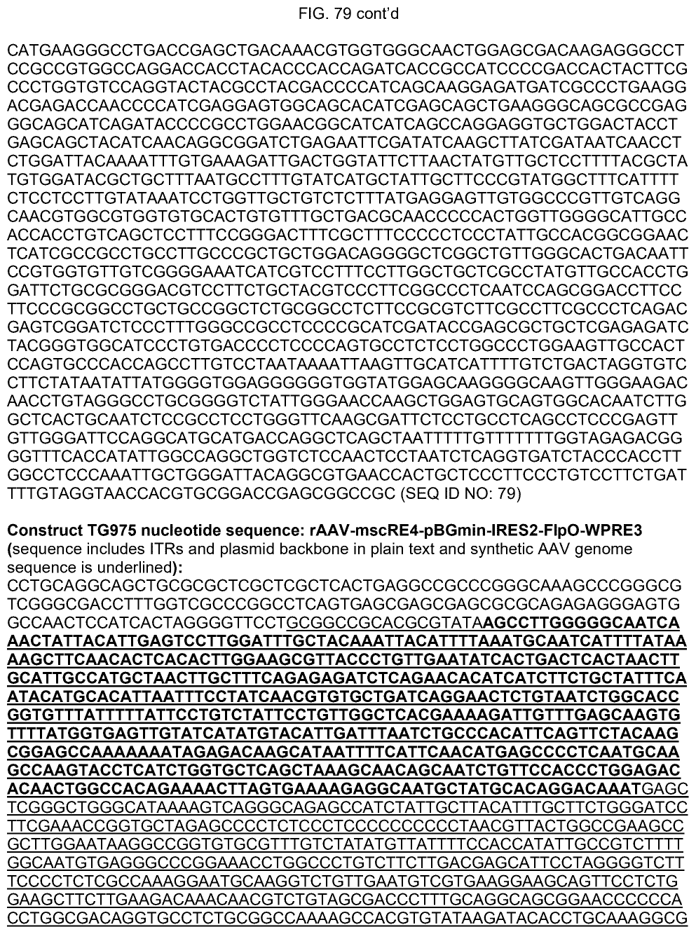

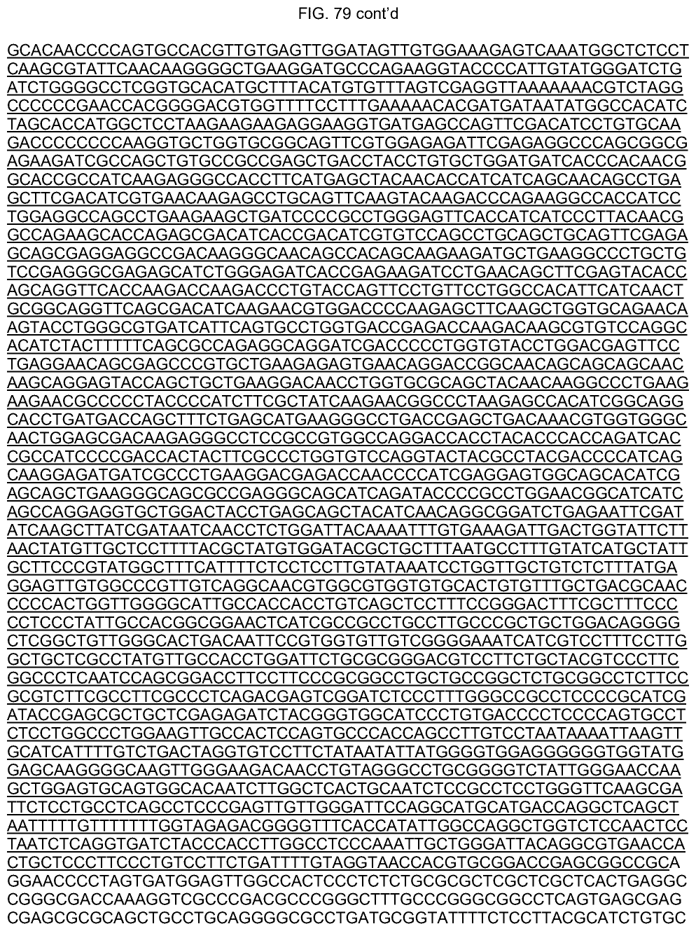

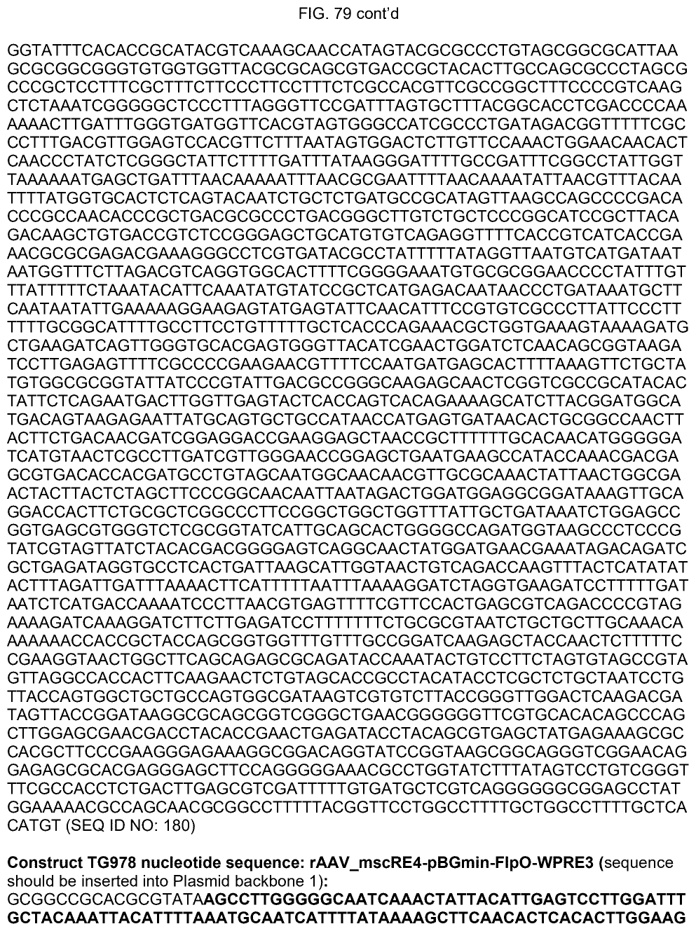

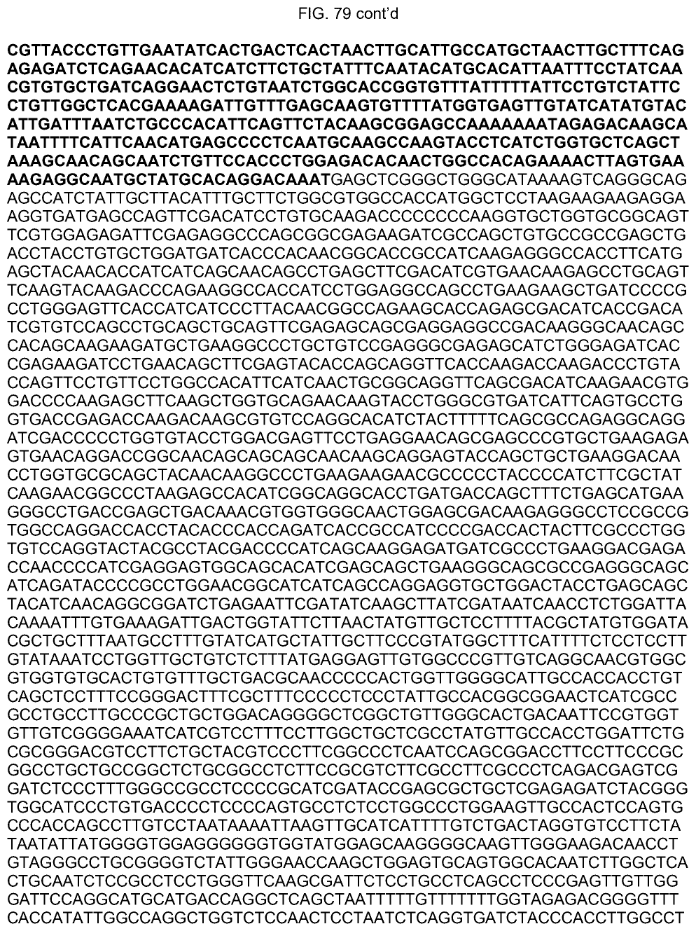

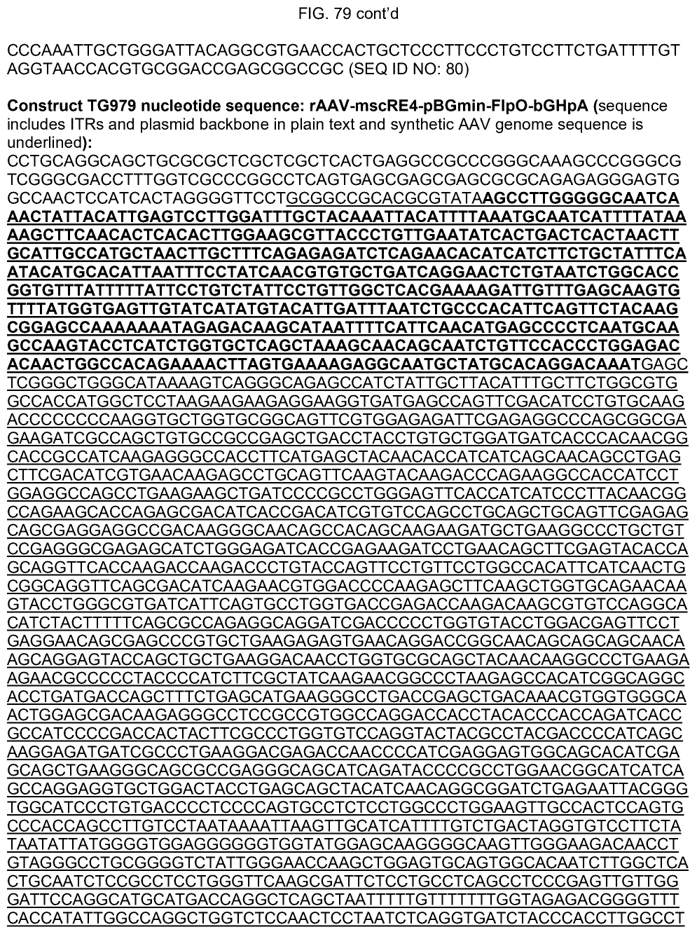

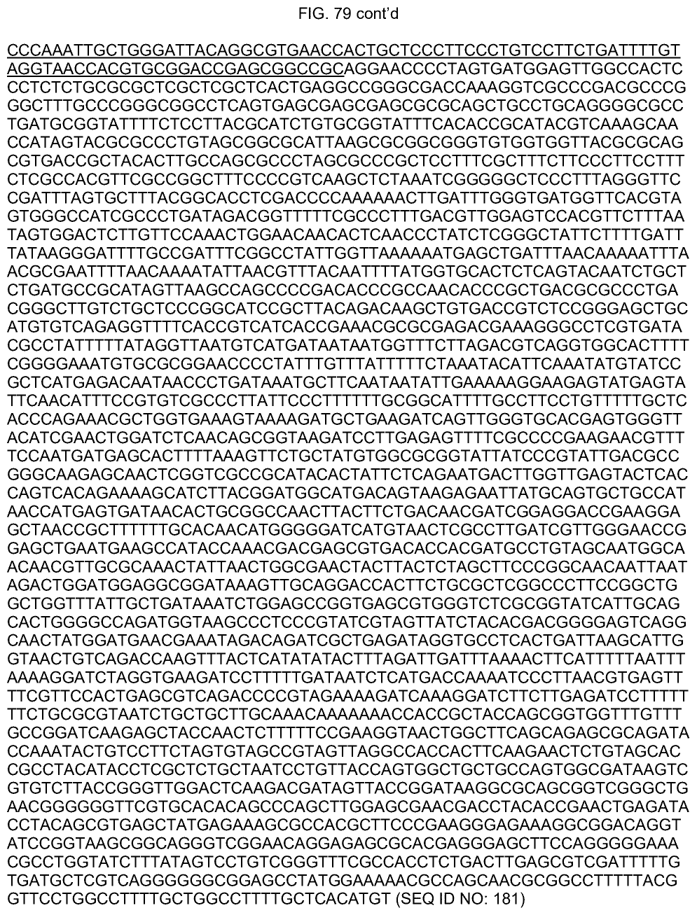

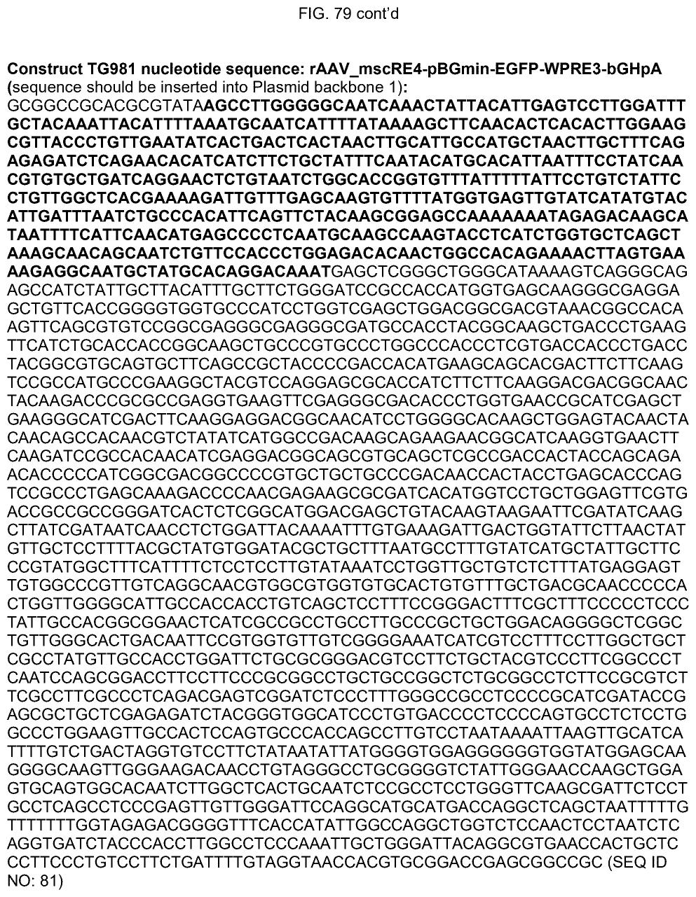

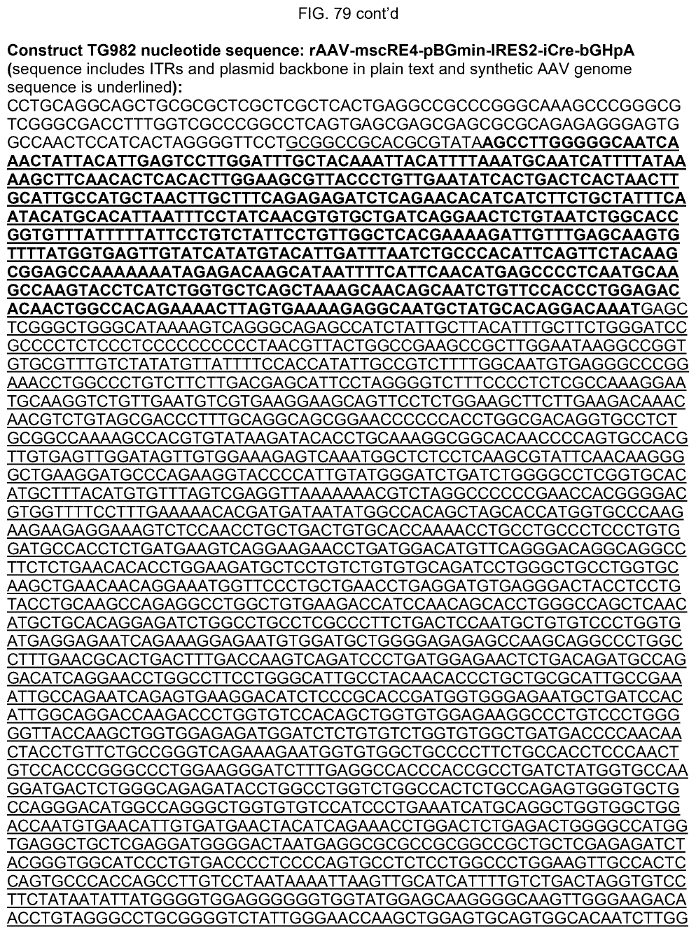

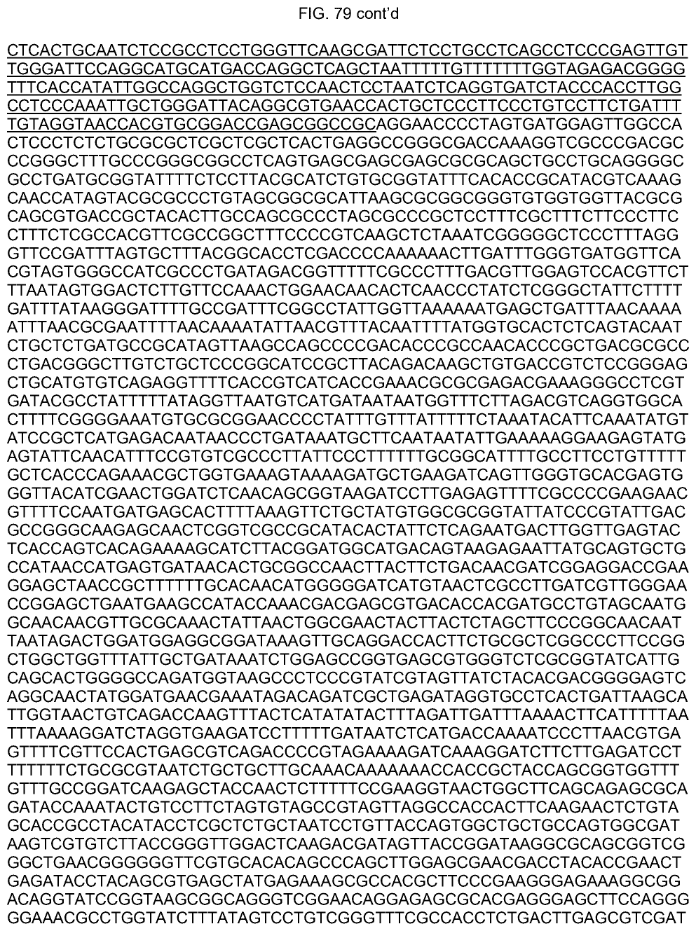

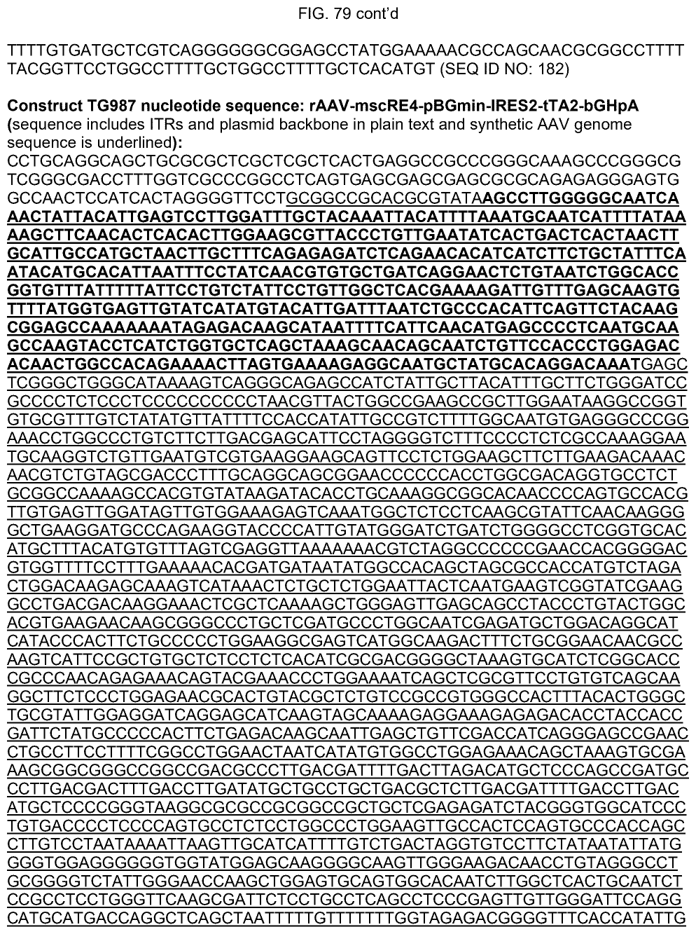

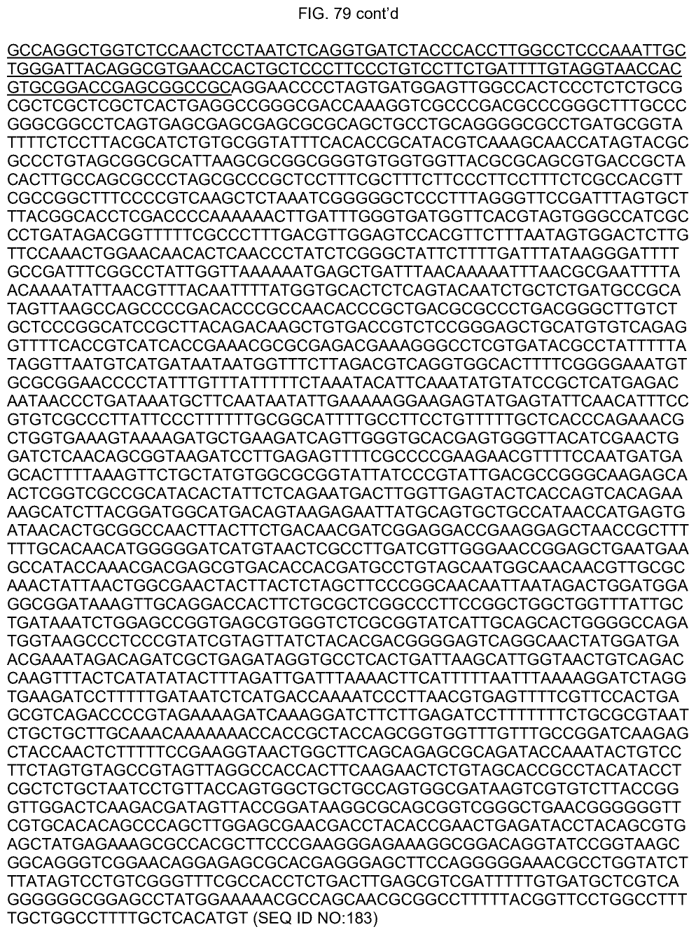

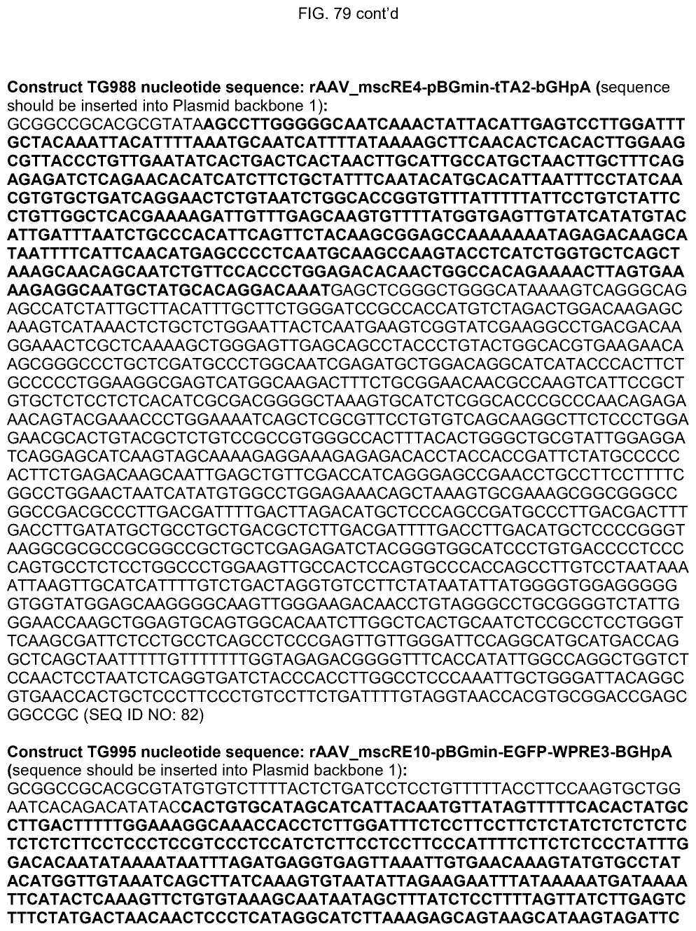

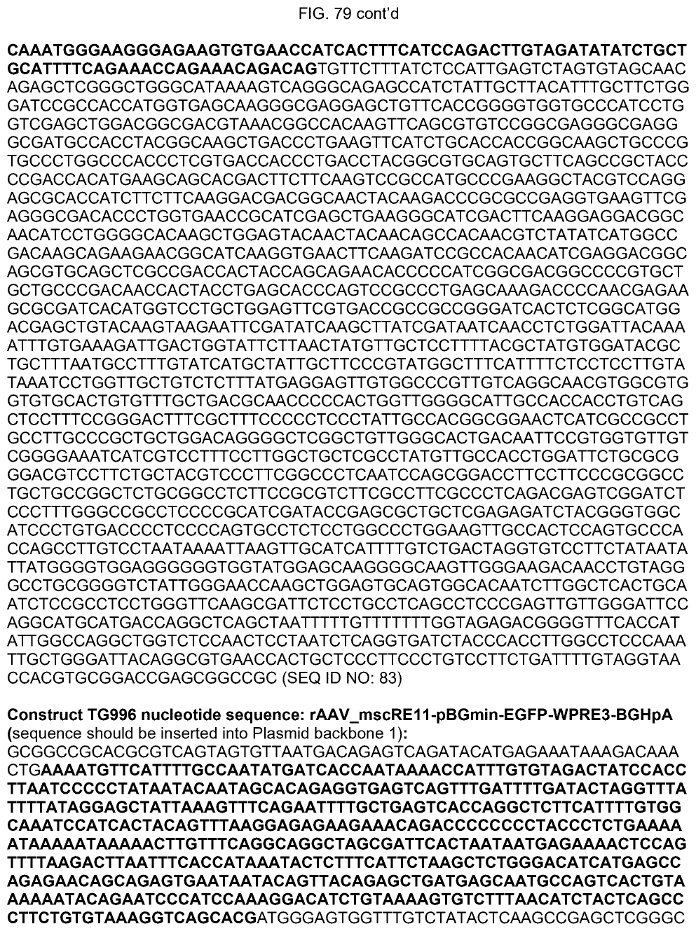

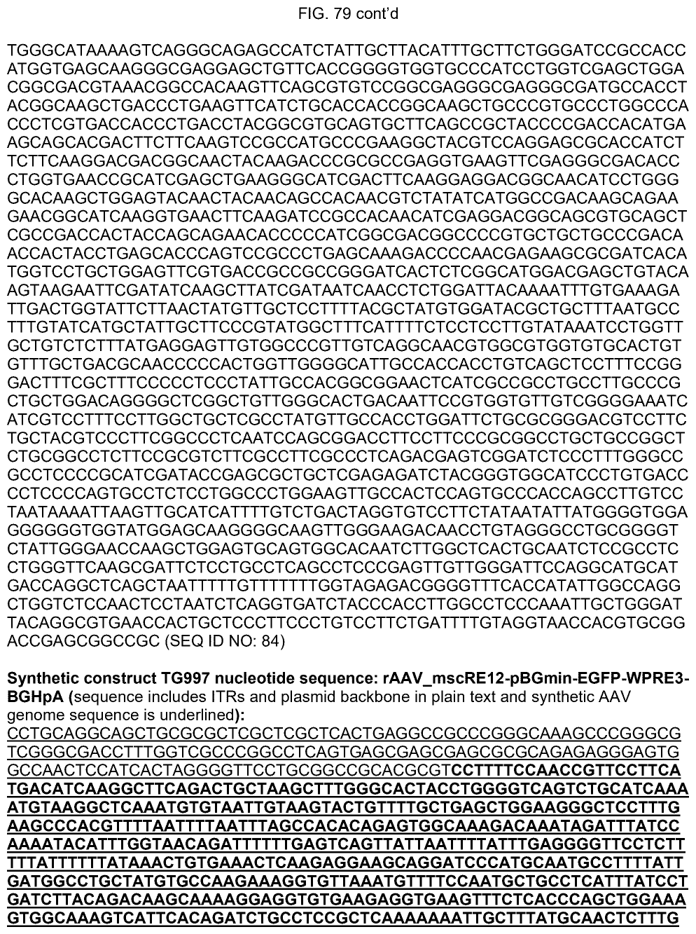

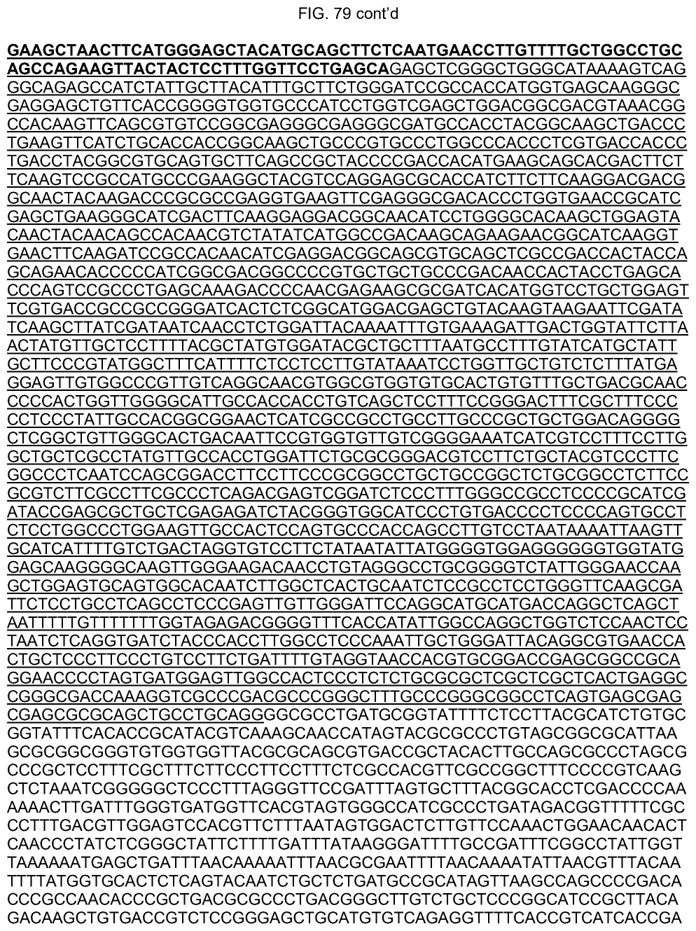

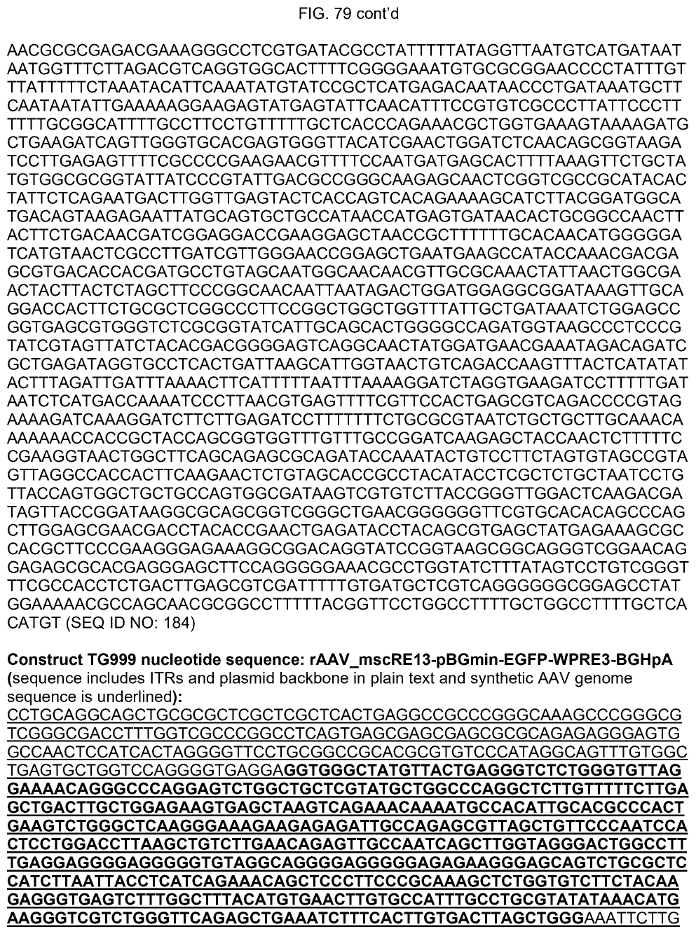

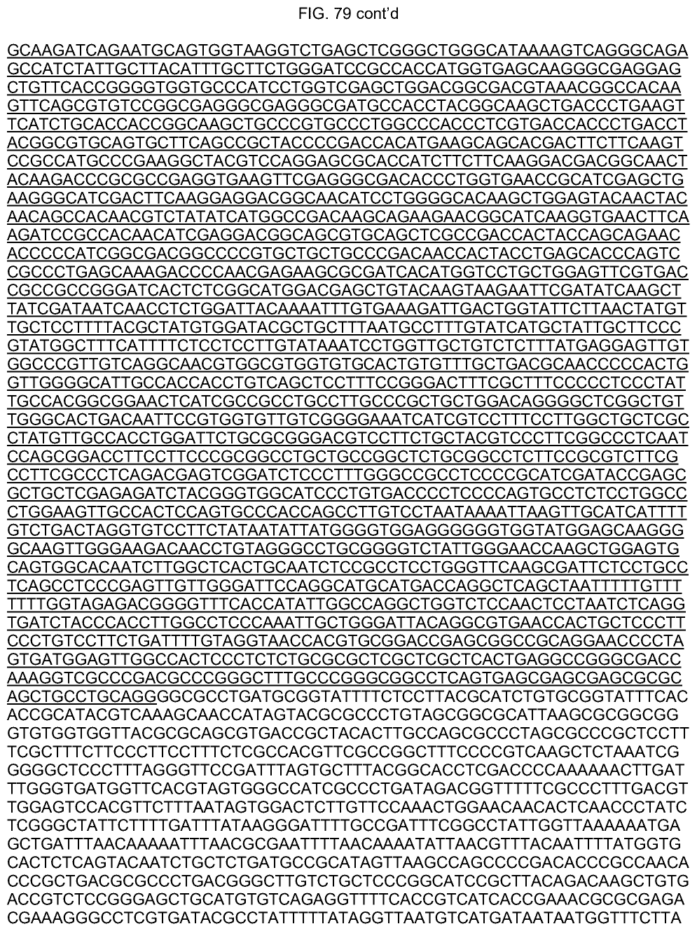

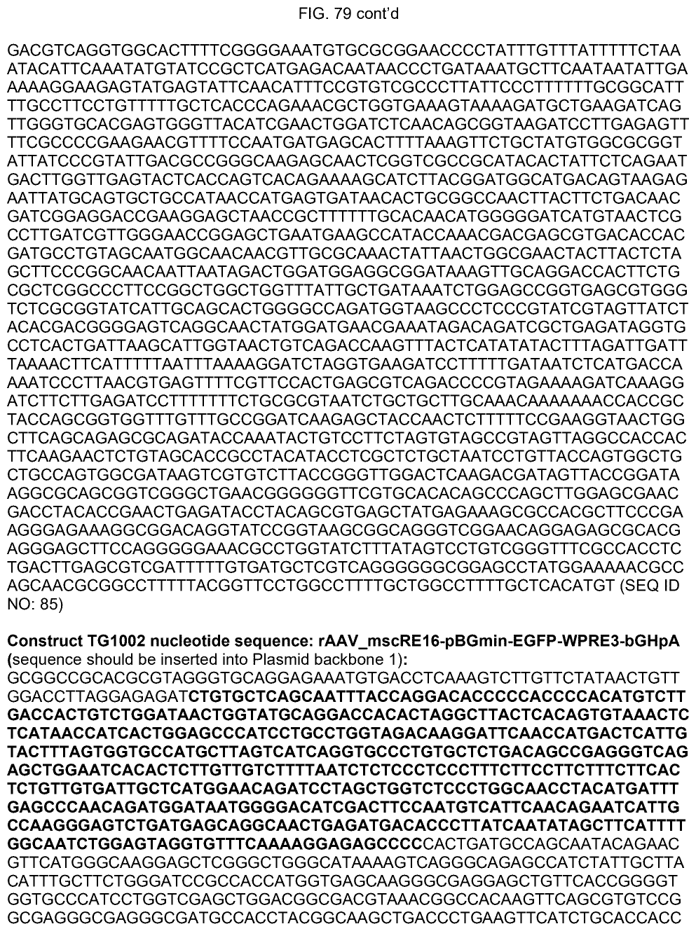

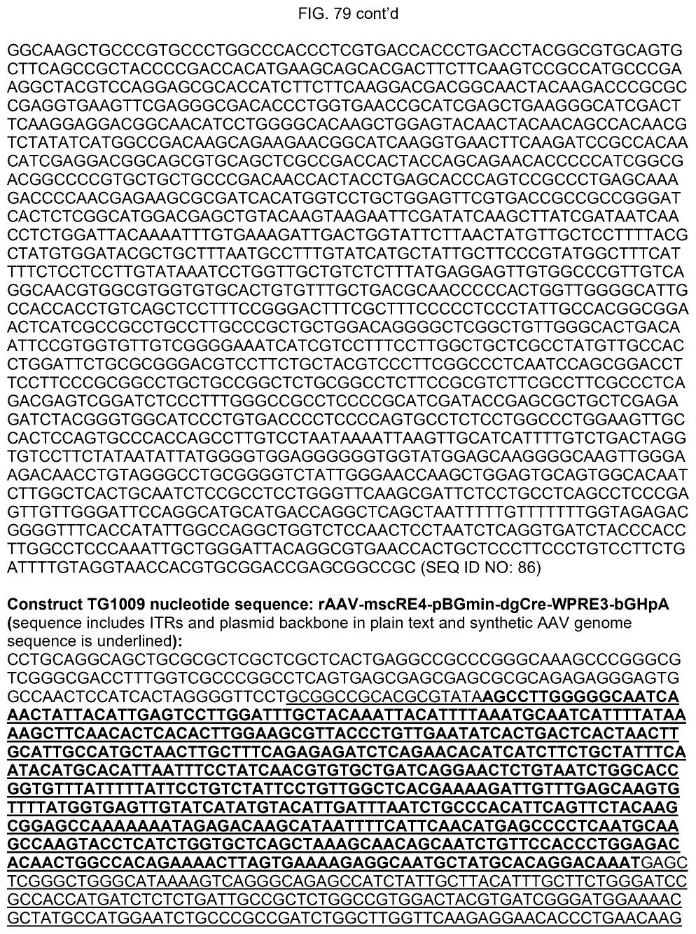

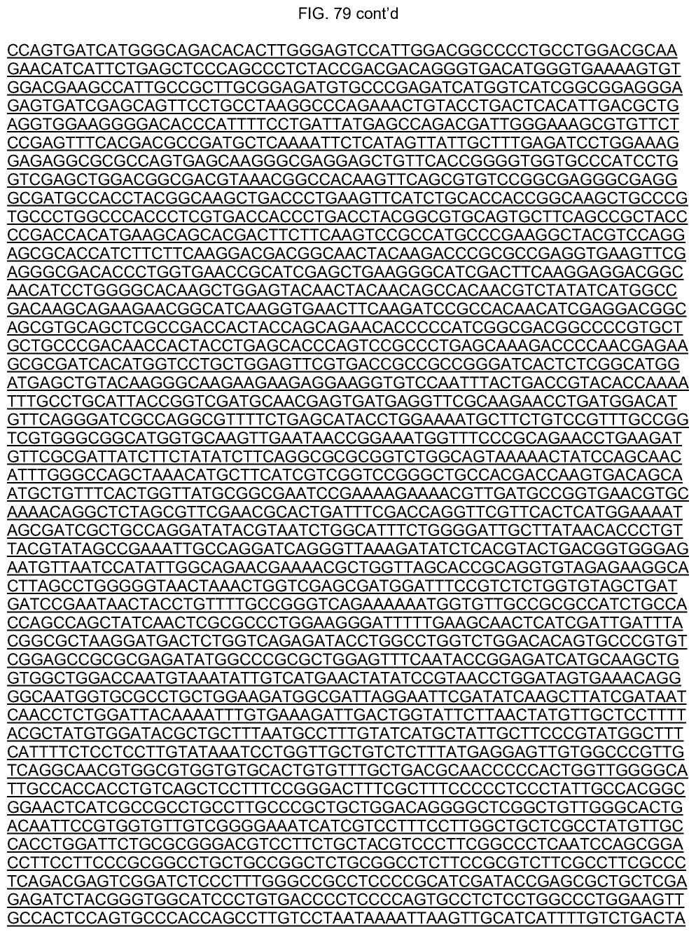

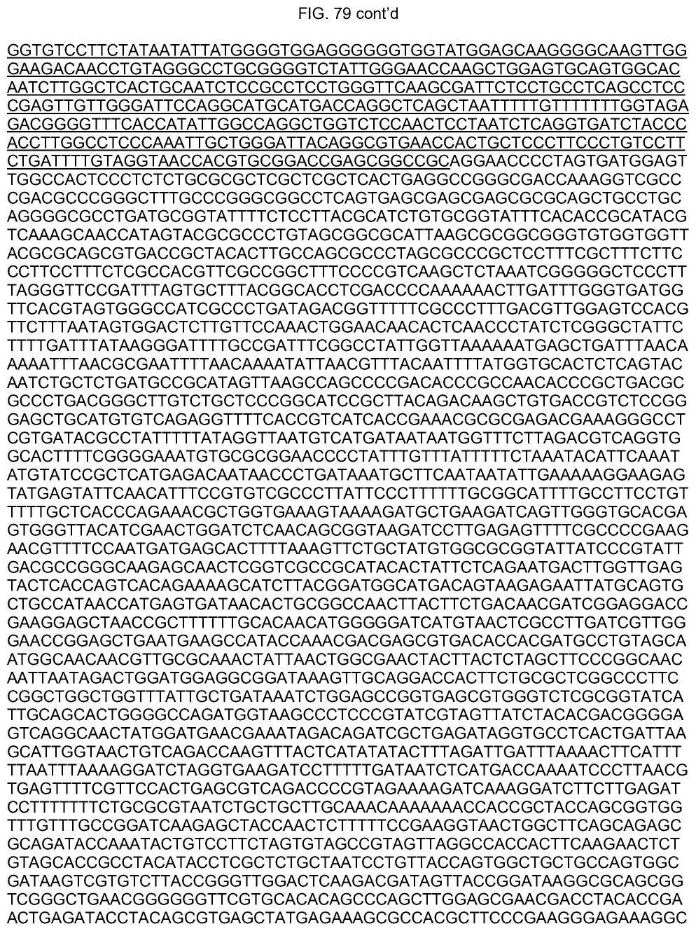

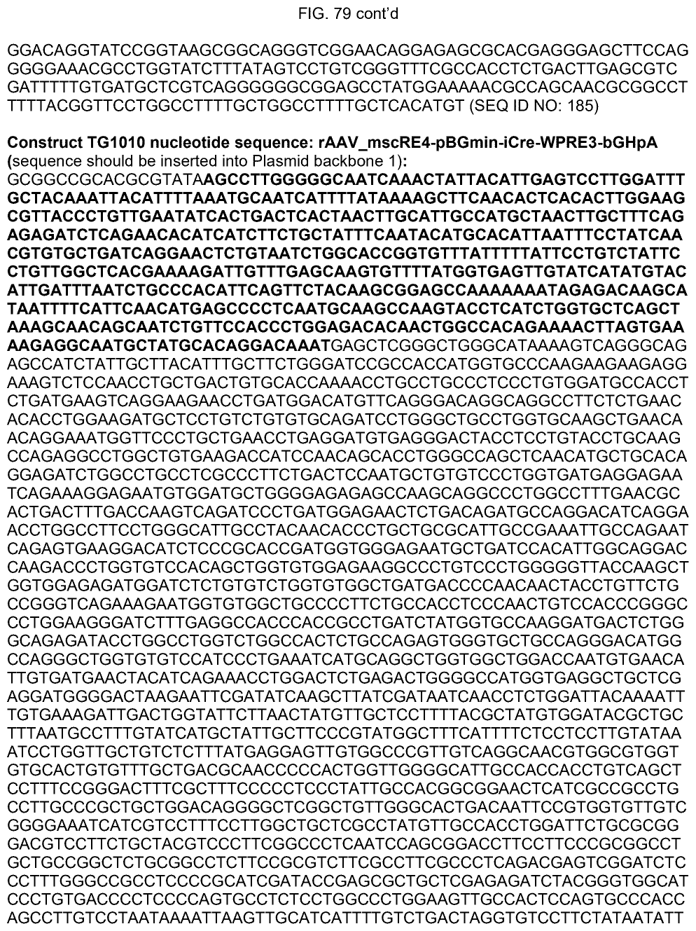

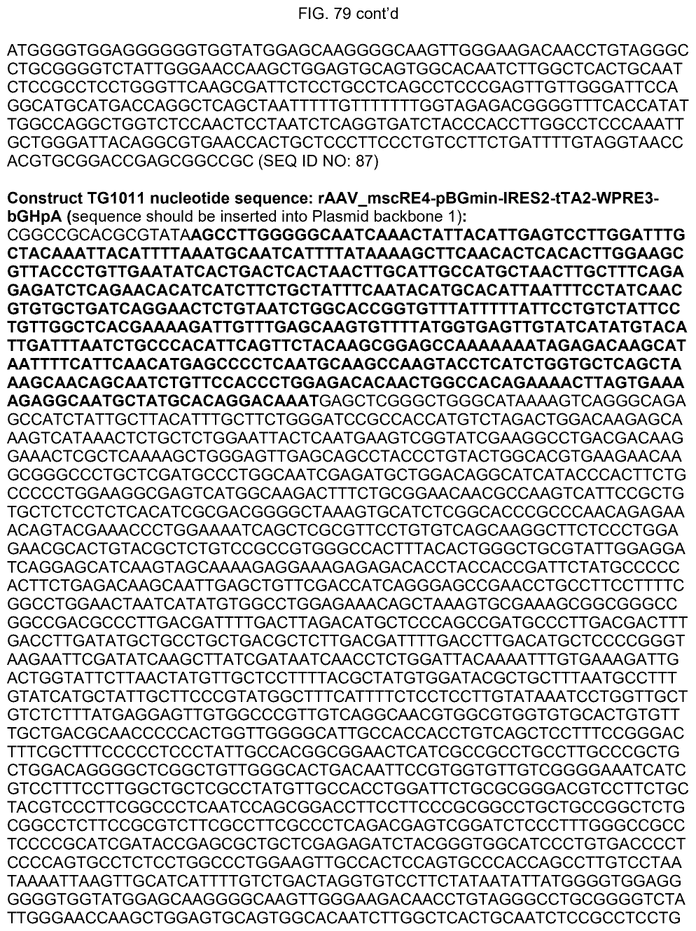

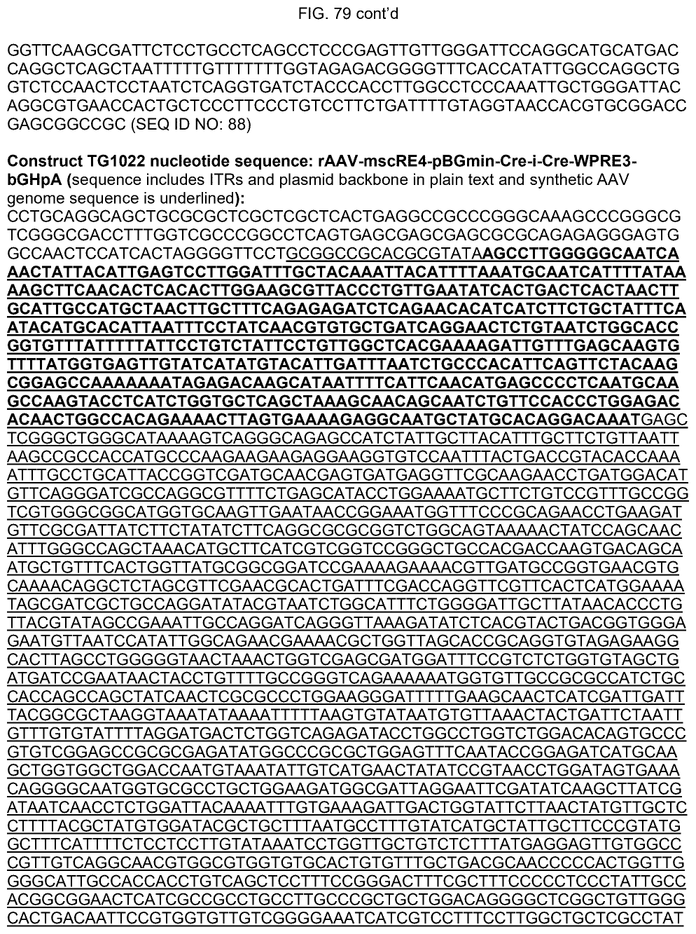

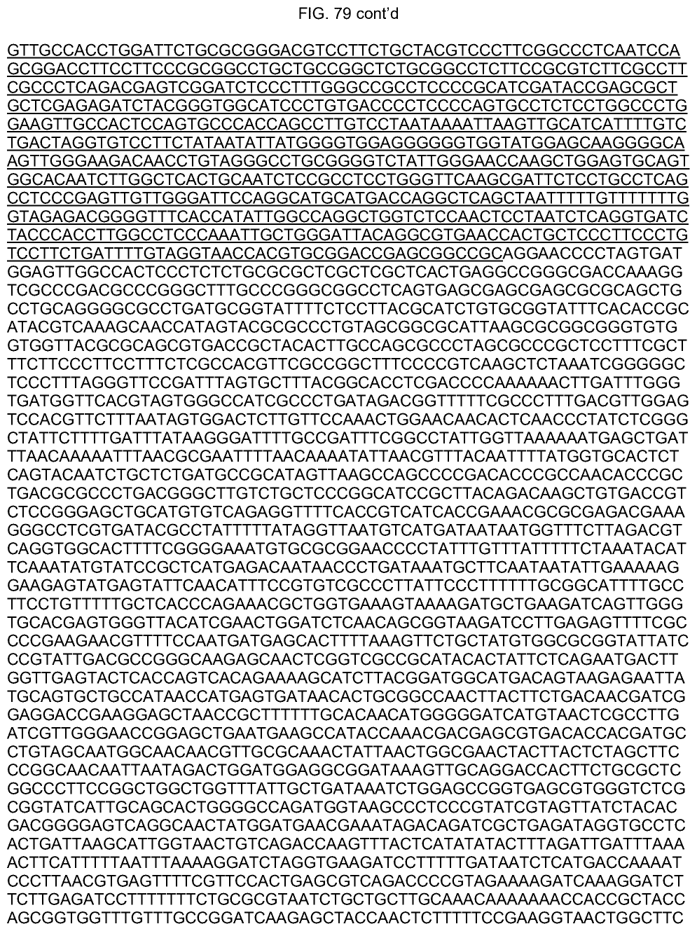

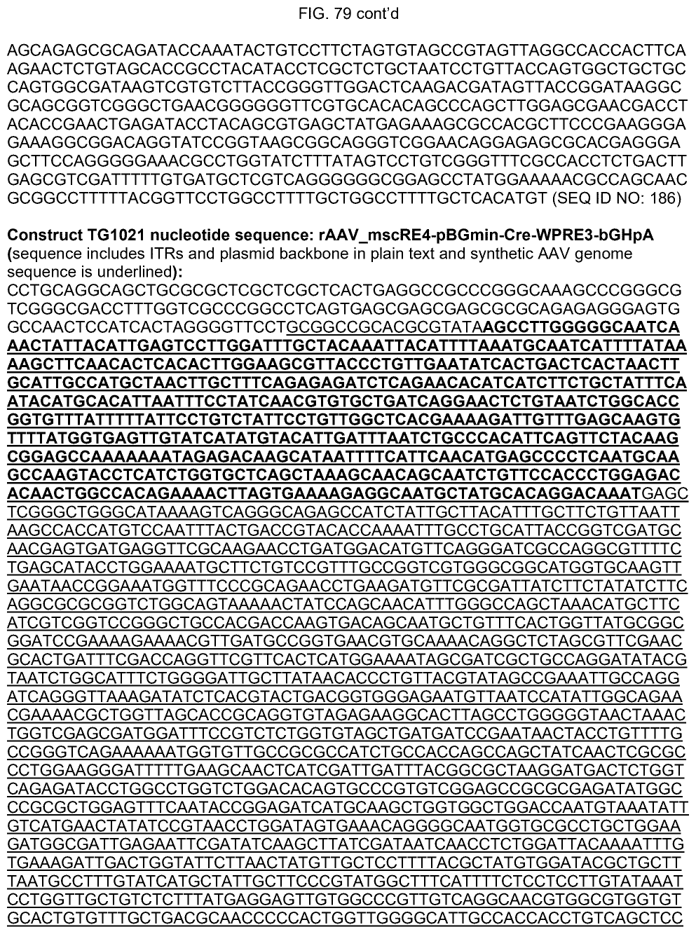

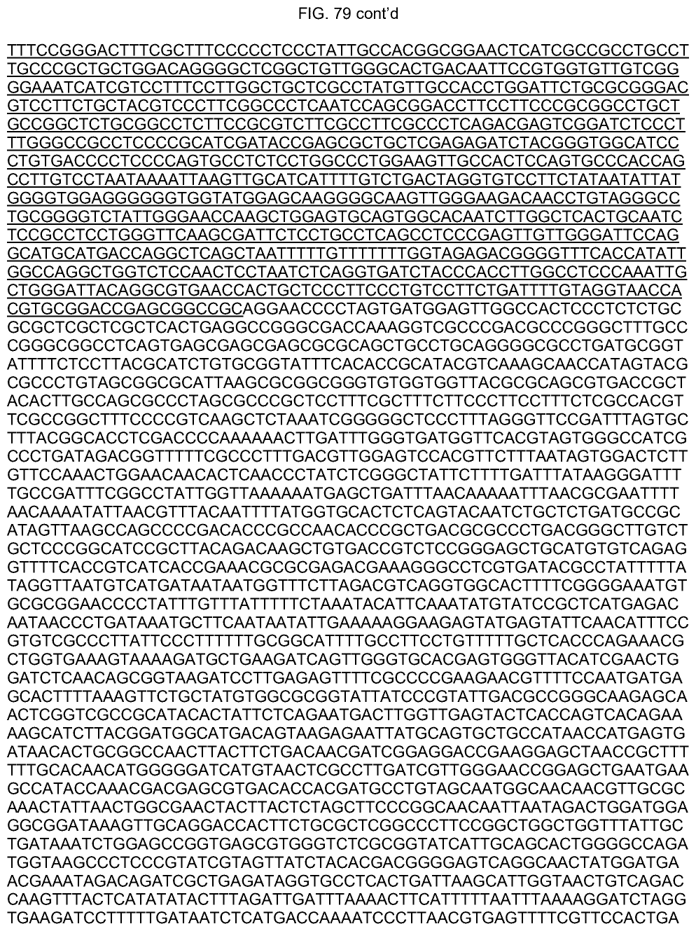

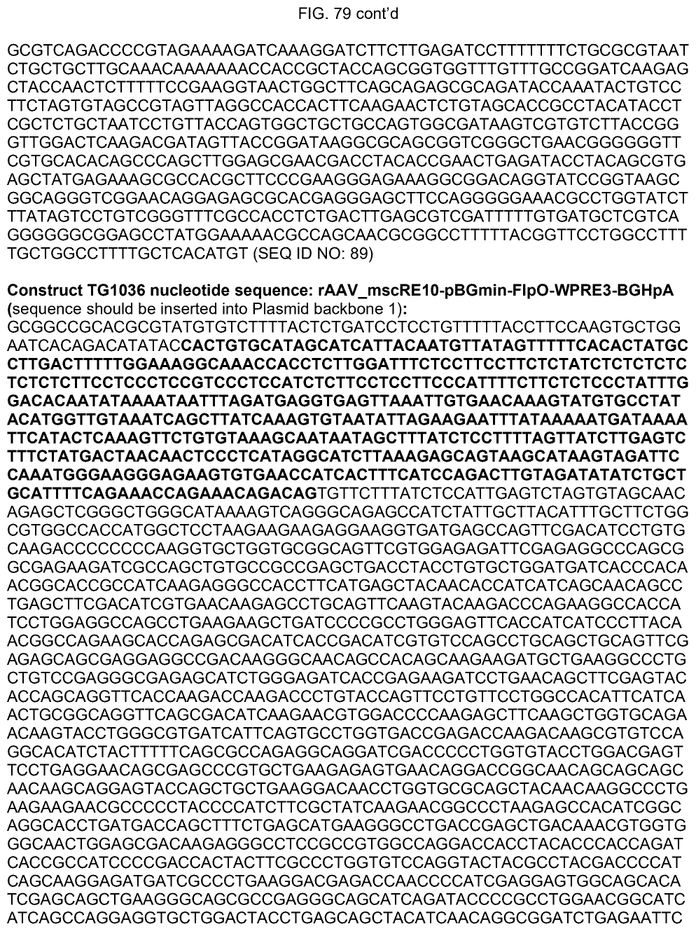

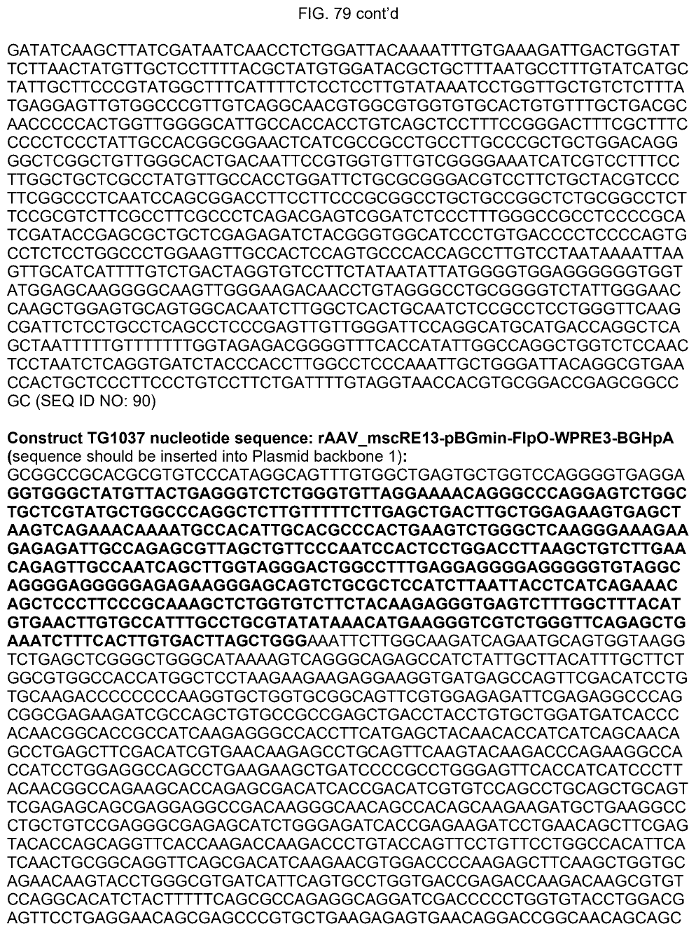

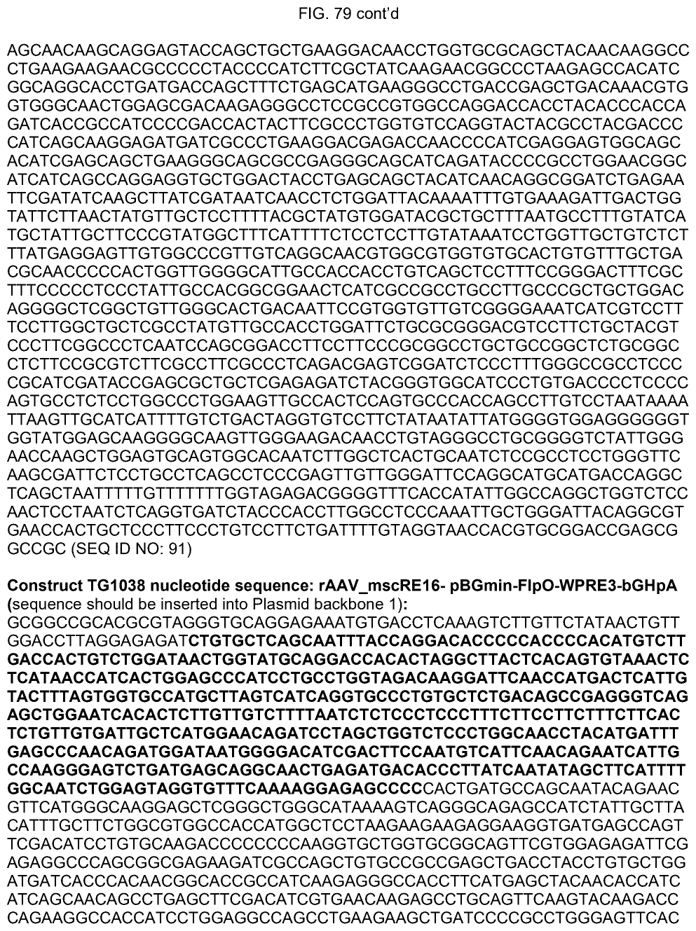

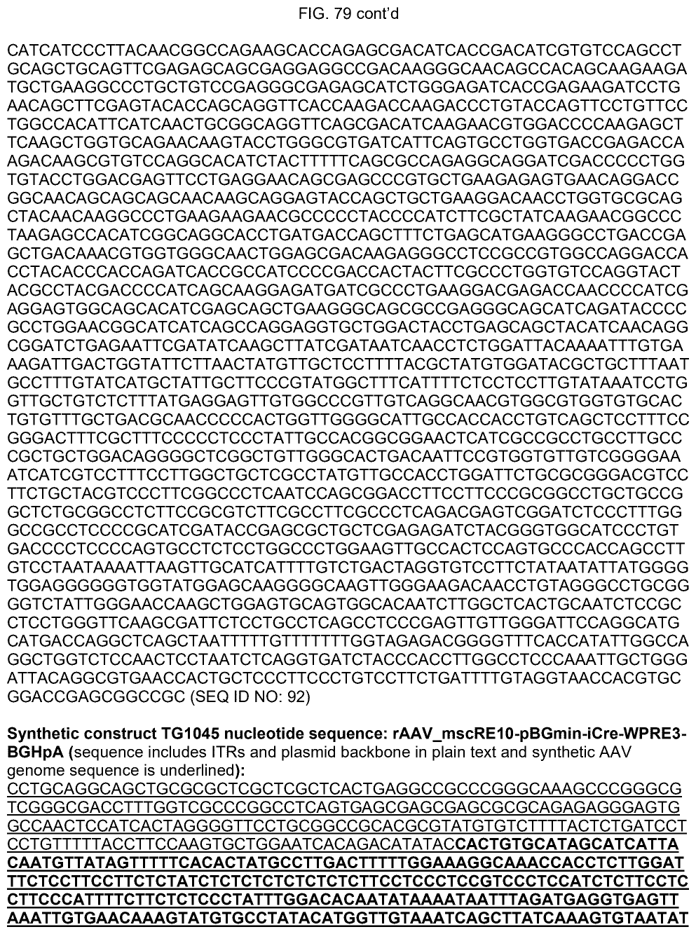

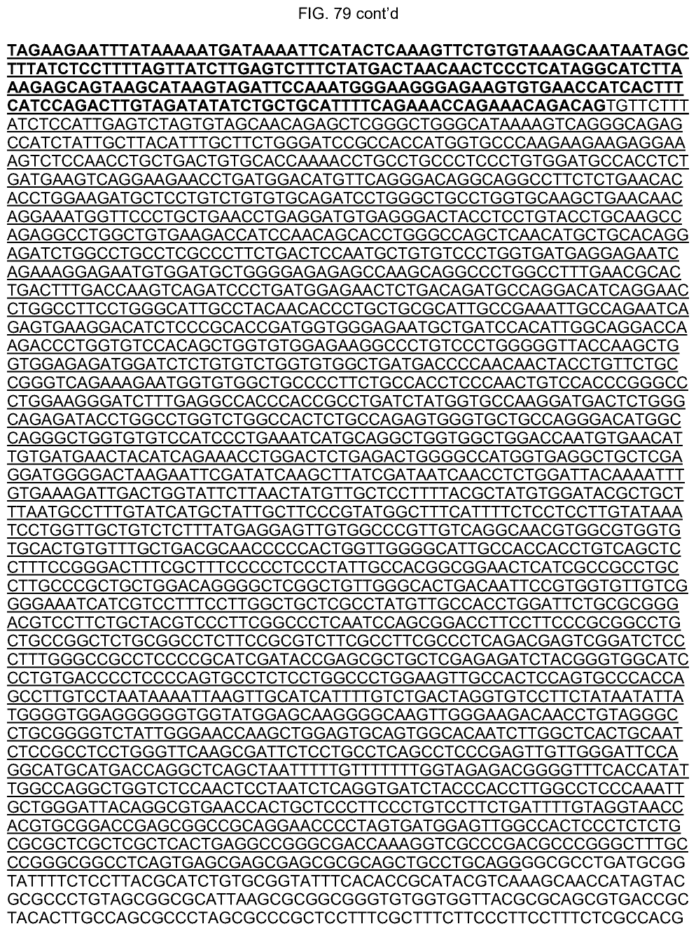

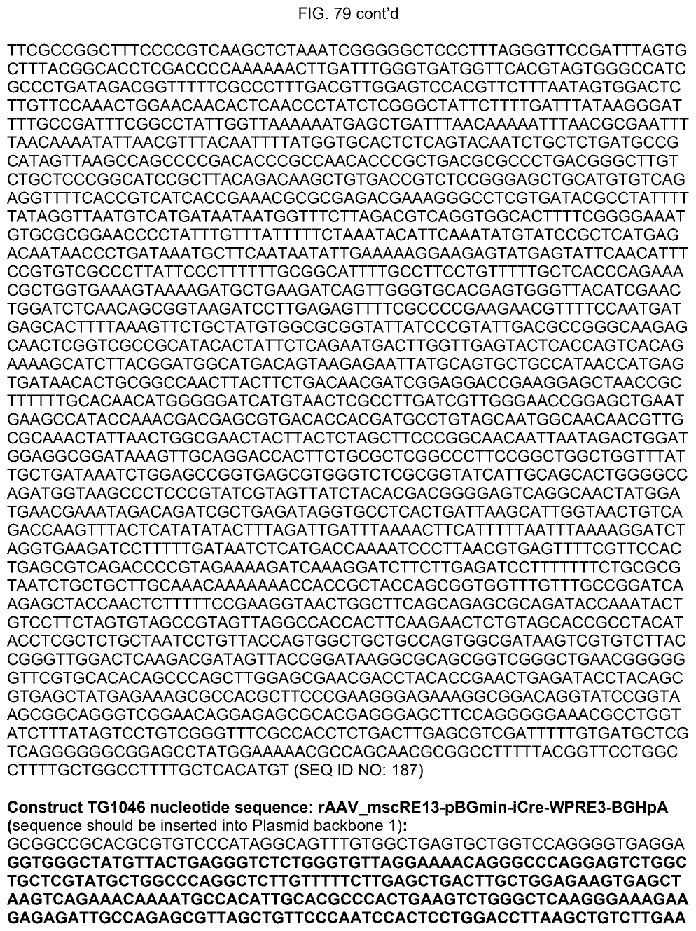

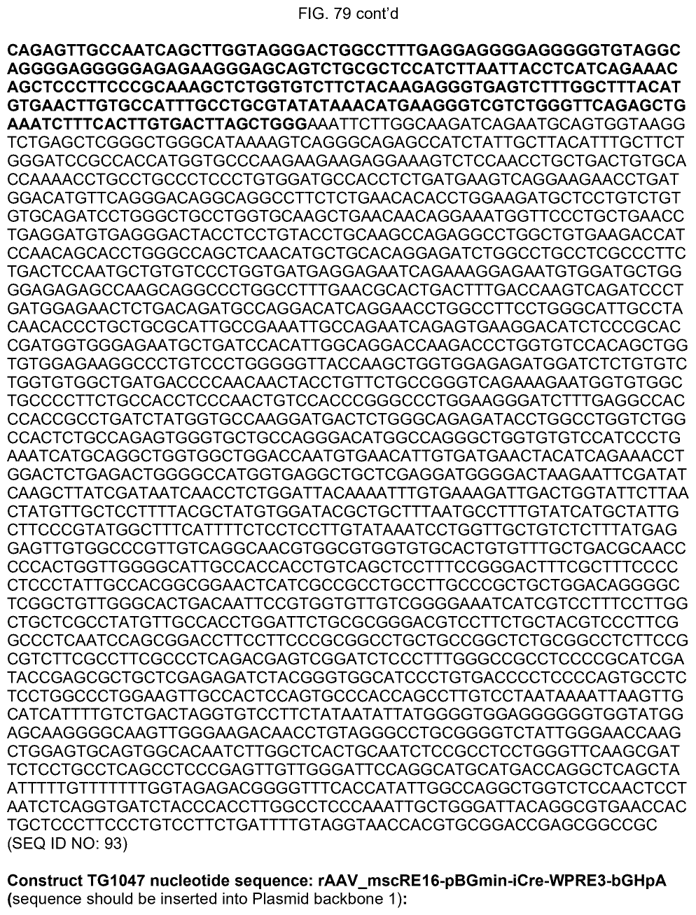

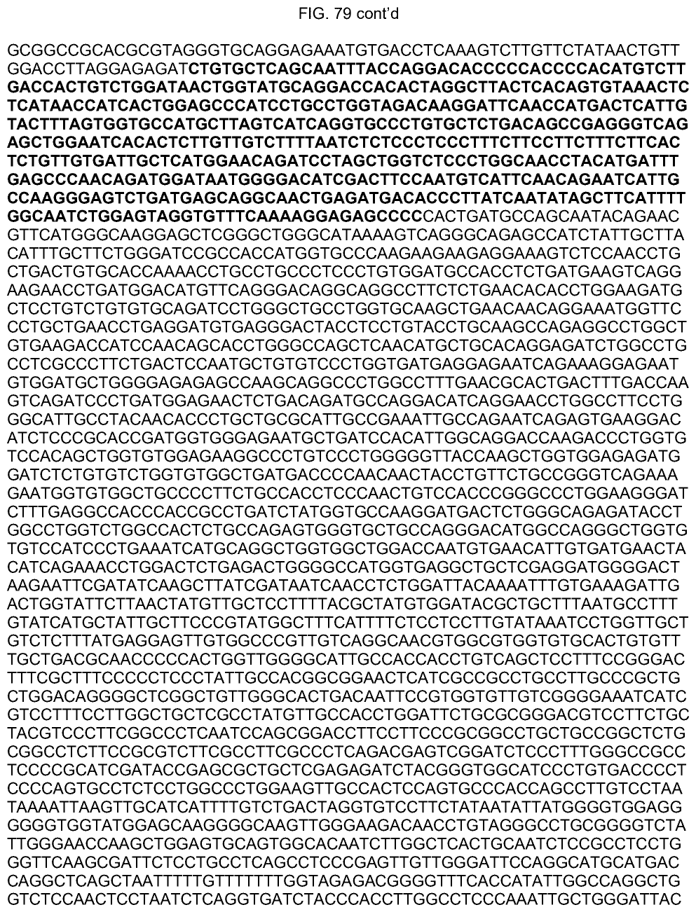

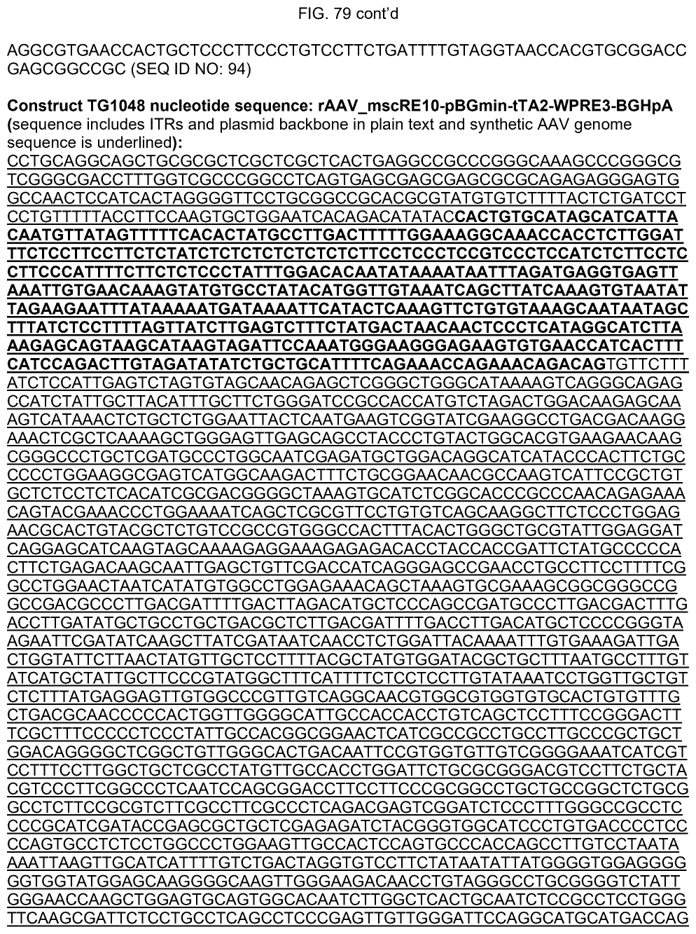

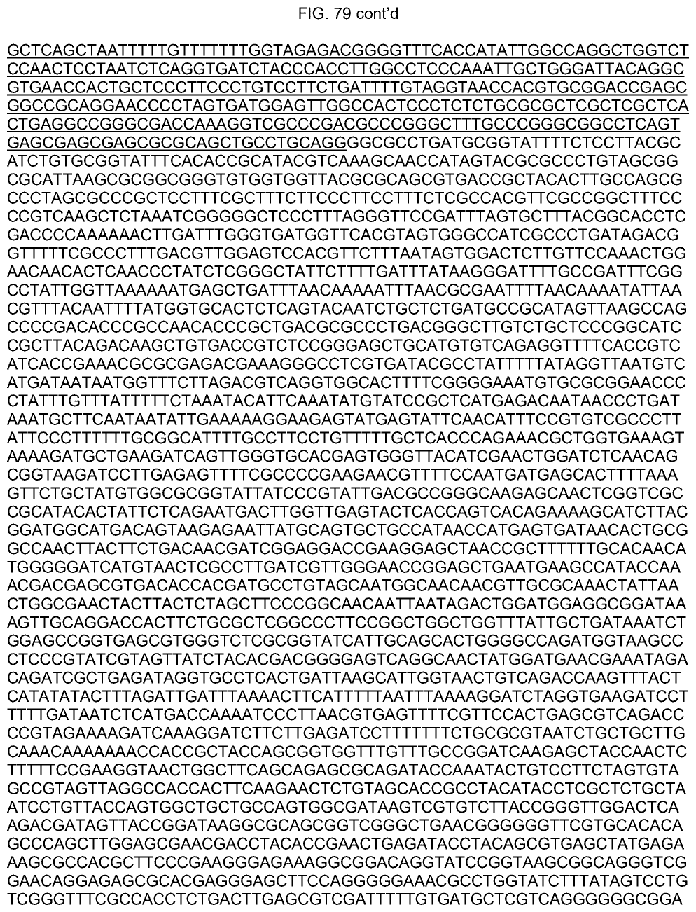

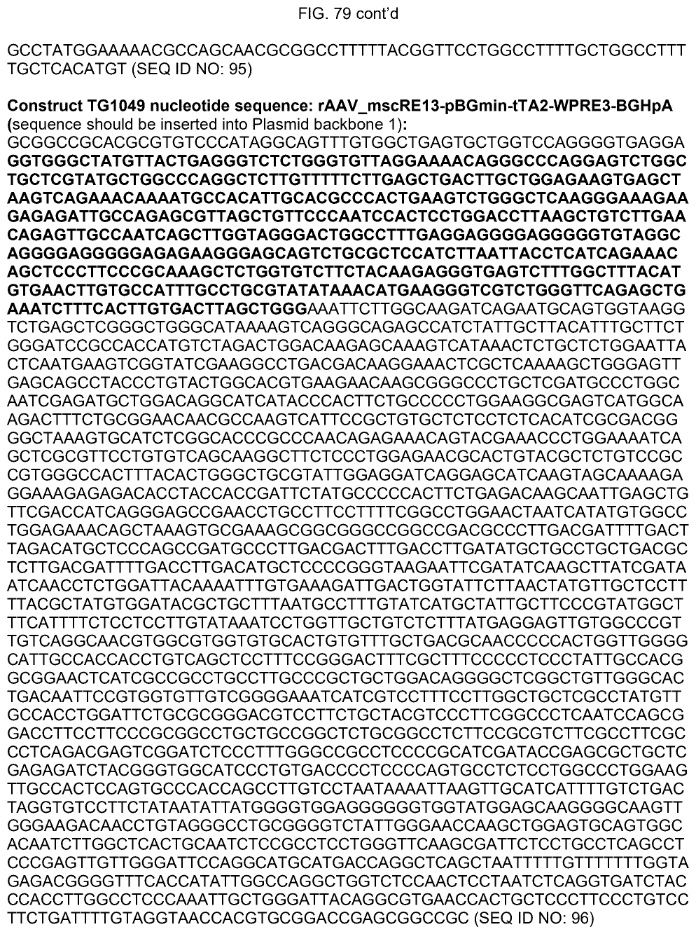

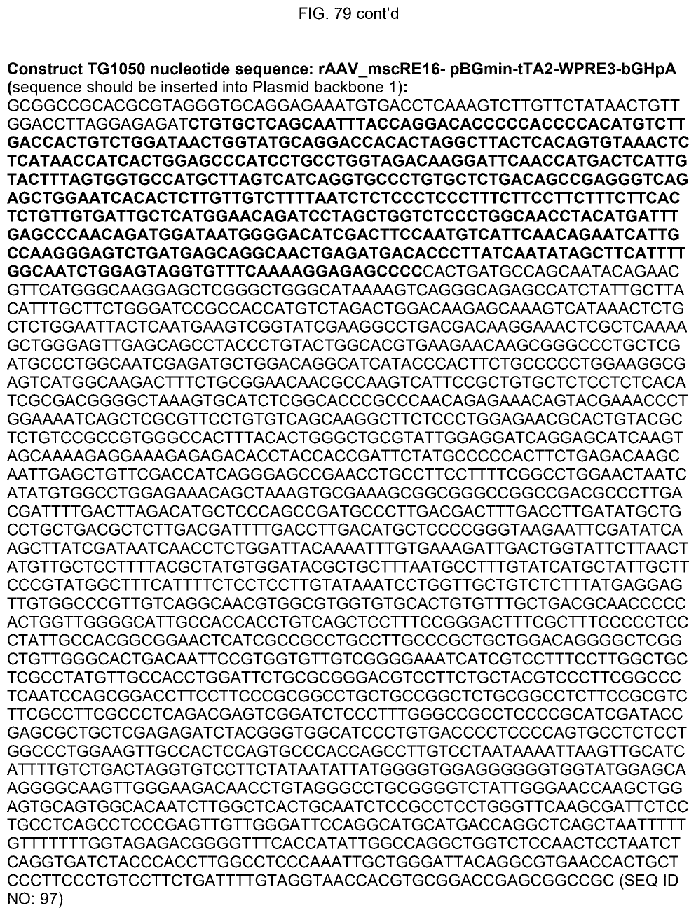

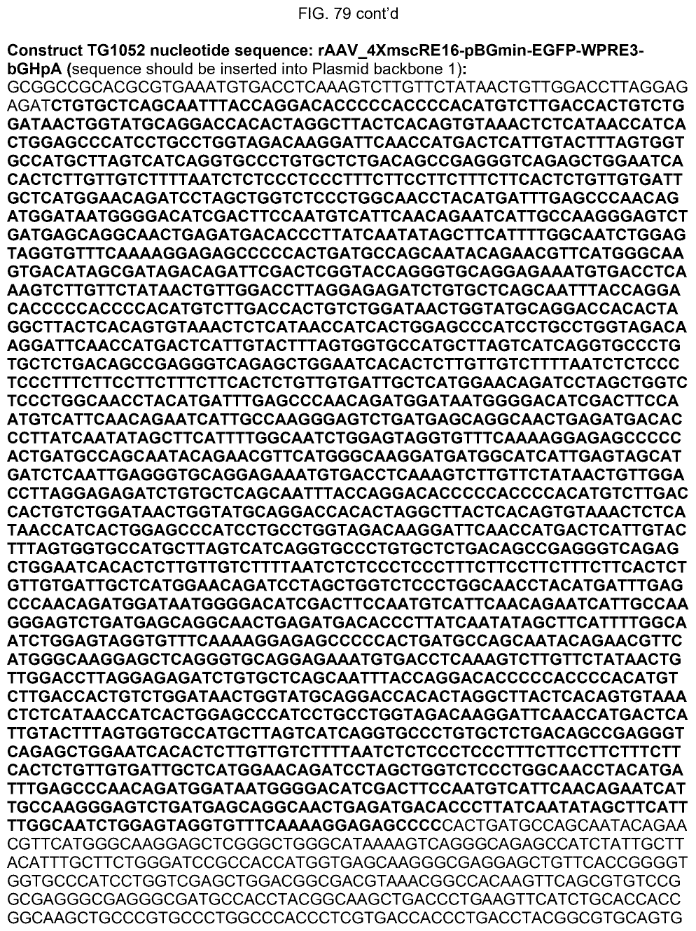

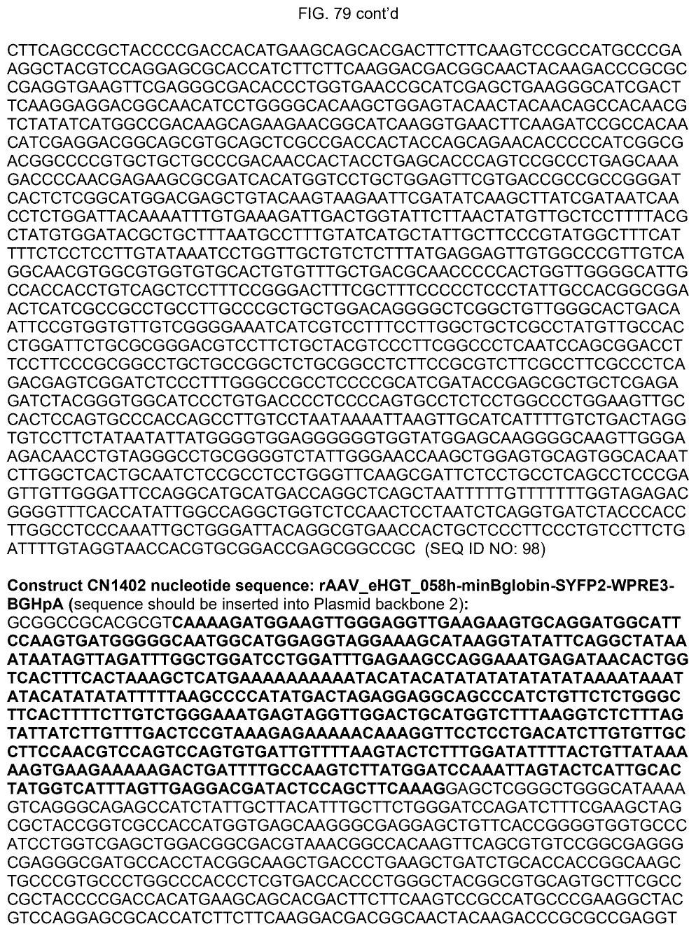

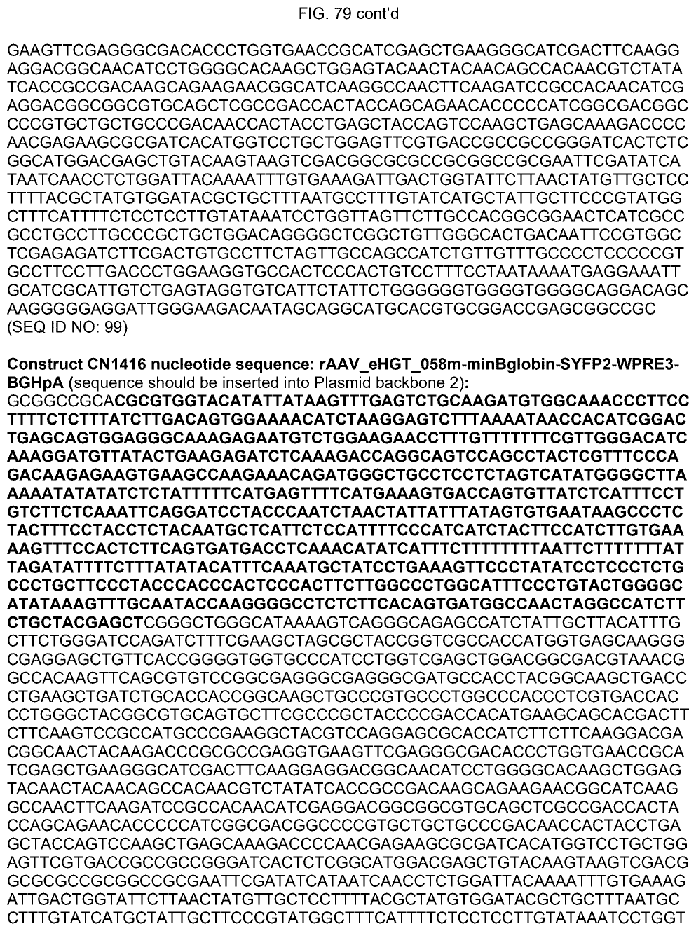

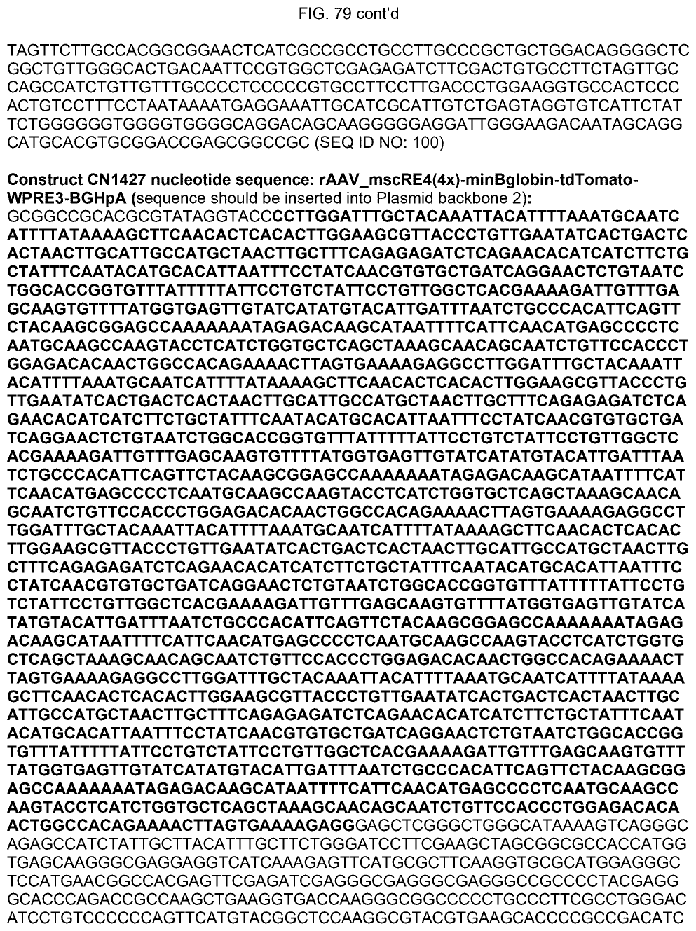

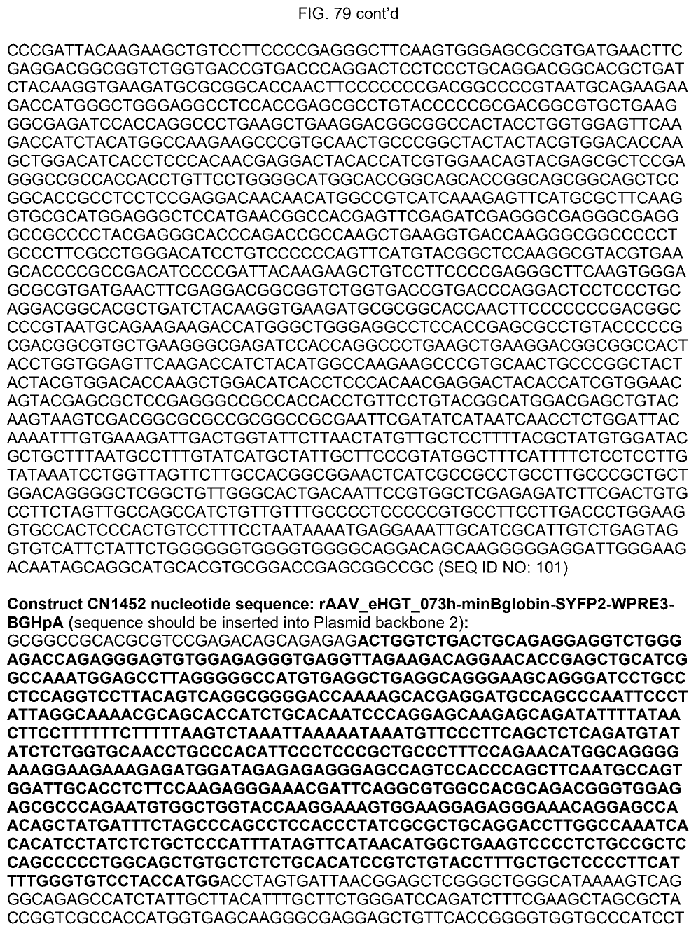

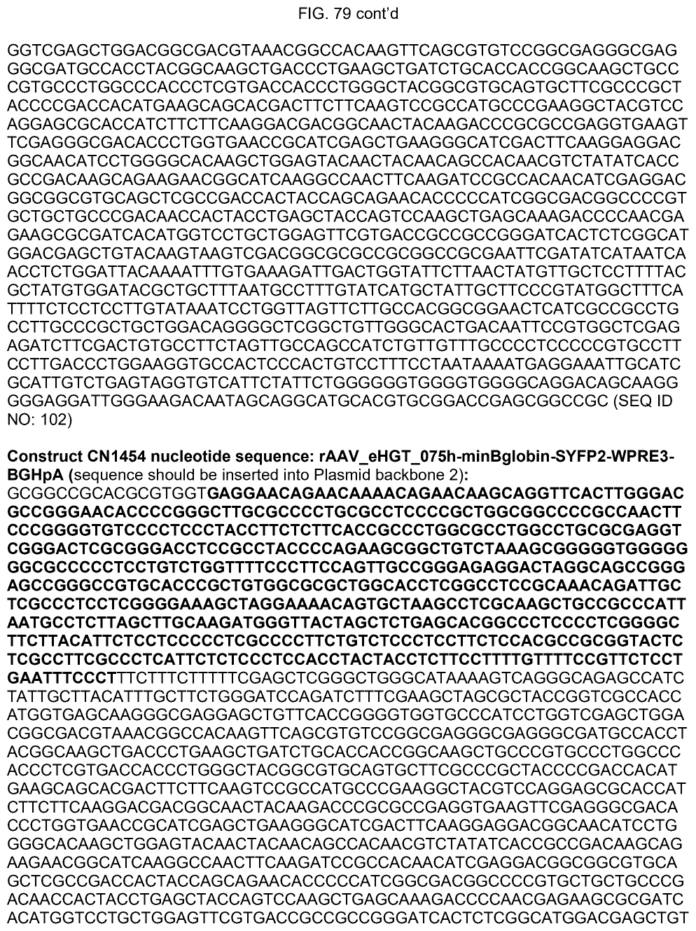

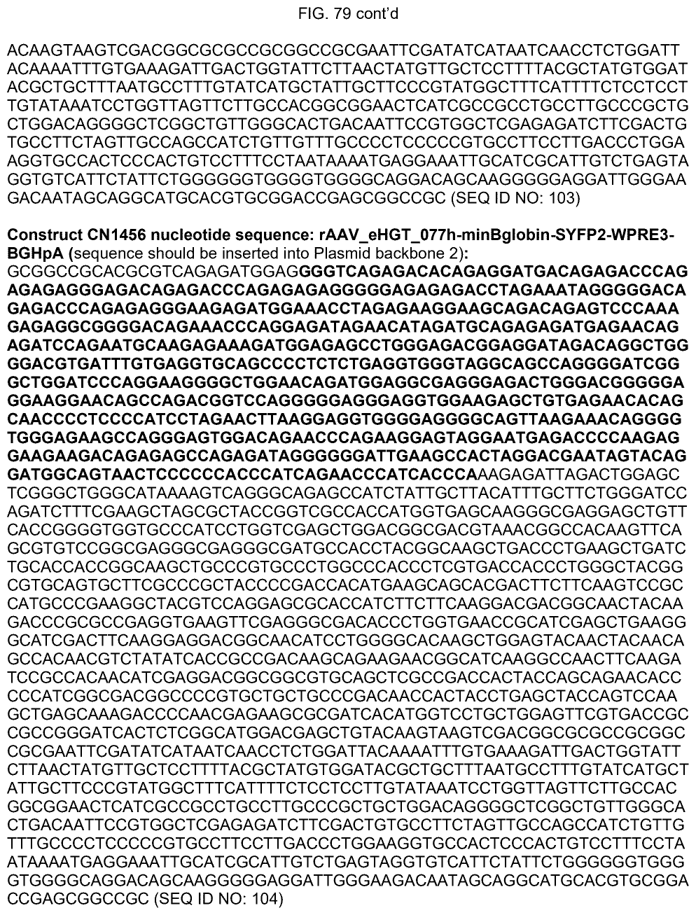

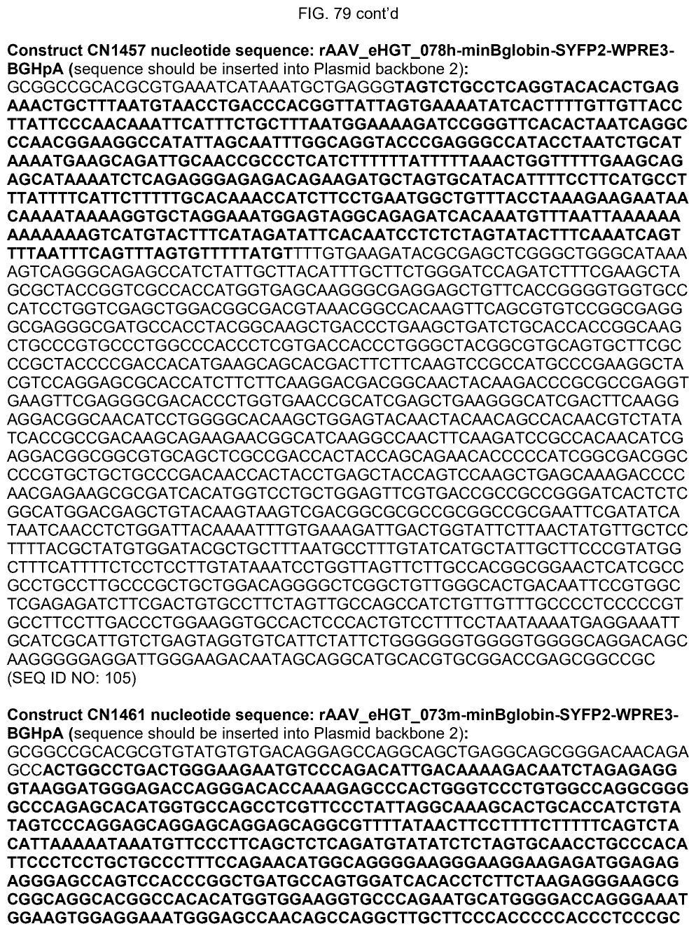

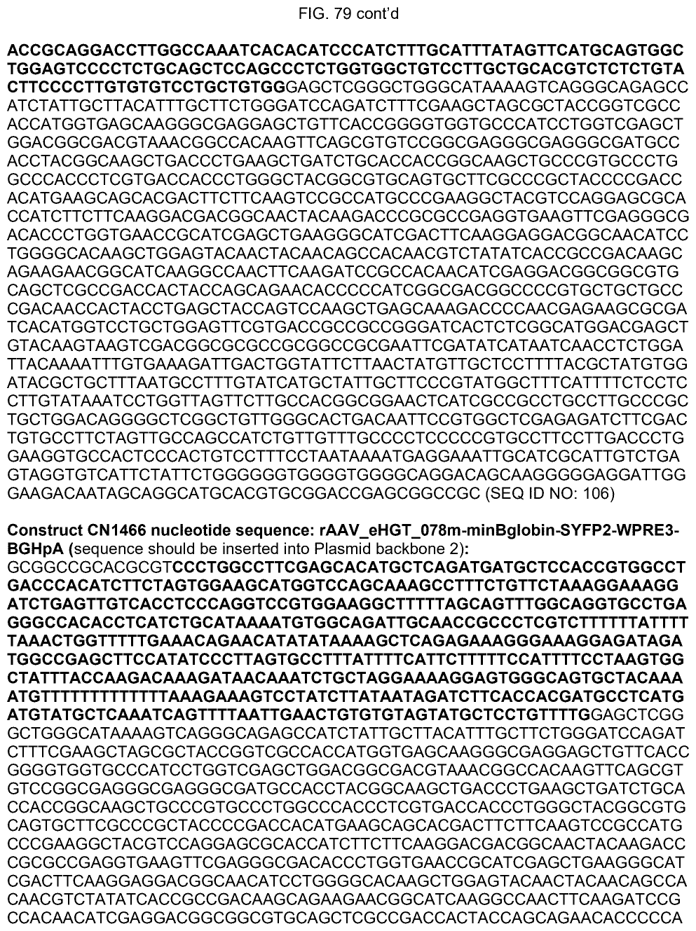

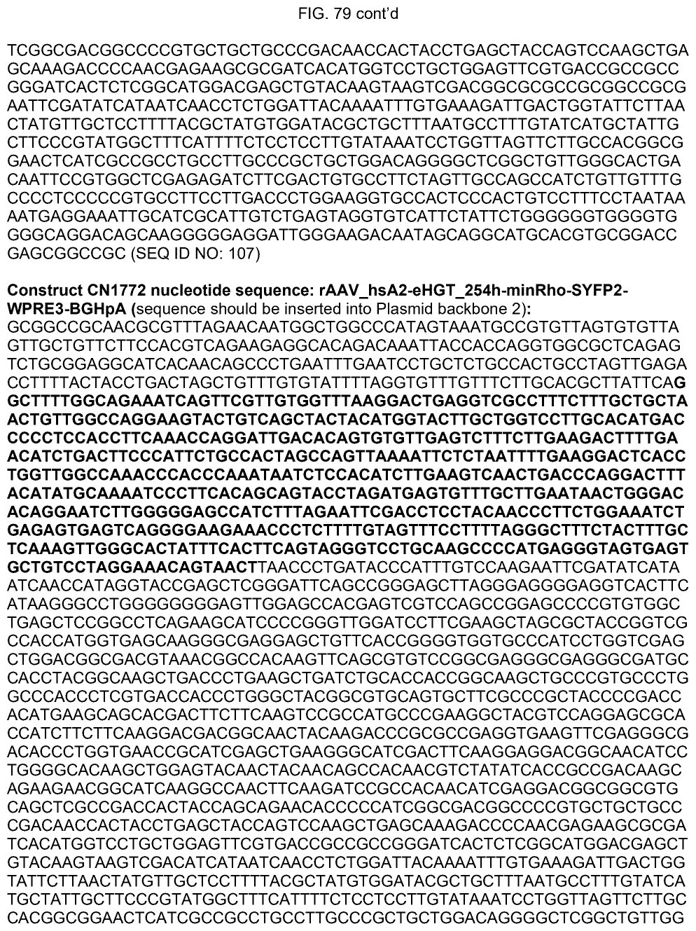

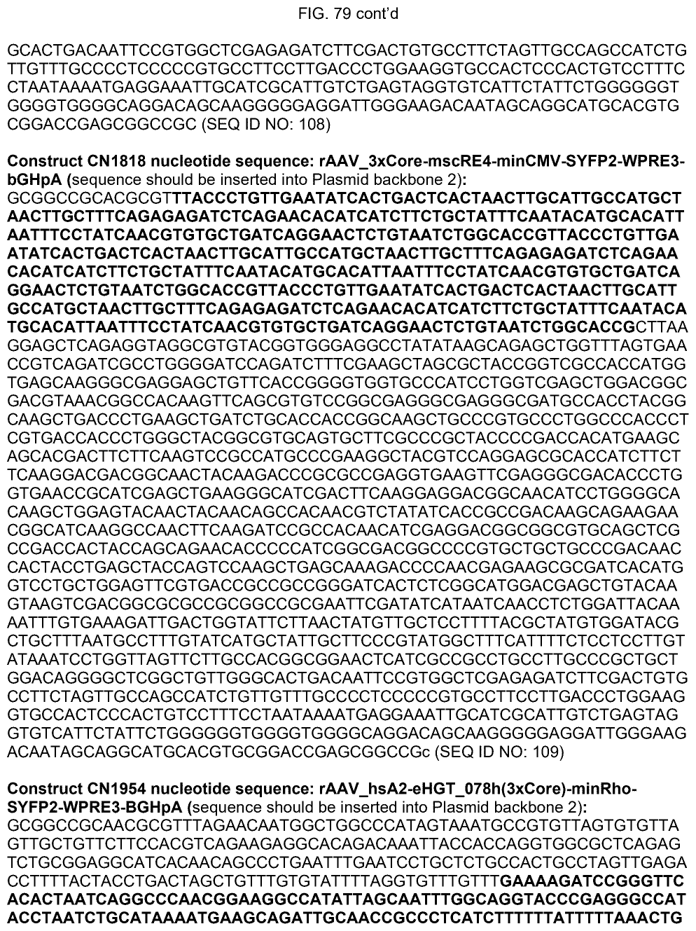

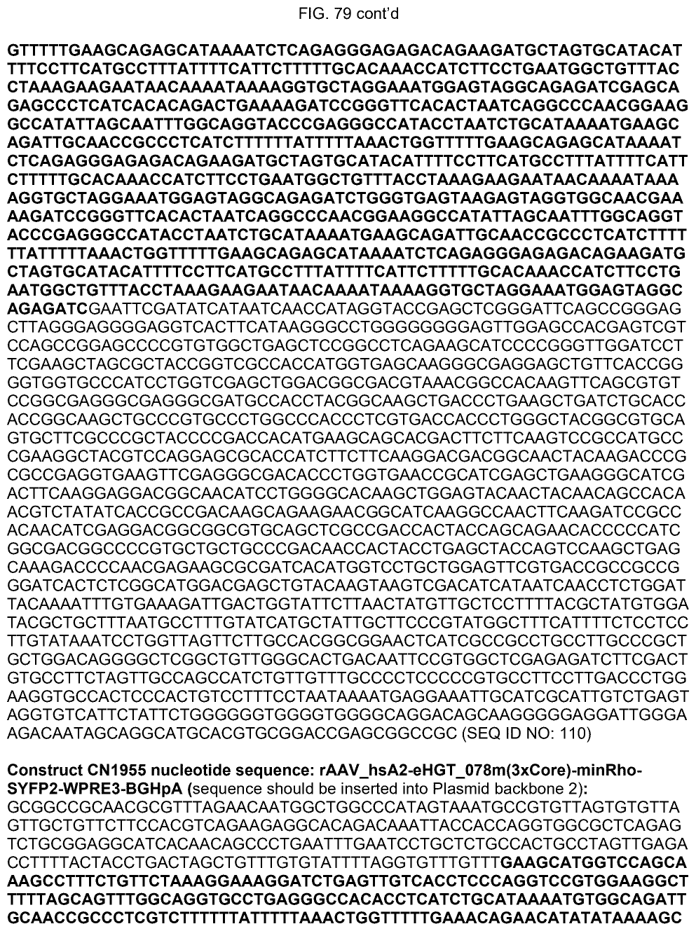

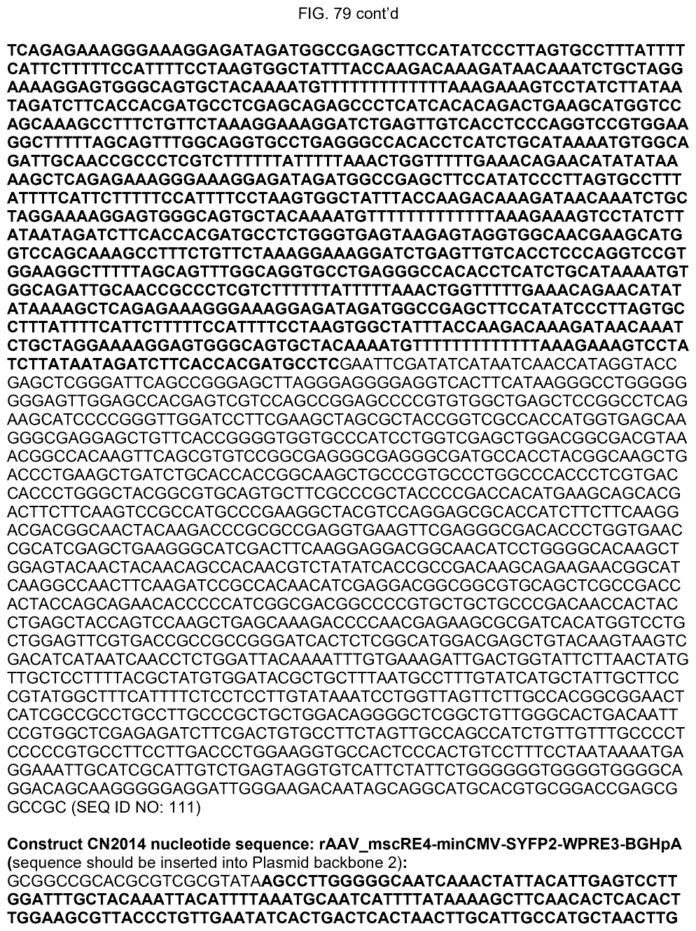

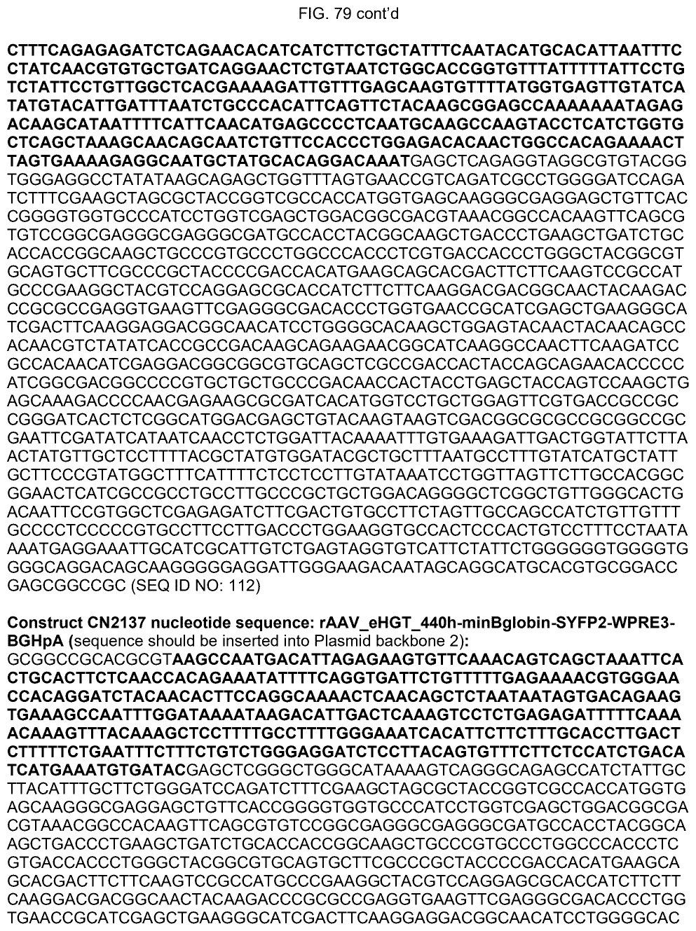

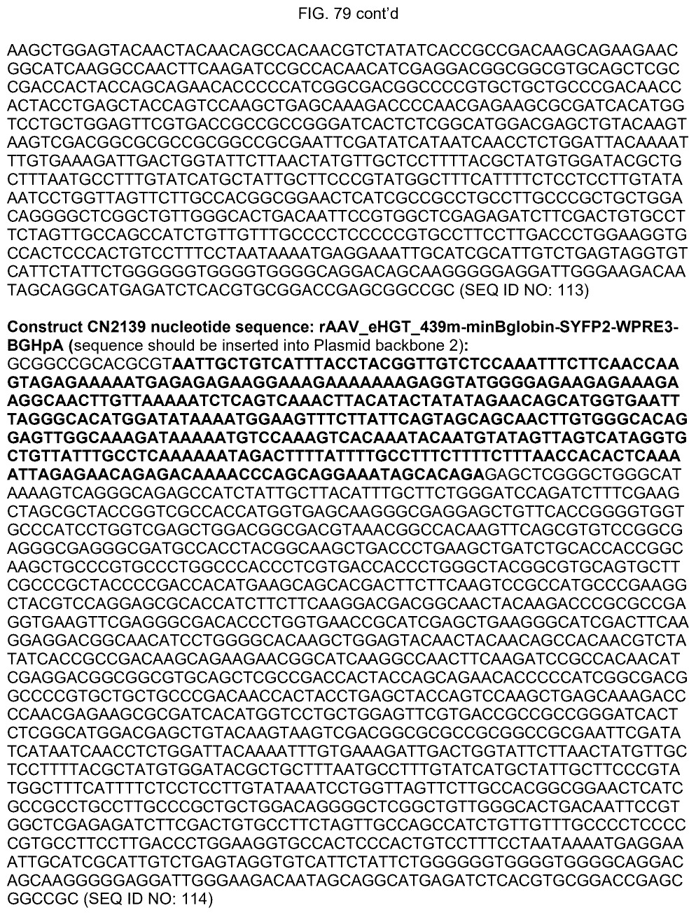

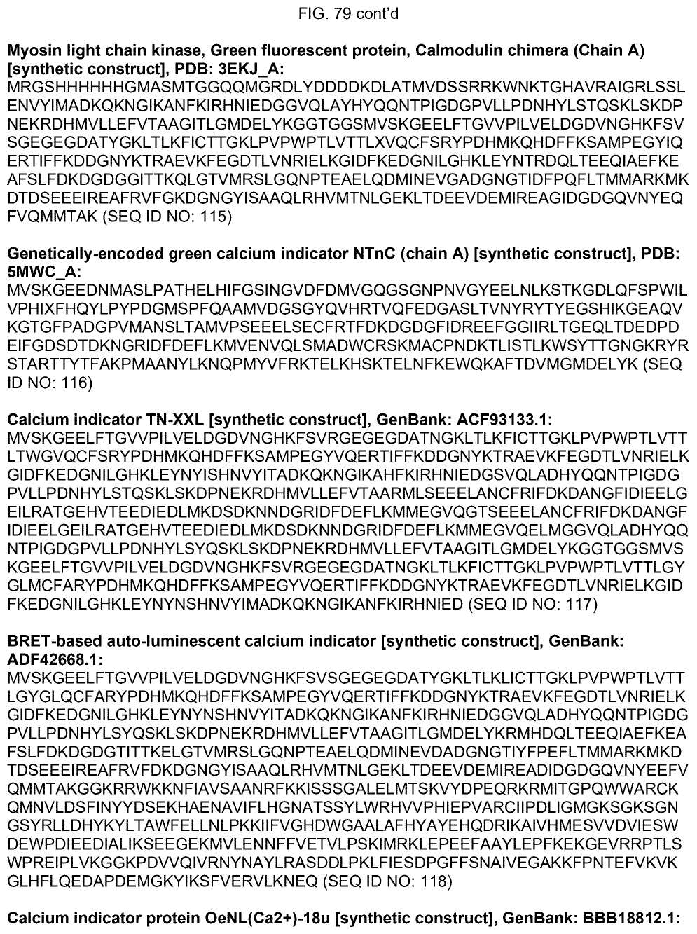

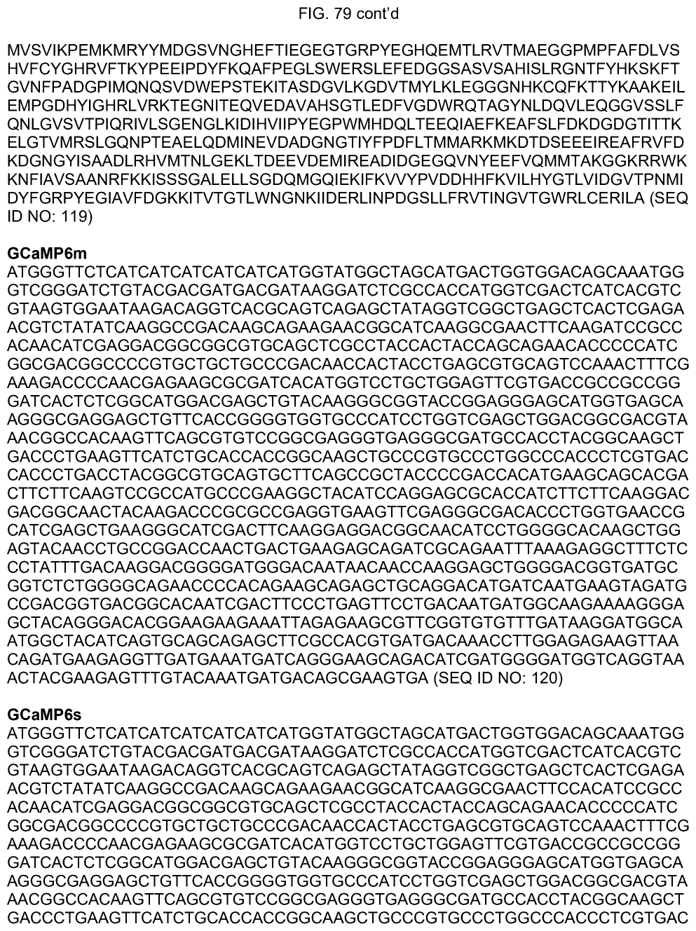

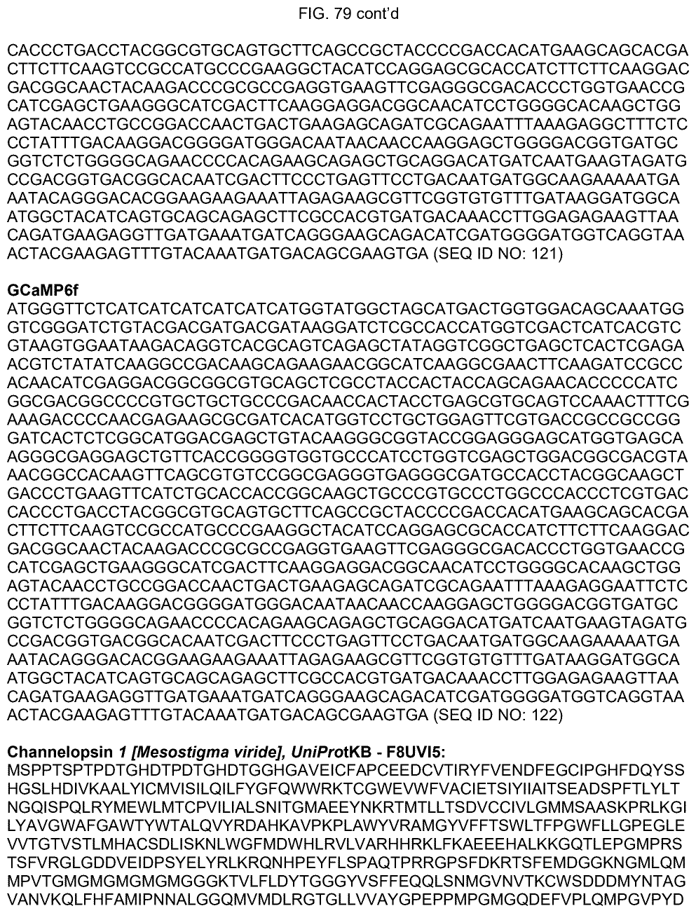

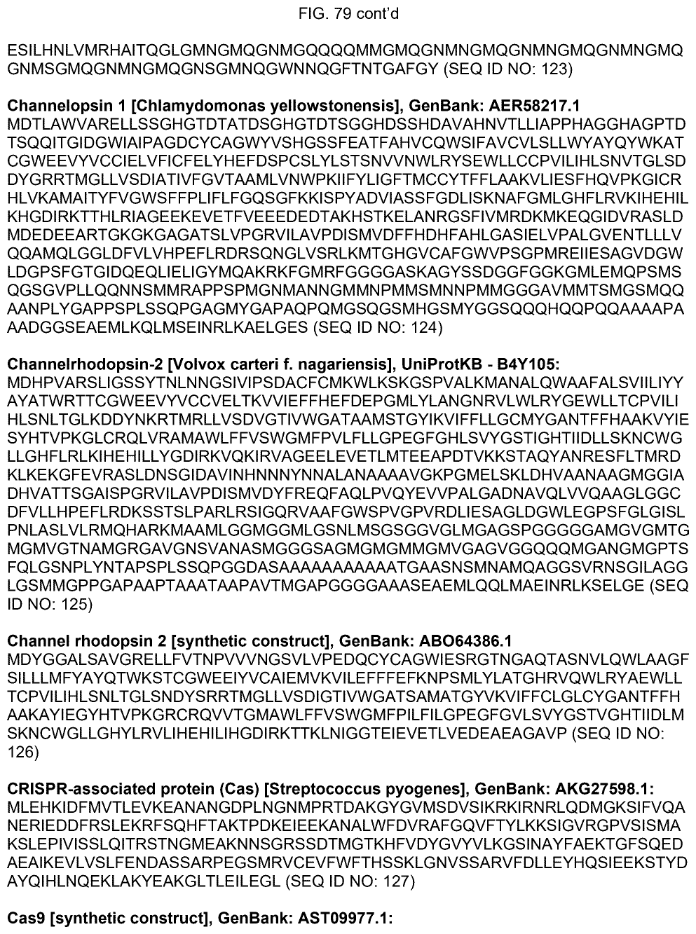

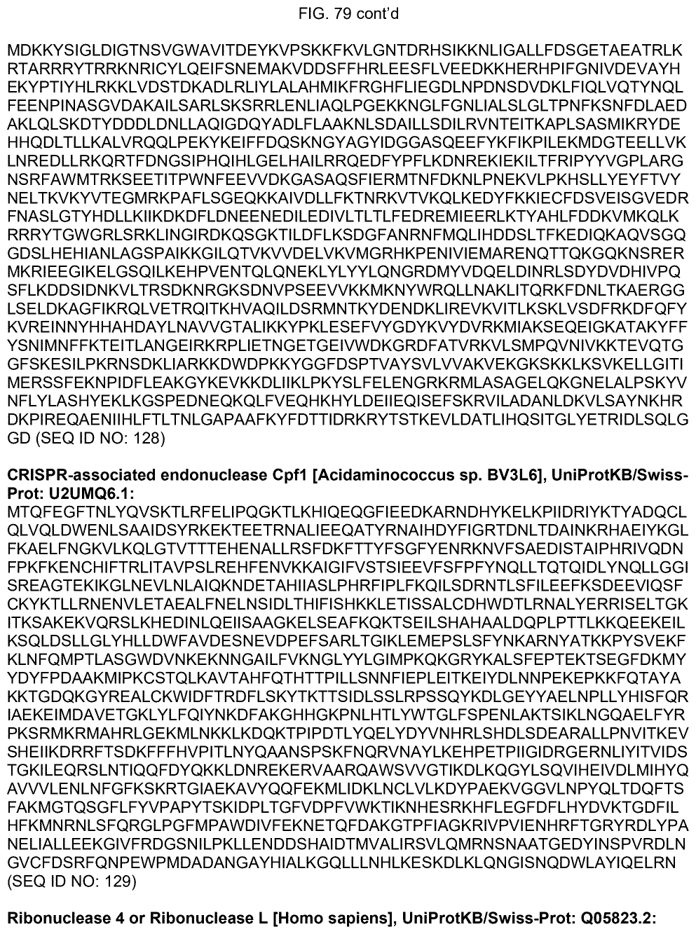

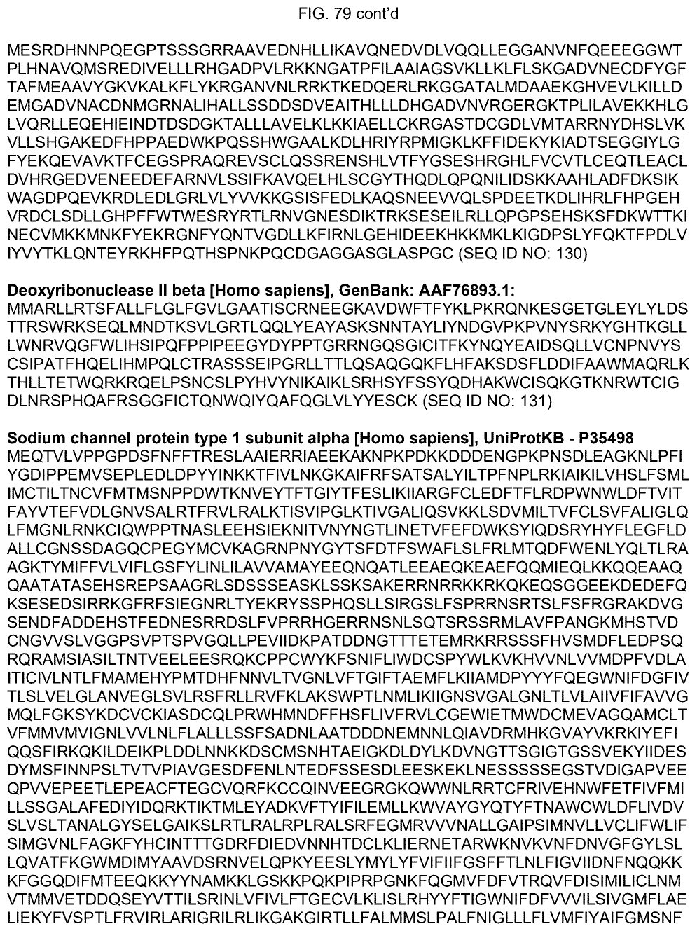

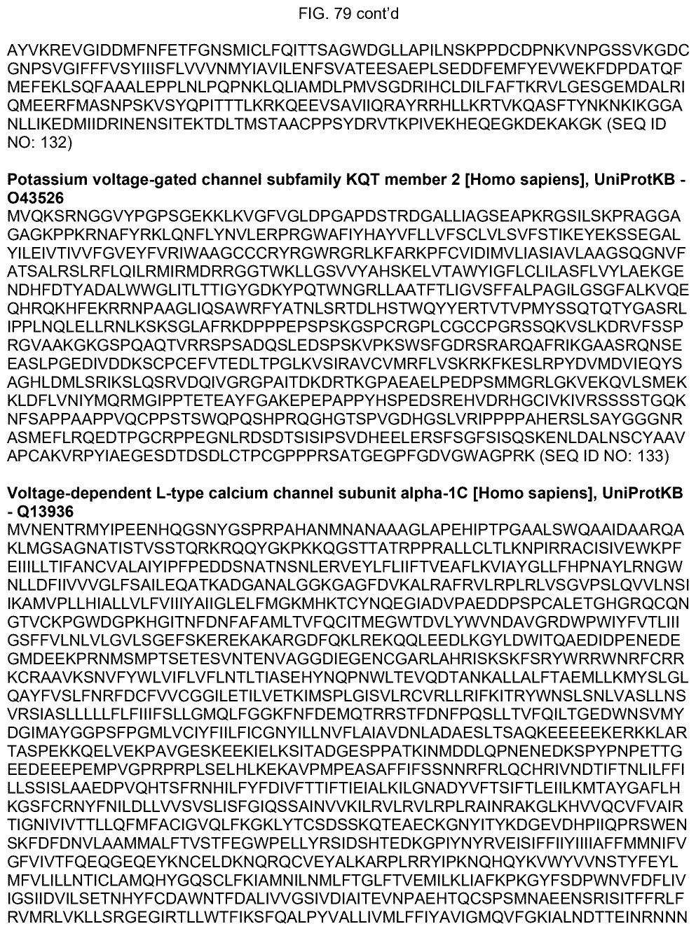

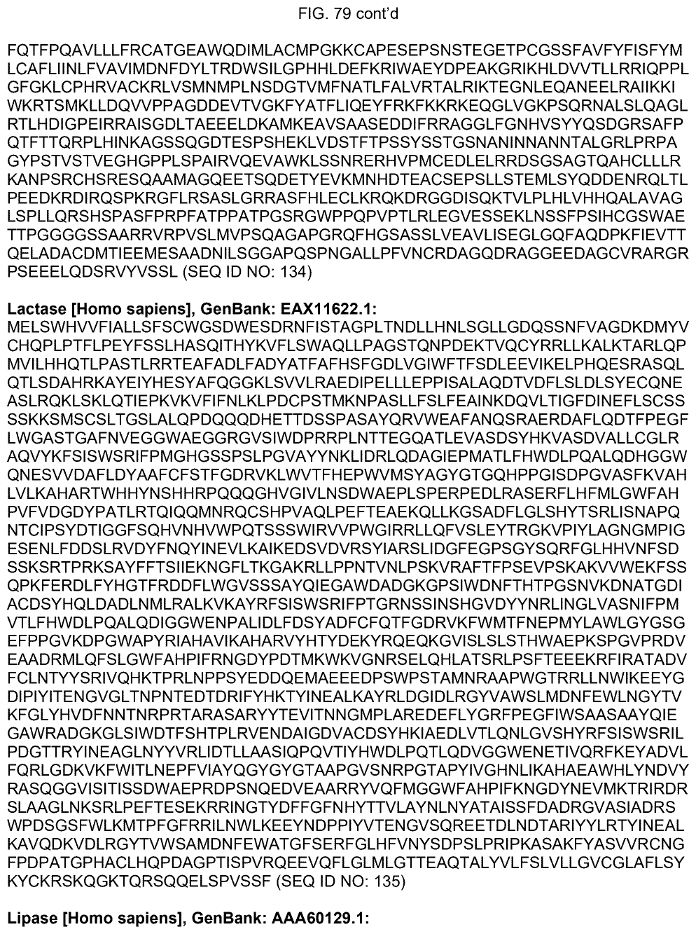

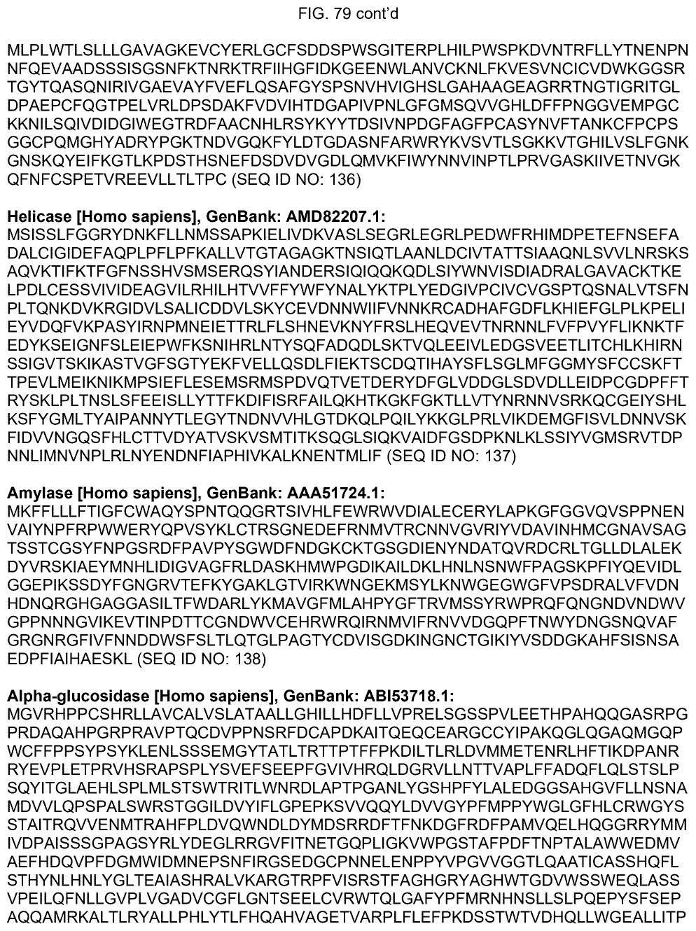

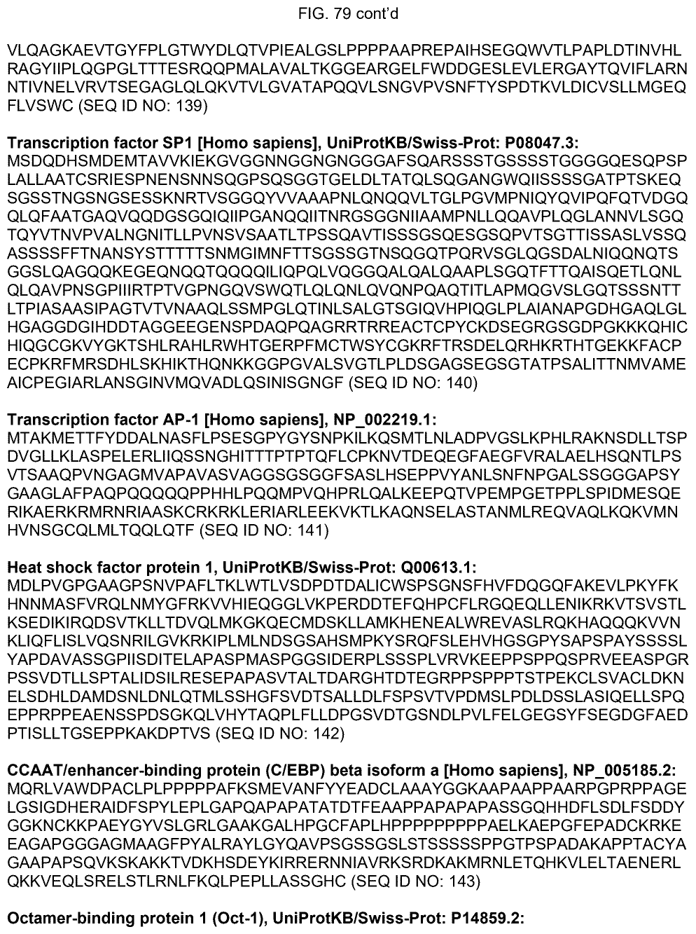

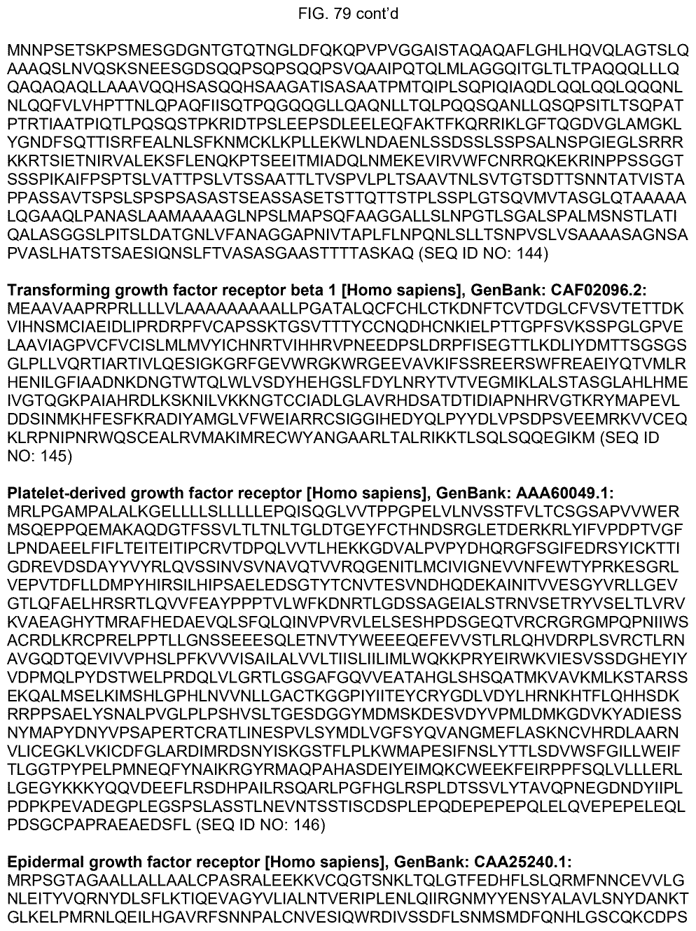

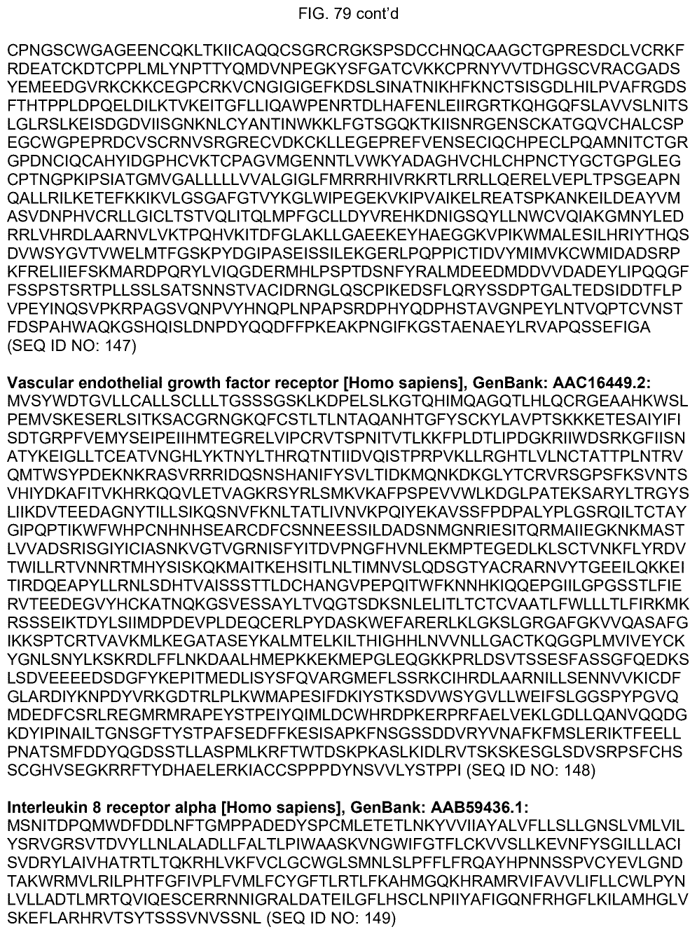

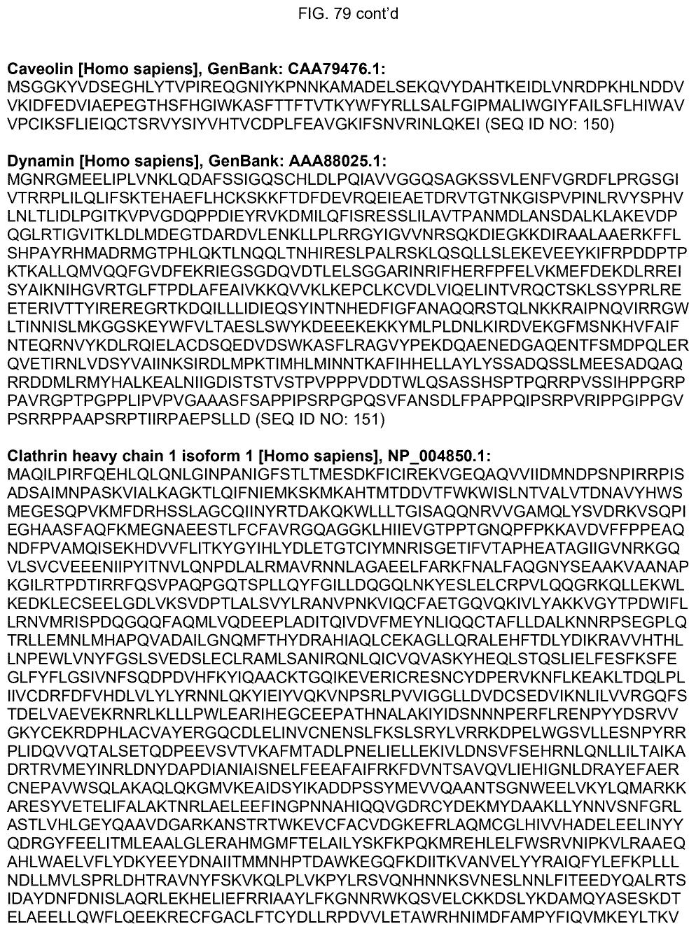

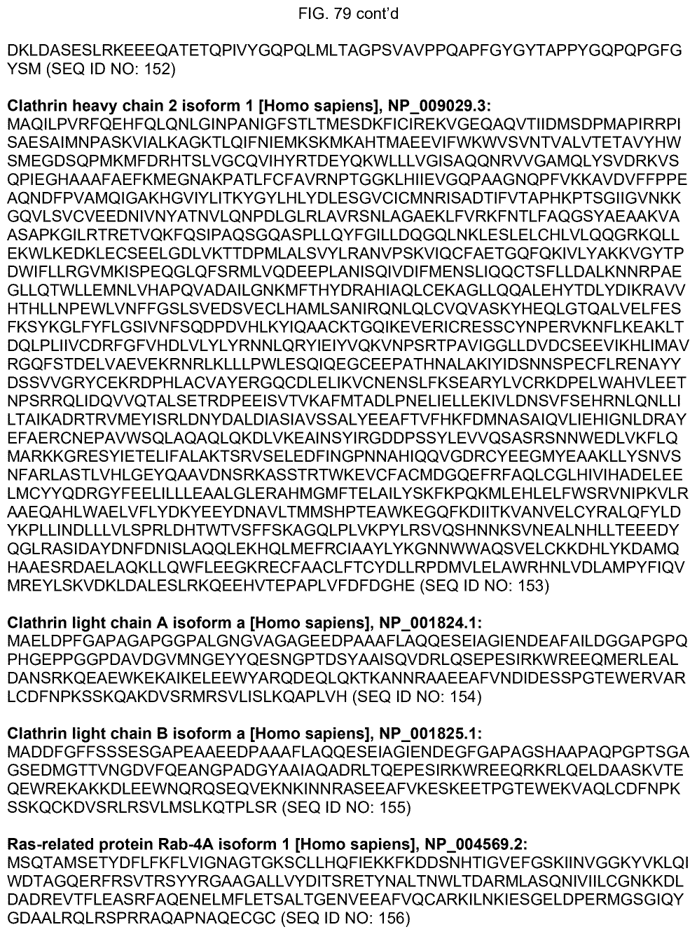

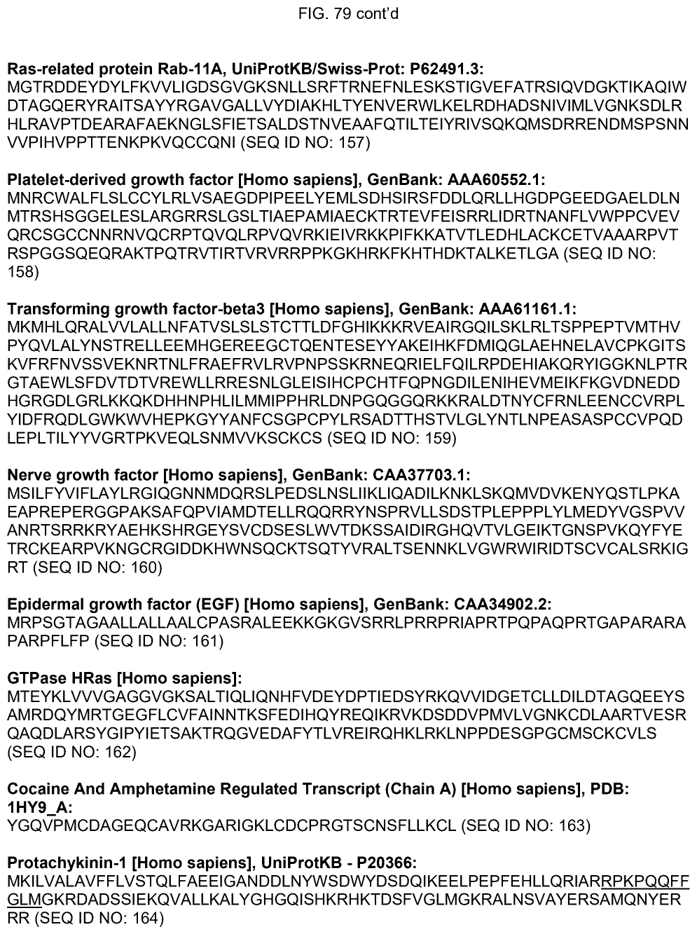

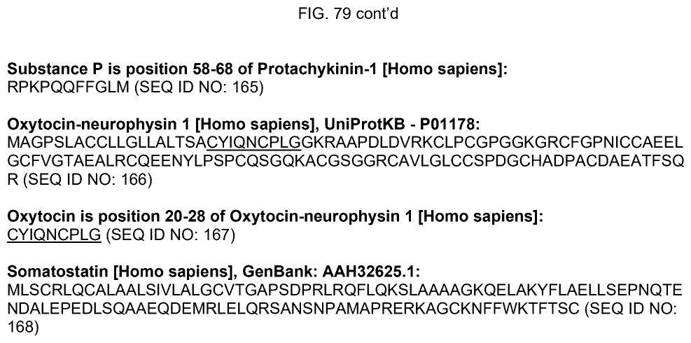

To determine whether these functional genomic elements could furnish useful genetic tools, several subclass-specific peaks were cloned into an adeno-associated virus (AAV) reporter expression vector to test for subclass-specific enhancer activity (Dimidschstein et al., Nature Neuroscience. 19, 1743-1749, 2016). Peaks were chosen to be nearby known subclass-specific marker genes from RNA-seq (Hodge et al., bioRxiv, 384826, 2018) and to exhibit subclass-specific accessibility. Several enhancers that drive distinct reporter expression patterns in mouse consistent with their expected subclass-specific accessibility profiles (Zerucha et al., J. Neurosci. 20, 709-721, 2000) were discovered (FIG. 78), suggesting that the herein described ATAC-seq enhancer discovery is a generalizable strategy to identify cell class-/type-specific genetic tools.

Since these tools are non-species restricted, research was focused on eHGT_022 near the LAMP5/VIP cell marker CXCL14, and which is conservedly accessible in LAMP5 and VIP neuron subclasses in human and mouse. It was found that AAV vectors driving either the human or mouse ortholog of eHGT_022 are both sufficient to drive expression in upper-layer-enriched interneurons in both mouse and human, and these reporter-positive cells specifically correspond to LAMP5 and VIP neurons in both mouse and human. These observations, coupled with those of the companion manuscript (Graybuck et al., bioRxiv, 525014, 2019), suggest that ATAC-seq can identify specific cell type and subclass enhancers that enable genetic tools useful in human and other species.

Human brain functions and diseases are often difficult to study because model organisms do not recapitulate human brain circuitry or display clear clinically relevant phenotypes. In particular, the functionally relevant cell types are unknown for many conditions, which leads to undertreatment of many debilitating brain disorders. It is thus critical to understand human brain-specific circuit components and their regulatory apparatus to furnish avenues for therapeutic intervention. In this work, human neocortical functional genomic elements were catalogued with cell type precision, furnishing the most high-resolution dataset of human brain chromatin accessibility so far. This deepens knowledge of human brain chromatin structure and uncovers a cell type-specific logic in gene regulation. It is expected that this knowledge will not only guide models of human cognitive circuitry, but also fuel gene therapy for unmet clinical needs.

Materials and Methods of Example 3. Neurosurgical tissue acquisition. From a network of surgeons in Seattle WA, a pipeline was established for regular delivery of fresh neurosurgical brain tissue to the Allen Institute for processing. These samples are excised as a matter of course to access the epileptic focus or tumor. Experiments are confined to temporal cortex, most frequently middle temporal gyrus. These samples are immersed in pre-carbogenated ACSF.7 (recipe in Table 3), transported to the Institute rapidly with carbogenation, and sliced on a vibratome into 350 um slices, and continuously carbogenated in ACSF.7 until dissociation.

Bulk tissue ATAC-seq. MTG tissue slices were harvested after bubbling in ACSF.7 for up to 16 hours, and they were treated with NeuroTrace 500/525 (catalog #N21480 from ThermoFisher Scientific, 1/100 in ACSF.7) to highlight layered cortex structure. With fine forceps, white matter and meningeal tissues were trimmed away, and then layers 1-6 were dissected into six different low-binding Eppendorf 1.5 mL tubes (MilliporeSigma catalog #Z666548) under a fluorescence microscope as in Hodge et al. (bioRxiv, 384826, 2018) The supernatant was discarded and replaced with 50-100 μL of Nextera DNA library reaction (#FC-121-1031 from Illumina) containing 0.1% IGEPAL-630 (NP-40 alternative), then it was pipetted up and down vigorously 25-50 times using a P200 pipette, and then incubated at 37° C. for one hour for transposition. Then, 1 mL of ice-cold nuclear isolation medium was added to quench the reaction, samples were pelleted at 1000 g for 5 minutes at 4° C., and resuspended in 1 mL fresh Homogenization Buffer (recipe in Table 3), nuclei were released from samples using 10-15 strokes of a loose-fitting dounce pestle followed by 10-15 strokes of a tight-fitting dounce pestle, then nuclei were filtered with a 70 um nylon mesh strainer, and nuclei were pelleted at 1000 g for 10 minutes at 4° C. To stain, nuclei were resuspended in 500 μL of ice-cold Blocking Buffer (recipe in Table 3) containing 1/500 PE-NeuN antibody (MilliporeSigma catalog #FCMAB317PE) and 1 μg/mL 4′-diamino-phenylindazole (DAPI, MilliporeSigma catalog #D9542), samples were rocked for 30 minutes at 4° C., then pelleted at 1000 g for 5 minutes at 4° C., and finally samples were resuspended in 500 μL fresh ice-cold blocking buffer before sorting cells on a FacsARIA III.

Using scatter profiles to eliminate debris and doublets, bulk samples were sorted as DAPI+NeuN+ from layers 1-6, or as DAPI+NeuN− from layer 1 and layer 5 samples, at 5000-10000 cells per sample, into 200 μL of blocking buffer in low-binding Eppendorf 1.5 mL tubes. Sorted nuclei were pelleted at 1000 g for 10 minutes at 4° C., followed by resuspension in 50 μL Proteinase K Cleanup Buffer (recipe in Table 3) and 37° C. incubation for 30 minutes, and then freezing at −20° C. until library prep and sequencing.

For library prep, tagmented DNA was purified with 1.8× vol/vol Ampure XP beads (Beckman-Coulter catalog #A63881), eluted in 11 μL of water, and then PCR-amplified with Nextera Index kit primers (#FC-121-1012 from Illumina) using KAPA HiFi HotStart ReadyMix (KAPA Biosystems #KK2602) in a 30 μL reaction (72° 3:00, 95° 1:00, cycle 17×[98°: 20, 65°: 15, 72°: 15], 72° 1:00). PCR products were purified using 1.8× Ampure XP beads, and libraries were quantified using Agilent BioAnalyzer High Sensitivity DNA Chips (catalog #5067-4626). Then sample libraries were pooled evenly and sequenced with paired-end 50 bp reads either on Illumina MiSeq (Allen Institute) or NextSeq machines (SeqMatic, Fremont CA USA). Fastq files were processed as described below.

Single Cell ATAC-seq. The single cell ATAC-seq workflow was modified from the bulk sample workflow in several ways, most notably performing transposition reactions following sorting rather than prior to sorting, and omitting DAPI except for non-neuronal samples (due to the uncertainty of DAPI possibly interfering with transposition).

Specific MTG tissue layers were collected and dissected as for bulk samples, but the layers were immediately dounced to release nuclei, and then stained in blocking buffer containing PE-NeuN antibody but not DAPI. Single NeuN+ nuclei from each layer were sorted into each well of a 96-well plate, using scatter profiles to exclude debris and doublets. Single nucleus-to-event correspondence was confirmed by test-sorting single NeuN+ events into flat-bottom 96 well plates with 40 μL blocking buffer containing DAPI followed by pelleting 1 min at 3000 g and microscopic examination. These tests routinely yielded >95% single nucleus-filled wells and undetectable doublets. In the cases where glial cells were sorted, neurons were first sorted from the sample using PE-NeuN+ staining, and then treated with DAPI (1 ug/uL) for 1-2 minutes prior to sorting glial cells as DAPI+NeuN-events.

Single NeuN+ cells were sorted into 1.5 μL of Nextera Tn5 transposition reaction (0.6 μL Tn5 enzyme, 0.75 μL tagmentation buffer, 0.15 μL 1% IGEPAL CA-630) in Eppendorf semi-skirted 96-well plates (MilliporeSigma catalog #EP0030129504). Immediately following sorting, plates were briefly spun down, briefly vortexed, spun down again, and then incubated at 37° C. for 30 minutes for transposition. After transposition 0.6 μL Proteinase K Cleanup Buffer were added, sample was briefly vortexed and spun down, and incubated at 40° C. for an additional 30 minutes, then plates were frozen until library prep. Library prep for single cell samples was the same as for bulk samples, except the number of amplification cycles was increased from 17 to 22 cycles due to the lower input DNA content.

Bulk ATAC-seq sample clustering. Peaks were called on all 39 bulk samples from 5 independent specimens using MACS2 (Zhang et al., Genome Biology. 9, R137, 2008), and then DiffBind (Ross-Innes et al., Nature. 481, 389-393, 2012) was used to identify 73742 differential peaks for all contrasts among the sample types (sort strategies and specimens). Of these, 1524 distinguished experimental specimens and were discarded for clustering. With 72218 remaining peaks found specifically to discriminate any pairwise combinations of sort strategies, correlation among bulk samples was reanalyzed using reads in these peaks. A correlation matrix revealed grouping of non-neuronal samples, upper layer neuronal samples, and lower layer neuronal samples. One sample was omitted from this analysis (H17.03.009 L1 NeuN+) because this sample appeared intermediate between NeuN+ and NeuN− cells, likely due to a sorting error.

ATAC-seq data preprocessing and quality control. Sample-specific fastq files were retrieved using standard built-in Illumina deindexing protocols. Each fastq file was mapped to human genome reference hg38 patch 7 using bowtie2 and the flags—no-mixed—no-discordant-×2000 to generate sample-specific bam files, which were then filtered for low-quality mappings, secondary mappings, and unmapped reads using samtools view-q 10-F 256-F 4, and then filtered for duplicate reads using samtools rmdup. Then, these filtered reads bam files were converted to bed files using bedTools bamToBed for quality control calculations of mean ENCODE overlap and TSS enrichment score. For mean ENCODE overlap bed files were converted to fragment format, the percentage of unique fragments that overlap with ENCODE project DNasel hypersensitivity peaks from adult human frontal cortex (studies ENCSR000EIK and ENCSROOOEIY; The ENCODE Project Consortium, Nature. 489, 57-74, 2012; Sloan et al., Nucleic Acids Res. 44, D726-D732, 2016) was assessed using bedTools intersectBed (Quinlan & Hall, Bioinformatics. 26, 841-842, 2010), and the mean of these two numbers was taken. For TSS enrichment score, the published technique of Chen et al (Chen et al., Nat Meth. 13, 1013-1020, 2016) was used. This technique sums the overlap of reads in 2 kb windows surrounding all human TSSs, then segments this 2 kb window into 40 50-bp bins, then normalizes the summed read counts to the outside four bins (first and last two), and finally reports the TSS enrichment score as the maximum height of that normalized read count graph. It was noticed that this technique worked well for all bulk samples but gave spurious abnormally high scores for some single cells having low read count; as a result a modification was made to set TSS enrichment score to 1 (no enrichment) for single cells having fewer than 500 reads or TSSs calculated to be greater than 20 (likely spurious events).

These quality control metrics were used to filter out low quality cells (ENCODE overlap <15% AND TSS score <4). Additionally, cells having fewer than 10000 unique read pairs were filtered out, since these many reads are required for the clustering approach. Of 3660 initial cells, analysis was confined to 2858 high quality nuclei for clustering.

Clustering single cells: bootstrapped clustering. Single cells were clustered using extended fragment Jaccard distance calculations among cells as implemented by the lowcat package (Graybuck et al., bioRxiv, 525014, 2019). To accomplish this, first, reads on chromosomes X, Y, and M were excluded to prevent differential chromosome-biased clustering. Then, it was randomly downsampled as described in Materials and Methods of Example 1 with fragments extended to a regularized length of 1000 bp with the same center. Then, Jaccard distances were calculated as described in Materials and Methods of Example 1.

Finally, this 2858×2858 Jaccard distance matrix was dimensionality reduced to a 2858×29 matrix of principal component scores 2 through 30 using princomp in R. Principal component 1 was omitted because it was highly correlated to quality control metrics, suggesting that this axis primarily reflected cell library quality. Principal components beyond 30 contain little cell type information, so excluding them represents a de-noising step. These resulting 29 PCs are used to call cell clusters and to visualize them using tSNE.

To call cell clusters on this 2858×29 principal component matrix, an iterated Jaccard-Louvain clustering technique was bootstrapped using k=15 nearest neighbors. Each bootstrapping round was repeated 200 times, each time including only 80% (2286) of the cells, and the frequency with which each cell co-clusters with every other cell was tabulated. This co-clustering frequency matrix was then hierarchically clustered by Euclidean distances, and 27 cell type clusters were called by cutting the tree to represent visually apparent co-clustered blocks of cells. Repeating this process with more stringent variable 50-90% cell inclusion resulted in similar cluster structure with similar cluster memberships, but randomizing the Jaccard distance matrix prior to principal component analysis and bootstrapped clustering yielded no clusters in the dataset. Together these analyses suggest that the identified clusters represent real and reproducible cell groups.

Clustering single cells: comparing choice of feature set. Clustering cells using other feature sets besides Jaccard distances among cells was also attempted. These feature sets included: 1) the list of all detected peaks from the entire aggregated dataset (236588 peaks called using Homer findPeaks (Heinz et al., Molecular Cell. 38, 576-589, 2010) with-region flag), 2) the list of all RefSeq gene TSS regions, extended+/−10 kb (27021 regions), 3) all 321184 non-overlapping 10 kb bins across the human genome, and 4) the list of “GeneBins” defined as the genomic region for each gene between the boundaries of midpoints between each RefSeq gene transcribed region. For each feature set, counts in regions for each cell were computed, then principal components were identified, and cell groupings were visualized by tSNE of principal components 2:50 in order to observe cell groupings. Jaccard distances disclosed the qualitatively cleanest separation among cells, and among cell clusters. Furthermore, a wide range of tSNE perplexity values maintained these separations.

Mapping clusters to transcriptomic cell types: assimilating epigenetic and transcriptomic information. The goal was to map the 2858 high quality ATAC-seq profiled cells to human brain cell types discovered by large-scale RNA-seq studies (Hodge et al., bioRxiv, 384826, 2018). To do this, first, the best technique to manufacture gene-level information from the ATAC-seq data was sought, in order to correlate with RNA-seq transcript counts. Four techniques were tried: 1) read counts in RefSeq gene bins, 2) read counts in RefSeq gene bodies, 3) read counts in RefSeg gene TSS regions extended+/−10 kb, and 4) Cicero gene activity scores (Cusanovich et al., Cell. 174, 1309-1324.e18, 2018; Pliner et al., Molecular Cell. 71, 858-871.e8, 2018). With these four sets of gene-level information computed for each cell, single cells were mapped to RNA-seq cell types using as the best correlated RNA-seq cluster median gene counts per million (CPM) with each epigenetic feature set (using a subset of 831 marker genes), resulting in four distinct mappings for each cell.

The 831 marker genes were chosen to be both informative marker genes for RNA-seq clustering and to contain abundant epigenetic information. This was accomplished by using the select_markers function with default parameters from the scrattch.hicat R package (Tasic et al., Nature. 563, 72, 2018) which yielded 2791 transcriptomic marker genes, which was further filtered by intersecting with the top ten percent of genes with the highest summed Cicero gene activity scores across all 2858 cells, to yield 831 combined transcriptomic and epigenetic marker genes for mapping.

The four sets of cellwise mappings yielded four tables of cell type abundances within the dataset. Next, taking the RNA-seq dataset as a true gold standard, the four cell type abundance tables were compared with the ‘expected’ cell type abundances, which were calculated as the sum of numbers of cells sorted in each sort strategy, times the expected cell type frequencies in each sort strategy. Correlating the four cell type abundance tables with the expected abundance table (Pearson correlations of log-transformed abundance values plus one) revealed that Cicero gene activity scores supply the most dependable gene-level information for the purpose of epigenetic to transcriptomic mapping.

Mapping clusters to transcriptomic cell types: bootstrapping mapping for final mapping calls. Using Cicero gene activity scores, the cellwise mapping procedure was bootstrapped 100 times with retention of a variable 50-90% of genes each round, and the most frequently mapped transcriptomic cell type was applied to each single ATAC-seq cell. Then, the percentage of each cluster's constituent cells mapping to each cell type was reported and summed by cell type subclass.

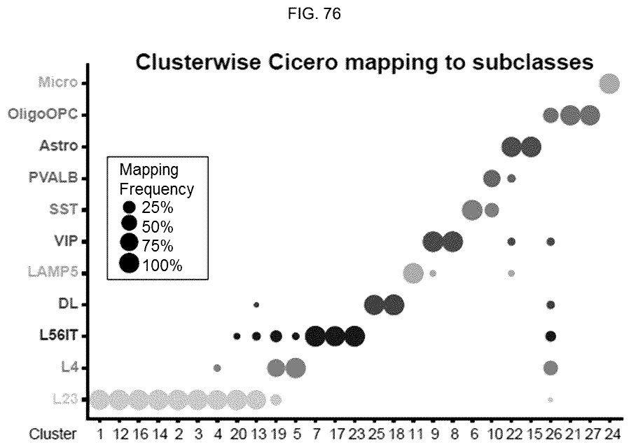

Clusterwise mapping was also performed for each of the 27 ATAC-seq clusters using the same bootstrapped mapping procedure, except that Cicero gene activity scores were aggregated by mean across cells within each cluster prior to mapping. The number of 100 times that each cluster is mapped to each cell type was reported and summed by transcriptomic subclass in FIG. 76.

Clusterwise mapping was observed to largely agree with, but to be cleaner than, cellwise mapping (FIG. 76); hence clusterwise mapping was elected as the final mapping procedure. Each cell is thus assigned a final mapped transcriptomic cell type and cell type subclass (shown in FIG. 76) as a result of its ATAC-seq cluster membership.

Peak calling. Peaks were called on both bulk and aggregated single-cell data using Homer findPeaks with-region flag (Heinz et al., Molecular Cell. 38, 576-589, 2010). This program was found to be superior to Hotspot, MACS2, and SICER to identify small regions corresponding to likely enhancers, while still capturing the peak boundaries. Peak sizes are median 400-500 bp across subclasses.

Identifying transcription factor motifs using chromVAR. ChromVAR (Schep et al., Nature Methods. 14, 975-978, 2017) was used to identify transcription factor motif accessibilities in the cells. Using Homer findPeaks, peaks were called on the aggregation of all single cell and bulk libraries (236588 peaks), and then they were resized to a standard 150 bp size with the same center. 452 transcription factor motifs from JASPAR (using JASPAR2018 R package; Tan, JASPAR2018: Data package for JASPAR 2018., 2017) and 1764 from cisBP (as included in the R package chromVARmotifs; Schep et al., Nature Methods. 14, 975-978, 2017) were downloaded, and chromVAR was used to aggregate and quantify motif accessibilities in all 2858 single cells. Cell type subclass-distinguishing motifs across were found by ranking subclass-averaged motif accessibilities by standard deviation across subclasses (including DLX1 and NEUROD6).

Global peak characterization by conservation. With peaks called for each subclass, peaks were subset into four sets. 1) All peaks (no subsetting). 2) Subclass-specific peaks which were detected in only that subclass and not in an outgroup subset of human keratinocyte or mouse E16.5 kidney ATAC-seq data downloaded from ENCODE (The ENCODE Project Consortium, Nature. 489, 57-74, 2012). 3) TSS-distal peaks which were not located less than 20 kb from any of 27021 RefSeq gene TSS sites, downloaded from UCSC table browser (Karolchik et al., Nucleic Acids Res. 32: D493-D496, 2004). 4) Subclass-specific AND TSS-distal peaks. Overlaps were calculated using bedtools intersectBed. In analyses that shuffle peak positions, for TSS-distal peaks randomly generated comparator peak positions were restricted to the same TSS-distal genomic regions.

For peak phyloP scores, bigWigSummary was used to lookup phyloP values from hg38.phyloP4way.bw or mm10.phyloP4way.bw. These files quantify the basepair conservation across four mammals: Homo sapiens, Mus musculus, Galeopterus variegatus (Malayan flying lemur), and Tupaia chinensis (Chinese tree shrew). Ten values distributed across each peak were returned, and the maximum mean of eight three-consecutive-value sets was calculated. This is done to find smaller regions on the order of 100 bp highly conserved regions within each peak and yields greater deviations between real and random phyloP scores than taking a single peak-wise average alone. Peak-wise phyloP scores were compared to those of randomly distributed peak regions throughout the genome by subtracting real peak phyloP mean minus random peak phyloP mean.

Identifying transcriptomic cell type matches for methylation data. Using the dataset of Luo et al. (Science. 357, 600-604, 2017 (Supplementary Table 3 containing 1012 human and 1016 mouse methylation marker genes)), the published mCH gene body marker genes were correlated with cluster-wise medians for transcriptomic human cell types identified by Hodge et al. (bioRxiv, 384826, 2018) and for mouse cell types by Tasic et al. (Nature. 563, 72, 2018). Pearson correlation coefficients were calculated between normalized gene body mCH and RNA-seq clusterwise median FPKM, and the best-correlated transcriptomic cell type was assigned to each methylation cell type. Specificity of matches was calculated as the difference between the best correlation and the second-best correlation. Importantly, all transcriptomic cell type assignments agree with the predicted subclasses by the original authors.

Quantifying ATAC-seq peak overlaps with DMRs. First, human DMRs from Luo et al. (Science. 341, 1237905, 2013) and Lister et al. (Science. 357, 600-604, 2017) were aggregated. For neuron types, DMRs were downloaded as calculated by the authors and then these DMRs were merged using bedtools mergeBed. For non-neuron types, raw fastq files were downloaded from the GEO submission of Lister et al corresponding to bulk NeuN-negative cells from two human replicates (GSM1173774 and GSM1173777) and converted these to allc files using the pipeline analysis method of Luo et al. (Science. 357, 600-604, 2017). These allc files were aggregated and used to find DMRs with methylpy DMRfind against allc files for all human subclasses from Luo et al., and an outgroup of human H1 cells from ENCODE (The ENCODE Project Consortium, Nature. 489, 57-74, 2012). The same set of bulk non-neuronal DMRs were used as one for comparison to Astrocytes, Oligodendrocytes/OPCs, and Microglia ATAC-seq classes (FIG. 77).

With bed files corresponding to each subclass ATAC-seq peakset and to each subclass DMR set, bedtools intersected were used to quantify the overlap between peaks and DMRs. Calculation of real peak overlaps 100× was bootstrapped by removing 20 percent of peaks each time and calculating percentage overlap, and the mean of these 100 measurements is reported.

Similarly, peak positions were randomized throughout the genome 100× using bedtools shuffleBed, percentage overlap was calculated each time, and the mean of these 100 measurements is reported. By definition, disjoint ranges of real versus randomized peak overlap percentages established false discovery rate <0.01. Enrichment of DMR overlaps for ATAC-seq peaksets, defined as the ratio of real peak-DMR overlap percentage to the overlap percentage of randomized peak positions, was also calculated.

Mouse to human cross-species comparisons. The sets of subclass-specific peaks were used to map between human and mouse subclasses, which are uniquely identified in only that subclass. First subclass-specific mouse peaks were mapped to hg38 using liftOver. Then calculation of human peak overlap was bootstrapped 100× against all mouse peaks with random retention of 80% of human peaks each time, and the mean of Jaccard similarity coefficients (intersection over union) over 100 runs was taken. In addition, genomic peak positions were shuffled 100×, and mean Jaccard similarity coefficients were calculated each time. The enrichment of Jaccard similarity coefficients was determined as the ratio of the real over random.

Characterization of human conserved and divergent peaks began with all human peaks and subset to those intersecting (“Conserved”) or not intersecting (“Divergent”) with mouse peaks identified within the same homologous subclass and mapped to hg38 by liftOver. To characterize mouse conserved and divergent peaks, all mouse peaks were intersected with reciprocal mm10-mapped human peaks. Then phyloP scores were calculated as above for these four sets of peaks.

Cloning enhancers. Enhancers were manually chosen from ATAC-seq and RNA-seq data for cloning by the following criteria: 1) adjacent to known subclass marker gene, and 2) specifically accessible peak in only the subclass of interest, and 3) contains region of high primary sequence conservation by phyloP score.

Chosen enhancers were cloned into AAV expression vectors that are derivatives of either pscAAV-MCS (Cell Biolabs catalog #VPK-430), including eHGT_019h, eHGT_017h, eHGT_022h, eHGT_022m, and eHGT_023h; or pAAV-GFP (Cell Biolabs catalog #VPK-410), including eHGT_078h, eHGT_058h, eHGT_060h, and hDLXI56i (Dimidschstein et al., Nature Neuroscience. 19, 1743-1749, 2016; Zerucha et al., J. Neurosci. 20, 709-721, 2000). Enhancers were inserted by standard Gibson assembly approaches, upstream of a minimal beta-globin promoter and SYFP2, a brighter EGFP alternative that is well tolerated in neurons (Kremers, et al., Biochemistry. 45, 6570-6580, 2006). NEB Stable cells (New England Biolabs #C30401) were used for transformations. scAAV plasmids were monitored by restriction analysis and sanger sequencing for occasional (10%) recombination of the left ITR.

Virus production. Enhancer AAV plasmids were maxiprepped and transfected with polyethylimine max into 1 plate of AAV-293 cells (Cell Biolabs catalog #AAV-100), along with helper plasmid and PHP.eB rep/cap packaging vector. The next day medium was changed to 1% FBS, and then after 5 days cells and supernatant were harvested and AAV particles released by three freeze-thaw cycles. Lysate was treated with benzonase after freeze thaw to degrade free DNA (2 μL benzonase, 30 min at 37 degrees, MilliporeSigma catalog #E8263-25KU), and then cell debris was precleared with low-speed spin (1500 g 10 min), and finally the crude virus was concentrated over a 100 kDa molecular weight cutoff Centricon column (MilliporeSigma catalog #Z648043) to a final volume of 150 μL. This crude virus prep was useful in both mouse and human virus testing.

Mouse virus testing. Mice were retro-orbitally injected at P42-P49 with 10 μL (1E11 genome copies) of crude virus prep diluted with 100 μL PBS, then sacrificed at 18-28 days post infection. For live epifluorescence, mice were perfused with ACSF.7 and live 350 um physiology sections were cut with a compresstome from one hemisphere to analyze reporter expression. For antibody staining the other hemisphere was drop-fixed in 4% PFA in PBS for 4-6 hours at 4 degrees, then cryoprotected in 30% sucrose in PBS 48-72 hours, then embedded in OCT for 3 hours at room temperature, then frozen on dry ice and sectioned at 10 μm thickness, prior to antibody stain using standard practice. Single-cell RNA-seq was accomplished as described previously (Tasic et al., Nat Neurosci. 19, 335-346, 2016; Tasic et al., Nature. 563, 72, 2018).

Human virus testing. Temporal cortex neurosurgical samples were bubbled in cold ACSF.7 and kept sterile throughout processing. Blocks of tissue were sliced at 350 μm thickness and then white matter and pial membranes were dissected away. Typically all layers are represented in a good cortical slice. Slices then underwent warm recovery (bubbled ACSF.7 at 30 degrees for 15 minutes) followed by reintroduction of sodium (bubbled ACSF.8 at room temperature for 30 minutes, recipe in Table 2; Ting et al., Scientific Reports. 8, 8407, 2018). Slices were then plated at the gas interface on Millicell PTFE cell culture inserts (MilliporeSigma #PICM03050) in a 6-well dish on 1 mL of Slice Culture Medium (recipe in Table 3). After 30 minutes, slices were infected by direct application of high-titer AAV2/PHP.eB viral prep to the surface of the slice, 1 μL per slice. Slice Culture Medium was replenished every 2 days and reporter expression was monitored.

Single cell RNA-seq was accomplished on human virus-infected neurons by 1 hr digestion at 30 degrees in carbogenated ACSF. 1/trehalose+blockers+papain (recipes in Table 3), followed by gentle trituration in Low-BSA Quench buffer, shallow spin gradient centrifugation (100 g 10 minutes at room temperature) into High-BSA Quench buffer, and resuspension into Cell Resuspension Buffer. Also, Myelin Bead Removal Kit II (Miltenyi catalog #130-096-733) at 1/20 was employed to remove myelin debris, and PE-anti CD9 clone eBioSN4 (Thermo Fisher catalog #12-0098-42) at 1/40 to sort away contaminating glial cells. Then, single SYFP2+ labeled human neurons were sorted for sequencing using SMARTer V4 as previously described (Tasic et al., Nat Neurosci. 19, 335-346, 2016; Tasic et al., Nature. 563, 72, 2018).

Inferring GWAS-cell subclass associations. Linkage disequilibrium score regression (LDSC; Bulik-Sullivan et al., Nature Genetics. 47, 291-295, 2015; Finucane et al., Nat Genet. 47, 1228-1235, 2015) was used to partition heritability of various brain conditions to regions associated with accessible chromatin in eleven human cortical cell subclasses, whose peaks are partitioned into Conserved and Divergent subsets. As outgroup comparators, heritability associated with outgroup populations of human keratinocytes downloaded from ENCODE was also investigated.

Summary statistics from 21 Genome Wide Association Studies (GWAS) were downloaded, including expected brain-related (schizophrenia, major depressive disorder, autism spectrum disorder, ADHD, Alzheimer's disease, Tourette's syndrome, bipolar disorder, eating disorder, obsessive-compulsive disorder, loneliness, BMI, PTSD) and expected non-brain-related diseases (Crohn's disease and asthma) from the PGC and EMBL/EBI GWAS repositories (see Table 2). Studies with log¹⁰(N*h²)<3.6 were excluded, where N is number of patients in the study and h² represents the sum of heritability across SNPs within the study, the effective power of the study (Finucane et al., Nat Genet. 47, 1228-1235, 2015). This exclusion removed asthma (Demenais et al., Nat. Genet. 50, 42-53, 2018; log¹⁰(N*h²)=3.5, PTSD (Duncan et al., Mol. Psychiatry. 23, 666-673, 2018 log¹⁰(N*h²)=2.9), eating disorder (Duncan et al., Am J Psychiatry. 174, 850-858, 2017; log¹⁰N*h²)=3.5), loneliness (Gao et al., Neuropsychopharmacology. 42, 811-821, 2017; log¹⁰(N*h²)=3.3), obsessive-compulsive disorder (IOCDF-GC & OCGAS, Mol. Psychiatry. 23, 1181-1188, 2018; log¹⁰(N*h²)=3.5), and one major depressive disorder study (Major Depressive Disorder Working Group of the Psychiatric GWAS Consortium et al., Mol. Psychiatry. 18, 497-511, 2013; log¹⁰(N*h²)=3.3). All 15 included studies were performed on a European descent population. Within these datasets, the analysis was confined to 1389227 high-confidence SNPs present in the HapMap3 list, and using linkage disequilibrium maps from the 1000 Genomes European descent individuals, the trait and disease enrichments of cell subclass-associated chromatin were analyzed along with the LDSC baseline model LDv2.0 with 75 enumerated genomic feature categories. For statistical testing to identify significant enrichments Bonferroni multiple hypothesis testing correction of LDSC's block jackknife-estimated p-values was used, as previously suggested (Skene et al., Nature Genetics. 50, 825, 2018). This correction is 0.05/345 disease/subclass combinations=1.45e⁻⁴ significance cutoff, and similarly 180 and 150 tests were used.

TABLE 2
Citations for GWAS studies

| Citation | Disease(s)/Condition(s) |
|---|---|
| Anney et al., Molecular Autism. 8, 21, 2017 | Autism |
| Autism Spectrum Disorder Working Group of the Psychiatry Genomics | Autism |
| Consortium, PGC- ASD summary statistics from a meta-analysis of 5,305 | spectrum |
| ASD-diagnosed cases and 5,305 pseudocontrols of European descent. | disorder |
| (2015), (available online at med.unc.edu/pgc/results-and-downloads). | |
| de Lange et al., Nat. Genet. 49, 256-261, 2017 | Inflammatory |
| | Bowel |
| | Disease |
| Demenais et al., Nat. Genet. 50, 42-53, 2018 | Asthma |
| Duncan et al., Mol. Psychiatry. 23, 666-673, 2018 | PTSD |
| Duncan et al., Am J Psychiatry. 174, 850-858, 2017 | Eating |
| | disorder |
| Gao et al., Neuropsychopharmacology. 42, 811-821, 2017 | Loneliness |
| International Obsessive Compulsive Disorder Foundation Genetics | OCD |
| Collaborative (IOCDF-GC) and OCD Collaborative Genetics Association | |
| Studies (OCGAS), Mol. Psychiatry. 23, 1181-1188, 2018 | |
| Lambert et al., Nat. Genet. 45, 1452-1458, 2013 | Alzheimer's |
| Lee et al., Nat. Genet. 50, 1112-1121, 2018 | Educational |
| | Attainment |
| Liu et al., Nat. Genet. 47, 979-986, 2015 | Inflammatory |
| | Bowel |
| | Disease |
| Major Depressive Disorder Working Group of the Psychiatric GWAS | Major |
| Consortium et al., Mol. Psychiatry. 18, 497-511, 2013 | Depressive |
| | Disorder |
| Marioni et al., Transl Psychiatry. 8, 99, 2018 | Alzheimer's |
| Okbay et al., Nature. 533, 539-542, 2016 | Educational |
| | Attainment |
| Psychiatric GWAS Consortium Bipolar Disorder Working Group, Nat. Genet. | Bipolar |
| 43, 977-983, 2011 | Disorder |
| Schizophrenia Psychiatric Genome-Wide Association Study (GWAS) | Schizophrenia |
| Consortium, Nat. Genet. 43, 969-976, 2011 | |
| Schizophrenia Working Group of the Psychiatric Genomics Consortium, | Schizophrenia |
| Nature. 511, 421-427, 2014 | |
| Tourette Association of America International Consortium for Genetics | Tourette |
| (TAAICG, Interrogating the genetic determinants of Tourette syndrome and | |
| other tic disorders through genome-wide association studies, 2018 | |
| Wray et al., Nat. Genet. 50, 668-681, 2018 | Major |
| | Depressive |
| | Disorder |
| Yang et al., Nat Meth. 14, 621-628, 2017 | |
| Demontis, Discovery Of The First Genome-Wide Significant Risk Loci For | ADHD |
| ADHD \| bioRxiv, (available online at biorxiv.org/content/10.1101/145581v1). | |

TABLE 3
Buffer Recipes

| Proteinase K | EDTA | 50 | mM |
|---|---|---|---|
| Cleanup Buffer | Sodium chloride | 5 | mM |
| | Sodium dodecyl sulfate | 1.25% | (w/v) |
| | Proteinase K (Qiagen # 19131) | 5 | mg/mL |
| Nuclei Isolation | Sucrose | 250 | mM |
| Medium | Potassium chloride | 25 | mM |
| | Magnesium chloride | 5 | mM |
| | Tris-HCl | 10 | mM |
| Homogenization | pH to 8.0 and sterile filter. Store refrigerated. | | |
| Buffer | 10 mL Nuclei Isolation Medium | | |
| | 0.1% (w/v) Triton X-100 | | |
| | One pellet Roche Mini cOmplete ™ EDTA-free | | |
| | (Sigma catalog # 4693159001) | | |
| | Prepare fresh on day of experiment. | | |
| Blocking Buffer | PBS | | |
| | BSA (catalog # A2058 from Millipore Sigma) | 0.5% | (w/v) |
| | Triton X-100 | 0.1% | (w/v) |
| ACSF.7 | HEPES | 20 | mM |
| | Sodium Pyruvate | 3 | mM |
| | Taurine | 10 | μM |
| | Thiourea | 2 | mM |
| | D-(+)-glucose | 25 | mM |
| | Myo-inositol | 3 | mM |
| | Sodium bicarbonate | 30 | mM |
| | Calcium chloride dihydrate | 0.5 | mM |
| | Magnesium sulfate | 10 | mM |
| | Potassium chloride | 2.5 | mM |
| | Monosodium Phosphate | 1.25 | mM |
| | HCl | 92 | mM |
| | N-methyl-D-(+)-glucamine | 92 | mM |
| | L-ascorbic acid | 5.0 | mM |
| | N-acetyl-L-cysteine | 12 | mM |
| | Adjust pH to 7.3-7.4 with HCl, then adjust osmolarity to 295-305. Sterile filter, | | |
| | and then make 100 mL aliquots and freeze them. The thawed aliquot keeps 2-3 months at 4 | | |
| | degrees, until it turns yellow. Bubble with carbogen at least 10-15 minutes before use, and | | |
| | continuously while in use. | | |
| ACSF.8 | HEPES | 20 | mM |
| | Taurine | 10 | μM |
| | Thiourea | 2 | mM |
| | D-(+)-glucose | 25 | mM |
| | Myo-inositol | 3 | mM |
| | Sodium bicarbonate | 30 | mM |
| | Calcium chloride dihydrate | 2.0 | mM |
| | Magnesium sulfate | 2.0 | mM |
| | Potassium chloride | 2.5 | mM |
| | Monosodium Phosphate | 1.25 | mM |
| | Sodium chloride | 92 | mM |
| | L-ascorbic acid | 5.0 | mM |
| | N-acetyl-L-cysteine | 12 | mM |
| | Adjust pH to 7.3-7.4 with HCl, then adjust osmolarity to 295-305. Sterile filter, | | |
| | and then make 100 mL aliquots and freeze them. The thawed aliquot keeps 2-3 months at 4 | | |
| | degrees, until it turns yellow. Bubble with carbogen at least 10-15 minutes before use, and | | |
| | continuously while in use. | | |
| Slice Culture | MEM Eagle medium powder | 1680 | mg |
| Medium | (MilliporeSigma catalog # M4642) | | |
| | L-ascorbic acid powder | 36 | mg |
| | CaCl₂, 2.0M | 100 | μL |
| | MgSO₄, 2.0M | 200 | μL |
| | HEPES, 1.0M | 6.0 | mL |
| | Sodium bicarbonate, 893 mM | 3.36 | mL |
| | D-(+)-glucose, 1.11M | 2.25 | mL |
| | Pen/Strep 100x (5k U/mL) | 1.0 | mL |
| | (Thermo catalog # 15070063) | | |
| | Tris base, 1.0M | 260 | μL |
| | GlutaMAX 200 mM | 0.5 | mL |
| | (Thermo catalog # 35050061) | | |
| | Bovine Pancreas Insulin, 10 mg/mL | 20 | μL |
| | (MilliporeSigma catalog # I0516) | | |
| | Heat-inactivated horse serum | 40 | mL |
| | (Thermo catalog # 26050088) | | |
| | Deionized water to 250 mL Adjust pH to 7.3-7.4 with HCl, then adjust osmolarity to 300-305. Sterile | | |
| | filter and store refrigerated for up to 1-2 months. | | |
| ACSF.1/trehalose | HEPES | 20 | mM |
| | Sodium Pyruvate | 3 | mM |
| | Taurine | 10 | μM |
| | Thiourea | 2 | mM |
| | D-(+)-glucose | 25 | mM |
| | Myo-inositol | 3 | mM |
| | Sodium bicarbonate | 25 | mM |
| | Calcium chloride dihydrate | 0.5 | mM |
| | Magnesium sulfate | 10 | mM |
| | Potassium chloride | 2.5 | mM |
| | Monosodium Phosphate | 1.25 | mM |
| | Trehalose dihydrate | 132 | mM |
| | N-methyl-D-(+)-glucamine | 30 | mM |
| | L-ascorbic acid | 5.0 | mM |
| | N-acetyl-L-cysteine 1 | 2 | mM |
| | Adjust pH to 7.3-7.4 with HCl and adjust osmolarity to 295-305. | | |
| | Sterile filter, and then make 100 mL aliquots and freeze them. The | | |
| | thawed aliquot keeps 2-3 months at 4 degrees, until it turns yellow. | | |
| ACSF.1/trehalose + | ACSF.1/trehalose | 50 | mL |
| blockers | 100 μM TTX (final 0.1 μM) | 50 | μL |
| | 25 mM DL-AP5 (final 50 μM) | 100 | μL |
| | 60 mM DNQX (final 20 μM) | 15 | μL |
| | 100 mM (+)-MK801 (final 10 μM) | 5 | μL |
| ACSF.1/trehalose + | ACSF.1/trehalose + blockers | 15 | mL |
| blockers + | One vial Worthington PAP2 reagent (150 U, final 10 U/mL) | | |
| papain | 10 kU/mL DNase I (Roche) | 15 | μL |
| Low-BSA Quench | ACSF.1/trehalose + blockers | 15 | mL |
| buffer | 10 kU/mL DNase I (Roche) | 15 | μL |
| | 20% BSA dissolved in water (final conc. 2 mg/mL) | 150 | μL |
| | 10 mg/mL ovomucoid inhibitor | 150 | μL |
| | (Sigma T9253, final conc. 0.1 mg/mL) | | |
| High-BSA Quench | ACSF.1/trehalose + blockers | 15 | mL |
| buffer | 10 kU/mL DNase I (Roche) | 15 | μL |
| | 20% BSA dissolved in water (final cone. 10 mg/mL) | 750 | μL |
| | 10 mg/mL ovomucoid inhibitor | 150 | μL |
| | (Sigma T9253, final cone. 0.1 mg/mL) | | |
| ACSF.1/trehalose + | HEPES | 20 | mM |
| EDTA | Sodium Pyruvate | 3 | mM |
| | Taurine | 10 | μM |
| | Thiourea | 2 | mM |
| | D-(+)-glucose | 25 | mM |
| | Myo-inositol | 3 | mM |
| | Sodium bicarbonate | 25 | mM |
| | Potassium chloride | 2.5 | mM |
| | Monosodium Phosphate | 1.25 | mM |
| | Trehalose | 132 | mM |
| | HCl | 2.9 | mM |
| | EDTA | 0.25 | mM |
| | N-methyl-D-(+)-glucamine | 30 | mM |
| | L-ascorbic acid | 5.0 | mM |
| | N-acetyl-L-cysteine | 12 | mM |
| | Adjust pH to 7.3-7.4 with HCl and adjust osmolarity to 295-305. | | |
| | Sterile filter, and then make 100 mL aliquots and freeze them (−20). The | | |
| | thawed aliquot keeps 2-3 months at 4 degrees, until it turns yellow. | | |
| Cell | ACSF.1/trehalose + EDTA | 50 | mL |
| Resuspension | 100 μM TTX (final 0.1 μM) | 50 | μL |
| Buffer | 25 mM DL-AP5 (final 50 μM) | 100 | μL |
| | 60 mM DNQX (final 20 μM) | 15 | μL |
| | 100 mM (+)-MK801 (final 10 μM) | 5 | μL |
| | 20% BSA dissolved in water (final conc. 2 mg/mL) | 150 | μL |
| | 4′-diamino-phenylindazole (DAPI) | 1 | μg/mL |

(ix) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within +2 is preferred, those within +1 are particularly preferred, and those within +0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N. Y . . . . Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The term concatenate is broadly used to describe linking together into a chain or series. It is used to describe the linking together of nucleotide or amino acid sequences into a single nucleotide or amino acid sequence, respectively. The term “concatamerize” should be interpreted to recite: “concatenate.”

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in selective expression in the targeted cell population as determined by scRNA-Seq and the selected artificial expression construct/targeted cell population pairing.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1.-66. (canceled)

67. An artificial expression construct comprising:
(i) an enhancer having the sequence of SEQ ID NO: 178, SEQ ID NO: 29, SEQ ID NO: 188, SEQ ID NO: 25, SEQ ID NO: 26, SEQ ID NO: 27, SEQ ID NO: 28, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, or SEQ ID NO: 51, or a sequence having at least 95% sequence identity to the sequence of SEQ ID NO: 178, SEQ ID NO: 29, SEQ ID NO: 188, SEQ ID NO: 25, SEQ ID NO: 26, SEQ ID NO: 27, SEQ ID NO: 28, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, or SEQ ID NO: 51;
(ii) a promoter, and
(iii) a coding sequence.

68. The artificial expression construct of claim 67, wherein the enhancer comprises 1, 2, 3, 4, 5, 6, 7, 8, 9, or 10 copies of a sequence having at least 95% sequence identity to the sequence of SEQ ID NO: 178, SEQ ID NO: 29, SEQ ID NO: 188, SEQ ID NO: 25, SEQ ID NO: 26, SEQ ID NO: 27, SEQ ID NO: 28, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, or SEQ ID NO: 51.

69. The artificial expression construct of claim 67, wherein the enhancer comprises 1, 2, 3, 4, 5, 6, 7, 8, 9, or 10 copies of the sequence of SEQ ID NO: 178, SEQ ID NO: 29, SEQ ID NO: 188, SEQ ID NO: 25, SEQ ID NO: 26, SEQ ID NO: 27, SEQ ID NO: 28, SEQ ID NO: 31, SEQ ID NO: 32, SEQ ID NO: 33, SEQ ID NO: 34, SEQ ID NO: 35, SEQ ID NO: 36, SEQ ID NO:
37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, or SEQ ID NO: 51.

70. The artificial expression construct of claim 67, wherein the enhancer comprises
three copies of the sequence of SEQ ID NO: 178,
three copies of the sequence of SEQ ID NO: 29,
four copies of the sequence of SEQ ID NO: 29,
three copies of a sequence having at least 95% sequence identity to the sequence of SEQ ID NO: 178,
three copies of a sequence having at least 95% sequence identity to the sequence of SEQ ID NO: 29, or
four copies of a sequence having at least 95% sequence identity to the sequence of SEQ ID NO: 29.

71. The artificial expression construct of claim 67, wherein the enhancer comprises
the sequence of SEQ ID NO: 49,
the sequence of SEQ ID NO: 30,
the sequence of SEQ ID NO: 31,
a sequence having at least 95% sequence identity with the sequence of SEQ ID NO: 49,
a sequence having at least 95% sequence identity with the sequence of SEQ ID NO: 30, or
a sequence having at least 95% sequence identity with the sequence of SEQ ID NO: 31.

72. The artificial expression construct of claim 67, wherein the coding sequence encodes an effector element or an expressible element.

73. The artificial expression construct of claim 72, wherein the effector element comprises a reporter protein or a functional molecule.

74. The artificial expression construct of claim 73, wherein the reporter protein comprises a fluorescent protein.

75. The artificial expression construct of claim 73, wherein the functional molecule comprises a functional ion transporter, enzyme, transcription factor, receptor, membrane protein, cellular trafficking protein, signaling molecule, neurotransmitter, calcium reporter, channel rhodopsin, CRISPR/Cas molecule, editase, guide RNA molecule, homologous recombination donor cassette, or a designer receptor exclusively activated by designer drug (DREADD).

76. The artificial expression construct of claim 67, wherein the artificial expression construct is associated with a capsid that crosses the blood brain barrier.

77. The artificial expression construct of claim 76, wherein the capsid comprises PHP.eB, AAV-BR1, AAV-PHP.S, AAV-PHP.B, AAV-PPS, AAV9, or AAVrh.10.

78. The artificial expression construct of claim 67, wherein the expression construct comprises or encodes a skipping element.

79. The artificial expression construct of claim 78, wherein the skipping element comprises a 2A peptide and/or an internal ribosome entry site (IRES).

80. The artificial expression construct of claim 79, wherein the 2A peptide is selected from T2A, P2A, E2A, or F2A.

81. The artificial expression construct of claim 67, wherein the artificial expression construct is within a viral vector.

82. The artificial expression construct of claim 81, wherein the viral vector comprises a recombinant adeno-associated viral (AAV) vector.

83. The artificial expression construct of claim 82, wherein the AAV vector is replication-competent.

84. The artificial expression construct of claim 67, comprising a sequence having 95% sequence identity to the sequence of SEQ ID NO: 111, SEQ ID NO: 101, SEQ ID NO: 73, SEQ ID NO: 179, SEQ ID NO: 78, SEQ ID NO: 77, SEQ ID NO: 79, SEQ ID NO: 76, SEQ ID NO: 74, SEQ ID NO: 75, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 105, SEQ ID NO: 109, SEQ ID NO: 100, SEQ ID NO: 102, SEQ ID NO: 106, SEQ ID NO: 103, SEQ ID NO: 104, SEQ ID NO: 108, SEQ ID NO: 107, SEQ ID NO: 110, SEQ ID NO: 113, SEQ ID NO: 114, or SEQ ID NO: 112.

85. The artificial expression construct of claim 67, comprising the sequence of SEQ ID NO: 111, SEQ ID NO: 101, SEQ ID NO: 73, SEQ ID NO: 179, SEQ ID NO: 78, SEQ ID NO: 77, SEQ ID NO: 79, SEQ ID NO: 76, SEQ ID NO: 74, SEQ ID NO: 75, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 105, SEQ ID NO: 109, SEQ ID NO: 100, SEQ ID NO: 102, SEQ ID NO: 106, SEQ ID NO: 103, SEQ ID NO: 104, SEQ ID NO: 108, SEQ ID NO: 107, SEQ ID NO: 110, SEQ ID NO: 113, SEQ ID NO: 114, or SEQ ID NO: 112.

86. A method for selectively expressing a coding sequence within a population of neural cells in vivo or in vitro, the method comprising providing an administrable composition comprising an artificial expression construct comprising artificial expression construct comprising:
an enhancer having the sequence of SEQ ID NO: 178, SEQ ID NO: 29, SEQ ID NO: 188, SEQ ID NO: 25, SEQ ID NO: 26, SEQ ID NO: 27, SEQ ID NO: 28, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, or SEQ ID NO: 51, or a sequence having at least 95% sequence identity to the sequence of SEQ ID NO: 178, SEQ ID NO: 29, SEQ ID NO: 188, SEQ ID NO: 25, SEQ ID NO: 26, SEQ ID NO: 27, SEQ ID NO: 28, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, or SEQ ID NO: 51;
a promoter, and
a coding sequence;
in a sufficient dosage and for a sufficient time to a sample or subject comprising the population of neural cells thereby selectively expressing the coding sequence within the population of neural cells.


SEQUENCE LISTING (188 sequences)

SEQ ID NO: 1 - DNA, 49 bp, synthetic construct
  misc_feature 1..49: note = 5' Cloning Primer
  source 1..49: mol_type = other DNA
ggtggagtcgtgacctaggacgcgtttctaatcatgaattcttttgtgg

SEQ ID NO: 2 - DNA, 46 bp, synthetic construct
  misc_feature 1..46: note = 5' Cloning Primer
  source 1..46: mol_type = other DNA
ggtggagtcgtgacctaggacgcgtctgattgtacagcaggcactc

SEQ ID NO: 3 - DNA, 45 bp, synthetic construct
  misc_feature 1..45: note = 5' Cloning Primer
  source 1..45: mol_type = other DNA
ggtggagtcgtgacctaggacgcgtacctttccagcctggcttac

SEQ ID NO: 4 - DNA, 44 bp, synthetic construct
  misc_feature 1..44: note = 5' Cloning Primer
  source 1..44: mol_type = other DNA
ggtggagtcgtgacctaggacgcgtataagccttgggggcaatc

SEQ ID NO: 5 - DNA, 35 bp, synthetic construct
  misc_feature 1..35: note = 5' Cloning Primer
  source 1..35: mol_type = other DNA
cgtacacgcgtatgtgtcttttactctgatcctcc

SEQ ID NO: 6 - DNA, 35 bp, synthetic construct
  misc_feature 1..35: note = 5' Cloning Primer
  source 1..35: mol_type = other DNA
cgtacacgcgtcagtagtgttaatgacagagtcag

SEQ ID NO: 7 - DNA, 31 bp, synthetic construct
  misc_feature 1..31: note = 5' Cloning Primer
  source 1..31: mol_type = other DNA
cgtacacgcgtccttttccaaccgttccttc

SEQ ID NO: 8 - DNA, 31 bp, synthetic construct
  misc_feature 1..31: note = 5' Cloning Primer
  source 1..31: mol_type = other DNA
cgtacacgcgtgtcccataggcagtttgtgg

SEQ ID NO: 9 - DNA, 31 bp, synthetic construct
  misc_feature 1..31: note = 5' Cloning Primer
  source 1..31: mol_type = other DNA
cgtacacgcgtagccgctctcaccttctata

SEQ ID NO: 10 - DNA, 31 bp, synthetic construct
  misc_feature 1..31: note = 5' Cloning Primer
  source 1..31: mol_type = other DNA
cgtacacgcgtgagctgggcatcatcacatc

SEQ ID NO: 11 - DNA, 31 bp, synthetic construct
  misc_feature 1..31: note = 5' Cloning Primer
  source 1..31: mol_type = other DNA
cgtacacgcgtagggtgcaggagaaatgtga

SEQ ID NO: 12 - DNA, 47 bp, synthetic construct
  misc_feature 1..47: note = 3' Cloning Primer
  source 1..47: mol_type = other DNA
cttttatgcccagcccgagctcaatgttcttgaacttaccaatcagg

SEQ ID NO: 13 - DNA, 43 bp, synthetic construct
  misc_feature 1..43: note = 3' Cloning Primer
  source 1..43: mol_type = other DNA
cttttatgcccagcccgagctcatgccgtgaagtaatccagtg

SEQ ID NO: 14 - DNA, 45 bp, synthetic construct
  misc_feature 1..45: note = 3' Cloning Primer
  source 1..45: mol_type = other DNA
cttttatgcccagcccgagctccggtggttgaaataaacaagaac

SEQ ID NO: 15 - DNA, 44 bp, synthetic construct
  misc_feature 1..44: note = 3' Cloning Primer
  source 1..44: mol_type = other DNA
cttttatgcccagcccgagctcatttgtcctgtgcatagcattg

SEQ ID NO: 16 - DNA, 34 bp, synthetic construct
  misc_feature 1..34: note = 3' Cloning Primer
  source 1..34: mol_type = other DNA
cgtacgagctctgttgctacactagactcaatgg

SEQ ID NO: 17 - DNA, 35 bp, synthetic construct
  misc_feature 1..35: note = 3' Cloning Primer
  source 1..35: mol_type = other DNA
cgtacgagctcggcttgagtatagacaaaccactc

SEQ ID NO: 18 - DNA, 30 bp, synthetic construct
  misc_feature 1..30: note = 3' Cloning Primer
  source 1..30: mol_type = other DNA
cgtacgagctctgctcaggaaccaaaggag

SEQ ID NO: 19 - DNA, 33 bp, synthetic construct
  misc_feature 1..33: note = 3' Cloning Primer
  source 1..33: mol_type = other DNA
cgtacgagctcagaccttaccactgcattctga

SEQ ID NO: 20 - DNA, 32 bp, synthetic construct
  misc_feature 1..32: note = 3' Cloning Primer
  source 1..32: mol_type = other DNA
cgtacgagctcctgggatgaacggaattgtgt

SEQ ID NO: 21 - DNA, 31 bp, synthetic construct
  misc_feature 1..31: note = 3' Cloning Primer
  source 1..31: mol_type = other DNA
cgtacgagctctgccgtctgatttgcatact

SEQ ID NO: 22 - DNA, 31 bp, synthetic construct
  misc_feature 1..31: note = 3' Cloning Primer
  source 1..31: mol_type = other DNA
cgtacgagctccttgcccatgaacgttctgt

SEQ ID NO: 23 - DNA, 4712 bp, synthetic construct
  misc_feature 1..4712: note = CN1818 top strand
  source 1..4712: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgcttaaggagctcagaggtaggcgtgtacggtgggaggcctatataagcagagctggtttagtgaaccgtcagatcgcctggggatccttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 4712 bp, synthetic construct
  misc_feature 1..4712: note = CN1818 bottom strand
  source 1..4712: mol_type = other DNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgtgcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaagatctctcgagccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatgatatcgaattcgcggccgcggcgcgccgtcgacttacttgtacagctcgtccatgccgagagtgatcccggcggcggtcacgaactccagcaggaccatgtgatcgcgcttctcgttggggtctttgctcagcttggactggtagctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccgtagcccagggtggtcacgagggtgggccagggcacgggcagcttgccggtggtgcagatcagcttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcttcgaaggatccccaggcgatctgacggttcactaaaccagctctgcttatataggcctcccaccgtacacgcctacctctgagctccttaagcggtgccagattacagagttcctgatcagcacacgttgataggaaattaatgtgcatgtattgaaatagcagaagatgatgtgttctgagatctctctgaaagcaagttagcatggcaatgcaagttagtgagtcagtgatattcaacagggtaacggtgccagattacagagttcctgatcagcacacgttgataggaaattaatgtgcatgtattgaaatagcagaagatgatgtgttctgagatctctctgaaagcaagttagcatggcaatgcaagttagtgagtcagtgatattcaacagggtaacggtgccagattacagagttcctgatcagcacacgttgataggaaattaatgtgcatgtattgaaatagcagaagatgatgtgttctgagatctctctgaaagcaagttagcatggcaatgcaagttagtgagtcagtgatattcaacagggtaaacgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 854 bp, Mus musculus
  source 1..854: mol_type = genomic DNA
ctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacagaaagcccacagaatcaaagacgtgttcaaacagccagctgaattcattgcacatttcaaccacagaaatattttcaggtgattctgttgtttgacaaaacgtgggaaccacaggatctacaacacttgcaagcaaaactcaacagctctaataatagttacagaagtgaaagccaatttggataaaataagacattgactcaagtcctctcagaagagttttgaaagcaaagtttacaaaagtctggtttgtcctttgggatttacagacctttcagccccttgattcatttttttttttttggatttcttcatcactgggagaattcccatgcattatttctcccctgcttcaaaatcatcaaatgtgaaacatttttcactcttttcttctgtatatagtgataaaatagctattggcttttggctaaatgtgctactttgagcccacccacacaagggagaaatgggggcagacatgagtttgggcatgagt

SEQ ID NO: 26 - DNA, 564 bp, Mus musculus
  source 1..564: mol_type = genomic DNA
ttctaatcatgaattcttttgtggtattttagattttcagtttgttctgtatcaattctctctcattcaaagaatatgatagtgaggtagatgaagctgcctaagccacaggaagaaacatcttcagtctgtcgtaaatgcacttcagatacagcaccttgcactgcacataaaaattcatagtcacctaagtgggaatagctatgaaaatctgagtatcgccatgctgttgactcagtgctatttataaaactcagttttaatgtttccaatttaaattctctgcacatatctctcctgcactaaagacttgagataccagtgctttaccctaaaatatcttgcttttatatcttgactcttatgttgagaatttattatttttaaaatatactttaaaacatgcattggtacaaaattagtcaaaacagcaaccagtgaattcaaagtaaattagtattattaatgctgtgtataattttggtgaattttactattaaattataaataaaaagtccttccaggtagtcatgttcactcctgattggtaagttcaagaacatt

SEQ ID NO: 27 - DNA, 562 bp, Mus musculus
  source 1..562: mol_type = genomic DNA
cctttccagcctggcttacaggcttttttcaccacagtaccaattgcccatgctctgctcattaatttaaatggcaatgactatctgggttttaaaatagagaagtgtcaggatgggaacacgcaatcatttggctttttgcgttccagcactgttttgaatagcagggttttcacttcctatgaaaccttagcaggaggaaaagcggaaactaaaccataaagtgagagggatgaggggagggagggacttgagtatttgtaaactcagggtggctggccctgcctaccaggctgctctctaccaccggaggctaggagtggaaaaacttgatttacgtgttgtgcctgccttttttttttcttcttcttcttcttcttcttcttcgtctccacaccaccttctgtacacctgactctgcataagcctatctgaagctggcttggtggcagggatagctggagaacagaagaatgtgcggagggagggagggaggaagggagggctggtacttttccattcacatctccacagtggctgttcttgtttatttcaaccaccg

SEQ ID NO: 28 - DNA, 555 bp, Mus musculus
  source 1..555: mol_type = genomic DNA
agccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaat

SEQ ID NO: 29 - DNA, 155 bp, Mus musculus
  source 1..155: mol_type = genomic DNA
ttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccg

SEQ ID NO: 30 - DNA, 465 bp, synthetic construct
  misc_feature 1..465: note = concatemer of Mus musculus core enhancer
  source 1..465: mol_type = other DNA
ttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccg

SEQ ID NO: 31 - DNA, 2000 bp, synthetic construct
  misc_feature 1..2000: note = concatamer of Mus musculus enhancer
  source 1..2000: mol_type = other DNA
ccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagagg

SEQ ID NO: 32 - DNA, 501 bp, Mus musculus
  source 1..501: mol_type = genomic DNA
cactgtgcatagcatcattacaatgttatagtttttcacactatgccttgactttttggaaaggcaaaccacctcttggatttctccttccttctctatctctctctctctctcttcctccctccgtccctccatctcttcctccttcccattttcttctctccctatttggacacaatataaaataatttagatgaggtgagttaaattgtgaacaaagtatgtgcctatacatggttgtaaatcagcttatcaaagtgtaatattagaagaatttataaaaatgataaaattcatactcaaagttctgtgtaaagcaataatagctttatctccttttagttatcttgagtctttctatgactaacaactccctcataggcatcttaaagagcagtaagcataagtagattccaaatgggaagggagaagtgtgaaccatcactttcatccagacttgtagatatatctgctgcattttcagaaaccagaaacagacag

SEQ ID NO: 33 - DNA, 501 bp, Mus musculus
  source 1..501: mol_type = genomic DNA
aaaatgttcattttgccaatatgatcaccaataaaaccatttgtgtagactatccaccttaatccccctataatacaatagcacagaggtgagtcagtttgattttgatactaggtttattttataggagctattaaagtttcagaattttgctgagtcaccaggctcttcattttgtggcaaatccatcactacagtttaaggagagaagaaacagacccccccctaccctctgaaaaataaaaataaaaacttgtttcaggcaggctagcgattcactaataatgagaaaactccagttttaagacttaatttcaccataaatactctttcattctaagctctgggacatcatgagccagagaacagcagagtgaataatacagttacagagctgatgagcaatgccagtcactgtaaaaaatacagaatcccatccaaaggacatctgtaaaagtgtctttaacatctactcagcccttctgtgtaaaggtcagcacg

SEQ ID NO: 34 - DNA, 598 bp, Mus musculus
  source 1..598: mol_type = genomic DNA
ccttttccaaccgttccttcatgacatcaaggcttcagactgctaagctttgggcactacctggggtcagtctgcatcaaaatgtaaggctcaaatgtgtaattgtaagtactgttttgctgagctggaagggctcctttgaagcccacgttttaattttaatttagccacacagagtggcaaagacaaatagatttatccaaaatacatttggtaacagattttttgagtcagttattaattttatttgaggggttcctctttttattttttataaactgtgaaactcaagaggaagcaggatcccatgcaatgccttttattgatggcctgctatgtgccaagaaaggtgttaaatgttttccaatgctgcctcatttatcctgatcttacagacaagcaaaaggaggtgtgaagaggtgaagtttctcacccagctggaaagtggcaaagtcattcacagatctgcctccgctcaaaaaaattgctttatgcaactctttggaagctaacttcatgggagctacatgcagcttctcaatgaaccttgttttgctggcctgcagccagaagttactactcctttggttcctgagca

SEQ ID NO: 35 - DNA, 501 bp, Mus musculus
  source 1..501: mol_type = genomic DNA
gactgctaagctttgggcactacctggggtcagtctgcatcaaaatgtaaggctcaaatgtgtaattgtaagtactgttttgctgagctggaagggctcctttgaagcccacgttttaattttaatttagccacacagagtggcaaagacaaatagatttatccaaaatacatttggtaacagattttttgagtcagttattaattttatttgaggggttcctctttttattttttataaactgtgaaactcaagaggaagcaggatcccatgcaatgccttttattgatggcctgctatgtgccaagaaaggtgttaaatgttttccaatgctgcctcatttatcctgatcttacagacaagcaaaaggaggtgtgaagaggtgaagtttctcacccagctggaaagtggcaaagtcattcacagatctgcctccgctcaaaaaaattgctttatgcaactctttggaagctaacttcatgggagctacatgcagcttct

SEQ ID NO: 36 - DNA, 501 bp, Mus musculus
  source 1..501: mol_type = genomic DNA
ggtgggctatgttactgagggtctctgggtgttaggaaaacagggcccaggagtctggctgctcgtatgctggcccaggctcttgtttttcttgagctgacttgctggagaagtgagctaagtcagaaacaaaatgccacattgcacgcccactgaagtctgggctcaagggaaagaagagagattgccagagcgttagctgttcccaatccactcctggaccttaagctgtcttgaacagagttgccaatcagcttggtagggactggcctttgaggaggggagggggtgtaggcaggggagggggagagaagggagcagtctgcgctccatcttaattacctcatcagaaacagctcccttcccgcaaagctctggtgtcttctacaagagggtgagtctttggctttacatgtgaacttgtgccatttgcctgcgtatataaacatgaagggtcgtctgggttcagagctgaaatctttcacttgtgacttagctggg

SEQ ID NO: 37 - DNA, 501 bp, Mus musculus
  source 1..501: mol_type = genomic DNA
ctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccc

SEQ ID NO: 38 - DNA, 2372 bp, synthetic construct
  misc_feature 1..2372: note = concatamer of Mus musculus enhancer
  source 1..2372: mol_type = other DNA
ctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaagtgacatagcgatagacagattcgactcggtaccagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggatgatggcatcattgagtagcatgatctcaattgagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccc

SEQ ID NO: 39 - DNA, 536 bp, Homo sapiens
  source 1..536: mol_type = genomic DNA
tagtctgcctcaggtacacactgagaaactgctttaatgtaacctgacccacggttattagtgaaaatatcacttttgttgttaccttattcccaacaaattcatttctgctttaatggaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcacaaatgtttaattaaaaaaaaaaaaagtcatgtactttcatagatattcacaatcctctctagtatactttcaaatcagttttaatttcagtttagtgtttttatgt

SEQ ID NO: 40 - DNA, 980 bp, synthetic construct
  misc_feature 1..980: note = concatamer of Homo sapiens enhancer core
  source 1..980: mol_type = other DNA
gaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcgagcagagccctcatcacacagactgaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatctgggtgagtaagagtaggtggcaacgaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatc

SEQ ID NO: 41 - DNA, 567 bp, Homo sapiens
  source 1..567: mol_type = genomic DNA
caaaagatggaagttgggaggttgaagaagtgcaggatggcattccaagtgatgggggcaatggcatggaggtaggaaagcataaggtatattcaggctataaataatagttagatttggctggatcctggatttgagaagccaggaaatgagataacactggtcactttcactaaagctcatgaaaaaaaaaatacatacatatatatatatataaaataaatatacatatatatttttaagccccatatgactagaggaggcagcccatctgttctctgggcttcacttttcttgtctgggaaatgagtaggttggactgcatggtctttaaggtctctttagtattatcttgtttgactccgtaaagagaaaaacaaaggttcctcctgacatcttgtgttgccttccaacgtccagtccagtgtgattgttttaagtactctttggatattttactgttataaaaagtgaagaaaaagactgattttgccaagtcttatggatccaaattagtactcattgcactatggtcatttagttgaggacgatactccagcttcaaag

SEQ ID NO: 42 - DNA, 732 bp, Mus musculus
  source 1..732: mol_type = genomic DNA
cgcgtggtacatattataagtttgagtctgcaagatgtggcaaacccttccttttctctttatcttgacagtggaaaacatctaaggagtctttaaaataaccacatcggactgagcagtggagggcaaagagaatgtctggaagaacctttgtttttttcgttgggacatcaaaggatgttatactgaagagatctcaaagaccaggcagtccagcctactcgtttcccagacaagagaagtgaagccaagaaacagatgggctgcctcctctagtcatatggggcttaaaaatatatatctctatttttcatgagttttcatgaaagtgaccagtgttatctcatttcctgtcttctcaaattcaggatcctacccaatctaactattatttatagtgtgaataagccctctactttcctacctctacaatgctcattctccattttcccatcatctacttccatcttgtgaaaagtttccactcttcagtgatgacctcaaacatatcatttcttttttttaattctttttttattagatattttctttatatacatttcaaatgctatcctgaaagttccctatatcctccctctgccctgcttccctacccacccactcccacttcttggccctggcatttccctgtactggggcatataaagtttgcaataccaaggggcctctcttcacagtgatggccaactaggccatcttctgctacgagct

SEQ ID NO: 43 - DNA, 756 bp, Homo sapiens
  source 1..756: mol_type = genomic DNA
actggtctgactgcagaggaggtctgggagaccagagggagtgtggagagggtgaggttagaagacaggaacaccgagctgcatcggccaaatggagccttagggggccatgtgaggctgaggcagggaagcagggatcctgccctccaggtccttacagtcaggcggggaccaaaagcacgaggatgccagcccaattccctattaggcaaaacgcagcaccatctgcacaatcccaggagcaagagcagatattttataacttccttttttctttttaagtctaaattaaaaataaatgttcccttcagctctcagatgtatatctctggtgcaacctgcccacattccctcccgctgccctttccagaacatggcaggggaaaggaagaaagagatggatagagagagggagccagtccacccagcttcaatgccagtggattgcacctcttccaagagggaaacgattcaggcgtggccacgcagacgggtggagagcgcccagaatgtggctggtaccaaggaaagtggaaggagagggaaacaggagccaacagctatgatttctagcccagcctccaccctatcgcgctgcaggaccttggccaaatcacacatcctatctctgctcccatttatagttcataacatggctgaagtcccctctgccgctccagccccctggcagctgtgctctctgcacatccgtctgtacctttgctgctccccttcattttgggtgtcctaccatgg

SEQ ID NO: 44 - DNA, 670 bp, Mus musculus
  source 1..670: mol_type = genomic DNA
actggcctgactgggaagaatgtcccagacattgacaaaagacaatctagagagggtaaggatgggagaccagggacaccaaagagcccactgggtccctgtggccaggcggggcccagagcacatggtgccagcctcgttccctattaggcaaagcactgcaccatctgtatagtcccaggagcaggagcaggagcaggcgttttataacttccttttctttttcagtctacattaaaaataaatgttcccttcagctctcagatgtatatctctagtgcaacctgcccacattccctcctgctgccctttccagaacatggcaggggaagggaaggaagagatggagagagggagccagtccacccggctgatgccagtggatcacacctcttctaagagggaagcgcggcaggcacggccacacatggtggaaggtgcccagaatgcatggggaccagggaaatggaagtggaggaaatgggagccaacagccaggcttgcttcccacccccaccctcccgcaccgcaggaccttggccaaatcacacatcccatctttgcatttatagttcatgcagtggctggagtcccctctgcagctccagccctctggtggctgtccttgctgcacgtctctctgtacttccccttgtgtgtcctgctgtgg

SEQ ID NO: 45 - DNA, 584 bp, Homo sapiens
  source 1..584: mol_type = genomic DNA
gaggaacagaacaaaacagaacaagcaggttcacttgggacgccgggaacaccccgggcttgcgcccctgcgcctccccgctggcggccccgccaacttcccggggtgtcccctccctaccttctcttcaccgccctggcgcctggcctgcgcgaggtcgggactcgcgggacctccgcctaccccagaagcggctgtctaaagcgggggtgggggggcgccccctcctgtctggttttcccttccagttgccgggagaggactaggcagccgggagccgggccgtgcacccgctgtggcgcgctggcacctcggcctccgcaaacagattgctcgccctcctcggggaaagctaggaaaacagtgctaagcctcgcaagctgccgcccattaatgcctcttagcttgcaagatgggttactagctctgagcacggccctcccctcggggcttcttacattctcctccccctcgccccttctgtctccctccttctccacgccgcggtactctcgccttcgccctcattctctccctccacctactacctcttccttttgttttccgttctcctgaatttccct

SEQ ID NO: 46 - DNA, 648 bp, Homo sapiens
  source 1..648: mol_type = genomic DNA
gggtcagagacacagaggatgacagagacccagagagagggagacagagacccagagagagggggagagagacctagaaatagggggacagagacccagagagggaagagatggaaacctagagaaggaagcagacagagtcccaaagagaggcggggacagaaacccaggagatagaacatagatgcagagagatgagaacagagatccagaatgcaagagaaagatggagagcctgggagacggaggatagacaggctggggacgtgatttgtgaggtgcagcccctctctgaggtgggtaggcagccaggggatcgggctggatcccaggaaggggctggaacagatggaggcgagggagactgggacgggggaggaaggaacagccagacggtccagggggagggaggtggaagagctgtgagaacacagcaacccctccccatcctagaacttaaggaggtggggaggggcagttaagaaacaggggtgggagaagccagggagtggacagaacccagaaggagtaggaatgagaccccaagaggaagaagacagagagccagagataggggggattgaagccactaggacgaatagtacaggatggcagtaactccccccacccatcagaacccatcaccca

SEQ ID NO: 47 - DNA, 566 bp, Homo sapiens
  source 1..566: mol_type = genomic DNA
ggcttttggcagaaatcagttcgttgtggtttaaggactgaggtcgcctttctttgctgctaactgttggccaggaagtactgtcagctactacatggtacttgctggtccttgcacatgaccccctccaccttcaaaccaggattgacacagtgtgttgagtctttcttgaagacttttgaacatctgacttcccattctgccactagccagttaaaattctctaattttgaaggactcacctggttggccaaacccacccaaataatctccacatcttgaagtcaactgacccaggactttacatatgcaaaatcccttcacagcagtacctagatgagtgtttgcttgaataactgggacacaggaatcttgggggagccatctttagaattcgacctcctacaacccttctggaaatctgagagtgagtcaggggaagaaaccctcttttgtagtttccttttagggctttctactttgctcaaagttgggcactatttcacttcagtagggtcctgcaagccccatgagggtagtgagtgctgtcctaggaaacagtaact

SEQ ID NO: 48 - DNA, 520 bp, Mus musculus
  source 1..520: mol_type = genomic DNA
ccctggccttcgagcacatgctcagatgatgctccaccgtggcctgacccacatcttctagtggaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctcatgatgtatgctcaaatcagttttaattgaactgtgtgtagtatgctcctgttttg

SEQ ID NO: 49 - DNA, 1253 bp, synthetic construct
  misc_feature 1..1253: note = concatamer of Mus musculus enhancer core
  source 1..1253: mol_type = other DNA
gaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctcgagcagagccctcatcacacagactgaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctctgggtgagtaagagtaggtggcaacgaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctc

SEQ ID NO: 50 - DNA, 386 bp, Mus musculus
  source 1..386: mol_type = genomic DNA
aattgctgtcatttacctacggttgtctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacaga

SEQ ID NO: 51 - DNA, 363 bp, Homo sapiens
  source 1..363: mol_type = genomic DNA
aagccaatgacattagagaagtgttcaaacagtcagctaaattcactgcacttctcaaccacagaaatattttcaggtgattctgtttttgagaaaacgtgggaaccacaggatctacaacacttccaggcaaaactcaacagctctaataatagtgacagaagtgaaagccaatttggataaaataagacattgactcaaagtcctctgagagatttttcaaaacaaagtttacaaagctccttttgccttttgggaaatcacattcttctttgcaccttgactctttttctgaatttctttctgtctgggaggatctccttacagtgtttcttctccatctgacatcatgaaatgtgatac

SEQ ID NO: 52 - DNA, 53 bp, synthetic construct
  misc_feature 1..53: note = beta globin minimal promoter
  source 1..53: mol_type = other DNA
gggctgggcataaaagtcagggcagagccatctattgcttacatttgcttctg

SEQ ID NO: 53 - DNA, 68 bp, synthetic construct
  misc_feature 1..68: note = minCMV
  source 1..68: mol_type = other DNA
gaggtaggcgtgtacggtgggaggcctatataagcagagctcgtttagtgaaccgtcagatcgcctgg

SEQ ID NO: 54 - DNA, 68 bp, synthetic construct
  misc_feature 1..68: note = mutated minCMV promoter
  source 1..68: mol_type = other DNA
gaggtaggcgtgtacggtgggaggcctatataagcagagctggtttagtgaaccgtcagatcgcctgg

SEQ ID NO: 55 - DNA, 867 bp, synthetic construct
  misc_feature 1..867: note = Hsp68 minimal Promoter
  source 1..867: mol_type = other DNA
caggaacatccaaactgagcagccggggtcccccccaccccccaccccgccccacgcggcaactttgagcctgtgctgggacagagcctctagttcctaaattagtccatgaggtcagaggcagcactgccattgtaacgcgattggagaggatcacgtcaccggacacgcccccaggcatctccctgggtctcctaaacttggcggggagaagttttagcccttaagttttagcctttaacccccatattcagaactgtgcgagttggcgaaaccccacaaatcacaacaaactgtacacaacaccgagctagaggtgatctttcttgtccattccacacaggccttagtaatgcgtcgccatagcaacagtgtcactagtagcaccagcacttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcaggtcaccagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgcc

SEQ ID NO: 56 - DNA, 720 bp, synthetic construct
  misc_feature 1..720: note = SYFP2
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 57 - DNA, 720 bp, synthetic construct
  misc_feature 1..720: note = EGFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 58 - DNA, 1299 bp, synthetic construct
  misc_feature 1..1299: note = Optimized Flp recombinase
  source 1..1299: mol_type = other DNA
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 59 - DNA, 1056 bp, synthetic construct
  misc_feature 1..1056: note = Improved Cre recombinase
  source 1..1056: mol_type = other DNA
atggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaa

SEQ ID NO: 60 - DNA, 384 bp, synthetic construct
  misc_feature 1..384: note = WPRE3
  source 1..384: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtgg

SEQ ID NO: 61 - DNA, 204 bp, synthetic construct
  misc_feature 1..204: note = BGHpA
  source 1..204: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 62 - protein, 33 aa, synthetic construct
  REGION 1..33: note = HA tag
MVYPYDVPDYAGSYPYDVPDYAGSYPYDVPDYA

SEQ ID NO: 63 - DNA, 99 bp, synthetic construct
  misc_feature 1..99: note = HA tag
  source 1..99: mol_type = other DNA
atggtttacccgtatgatgtcccggattacgctggcagctacccatacgatgtacccgactatgccggcagttatccctacgacgtccctgactacgca

SEQ ID NO: 64 - DNA, 78 bp, synthetic construct
  misc_feature 1..78: note = P2A
  source 1..78: mol_type = other DNA
ggcagcggcgccaccaacttcagcctgctgaagcaggccggcgacgtggaggagaaccccggccccggagctagcgga

SEQ ID NO: 65 - protein, 21 aa, synthetic construct
  REGION 1..21: note = T2A
  REGION 1..3: note = MISC_FEATURE - (GlySerGly) residues can be added to the 5' end of the peptide to improve cleavage efficiency
GSGEGRGSLLTCGDVEENPGP

SEQ ID NO: 66 - protein, 24 aa, synthetic construct
  REGION 1..24: note = E2A
  REGION 1..3: note = MISC_FEATURE - (GlySerGly) residues can be added to the 5' end of the peptide to improve cleavage efficiency
GSGQCTNYALLKLAGDVESNPGPP

SEQ ID NO: 67 - protein, 25 aa, synthetic construct
  REGION 1..25: note = F2A
  REGION 1..3: note = MISC_FEATURE - (GlySerGly) residues can be added to the 5' end of the peptide to improve cleavage efficiency
GSGVKQTLNFDLLKLAGDVESNPGP

SEQ ID NO: 68 - DNA, 747 bp, synthetic construct
  misc_feature 1..747: note = tet-Transactivator
  source 1..747: mol_type = other DNA
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 69 - protein, 743 aa, synthetic construct
  REGION 1..743: note = PHP.eB capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 70 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - DNA, 2868 bp, synthetic construct
  misc_feature 1..2868: note = Plasmid backbone
  source 1..2868: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 72 - DNA, 2879 bp, synthetic construct
  misc_feature 1..2879: note = Plasmid backbone
  source 1..2879: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 73 - DNA, 3034 bp, synthetic construct
  misc_feature 1..3034: note = synthetic construct
  source 1..3034: mol_type = other DNA
ggccgcacgcgttctagacaggaacatccaaactgagcagccggggtcccccccaccccccaccccgccccacgcggcaactttgagcctgtgctgggacagagcctctagttcctaaattagtccatgaggtcagaggcagcactgccattgtaacgcgattggagaggatcacgtcaccggacacgcccccaggcatctccctgggtctcctaaacttggcggggagaagttttagcccttaagttttagcctttaacccccatattcagaactgtgcgagttggcgaaaccccacaaatcacaacaaactgtacacaacaccgagctagaggtgatctttcttgtccattccacacaggccttagtaatgcgtcgccatagcaacagtgtcactagtagcaccagcacttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcaggtcaccagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgccacatatgccgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaatctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgacttgctgctatcccctaggtgctgtcatttacctacggttgtctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacagaaagcccacagaatcaaagacgtgttcaaacagccagctgaattcattgcacatttcaaccacagaaatattttcaggtgattctgttgtttgacaaaacgtgggaaccacaggatctacaacacttgcaagcaaaactcaacagctctaataatagttacagaagtgaaagccaatttggataaaataagacattgactcaagtcctctcagaagagttttgaaagcaaagtttacaaaagtctggtttgtcctttgggatttacagacctttcagccccttgattcatttttttttttttggatttcttcatcactgggagaattcccatgcattatttctcccctgcttcaaaatcatcaaatgtgaaacatttttcactcttttcttctgtatatagtgataaaatagctattggcttttggctaaatgtgctactttgagcccacccacacaagggagaaatgggggcagacatgagtttgggcatgagtgagctctgccttctcagaggtgagccacgtggtgcggaccgagcggccg

SEQ ID NO: 74 - DNA, 4856 bp, synthetic construct
  misc_feature 1..4856: note = synthetic construct
  source 1..4856: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 75 - DNA, 4861 bp, synthetic construct
  misc_feature 1..4861: note = synthetic construct
  source 1..4861: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtacctttccagcctggcttacaggcttttttcaccacagtaccaattgcccatgctctgctcattaatttaaatggcaatgactatctgggttttaaaatagagaagtgtcaggatgggaacacgcaatcatttggctttttgcgttccagcactgttttgaatagcagggttttcacttcctatgaaaccttagcaggaggaaaagcggaaactaaaccataaagtgagagggatgaggggagggagggacttgagtatttgtaaactcagggtggctggccctgcctaccaggctgctctctaccaccggaggctaggagtggaaaaacttgatttacgtgttgtgcctgccttttttttttcttcttcttcttcttcttcttcttcgtctccacaccaccttctgtacacctgactctgcataagcctatctgaagctggcttggtggcagggatagctggagaacagaagaatgtgcggagggagggagggaggaagggagggctggtacttttccattcacatctccacagtggctgttcttgtttatttcaaccaccggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 76 - DNA, 4862 bp, synthetic construct
  misc_feature 1..4862: note = synthetic construct
  source 1..4862: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctaggacgcgtttctaatcatgaattcttttgtggtattttagattttcagtttgttctgtatcaattctctctcattcaaagaatatgatagtgaggtagatgaagctgcctaagccacaggaagaaacatcttcagtctgtcgtaaatgcacttcagatacagcaccttgcactgcacataaaaattcatagtcacctaagtgggaatagctatgaaaatctgagtatcgccatgctgttgactcagtgctatttataaaactcagttttaatgtttccaatttaaattctctgcacatatctctcctgcactaaagacttgagataccagtgctttaccctaaaatatcttgcttttatatcttgactcttatgttgagaatttattatttttaaaatatactttaaaacatgcattggtacaaaattagtcaaaacagcaaccagtgaattcaaagtaaattagtattattaatgctgtgtataattttggtgaattttactattaaattataaataaaaagtccttccaggtagtcatgttcactcctgattggtaagttcaagaacattgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 77 - DNA, 3116 bp, synthetic construct
  misc_feature 1..3116: note = synthetic construct
  source 1..3116: mol_type = other DNA
gcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttgctgtcatttacctacggttgtctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacagaaagcccacagaatcaaagacgtgttcaaacagccagctgaattcattgcacatttcaaccacagaaatattttcaggtgattctgttgtttgacaaaacgtgggaaccacaggatctacaacacttgcaagcaaaactcaacagctctaataatagttacagaagtgaaagccaatttggataaaataagacattgactcaagtcctctcagaagagttttgaaagcaaagtttacaaaagtctggtttgtcctttgggatttacagacctttcagccccttgattcatttttttttttttggatttcttcatcactgggagaattcccatgcattatttctcccctgcttcaaaatcatcaaatgtgaaacatttttcactcttttcttctgtatatagtgataaaatagctattggcttttggctaaatgtgctactttgagcccacccacacaagggagaaatgggggcagacatgagtttgggcatgagtcttaaggagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 78 - DNA, 3695 bp, synthetic construct
  misc_feature 1..3695: note = synthetic construct
  source 1..3695: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttgctgtcatttacctacggttgtctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacagaaagcccacagaatcaaagacgtgttcaaacagccagctgaattcattgcacatttcaaccacagaaatattttcaggtgattctgttgtttgacaaaacgtgggaaccacaggatctacaacacttgcaagcaaaactcaacagctctaataatagttacagaagtgaaagccaatttggataaaataagacattgactcaagtcctctcagaagagttttgaaagcaaagtttacaaaagtctggtttgtcctttgggatttacagacctttcagccccttgattcatttttttttttttggatttcttcatcactgggagaattcccatgcattatttctcccctgcttcaaaatcatcaaatgtgaaacatttttcactcttttcttctgtatatagtgataaaatagctattggcttttggctaaatgtgctactttgagcccacccacacaagggagaaatgggggcagacatgagtttgggcatgagtcttaaggagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgca

SEQ ID NO: 79 - DNA, 3044 bp, synthetic construct
  misc_feature 1..3044: note = synthetic construct
  source 1..3044: mol_type = other DNA
gcggccgcacgcgtgactgctaagctttgggcactacctggggtcagtctgcatcaaaatgtaaggctcaaatgtgtaattgtaagtactgttttgctgagctggaagggctcctttgaagcccacgttttaattttaatttagccacacagagtggcaaagacaaatagatttatccaaaatacatttggtaacagattttttgagtcagttattaattttatttgaggggttcctctttttattttttataaactgtgaaactcaagaggaagcaggatcccatgcaatgccttttattgatggcctgctatgtgccaagaaaggtgttaaatgttttccaatgctgcctcatttatcctgatcttacagacaagcaaaaggaggtgtgaagaggtgaagtttctcacccagctggaaagtggcaaagtcattcacagatctgcctccgctcaaaaaaattgctttatgcaactctttggaagctaacttcatgggagctacatgcagcttctgagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 80 - DNA, 3101 bp, synthetic construct
  misc_feature 1..3101: note = synthetic construct
  source 1..3101: mol_type = other DNA
gcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 81 - DNA, 2523 bp, synthetic construct
  misc_feature 1..2523: note = synthetic construct
  source 1..2523: mol_type = other DNA
gcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 82 - DNA, 1938 bp, synthetic construct
  misc_feature 1..1938: note = synthetic construct
  source 1..1938: mol_type = other DNA
gcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaggcgcgccgcggccgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 83 - DNA, 2564 bp, synthetic construct
  misc_feature 1..2564: note = synthetic construct
  source 1..2564: mol_type = other DNA
gcggccgcacgcgtatgtgtcttttactctgatcctcctgtttttaccttccaagtgctggaatcacagacatataccactgtgcatagcatcattacaatgttatagtttttcacactatgccttgactttttggaaaggcaaaccacctcttggatttctccttccttctctatctctctctctctctcttcctccctccgtccctccatctcttcctccttcccattttcttctctccctatttggacacaatataaaataatttagatgaggtgagttaaattgtgaacaaagtatgtgcctatacatggttgtaaatcagcttatcaaagtgtaatattagaagaatttataaaaatgataaaattcatactcaaagttctgtgtaaagcaataatagctttatctccttttagttatcttgagtctttctatgactaacaactccctcataggcatcttaaagagcagtaagcataagtagattccaaatgggaagggagaagtgtgaaccatcactttcatccagacttgtagatatatctgctgcattttcagaaaccagaaacagacagtgttctttatctccattgagtctagtgtagcaacagagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 84 - DNA, 2543 bp, synthetic construct
  misc_feature 1..2543: note = synthetic construct
  source 1..2543: mol_type = other DNA
gcggccgcacgcgtcagtagtgttaatgacagagtcagatacatgagaaataaagacaaactgaaaatgttcattttgccaatatgatcaccaataaaaccatttgtgtagactatccaccttaatccccctataatacaatagcacagaggtgagtcagtttgattttgatactaggtttattttataggagctattaaagtttcagaattttgctgagtcaccaggctcttcattttgtggcaaatccatcactacagtttaaggagagaagaaacagacccccccctaccctctgaaaaataaaaataaaaacttgtttcaggcaggctagcgattcactaataatgagaaaactccagttttaagacttaatttcaccataaatactctttcattctaagctctgggacatcatgagccagagaacagcagagtgaataatacagttacagagctgatgagcaatgccagtcactgtaaaaaatacagaatcccatccaaaggacatctgtaaaagtgtctttaacatctactcagcccttctgtgtaaaggtcagcacgatgggagtggtttgtctatactcaagccgagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 85 - DNA, 5427 bp, synthetic construct
  misc_feature 1..5427: note = synthetic construct
  source 1..5427: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgtcccataggcagtttgtggctgagtgctggtccaggggtgaggaggtgggctatgttactgagggtctctgggtgttaggaaaacagggcccaggagtctggctgctcgtatgctggcccaggctcttgtttttcttgagctgacttgctggagaagtgagctaagtcagaaacaaaatgccacattgcacgcccactgaagtctgggctcaagggaaagaagagagattgccagagcgttagctgttcccaatccactcctggaccttaagctgtcttgaacagagttgccaatcagcttggtagggactggcctttgaggaggggagggggtgtaggcaggggagggggagagaagggagcagtctgcgctccatcttaattacctcatcagaaacagctcccttcccgcaaagctctggtgtcttctacaagagggtgagtctttggctttacatgtgaacttgtgccatttgcctgcgtatataaacatgaagggtcgtctgggttcagagctgaaatctttcacttgtgacttagctgggaaattcttggcaagatcagaatgcagtggtaaggtctgagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 2564 bp, synthetic construct
  misc_feature 1..2564: note = synthetic construct
  source 1..2564: mol_type = other DNA
gcggccgcacgcgtagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 87 - DNA, 2859 bp, synthetic construct
  misc_feature 1..2859: note = synthetic construct
  source 1..2859: mol_type = other DNA
gcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 88 - DNA, 2549 bp, synthetic construct
  misc_feature 1..2549: note = synthetic construct
  source 1..2549: mol_type = other DNA
cggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 89 - DNA, 5716 bp, synthetic construct
  misc_feature 1..5716: note = synthetic construct
  source 1..5716: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgttaattaagccaccatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 90 - DNA, 3142 bp, synthetic construct
  misc_feature 1..3142: note = synthetic construct
  source 1..3142: mol_type = other DNA
gcggccgcacgcgtatgtgtcttttactctgatcctcctgtttttaccttccaagtgctggaatcacagacatataccactgtgcatagcatcattacaatgttatagtttttcacactatgccttgactttttggaaaggcaaaccacctcttggatttctccttccttctctatctctctctctctctcttcctccctccgtccctccatctcttcctccttcccattttcttctctccctatttggacacaatataaaataatttagatgaggtgagttaaattgtgaacaaagtatgtgcctatacatggttgtaaatcagcttatcaaagtgtaatattagaagaatttataaaaatgataaaattcatactcaaagttctgtgtaaagcaataatagctttatctccttttagttatcttgagtctttctatgactaacaactccctcataggcatcttaaagagcagtaagcataagtagattccaaatgggaagggagaagtgtgaaccatcactttcatccagacttgtagatatatctgctgcattttcagaaaccagaaacagacagtgttctttatctccattgagtctagtgtagcaacagagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 91 - DNA, 3126 bp, synthetic construct
  misc_feature 1..3126: note = synthetic construct
  source 1..3126: mol_type = other DNA
gcggccgcacgcgtgtcccataggcagtttgtggctgagtgctggtccaggggtgaggaggtgggctatgttactgagggtctctgggtgttaggaaaacagggcccaggagtctggctgctcgtatgctggcccaggctcttgtttttcttgagctgacttgctggagaagtgagctaagtcagaaacaaaatgccacattgcacgcccactgaagtctgggctcaagggaaagaagagagattgccagagcgttagctgttcccaatccactcctggaccttaagctgtcttgaacagagttgccaatcagcttggtagggactggcctttgaggaggggagggggtgtaggcaggggagggggagagaagggagcagtctgcgctccatcttaattacctcatcagaaacagctcccttcccgcaaagctctggtgtcttctacaagagggtgagtctttggctttacatgtgaacttgtgccatttgcctgcgtatataaacatgaagggtcgtctgggttcagagctgaaatctttcacttgtgacttagctgggaaattcttggcaagatcagaatgcagtggtaaggtctgagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 92 - DNA, 3142 bp, synthetic construct
  misc_feature 1..3142: note = synthetic construct
  source 1..3142: mol_type = other DNA
gcggccgcacgcgtagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 93 - DNA, 2884 bp, synthetic construct
  misc_feature 1..2884: note = synthetic construct
  source 1..2884: mol_type = other DNA
gcggccgcacgcgtgtcccataggcagtttgtggctgagtgctggtccaggggtgaggaggtgggctatgttactgagggtctctgggtgttaggaaaacagggcccaggagtctggctgctcgtatgctggcccaggctcttgtttttcttgagctgacttgctggagaagtgagctaagtcagaaacaaaatgccacattgcacgcccactgaagtctgggctcaagggaaagaagagagattgccagagcgttagctgttcccaatccactcctggaccttaagctgtcttgaacagagttgccaatcagcttggtagggactggcctttgaggaggggagggggtgtaggcaggggagggggagagaagggagcagtctgcgctccatcttaattacctcatcagaaacagctcccttcccgcaaagctctggtgtcttctacaagagggtgagtctttggctttacatgtgaacttgtgccatttgcctgcgtatataaacatgaagggtcgtctgggttcagagctgaaatctttcacttgtgacttagctgggaaattcttggcaagatcagaatgcagtggtaaggtctgagctcgggctgggcataaaagtcagggcagagccatctattgcttacatttgcttctgggatccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 94 - DNA, 2900 bp, synthetic construct
  misc_feature 1..2900: note = synthetic construct
  source 1..2900: mol_type = other DNA
gcggccgcacgcgtagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcgggctgggcataaaagtcagggcagagccatctattgcttacatttgcttctgggatccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 95 - DNA, 5470 bp, synthetic construct
  misc_feature 1..5470: note = synthetic construct
  source 1..5470: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtatgtgtcttttactctgatcctcctgtttttaccttccaagtgctggaatcacagacatataccactgtgcatagcatcattacaatgttatagtttttcacactatgccttgactttttggaaaggcaaaccacctcttggatttctccttccttctctatctctctctctctctcttcctccctccgtccctccatctcttcctccttcccattttcttctctccctatttggacacaatataaaataatttagatgaggtgagttaaattgtgaacaaagtatgtgcctatacatggttgtaaatcagcttatcaaagtgtaatattagaagaatttataaaaatgataaaattcatactcaaagttctgtgtaaagcaataatagctttatctccttttagttatcttgagtctttctatgactaacaactccctcataggcatcttaaagagcagtaagcataagtagattccaaatgggaagggagaagtgtgaaccatcactttcatccagacttgtagatatatctgctgcattttcagaaaccagaaacagacagtgttctttatctccattgagtctagtgtagcaacagagctcgggctgggcataaaagtcagggcagagccatctattgcttacatttgcttctgggatccgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 96 - DNA, 2575 bp, synthetic construct
  misc_feature 1..2575: note = synthetic construct
  source 1..2575: mol_type = other DNA
gcggccgcacgcgtgtcccataggcagtttgtggctgagtgctggtccaggggtgaggaggtgggctatgttactgagggtctctgggtgttaggaaaacagggcccaggagtctggctgctcgtatgctggcccaggctcttgtttttcttgagctgacttgctggagaagtgagctaagtcagaaacaaaatgccacattgcacgcccactgaagtctgggctcaagggaaagaagagagattgccagagcgttagctgttcccaatccactcctggaccttaagctgtcttgaacagagttgccaatcagcttggtagggactggcctttgaggaggggagggggtgtaggcaggggagggggagagaagggagcagtctgcgctccatcttaattacctcatcagaaacagctcccttcccgcaaagctctggtgtcttctacaagagggtgagtctttggctttacatgtgaacttgtgccatttgcctgcgtatataaacatgaagggtcgtctgggttcagagctgaaatctttcacttgtgacttagctgggaaattcttggcaagatcagaatgcagtggtaaggtctgagctcgggctgggcataaaagtcagggcagagccatctattgcttacatttgcttctgggatccgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 97 - DNA, 2591 bp, synthetic construct
  misc_feature 1..2591: note = synthetic construct
  source 1..2591: mol_type = other DNA
gcggccgcacgcgtagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcgggctgggcataaaagtcagggcagagccatctattgcttacatttgcttctgggatccgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 98 - DNA, 4424 bp, synthetic construct
  misc_feature 1..4424: note = synthetic construct
  source 1..4424: mol_type = other DNA
gcggccgcacgcgtgaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaagtgacatagcgatagacagattcgactcggtaccagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggatgatggcatcattgagtagcatgatctcaattgagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcagggtgcaggagaaatgtgacctcaaagtcttgttctataactgttggaccttaggagagatctgtgctcagcaatttaccaggacacccccaccccacatgtcttgaccactgtctggataactggtatgcaggaccacactaggcttactcacagtgtaaactctcataaccatcactggagcccatcctgcctggtagacaaggattcaaccatgactcattgtactttagtggtgccatgcttagtcatcaggtgccctgtgctctgacagccgagggtcagagctggaatcacactcttgttgtcttttaatctctccctccctttcttccttctttcttcactctgttgtgattgctcatggaacagatcctagctggtctccctggcaacctacatgatttgagcccaacagatggataatggggacatcgacttccaatgtcattcaacagaatcattgccaagggagtctgatgagcaggcaactgagatgacacccttatcaatatagcttcattttggcaatctggagtaggtgtttcaaaaggagagcccccactgatgccagcaatacagaacgttcatgggcaaggagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 99 - DNA, 1919 bp, synthetic construct
  misc_feature 1..1919: note = synthetic construct
  source 1..1919: mol_type = other DNA
gcggccgcacgcgtcaaaagatggaagttgggaggttgaagaagtgcaggatggcattccaagtgatgggggcaatggcatggaggtaggaaagcataaggtatattcaggctataaataatagttagatttggctggatcctggatttgagaagccaggaaatgagataacactggtcactttcactaaagctcatgaaaaaaaaaatacatacatatatatatatataaaataaatatacatatatatttttaagccccatatgactagaggaggcagcccatctgttctctgggcttcacttttcttgtctgggaaatgagtaggttggactgcatggtctttaaggtctctttagtattatcttgtttgactccgtaaagagaaaaacaaaggttcctcctgacatcttgtgttgccttccaacgtccagtccagtgtgattgttttaagtactctttggatattttactgttataaaaagtgaagaaaaagactgattttgccaagtcttatggatccaaattagtactcattgcactatggtcatttagttgaggacgatactccagcttcaaaggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 100 - DNA, 2074 bp, synthetic construct
  misc_feature 1..2074: note = synthetic construct
  source 1..2074: mol_type = other DNA
gcggccgcacgcgtggtacatattataagtttgagtctgcaagatgtggcaaacccttccttttctctttatcttgacagtggaaaacatctaaggagtctttaaaataaccacatcggactgagcagtggagggcaaagagaatgtctggaagaacctttgtttttttcgttgggacatcaaaggatgttatactgaagagatctcaaagaccaggcagtccagcctactcgtttcccagacaagagaagtgaagccaagaaacagatgggctgcctcctctagtcatatggggcttaaaaatatatatctctatttttcatgagttttcatgaaagtgaccagtgttatctcatttcctgtcttctcaaattcaggatcctacccaatctaactattatttatagtgtgaataagccctctactttcctacctctacaatgctcattctccattttcccatcatctacttccatcttgtgaaaagtttccactcttcagtgatgacctcaaacatatcatttcttttttttaattctttttttattagatattttctttatatacatttcaaatgctatcctgaaagttccctatatcctccctctgccctgcttccctacccacccactcccacttcttggccctggcatttccctgtactggggcatataaagtttgcaataccaaggggcctctcttcacagtgatggccaactaggccatcttctgctacgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 101 - DNA, 4059 bp, synthetic construct
  misc_feature 1..4059: note = synthetic construct
  source 1..4059: mol_type = other DNA
gcggccgcacgcgtataggtaccccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagagggagctcgggctgggcataaaagtcagggcagagccatctattgcttacatttgcttctgggatccttcgaagctagcggcgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 102 - DNA, 2139 bp, synthetic construct
  misc_feature 1..2139: note = synthetic construct
  source 1..2139: mol_type = other DNA
gcggccgcacgcgtccgagacagcagagagactggtctgactgcagaggaggtctgggagaccagagggagtgtggagagggtgaggttagaagacaggaacaccgagctgcatcggccaaatggagccttagggggccatgtgaggctgaggcagggaagcagggatcctgccctccaggtccttacagtcaggcggggaccaaaagcacgaggatgccagcccaattccctattaggcaaaacgcagcaccatctgcacaatcccaggagcaagagcagatattttataacttccttttttctttttaagtctaaattaaaaataaatgttcccttcagctctcagatgtatatctctggtgcaacctgcccacattccctcccgctgccctttccagaacatggcaggggaaaggaagaaagagatggatagagagagggagccagtccacccagcttcaatgccagtggattgcacctcttccaagagggaaacgattcaggcgtggccacgcagacgggtggagagcgcccagaatgtggctggtaccaaggaaagtggaaggagagggaaacaggagccaacagctatgatttctagcccagcctccaccctatcgcgctgcaggaccttggccaaatcacacatcctatctctgctcccatttatagttcataacatggctgaagtcccctctgccgctccagccccctggcagctgtgctctctgcacatccgtctgtacctttgctgctccccttcattttgggtgtcctaccatggacctagtgattaacggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 103 - DNA, 1952 bp, synthetic construct
  misc_feature 1..1952: note = synthetic construct
  source 1..1952: mol_type = other DNA
gcggccgcacgcgtggtgaggaacagaacaaaacagaacaagcaggttcacttgggacgccgggaacaccccgggcttgcgcccctgcgcctccccgctggcggccccgccaacttcccggggtgtcccctccctaccttctcttcaccgccctggcgcctggcctgcgcgaggtcgggactcgcgggacctccgcctaccccagaagcggctgtctaaagcgggggtgggggggcgccccctcctgtctggttttcccttccagttgccgggagaggactaggcagccgggagccgggccgtgcacccgctgtggcgcgctggcacctcggcctccgcaaacagattgctcgccctcctcggggaaagctaggaaaacagtgctaagcctcgcaagctgccgcccattaatgcctcttagcttgcaagatgggttactagctctgagcacggccctcccctcggggcttcttacattctcctccccctcgccccttctgtctccctccttctccacgccgcggtactctcgccttcgccctcattctctccctccacctactacctcttccttttgttttccgttctcctgaatttccctttctttctttttcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 104 - DNA, 2025 bp, synthetic construct
  misc_feature 1..2025: note = synthetic construct
  source 1..2025: mol_type = other DNA
gcggccgcacgcgtcagagatggaggggtcagagacacagaggatgacagagacccagagagagggagacagagacccagagagagggggagagagacctagaaatagggggacagagacccagagagggaagagatggaaacctagagaaggaagcagacagagtcccaaagagaggcggggacagaaacccaggagatagaacatagatgcagagagatgagaacagagatccagaatgcaagagaaagatggagagcctgggagacggaggatagacaggctggggacgtgatttgtgaggtgcagcccctctctgaggtgggtaggcagccaggggatcgggctggatcccaggaaggggctggaacagatggaggcgagggagactgggacgggggaggaaggaacagccagacggtccagggggagggaggtggaagagctgtgagaacacagcaacccctccccatcctagaacttaaggaggtggggaggggcagttaagaaacaggggtgggagaagccagggagtggacagaacccagaaggagtaggaatgagaccccaagaggaagaagacagagagccagagataggggggattgaagccactaggacgaatagtacaggatggcagtaactccccccacccatcagaacccatcacccaaagagattagactggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 105 - DNA, 1923 bp, synthetic construct
  misc_feature 1..1923: note = synthetic construct
  source 1..1923: mol_type = other DNA
gcggccgcacgcgtgaaatcataaatgctgagggtagtctgcctcaggtacacactgagaaactgctttaatgtaacctgacccacggttattagtgaaaatatcacttttgttgttaccttattcccaacaaattcatttctgctttaatggaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcacaaatgtttaattaaaaaaaaaaaaagtcatgtactttcatagatattcacaatcctctctagtatactttcaaatcagttttaatttcagtttagtgtttttatgttttgtgaagatacgcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 106 - DNA, 2070 bp, synthetic construct
  misc_feature 1..2070: note = synthetic construct
  source 1..2070: mol_type = other DNA
gcggccgcacgcgtgtatgtgtgacaggagccaggcagctgaggcagcgggacaacagagccactggcctgactgggaagaatgtcccagacattgacaaaagacaatctagagagggtaaggatgggagaccagggacaccaaagagcccactgggtccctgtggccaggcggggcccagagcacatggtgccagcctcgttccctattaggcaaagcactgcaccatctgtatagtcccaggagcaggagcaggagcaggcgttttataacttccttttctttttcagtctacattaaaaataaatgttcccttcagctctcagatgtatatctctagtgcaacctgcccacattccctcctgctgccctttccagaacatggcaggggaagggaaggaagagatggagagagggagccagtccacccggctgatgccagtggatcacacctcttctaagagggaagcgcggcaggcacggccacacatggtggaaggtgcccagaatgcatggggaccagggaaatggaagtggaggaaatgggagccaacagccaggcttgcttcccacccccaccctcccgcaccgcaggaccttggccaaatcacacatcccatctttgcatttatagttcatgcagtggctggagtcccctctgcagctccagccctctggtggctgtccttgctgcacgtctctctgtacttccccttgtgtgtcctgctgtgggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 107 - DNA, 1872 bp, synthetic construct
  misc_feature 1..1872: note = synthetic construct
  source 1..1872: mol_type = other DNA
gcggccgcacgcgtccctggccttcgagcacatgctcagatgatgctccaccgtggcctgacccacatcttctagtggaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctcatgatgtatgctcaaatcagttttaattgaactgtgtgtagtatgctcctgttttggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 108 - DNA, 2245 bp, synthetic construct
  misc_feature 1..2245: note = synthetic construct
  source 1..2245: mol_type = other DNA
gcggccgcaacgcgtttagaacaatggctggcccatagtaaatgccgtgttagtgtgttagttgctgttcttccacgtcagaagaggcacagacaaattaccaccaggtggcgctcagagtctgcggaggcatcacaacagccctgaatttgaatcctgctctgccactgcctagttgagaccttttactacctgactagctgtttgtgtattttaggtgtttgtttcttgcacgcttattcaggcttttggcagaaatcagttcgttgtggtttaaggactgaggtcgcctttctttgctgctaactgttggccaggaagtactgtcagctactacatggtacttgctggtccttgcacatgaccccctccaccttcaaaccaggattgacacagtgtgttgagtctttcttgaagacttttgaacatctgacttcccattctgccactagccagttaaaattctctaattttgaaggactcacctggttggccaaacccacccaaataatctccacatcttgaagtcaactgacccaggactttacatatgcaaaatcccttcacagcagtacctagatgagtgtttgcttgaataactgggacacaggaatcttgggggagccatctttagaattcgacctcctacaacccttctggaaatctgagagtgagtcaggggaagaaaccctcttttgtagtttccttttagggctttctactttgctcaaagttgggcactatttcacttcagtagggtcctgcaagccccatgagggtagtgagtgctgtcctaggaaacagtaacttaaccctgatacccatttgtccaagaattcgatatcataatcaaccataggtaccgagctcgggattcagccgggagcttagggaggggaggtcacttcataagggcctggggggggagttggagccacgagtcgtccagccggagccccgtgtggctgagctccggcctcagaagcatccccgggttggatccttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 109 - DNA, 1839 bp, synthetic construct
  misc_feature 1..1839: note = synthetic construct
  source 1..1839: mol_type = other DNA
gcggccgcacgcgtttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccgcttaaggagctcagaggtaggcgtgtacggtgggaggcctatataagcagagctggtttagtgaaccgtcagatcgcctggggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 110 - DNA, 2619 bp, synthetic construct
  misc_feature 1..2619: note = synthetic construct
  source 1..2619: mol_type = other DNA
gcggccgcaacgcgtttagaacaatggctggcccatagtaaatgccgtgttagtgtgttagttgctgttcttccacgtcagaagaggcacagacaaattaccaccaggtggcgctcagagtctgcggaggcatcacaacagccctgaatttgaatcctgctctgccactgcctagttgagaccttttactacctgactagctgtttgtgtattttaggtgtttgtttgaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcgagcagagccctcatcacacagactgaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatctgggtgagtaagagtaggtggcaacgaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatcgaattcgatatcataatcaaccataggtaccgagctcgggattcagccgggagcttagggaggggaggtcacttcataagggcctggggggggagttggagccacgagtcgtccagccggagccccgtgtggctgagctccggcctcagaagcatccccgggttggatccttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 111 - DNA, 2892 bp, synthetic construct
  misc_feature 1..2892: note = synthetic construct
  source 1..2892: mol_type = other DNA
gcggccgcaacgcgtttagaacaatggctggcccatagtaaatgccgtgttagtgtgttagttgctgttcttccacgtcagaagaggcacagacaaattaccaccaggtggcgctcagagtctgcggaggcatcacaacagccctgaatttgaatcctgctctgccactgcctagttgagaccttttactacctgactagctgtttgtgtattttaggtgtttgtttgaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctcgagcagagccctcatcacacagactgaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctctgggtgagtaagagtaggtggcaacgaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctcgaattcgatatcataatcaaccataggtaccgagctcgggattcagccgggagcttagggaggggaggtcacttcataagggcctggggggggagttggagccacgagtcgtccagccggagccccgtgtggctgagctccggcctcagaagcatccccgggttggatccttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 112 - DNA, 1931 bp, synthetic construct
  misc_feature 1..1931: note = synthetic construct
  source 1..1931: mol_type = other DNA
gcggccgcacgcgtcgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcagaggtaggcgtgtacggtgggaggcctatataagcagagctggtttagtgaaccgtcagatcgcctggggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 113 - DNA, 1721 bp, synthetic construct
  misc_feature 1..1721: note = synthetic construct
  source 1..1721: mol_type = other DNA
gcggccgcacgcgtaagccaatgacattagagaagtgttcaaacagtcagctaaattcactgcacttctcaaccacagaaatattttcaggtgattctgtttttgagaaaacgtgggaaccacaggatctacaacacttccaggcaaaactcaacagctctaataatagtgacagaagtgaaagccaatttggataaaataagacattgactcaaagtcctctgagagatttttcaaaacaaagtttacaaagctccttttgccttttgggaaatcacattcttctttgcaccttgactctttttctgaatttctttctgtctgggaggatctccttacagtgtttcttctccatctgacatcatgaaatgtgatacgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 114 - DNA, 1744 bp, synthetic construct
  misc_feature 1..1744: note = synthetic construct
  source 1..1744: mol_type = other DNA
gcggccgcacgcgtaattgctgtcatttacctacggttgtctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacagagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 115 - protein, 449 aa, synthetic construct
  REGION 1..449: note = Myosin light chain kinase, Green fluorescent protein, Calmodulin chimera
  SITE 223: note = misc_feature - Xaa can be any naturally occurring amino acid
MRGSHHHHHHGMASMTGGQQMGRDLYDDDDKDLATMVDSSRRKWNKTGHAVRAIGRLSSLENVYIMADKQKNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLXVQCFSRYPDHMKQHDFFKSAMPEGYIQERTIFFKDDGNYKTRAEVKFEGDTLVNRIELKGIDFKEDGNILGHKLEYNTRDQLTEEQIAEFKEAFSLFDKDGDGGITTKQLGTVMRSLGQNPTEAELQDMINEVGADGNGTIDFPQFLTMMARKMKDTDSEEEIREAFRVFGKDGNGYISAAQLRHVMTNLGEKLTDEEVDEMIREAGIDGDGQVNYEQFVQMMTAK

SEQ ID NO: 116 - protein, 309 aa, synthetic construct
  REGION 1..309: note = Genetically-encoded green calcium indicator NTnC
  SITE 68: note = misc_feature - Xaa can be any naturally occurring amino acid
MVSKGEEDNMASLPATHELHIFGSINGVDFDMVGQGSGNPNVGYEELNLKSTKGDLQFSPWILVPHIXFHQYLPYPDGMSPFQAAMVDGSGYQVHRTVQFEDGASLTVNYRYTYEGSHIKGEAQVKGTGFPADGPVMANSLTAMVPSEEELSECFRTFDKDGDGFIDREEFGGIIRLTGEQLTDEDPDEIFGDSDTDKNGRIDFDEFLKMVENVQLSMADWCRSKMACPNDKTLISTLKWSYTTGNGKRYRSTARTTYTFAKPMAANYLKNQPMYVFRKTELKHSKTELNFKEWQKAFTDVMGMDELYK

SEQ ID NO: 117 - protein, 619 aa, synthetic construct
  REGION 1..619: note = Calcium indicator TN-XXL
MVSKGEELFTGVVPILVELDGDVNGHKFSVRGEGEGDATNGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSKLSKDPNEKRDHMVLLEFVTAARMLSEEELANCFRIFDKDANGFIDIEELGEILRATGEHVTEEDIEDLMKDSDKNNDGRIDFDEFLKMMEGVQGTSEEELANCFRIFDKDANGFIDIEELGEILRATGEHVTEEDIEDLMKDSDKNNDGRIDFDEFLKMMEGVQELMGGVQLADHYQQNTPIGDGPVLLPDNHYLSYQSKLSKDPNEKRDHMVLLEFVTAAGITLGMDELYKGGTGGSMVSKGEELFTGVVPILVELDGDVNGHKFSVRGEGEGDATNGKLTLKFICTTGKLPVPWPTLVTTLGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIED

SEQ ID NO: 118 - protein, 730 aa, synthetic construct
  REGION 1..730: note = BRET-based auto-luminescent calcium indicator
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKRMHDQLTEEQIAEFKEAFSLFDKDGDGTITTKELGTVMRSLGQNPTEAELQDMINEVDADGNGTIYFPEFLTMMARKMKDTDSEEEIREAFRVFDKDGNGYISAAQLRHVMTNLGEKLTDEEVDEMIREADIDGDGQVNYEEFVQMMTAKGGKRRWKKNFIAVSAANRFKKISSSGALELMTSKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGAALAFHYAYEHQDRIKAIVHMESVVDVIESWDEWPDIEEDIALIKSEEGEKMVLENNFFVETVLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 119 - protein, 568 aa, synthetic construct
  REGION 1..568: note = Calcium indicator protein OeNL(Ca2+)-18u
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITEQVEDAVAHSGTLEDFVGDWRQTAGYNLDQVLEQGGVSSLFQNLGVSVTPIQRIVLSGENGLKIDIHVIIPYEGPWMHDQLTEEQIAEFKEAFSLFDKDGDGTITTKELGTVMRSLGQNPTEAELQDMINEVDADGNGTIYFPDFLTMMARKMKDTDSEEEIREAFRVFDKDGNGYISAADLRHVMTNLGEKLTDEEVDEMIREADIDGEGQVNYEEFVQMMTAKGGKRRWKKNFIAVSAANRFKKISSSGALELLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 120 - DNA, 1353 bp, synthetic construct
  misc_feature 1..1353: note = GCaMP6m
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaagggagctacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 121 - DNA, 1353 bp, synthetic construct
  misc_feature 1..1353: note = GCaMP6s
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 122 - DNA, 1353 bp, synthetic construct
  misc_feature 1..1353: note = GCaMP6f
  source 1..1353: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 123 - protein, 593 aa, Mesostigma viride
MSPPTSPTPDTGHDTPDTGHDTGGHGAVEICFAPCEEDCVTIRYFVENDFEGCIPGHFDQYSSHGSLHDIVKAALYICMVISILQILFYGFQWWRKTCGWEVWFVACIETSIYIIAITSEADSPFTLYLTNGQISPQLRYMEWLMTCPVILIALSNITGMAEEYNKRTMTLLTSDVCCIVLGMMSAASKPRLKGILYAVGWAFGAWTYWTALQVYRDAHKAVPKPLAWYVRAMGYVFFTSWLTFPGWFLLGPEGLEVVTGTVSTLMHACSDLISKNLWGFMDWHLRVLVARHHRKLFKAEEEHALKKGQTLEPGMPRSTSFVRGLGDDVEIDPSYELYRLKRQNHPEYFLSPAQTPRRGPSFDKRTSFEMDGGKNGMLQMMPVTGMGMGMGMGMGGGKTVLFLDYTGGGYVSFFEQQLSNMGVNVTKCWSDDDMYNTAGVANVKQLFHFAMIPNNALGGQMVMDLRGTGLLVVAYGPEPPMPGMGQDEFVPLQMPGVPYDESILHNLVMRHAITQGLGMNGMQGNMGQQQQMMGMQGNMNGMQGNMNGMQGNMNGMQGNMSGMQGNMNGMQGNSGMNQGWNNQGFTNTGAFGY

SEQ ID NO: 124 - protein, 717 aa, Chlamydomonas yellowstonensis
MDTLAWVARELLSSGHGTDTATDSGHGTDTSGGHDSSHDAVAHNVTLLIAPPHAGGHAGPTDTSQQITGIDGWIAIPAGDCYCAGWYVSHGSSFEATFAHVCQWSIFAVCVLSLLWYAYQYWKATCGWEEVYVCCIELVFICFELYHEFDSPCSLYLSTSNVVNWLRYSEWLLCCPVILIHLSNVTGLSDDYGRRTMGLLVSDIATIVFGVTAAMLVNWPKIIFYLIGFTMCCYTFFLAAKVLIESFHQVPKGICRHLVKAMAITYFVGWSFFPLIFLFGQSGFKKISPYADVIASSFGDLISKNAFGMLGHFLRVKIHEHILKHGDIRKTTHLRIAGEEKEVETFVEEEDEDTAKHSTKELANRGSFIVMRDKMKEQGIDVRASLDMDEDEEARTGKGKGAGATSLVPGRVILAVPDISMVDFFHDHFAHLGASIELVPALGVENTLLLVQQAMQLGGLDFVLVHPEFLRDRSQNGLVSRLKMTGHGVCAFGWVPSGPMREIIESAGVDGWLDGPSFGTGIDQEQLIELIGYMQAKRKFGMRFGGGGASKAGYSSDGGFGGKGMLEMQPSMSQGSGVPLLQQNNSMMRAPPSPMGNMANNGMMNPMMSMNNPMMGGGAVMMTSMGSMQQAANPLYGAPPSPLSSQPGAGMYGAPAQPQMGSQGSMHGSMYGGSQQQHQQPQQAAAAPAAADGGSEAEMLKQLMSEINRLKAELGES

SEQ ID NO: 125 - protein, 747 aa, Volvox carteri
  source 1..747: note = Volvox carteri f. nagariensis
MDHPVARSLIGSSYTNLNNGSIVIPSDACFCMKWLKSKGSPVALKMANALQWAAFALSVIILIYYAYATWRTTCGWEEVYVCCVELTKVVIEFFHEFDEPGMLYLANGNRVLWLRYGEWLLTCPVILIHLSNLTGLKDDYNKRTMRLLVSDVGTIVWGATAAMSTGYIKVIFFLLGCMYGANTFFHAAKVYIESYHTVPKGLCRQLVRAMAWLFFVSWGMFPVLFLLGPEGFGHLSVYGSTIGHTIIDLLSKNCWGLLGHFLRLKIHEHILLYGDIRKVQKIRVAGEELEVETLMTEEAPDTVKKSTAQYANRESFLTMRDKLKEKGFEVRASLDNSGIDAVINHNNNYNNALANAAAAVGKPGMELSKLDHVAANAAGMGGIADHVATTSGAISPGRVILAVPDISMVDYFREQFAQLPVQYEVVPALGADNAVQLVVQAAGLGGCDFVLLHPEFLRDKSSTSLPARLRSIGQRVAAFGWSPVGPVRDLIESAGLDGWLEGPSFGLGISLPNLASLVLRMQHARKMAAMLGGMGGMLGSNLMSGSGGVGLMGAGSPGGGGGAMGVGMTGMGMVGTNAMGRGAVGNSVANASMGGGSAGMGMGMMGMVGAGVGGQQQMGANGMGPTSFQLGSNPLYNTAPSPLSSQPGGDASAAAAAAAAAAATGAASNSMNAMQAGGSVRNSGILAGGLGSMMGPPGAPAAPTAAATAAPAVTMGAPGGGGAAASEAEMLQQLMAEINRLKSELGE

SEQ ID NO: 126 - protein, 310 aa, synthetic construct
  REGION 1..310: note = Channel rhodopsin 2
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVP

SEQ ID NO: 127 - protein, 282 aa, Streptococcus pyogenes
MLEHKIDFMVTLEVKEANANGDPLNGNMPRTDAKGYGVMSDVSIKRKIRNRLQDMGKSIFVQANERIEDDFRSLEKRFSQHFTAKTPDKEIEEKANALWFDVRAFGQVFTYLKKSIGVRGPVSISMAKSLEPIVISSLQITRSTNGMEAKNNSGRSSDTMGTKHFVDYGVYVLKGSINAYFAEKTGFSQEDAEAIKEVLVSLFENDASSARPEGSMRVCEVFWFTHSSKLGNVSSARVFDLLEYHQSIEEKSTYDAYQIHLNQEKLAKYEAKGLTLEILEGL

SEQ ID NO: 128 - protein, 1368 aa, synthetic construct [sequence omitted]

SEQ ID NO: 129 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 130 - protein, 741 aa, Homo sapiens
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 131 - protein, 357 aa, Homo sapiens
MMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 132 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 133 - protein, 872 aa, Homo sapiens [sequence omitted]

SEQ ID NO: 134 - protein, 2221 aa, Homo sapiens
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIALKILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKGYFSDPWNVFDFLIVIGSIIDVILSETNHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRERHVPMCEDLELRRDSGSAGTQAHCLLLRKANPSRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGRPSEEELQDSRVYVSSL

SEQ ID NO: 135 - protein, 1927 aa, Homo sapiens [sequence omitted]

SEQ ID NO: 136 - protein, 465 aa, Homo sapiens
MLPLWTLSLLLGAVAGKEVCYERLGCFSDDSPWSGITERPLHILPWSPKDVNTRFLLYTNENPNNFQEVAADSSSISGSNFKTNRKTRFIIHGFIDKGEENWLANVCKNLFKVESVNCICVDWKGGSRTGYTQASQNIRIVGAEVAYFVEFLQSAFGYSPSNVHVIGHSLGAHAAGEAGRRTNGTIGRITGLDPAEPCFQGTPELVRLDPSDAKFVDVIHTDGAPIVPNLGFGMSQVVGHLDFFPNGGVEMPGCKKNILSQIVDIDGIWEGTRDFAACNHLRSYKYYTDSIVNPDGFAGFPCASYNVFTANKCFPCPSGGCPQMGHYADRYPGKTNDVGQKFYLDTGDASNFARWRYKVSVTLSGKKVTGHILVSLFGNKGNSKQYEIFKGTLKPDSTHSNEFDSDVDVGDLQMVKFIWYNNVINPTLPRVGASKIIVETNVGKQFNFCSPETVREEVLLTLTPC

SEQ ID NO: 137 - protein, 824 aa, Homo sapiens
MSISSLFGGRYDNKFLLNMSSAPKIELIVDKVASLSEGRLEGRLPEDWFRHIMDPETEFNSEFADALCIGIDEFAQPLPFLPFKALLVTGTAGAGKTNSIQTLAANLDCIVTATTSIAAQNLSVVLNRSKSAQVKTIFKTFGFNSSHVSMSERQSYIANDERSIQIQQKQDLSIYWNVISDIADRALGAVACKTKELPDLCESSVIVIDEAGVILRHILHTVVFFYWFYNALYKTPLYEDGIVPCIVCVGSPTQSNALVTSFNPLTQNKDVKRGIDVLSALICDDVLSKYCEVDNNWIIFVNNKRCADHAFGDFLKHIEFGLPLKPELIEYVDQFVKPASYIRNPMNEIETTRLFLSHNEVKNYFRSLHEQVEVTNRNNLFVFPVYFLIKNKTFEDYKSEIGNFSLEIEPWFKSNIHRLNTYSQFADQDLSKTVQLEEIVLEDGSVEETLITCHLKHIRNSSIGVTSKIKASTVGFSGTYEKFVELLQSDLFIEKTSCDQTIHAYSFLSGLMFGGMYSFCCSKFTTPEVLMEIKNIKMPSIEFLESEMSRMSPDVQTVETDERYDFGLVDDGLSDVDLLEIDPCGDPFFTRYSKLPLTNSLSFEEISLLYTTFKDIFISRFAILQKHTKGKFGKTLLVTYNRNNVSRKQCGEIYSHLKSFYGMLTYAIPANNYTLEGYTNDNVVHLGTDKQLPQILYKKGLPRLVIKDEMGFISVLDNNVSKFIDVVNGQSFHLCTTVDYATVSKVSMTITKSQGLSIQKVAIDFGSDPKNLKLSSIYVGMSRVTDPNNLIMNVNPLRLNYENDNFIAPHIVKALKNENTMLIF

SEQ ID NO: 138 - protein, 511 aa, Homo sapiens
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 139 - protein, 952 aa, Homo sapiens [sequence omitted]

SEQ ID NO: 140 - protein, 785 aa, Homo sapiens
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 141 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 142 - protein, 529 aa, Homo sapiens
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 143 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 144 - protein, 743 aa, Homo sapiens
MNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSASQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 145 - protein, 507 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 146 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 147 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 148 - protein, 1338 aa, Homo sapiens [sequence omitted]

SEQ ID NO: 149 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 150 - protein, 178 aa, Homo sapiens [sequence omitted]

SEQ ID NO: 151 - protein, 866 aa, Homo sapiens
MGNRGMEELIPLVNKLQDAFSSIGQSCHLDLPQIAVVGGQSAGKSSVLENFVGRDFLPRGSGIVTRRPLILQLIFSKTEHAEFLHCKSKKFTDFDEVRQEIEAETDRVTGTNKGISPVPINLRVYSPHVLNLTLIDLPGITKVPVGDQPPDIEYRVKDMILQFISRESSLILAVTPANMDLANSDALKLAKEVDPQGLRTIGVITKLDLMDEGTDARDVLENKLLPLRRGYIGVVNRSQKDIEGKKDIRAALAAERKFFLSHPAYRHMADRMGTPHLQKTLNQQLTNHIRESLPALRSKLQSQLLSLEKEVEEYKIFRPDDPTPKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELINTVRQCTSKLSSYPRLREETERIVTTYIREREGRTKDQILLLIDIEQSYINTNHEDFIGFANAQQRSTQLNKKRAIPNQVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESADQAQRRDDMLRMYHALKEALNIIGDISTSTVSTPVPPPVDDTWLQSASSHSPTPQRRPVSSIHPPGRPPAVRGPTPGPPLIPVPVGAAASFSAPPIPSRPGPQSVFANSDLFPAPPQIPSRPVRIPPGIPPGVPSRRPPAAPSRPTIIRPAEPSLLD

SEQ ID NO: 152 - protein, 1675 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 153 - protein, 1640 aa, Homo sapiens
MAQILPVRFQEHFQLQNLGINPANIGFSTLTMESDKFICIREKVGEQAQVTIIDMSDPMAPIRRPISAESAIMNPASKVIALKAGKTLQIFNIEMKSKMKAHTMAEEVIFWKWVSVNTVALVTETAVYHWSMEGDSQPMKMFDRHTSLVGCQVIHYRTDEYQKWLLLVGISAQQNRVVGAMQLYSVDRKVSQPIEGHAAAFAEFKMEGNAKPATLFCFAVRNPTGGKLHIIEVGQPAAGNQPFVKKAVDVFFPPEAQNDFPVAMQIGAKHGVIYLITKYGYLHLYDLESGVCICMNRISADTIFVTAPHKPTSGIIGVNKKGQVLSVCVEEDNIVNYATNVLQNPDLGLRLAVRSNLAGAEKLFVRKFNTLFAQGSYAEAAKVAASAPKGILRTRETVQKFQSIPAQSGQASPLLQYFGILLDQGQLNKLESLELCHLVLQQGRKQLLEKWLKEDKLECSEELGDLVKTTDPMLALSVYLRANVPSKVIQCFAETGQFQKIVLYAKKVGYTPDWIFLLRGVMKISPEQGLQFSRMLVQDEEPLANISQIVDIFMENSLIQQCTSFLLDALKNNRPAEGLLQTWLLEMNLVHAPQVADAILGNKMFTHYDRAHIAQLCEKAGLLQQALEHYTDLYDIKRAVVHTHLLNPEWLVNFFGSLSVEDSVECLHAMLSANIRQNLQLCVQVASKYHEQLGTQALVELFESFKSYKGLFYFLGSIVNFSQDPDVHLKYIQAACKTGQIKEVERICRESSCYNPERVKNFLKEAKLTDQLPLIIVCDRFGFVHDLVLYLYRNNLQRYIEIYVQKVNPSRTPAVIGGLLDVDCSEEVIKHLIMAVRGQFSTDELVAEVEKRNRLKLLLPWLESQIQEGCEEPATHNALAKIYIDSNNSPECFLRENAYYDSSVVGRYCEKRDPHLACVAYERGQCDLELIKVCNENSLFKSEARYLVCRKDPELWAHVLEETNPSRRQLIDQVVQTALSETRDPEEISVTVKAFMTADLPNELIELLEKIVLDNSVFSEHRNLQNLLILTAIKADRTRVMEYISRLDNYDALDIASIAVSSALYEEAFTVFHKFDMNASAIQVLIEHIGNLDRAYEFAERCNEPAVWSQLAQAQLQKDLVKEAINSYIRGDDPSSYLEVVQSASRSNNWEDLVKFLQMARKKGRESYIETELIFALAKTSRVSELEDFINGPNNAHIQQVGDRCYEEGMYEAAKLLYSNVSNFARLASTLVHLGEYQAAVDNSRKASSTRTWKEVCFACMDGQEFRFAQLCGLHIVIHADELEELMCYYQDRGYFEELILLLEAALGLERAHMGMFTELAILYSKFKPQKMLEHLELFWSRVNIPKVLRAAEQAHLWAELVFLYDKYEEYDNAVLTMMSHPTEAWKEGQFKDIITKVANVELCYRALQFYLDYKPLLINDLLLVLSPRLDHTWTVSFFSKAGQLPLVKPYLRSVQSHNNKSVNEALNHLLTEEEDYQGLRASIDAYDNFDNISLAQQLEKHQLMEFRCIAAYLYKGNNWWAQSVELCKKDHLYKDAMQHAAESRDAELAQKLLQWFLEEGKRECFAACLFTCYDLLRPDMVLELAWRHNLVDLAMPYFIQVMREYLSKVDKLDALESLRKQEEHVTEPAPLVFDFDGHE

SEQ ID NO: 154 - protein, 218 aa, Homo sapiens
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRAAEEAFVNDIDESSPGTEWERVARLCDFNPKSSKQAKDVSRMRSVLISLKQAPLVH

SEQ ID NO: 155 - protein, 211 aa, Homo sapiens
MADDFGFFSSSESGAPEAAEEDPAAAFLAQQESEIAGIENDEGFGAPAGSHAAPAQPGPTSGAGSEDMGTTVNGDVFQEANGPADGYAAIAQADRLTQEPESIRKWREEQRKRLQELDAASKVTEQEWREKAKKDLEEWNQRQSEQVEKNKINNRASEEAFVKESKEETPGTEWEKVAQLCDFNPKSSKQCKDVSRLRSVLMSLKQTPLSR

SEQ ID NO: 156 - protein, 218 aa, Homo sapiens
MSQTAMSETYDFLFKFLVIGNAGTGKSCLLHQFIEKKFKDDSNHTIGVEFGSKIINVGGKYVKLQIWDTAGQERFRSVTRSYYRGAAGALLVYDITSRETYNALTNWLTDARMLASQNIVIILCGNKKDLDADREVTFLEASRFAQENELMFLETSALTGENVEEAFVQCARKILNKIESGELDPERMGSGIQYGDAALRQLRSPRRAQAPNAQECGC

SEQ ID NO: 157 - protein, 216 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 158 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 159 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 160 - protein, 257 aa, Homo sapiens
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 161 - protein, 71 aa, Homo sapiens
MRPSGTAGAALLALLAALCPASRALEEKKGKGVSRRLPRRPRIAPRTPQPAQPRTGAPARARAPARPFLFP

SEQ ID NO: 162 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 163 - protein, 41 aa, Homo sapiens
YGQVPMCDAGEQCAVRKGARIGKLCDCPRGTSCNSFLLKCL

SEQ ID NO: 164 - protein, 129 aa, Homo sapiens
MKILVALAVFFLVSTQLFAEEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 165 - protein, 11 aa, Homo sapiens
RPKPQQFFGLM

SEQ ID NO: 166 - protein, 125 aa, Homo sapiens
MAGPSLACCLLGLLALTSACYIQNCPLGGKRAAPDLDVRKCLPCGPGGKGRCFGPNICCAEELGCFVGTAEALRCQEENYLPSPCQSGQKACGSGGRCAVLGLCCSPDGCHADPACDAEATFSQR

SEQ ID NO: 167 - protein, 9 aa, Homo sapiens
CYIQNCPLG

SEQ ID NO: 168 - protein, 116 aa, Homo sapiens
MLSCRLQCALAALSIVLALGCVTGAPSDPRLRQFLQKSLAAAAGKQELAKYFLAELLSEPNQTENDALEPEDLSQAAEQDEMRLELQRSANSNPAMAPRERKAGCKNFFWKTFTSC

SEQ ID NO: 169 - protein, 4 aa, synthetic construct
  REGION 1..4: note = AAV9 residues at residue 586
SAQA

SEQ ID NO: 170 - protein, 11 aa, synthetic construct
  REGION 1..11: note = Substitution at residue 586 to form PHP.eB capsid
SDGTLAVPFKA

SEQ ID NO: 171 - protein, 7 aa, synthetic construct
  REGION 1..7: note = AAV-BR1
NRGTEWD

SEQ ID NO: 172 - protein, 7 aa, synthetic construct
  REGION 1..7: note = AAV-PHP.S
QAVRTSL

SEQ ID NO: 173 - protein, 7 aa, synthetic construct
  REGION 1..7: note = AAV-PHP.B
TLAVPFK

SEQ ID NO: 174 - protein, 7 aa, synthetic construct
  REGION 1..7: note = AAV-PPS
DSPAHPS

SEQ ID NO: 175 - DNA, 21 bp, synthetic construct
  misc_feature 1..21: note = forward primer
  source 1..21: mol_type = other DNA
ggaacccctagtgatggagtt

SEQ ID NO: 176 - DNA, 16 bp, synthetic construct
  misc_feature 1..16: note = reverse primer
  source 1..16: mol_type = other DNA
cggcctcagtgagcga

SEQ ID NO: 177 - DNA, 310 bp, Homo sapiens
  source 1..310: mol_type = genomic DNA
gaaaagatccgggttcacactaatcaggcccaacggaaggccatattagcaatttggcaggtacccgagggccatacctaatctgcataaaatgaagcagattgcaaccgccctcatcttttttatttttaaactggtttttgaagcagagcataaaatctcagagggagagacagaagatgctagtgcatacattttccttcatgcctttattttcattctttttgcacaaaccatcttcctgaatggctgtttacctaaagaagaataacaaaataaaaggtgctaggaaatggagtaggcagagatc

SEQ ID NO: 178 - DNA, 401 bp, Mus musculus
  source 1..401: mol_type = genomic DNA
gaagcatggtccagcaaagcctttctgttctaaaggaaaggatctgagttgtcacctcccaggtccgtggaaggctttttagcagtttggcaggtgcctgagggccacacctcatctgcataaaatgtggcagattgcaaccgccctcgtcttttttatttttaaactggtttttgaaacagaacatatataaaagctcagagaaagggaaaggagatagatggccgagcttccatatcccttagtgcctttattttcattctttttccattttcctaagtggctatttaccaagacaaagataacaaatctgctaggaaaaggagtgggcagtgctacaaaatgtttttttttttttaaagaaagtcctatcttataatagatcttcaccacgatgcctc

SEQ ID NO: 179 - DNA, 5102 bp, synthetic construct
  misc_feature 1..5102: note = synthetic construct
  source 1..5102: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctagagggctgggcataaaagtcagggcagagccatctattgcttacatttgcttctgacatatgccgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaatctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgacttgctgctatcccctaggtgctgtcatttacctacggttgtctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcataggtgctgttatttgcctcaaaaaatagacttttattttgcctttcttttctttaaccacactcaaaattagagaacagagacaaaacccagcaggaaatagcacagaaagcccacagaatcaaagacgtgttcaaacagccagctgaattcattgcacatttcaaccacagaaatattttcaggtgattctgttgtttgacaaaacgtgggaaccacaggatctacaacacttgcaagcaaaactcaacagctctaataatagttacagaagtgaaagccaatttggataaaataagacattgactcaagtcctctcagaagagttttgaaagcaaagtttacaaaagtctggtttgtcctttgggatttacagacctttcagccccttgattcatttttttttttttggatttcttcatcactgggagaattcccatgcattatttctcccctgcttcaaaatcatcaaatgtgaaacatttttcactcttttcttctgtatatagtgataaaatagctattggcttttggctaaatgtgctactttgagcccacccacacaagggagaaatgggggcagacatgagtttgggcatgagtgagctctgccttctcagaggtgagccacgtggtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 180 - DNA, 6587 bp, synthetic construct
  misc_feature 1..6587: note = synthetic construct
  source 1..6587: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccttcgaaaccggtgctagagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacatctagcaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 181 - DNA, 5343 bp, synthetic construct
  misc_feature 1..5343: note = synthetic construct
  source 1..5343: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctggcgtggccaccatggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 182 - DNA, 5717 bp, synthetic construct
  misc_feature 1..5717: note = synthetic construct
  source 1..5717: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagctagcaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaatgaggcgcgccgcggccgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 183 - DNA, 5408 bp, synthetic construct
  misc_feature 1..5408: note = synthetic construct
  source 1..5408: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacagctagcgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaggcgcgccgcggccgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 184 - DNA, 5442 bp, synthetic construct
  misc_feature 1..5442: note = synthetic construct
  source 1..5442: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccttttccaaccgttccttcatgacatcaaggcttcagactgctaagctttgggcactacctggggtcagtctgcatcaaaatgtaaggctcaaatgtgtaattgtaagtactgttttgctgagctggaagggctcctttgaagcccacgttttaattttaatttagccacacagagtggcaaagacaaatagatttatccaaaatacatttggtaacagattttttgagtcagttattaattttatttgaggggttcctctttttattttttataaactgtgaaactcaagaggaagcaggatcccatgcaatgccttttattgatggcctgctatgtgccaagaaaggtgttaaatgttttccaatgctgcctcatttatcctgatcttacagacaagcaaaaggaggtgtgaagaggtgaagtttctcacccagctggaaagtggcaaagtcattcacagatctgcctccgctcaaaaaaattgctttatgcaactctttggaagctaacttcatgggagctacatgcagcttctcaatgaaccttgttttgctggcctgcagccagaagttactactcctttggttcctgagcagagctcgggctgggcataaaagtcagggcagagccatctattgcttacatttgcttctgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 185 - DNA, 6932 bp, synthetic construct
  misc_feature 1..6932: note = synthetic construct
  source 1..6932: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgggatccgccaccatgatctctctgattgccgctctggccgtggactacgtgatcgggatggaaaacgctatgccatggaatctgcccgccgatctggcttggttcaagaggaacaccctgaacaagccagtgatcatgggcagacacacttgggagtccattggacggcccctgcctggacgcaagaacatcattctgagctcccagccctctaccgacgacagggtgacatgggtgaaaagtgtggacgaagccattgccgcttgcggagatgtgcccgagatcatggtcatcggcggagggagagtgatcgagcagttcctgcctaaggcccagaaactgtacctgactcacattgacgctgaggtggaaggggacacccattttcctgattatgagccagacgattgggaaagcgtgttctccgagtttcacgacgccgatgctcaaaattctcatagttattgctttgagatcctggaaaggagaggcgcgccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggatgagctgtacaagggcaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcgaatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 186 - DNA, 5806 bp, synthetic construct
  misc_feature 1..5806: note = synthetic construct
  source 1..5806: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtataagccttgggggcaatcaaactattacattgagtccttggatttgctacaaattacattttaaatgcaatcattttataaaagcttcaacactcacacttggaagcgttaccctgttgaatatcactgactcactaacttgcattgccatgctaacttgctttcagagagatctcagaacacatcatcttctgctatttcaatacatgcacattaatttcctatcaacgtgtgctgatcaggaactctgtaatctggcaccggtgtttatttttattcctgtctattcctgttggctcacgaaaagattgtttgagcaagtgttttatggtgagttgtatcatatgtacattgatttaatctgcccacattcagttctacaagcggagccaaaaaaatagagacaagcataattttcattcaacatgagcccctcaatgcaagccaagtacctcatctggtgctcagctaaagcaacagcaatctgttccaccctggagacacaactggccacagaaaacttagtgaaaagaggcaatgctatgcacaggacaaatgagctcgggctgggcataaaagtcagggcagagccatctattgcttacatttgcttctgttaattaagccgccaccatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 187 - DNA, 5779 bp, synthetic construct
  misc_feature 1..5779: note = synthetic construct
  source 1..5779: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtatgtgtcttttactctgatcctcctgtttttaccttccaagtgctggaatcacagacatataccactgtgcatagcatcattacaatgttatagtttttcacactatgccttgactttttggaaaggcaaaccacctcttggatttctccttccttctctatctctctctctctctcttcctccctccgtccctccatctcttcctccttcccattttcttctctccctatttggacacaatataaaataatttagatgaggtgagttaaattgtgaacaaagtatgtgcctatacatggttgtaaatcagcttatcaaagtgtaatattagaagaatttataaaaatgataaaattcatactcaaagttctgtgtaaagcaataatagctttatctccttttagttatcttgagtctttctatgactaacaactccctcataggcatcttaaagagcagtaagcataagtagattccaaatgggaagggagaagtgtgaaccatcactttcatccagacttgtagatatatctgctgcattttcagaaaccagaaacagacagtgttctttatctccattgagtctagtgtagcaacagagctcgggctgggcataaaagtcagggcagagccatctattgcttacatttgcttctgggatccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 188 - DNA, 255 bp, Mus musculus
  source 1..255: mol_type = genomic DNA
ctccaaatttcttcaaccaagtagagaaaaatgagagagaaggaaagaaaaaaagaggtatggggagaagagaaagaaggcaacttgttaaaaatctcagtcaaacttacatactatatagaacagcatggtgaatttagggcacatggatataaaatggaagtttcttattcagtagcagcaacttgtgggcacaggagttggcaaagataaaaatgtccaaagtcacaaatacaatgtatagttagtcatagg